(19) United States
(12) Patent Application Publication
(10) Pub. No.: US 2025/0146001 A1
(43) Pub. Date: May 8, 2025

(54) Compositions and Methods for Enhancing AAV Therapy and Decreasing Tropism of AAV to the Liver

(71) Applicant: Vertex Pharmaceuticals Incorporated, Boston, MA (US)

(72) Inventors: Gregoriy Aleksandrovich Dokshin, Boston, MA (US); Jishnu Saha, Boston, MA (US)

(73) Assignee: Vertex Pharmaceuticals Incorporated, Boston, MA (US)

(21) Appl. No.: 18/919,091

(22) Filed: Oct. 17, 2024

Related U.S. Application Data
(63) Continuation of application No. PCT/US23/65850, filed on Apr. 17, 2023.
(60) Provisional application No. 63/331,968, filed on Apr. 18, 2022.

(51) Int. Cl. C12N 15/113; C07K 16/28; C12N 9/22; C12N 15/11; C12N 15/86; C12N 15/88

(52) U.S. Cl. CPC C12N 15/1138; C07K 16/28; C12N 9/22; C12N 15/111; C12N 15/86; C12N 15/88; C12N 2310/11; C12N 2310/14; C12N 2310/20; C12N 2310/351; C12N 2750/14143

(57) ABSTRACT

Compositions and methods for enhancing AAV therapy by increasing the percentage of AAV delivered to a non-liver target in a subject by blocking binding of binding of AAVs to AAV receptors in the liver and then administering an AAV therapy targeting a non-liver tissue.

This application is a continuation of International Application No. PCT/US2023/065850, filed on Apr. 17, 2023, which claims the benefit of priority to U.S. Provisional Application No. 63/331,968, filed Apr. 18, 2022, both of which are incorporated herein by reference in their entireties.

The instant application contains a Sequence Listing which has been submitted electronically in XML format and is hereby incorporated by reference in its entirety. Said XML copy, created on Apr. 15, 2023, is named 01245-0037-00PCT_Sequence_Listing and is 8,090,000 bytes in size.

### INTRODUCTION AND SUMMARY

Genetic drugs, oligonucleotides such as small interfering RNA (siRNA), messenger RNA (mRNA), antisense oligonucleotides (ASOs) and plasmid DNA, provide the potential for regulating and editing gene expression. ASOs may work by downregulation of a molecular target, usually achieved by induction of RNase H endonuclease activity that cleaves an RNA-DNA heteroduplex with a significant reduction of the target gene translation. Other ASO-driven mechanisms may include inhibition of 5′ cap formation, splice-switching, or steric hindrance of ribosomal activity. siRNAs in particular are short (19-21 nucleotide), double-stranded RNAs that use the natural RNA interference (RNAi) mechanisms and degrade complementary mRNAs through the use of complicated protein machinery. As a result, siRNA can lead to the reversible knock down in vivo of a protein of interest. However, naked RNA or DNA molecules face rapid degradation in vivo, complex immune responses, and impermissible cellular uptake. Thus, delivery of these drugs requires a sophisticated delivery system.

Adeno-associated viruses (AAV) vectors have traditionally been a leading candidate for in vivo virus-based gene therapy because of their broad tissue tropism, non-pathogenic nature and low immunogenicity. Currently 12 AAV serotypes and over 100 variants have been identified in human and nonhuman primate populations. Gene therapy vectors using AAV in vivo can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. See S. Pillay, Nature, 530(7588): 108-112 (2016).

AAV variants have or have been engineered with specific tropisms to allow for efficacious localized or systemic administration with targeted gene therapy delivery. Subtle variations in primary and secondary receptor interactions for AAV variants can yield variants or serotypes possessing particular tropisms where the AAV preferentially infects one tissue or cell type over others. See Naso et al., BioDrugs, 31:317-334 (2017). As a non-limiting example, skeletal muscle has been shown to be a target tissue effectively transduced by many AAV serotypes and variants. Targeted delivery of gene therapy to reconstitute deficient muscle structural proteins or enzymes can be used to treat many diseases that disable muscle fibers throughout the body. Additionally, once transduced, muscle serves as a production site for protein products. Muscle can be targeted as a biofactory to synthesize and secrete therapeutic agents or secretory proteins, e.g., factor VIII and IX and erythropoietin, that can act locally or systemically to treat, e.g., diabetes, atherosclerosis, hemophilia, cancer and other infectious diseases. See Wang, Expert Opin Drug Deliv., 11(3): 345-364 (2014). Direct central nervous system delivery, local delivery of AAV to cardiac muscles, and inhaled pulmonary delivery are other non-limiting exemplary AAV gene therapy applications.

However, AAV vectors are limited by several factors, including their small packaging size. Moreover, current limitations of using AAVs for gene transfer include potential safety concerns, including off-target toxicity. Following administration, in addition to localized and targeted delivery, most serotypes of AAV may also achieve off-target gene transfer, which can result in transduction and expression of the gene of interest in unwanted cells or tissues. When AAV vectors reach the bloodstream, the circulatory system carries the vectors to the whole body, including to the liver, skeletal and cardiac muscles, pancreas and adrenal glands. While different AAV serotypes can have distinct tissue distribution patterns after administration, the liver is the most common organ harboring a large amount of mis-targeted AAV vectors. A recent biodistribution animal study found that, despite direct cerebrospinal fluid administration, biodistribution of vector DNA and green fluorescent protein (GFP) expression was widespread. See Meseck et al., doi.org/10.1101/2021.11.28.470258, BioRxiv.org (posted Nov. 28, 2021). In a portion of that study, the transduction and expression of scAAV9-CB-GFP in the CNS and peripheral tissues following a single intrathecal infusion into the cerebrospinal fluid was assessed. In that study, vector DNA and GFP expression was found to be the greatest in the spinal cord, dorsal root ganglia, and systemic tissue (e.g., liver) with lower concentrations in many brain regions. Recently in 2020-2021, a gene therapy clinical trial using AAV gene therapy was paused due in part to deaths of subjects who developed complications from liver failure. See NCT03199469 (using an AAV serotype 8 vector). There is an urgent need to address the issue of secondary liver toxicity in AAV-gene therapy.

Current methods of addressing the issue of secondary liver toxicity include trying to reduce liver tropism by modifying the AAVs to promote tropism to the intended targets. This includes using recombinant techniques to engineer the makeup of the AAV to enhance specific tropism to the intended target, such as capsid shuffling, directed evolution, and random peptide library insertions, in addition to inserting larger binding proteins into different regions of AAV capsid proteins to derive variants and confer selectivity. See Naso et al., BioDrugs, 31:317-334 (2017). One other such method is using tissue-specific promotors to drive transcription in the intended targets and not in the mis-targeted tissue. Another such method is to design the transgene to carry a target sequence of microRNAs (miRNAs) that are expressed specifically in the target tissue or cell type, for example, incorporating target sequences into the 3′-UTRs to reduce transgene expression in undesired tissues, while maintaining transgene expression in the target. Wang, Expert Opin Drug Deliv., 11(3): 345-364 (2014). Notably, however, each of these methods is individualized for the particular therapy. Namely, they involve designing aspects of particular AAV serotypes or variants and/or transgenes and/or using promoters to enhance tropism to the specific target. These modifications to the specific vectors, transgenes, and/or promoters tend to be therapy-specific and cannot necessarily be applied to all AAV-gene therapy systems for reducing secondary liver toxicity.

Thus, there exists a need in the art to reduce hepatotoxicity from AAVs that can apply to all AAVs and administration of all transgenes regardless of the intended target and type of delivery.

Rather than enhance tropism to the intended non-liver target, the present invention, in part, seeks to de-target the liver by knocking down or blocking the receptors for AAVs in the liver, for example, as a pre-conditioning therapy prior to receiving the AAV-gene therapy directed to the non-liver target. Administration of RNA interference (RNAi)-based therapeutics, including using small interfering RNA (siRNA) or GalNAc-conjugated RNAi or LNP-assisted RNAi, or antisense oligonucleotides (ASOs), for example, prior to AAV administration could effectively and temporarily block subsequent binding of the AAV capsid to AAVR in the liver. Using ASOs or siRNAs in vivo is known to generally be transient. Because RNAi-based pre-conditioning can be administered prior to the AAV, and can later be cleared from the body, it can effectively and temporarily block binding to AAVR in the liver, thereby enhancing the tropism of the AAVs to the intended non-liver target without long term effects. The present compositions and methods can be used as a platform to enhance the tropism and thus efficacy and safety of non-liver AAV-mediated gene therapies.

Accordingly, the following non-limiting embodiments are provided:
Embodiment 1 is a composition comprising an agent that blocks AAV binding to an AAV receptor (AAVR) and a delivery molecule that delivers the agent to the liver.
Embodiment 2 is the composition of embodiment 1, wherein the agent comprises a small-interfering RNA (siRNA).
Embodiment 3 is the composition of embodiment 2, wherein the siRNA comprises at least 19 contiguous nucleotides of any of SEQ ID Nos: 4300-9527.
Embodiment 4 is the composition of embodiment 3, wherein the siRNA comprises any of the sequences of SEQ ID Nos: 4300-9527.
Embodiment 5 is the composition of embodiment 4, wherein the siRNA is no more than 21, 25, or 31 nucleotides in length.
Embodiment 6 is the composition of embodiment 1, wherein the agent comprises an anti-AAVR antibody.
Embodiment 7 is the composition of embodiment 1, wherein the agent comprises a small molecule.
Embodiment 8 is the composition of embodiment 1, wherein the agent comprises an antisense oligonucleotide (ASO).
Embodiment 9 is the composition of embodiment 8, wherein the ASO comprises any of the sequences of SEQ ID Nos: 9600-9623, or comprises at least 14 consecutive nucleotides of any of SEQ ID Nos: 9600-9623.
Embodiment 10 is the composition of any one of embodiments 1-9, wherein the delivery molecule comprises one or more of the following: lactose, galactose, N-acetylgalactosamine (GalNAc), galactosamine, N-formylgalactosamine, N-acetylgalactosamine, N-propionylgalactosamine, N-butanoylgalactosamine, N-isobutanoyl-galactosamine, and cholesterol, or a derivative thereof.
Embodiment 11 is the composition of embodiment 10, wherein the delivery molecule comprises N-acetylgalactosamine (GalNAc).
Embodiment 12 is the composition of any one of embodiments 1-9, wherein the delivery molecule comprises a lipid nanoparticle (LNP).
Embodiment 13 is the composition of any one of embodiments 1-9, wherein the delivery molecule comprises an AAV.
Embodiment 14 is a method comprising: (a) administering to a subject an agent that blocks AAV binding to an AAV receptor (AAVR) in the liver, and then (b) administering an AAV vector to the subject.
Embodiment 15 is the method of embodiment 14, wherein the AAV vector further comprises a payload.
Embodiment 16 is the method of embodiment 15, wherein the payload comprises a therapeutic agent to prevent or treat disease.
Embodiment 17 is the method of embodiment 15, wherein the payload comprises a guide RNA, an endonuclease, a tRNA, a small molecule, an ASO, an antibody, an siRNA, or an RNAi agent.
Embodiment 18 is a method of increasing the percentage of AAV delivered to a non-liver target in a subject, comprising (a) a pre-conditioning step comprising administering to the subject a composition comprising an agent that blocks AAV binding to an AAV receptor (AAVR) in the liver, and then (b) administering an AAV vector targeting a non-liver tissue.
Embodiment 19 is the method of any one of embodiments 14-18, wherein step (a) comprises administering to the subject the composition of any one of embodiments 1-13.
Embodiment 20 is a method of decreasing tropism of AAV to the liver in a subject comprising (a) administering to the subject a composition of any one of embodiments 1-13, and then (b) administering an AAV vector targeting a non-liver tissue.
Embodiment 21 is the method of any of embodiments 14-20, wherein administering to the subject the composition and/or agent that blocks AAV binding to an AAV receptor (AAVR) in the liver temporarily blocks AAV binding to AAV receptors in the liver.
Embodiment 22 is the method of any of embodiments 14-20, wherein administering to the subject the composition and/or agent that blocks AAV binding to an AAV receptor (AAVR) in the liver is part of the pre-conditioning step.
Embodiment 23 is the method of any of embodiments 14-20, wherein administering to the subject the composition and/or agent that blocks AAV binding to an AAV receptor (AAVR) in the liver occurs about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the AAV vector.
Embodiment 24 is the method of any of embodiments 14-20, wherein administering to the subject the composition and/or agent that blocks AAV binding to an AAV receptor (AAVR) in the liver occurs about 1 day, about 2 days, about 3 days, about 4 days, about 5 days, about 6 days, about 7 days prior to administering the AAV vector.
Embodiment 25 is the method of any of embodiments 14-20, wherein administering to the subject the composition and/or agent that blocks AAV binding to an AAV receptor (AAVR) in the liver occurs about 7 days, about 8 days, about 9 days, about 10 days, about 11 days, about 12 days, about 13 days, or about 14 days prior to administering the AAV vector.
Embodiment 26 is the method of any of embodiments 14-20, wherein administering to the subject the composition of any one of embodiments 1-13 immediately precedes administering the AAV vector.
Embodiment 27 is the method of any of embodiments 14-20, wherein the composition of any one of embodiments 1-10 and the AAV vector are co-administered.
Embodiment 28 is the method of any of embodiments 14-20, wherein step (a) comprises administering to the subject a composition comprising a small-interfering RNA (siRNA) or an antisense oligonucleotide (ASO).
Embodiment 29 is the method of any of embodiment 28, wherein the composition comprises small-interfering RNA (siRNA) that is conjugated to a liver-targeting moiety.
Embodiment 30 is the method of embodiment 29, wherein the composition comprises N-acetylgalactosamine (GalNAc)-conjugated siRNA.
Embodiment 31 is the method of embodiment 28, wherein the composition comprises small-interfering RNA (siRNA) encapsulated in a lipid nanoparticle (LNP).
Embodiment 32 is the method of embodiment 28, wherein the composition comprises an ASO.
Embodiment 33 is the method of any one of embodiments 14-32, wherein the composition comprises a pharmaceutically acceptable carrier.
Embodiment 34 is the method of embodiment 33, wherein the method comprises administering by intraperitoneal injection.
Embodiment 35 is the method of embodiment 34, wherein the ASO is administered about 1 day, about 2 days, about 3 days, about 4 days, about 5 days, about 6 days, about 7 days prior to administering the AAV vector.
Embodiment 36 is the method of embodiment 34, wherein the ASO is administered between about 24 and about 48 hours prior to administering the AAV vector.
Embodiment 37 is the method of embodiment 34, wherein the ASO is administered about 24 hours prior to administering the AAV vector.
Embodiment 38 is the method of embodiment 34, wherein the ASO is administered about 48 hours prior to administering the AAV vector.
Embodiment 39 is the method of any of embodiments 31-38, wherein the method comprises co-administering other drugs that facilitate increased uptake of the siRNA or ASO in the liver.
Embodiment 40 is the method of any of any of embodiments 14-39, wherein the blocking of AAV binding to AAV receptors in the liver in step (a) is not temporary.
Embodiment 41 is the method of embodiment 39, wherein the composition comprises an anti-AAV antibody that blocks AAV binding to AAV receptors.
Embodiment 42 is the method of any of embodiments 14-41, wherein the AAV vector is intended for the brain; central nervous system; spinal cord; eye; retina; bone; cardiac muscle, skeletal muscle, and/or smooth muscle; lung; pancreas; heart; and/or kidney.
Embodiment 43 is the method of any of embodiments 14-42, wherein the AAV vector is intended for cardiac muscle, skeletal muscle, and/or smooth muscle.
Embodiment 44 is the method of any one of embodiments 14-43, wherein administering the composition in step (a) increases the percentage of AAV delivered to the non-liver target.
Embodiment 45 is the method of any one of embodiments 14-44, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of AAV in brain; central nervous system; spinal cord; eye; retina; bone; cardiac muscle, skeletal muscle, and/or smooth muscle; lung; pancreas; heart; and/or kidney as compared to the AAV in the corresponding tissue of a control subject that received the AAV but did not receive the agent that blocks AAV binding to an AAV receptor (AAVR) in the liver.
Embodiment 46 is the method of embodiment 45, the method results in at least a 10%, 30%, 50%, 75%, 100%, 125%, 150%, 200%, 250%, 300%, 350%, 400%, 450%, 500%, 600%, 700%, 800%, 900%, or 1000% increase of AAV in skeletal muscle as compared to the AAV in the corresponding muscle of a control subject that received the AAV but did not receive the agent.
Embodiment 47 is the method of any one of embodiments 14-46, wherein the AAV vector further comprises molecules for enhancing tropism for the target host cells or tissue.
Embodiment 48 is the method of any of embodiments 14-47, wherein the subject is a human subject.
Embodiment 49 is the method of any one of embodiments 14-48, wherein the AAV vector comprises a single nucleic acid molecule encoding one or more guide RNAs and a Cas9, wherein the single nucleic acid molecule comprises:
   a. a first nucleic acid encoding one or more spacer sequences selected from any one of SEQ ID NOs: 1-35, 1000-1078, or 3000-3069 and a second nucleic acid encoding a Staphylococcus aureus Cas9 (SaCas9); or
   b. a first nucleic acid encoding one or more spacer sequences selected from any one of SEQ ID NOs: 100-225, 2000-2116, or 4000-4251 and a second nucleic acid encoding a Staphylococcus lugdunensis (SluCas9); or
   c. a first nucleic acid encoding one or more spacer sequences comprising at least 20 contiguous nucleotides of a spacer sequence selected from any one of SEQ ID NOs: 1-35, 1000-1078, or 3000-3069 and a second nucleic acid encoding a Staphylococcus aureus Cas9 (SaCas9); or
   d. a first nucleic acid encoding one or more spacer sequences comprising at least 20 contiguous nucleotides of a spacer sequence selected from any one of SEQ ID NOs: 100-225, 2000-2116, or 4000-4251 and a second nucleic acid encoding a Staphylococcus lugdunensis (SluCas9); or
   e. a first nucleic acid encoding one or more spacer sequences that is at least 90% identical to any one of SEQ ID NOs: 1-35, 1000-1078, or 3000-3069 and a second nucleic acid encoding a Staphylococcus aureus Cas9 (SaCas9); or
   f. a first nucleic acid encoding one or more spacer sequences that is at least 90% identical to any one of SEQ ID NOs: 100-225, 2000-2116, or 4000-4251 and a second nucleic acid encoding a Staphylococcus lugdunensis (SluCas9); or
   g. a first nucleic acid encoding a pair of guide RNAs comprising a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   h. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   i. a first nucleic acid encoding a pair of guide RNAs that is at least 90% identical to a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   j. a first nucleic acid encoding a pair of guide RNAs comprising a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   k. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   l. a first nucleic acid encoding a pair of guide RNAs that is at least 90% identical to a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   m. a first nucleic acid encoding a pair of guide RNAs that is at least 90% identical to a first and second spacer sequence selected from any one of the following pairs of spacer sequences:
      i. SEQ ID NOS: 148 and 134,
      ii. SEQ ID Nos: 145 and 131,
      iii. SEQ ID Nos: 144 and 149;
      iv. SEQ ID Nos: 144 and 150;
      v. SEQ ID Nos: 146 and 148;
and a second nucleic acid encoding a Staphylococcus lugdunensis (SluCas9); or
   n. a first nucleic acid encoding a pair of guide RNAs that is at least 90% identical to a first and second spacer sequence selected from any one of the following pairs of spacer sequences:
      i. SEQ ID NOs: 12 and 1013; and
      ii. SEQ ID Nos: 12 and 1016;
and a second nucleic acid encoding a SaCas9-KKH.
Embodiment 50 is the method of any one of embodiments 14-49, wherein the AAV vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs.
Embodiment 51 is the method of any one of embodiments 14-50, wherein the AAV vector is an AAV9 vector.
Embodiment 52 is the method of any one of embodiments 14-51, wherein the agent is an siRNA.
Embodiment 53 is the method of embodiment 52, wherein the siRNA comprises at least 19 contiguous nucleotides of any of SEQ ID Nos: 4300-9527.
Embodiment 54 is the method of embodiment 52, wherein the siRNA comprises any of the sequences of SEQ ID Nos: 4300-9527.
Embodiment 55 is The method of embodiment 52, wherein the siRNA comprises at least 19 contiguous nucleotides of any of SEQ ID Nos: 9508-9531.
Embodiment 56 is The method of embodiment 52, wherein the siRNA comprises any of the sequences of SEQ ID Nos: 9508-9531.
Embodiment 57 is the method of any one of embodiments 52-56 wherein the siRNA is no more than 21, 25, or 31 nucleotides in length.
Embodiment 58 is the method of any one of embodiments 14-51, wherein the agent comprises an anti-AAVR antibody.
Embodiment 59 is the method of any one of embodiments 14-51, wherein the agent comprises a small molecule.
Embodiment 60 is the method of any one of embodiments 14-51, wherein the agent comprises an antisense oligonucleotide (ASO).
Embodiment 61 is the method of embodiment 60, wherein the ASO comprises any of the sequences of SEQ ID Nos: 9600-9623, or comprises at least 14 consecutive nucleotides of any of SEQ ID Nos: 9600-9623.
Embodiment 62 is the method of embodiment 60, wherein the ASO is between 14-35, 15-30, or 15-25 nucleotides in length.

### BRIEF DESCRIPTION OF THE DRAWINGS

FIGS. 1A-1J show knockdown of AAVR mRNA by 10 different siRNAs in Hepa1-6 cells. Plots show 10-point dose response for each of the 10 siRNAs evaluated. FIG. 1K provides a representative structure for a modified siRNA sequence used in the assessment.

FIG. 2 shows knockdown of AAVR mRNA with different MOE- or LNA-based ASOs in C2C12 cells. The plot shows fold change in AAVR mRNA levels relative to untreated samples, as measured by qPCR 48 hours after treatment with 50 nM ASO. The dashed line indicates the level of mRNA expression for the untreated samples. Error bars indicate standard deviation. On the horizonal axis, “siRNA” refers to siRNAVT011 targeting AAVR (positive control); “lipo” refers to RNAiMax Lipofectamine only (negative control); “untreated”—refers to samples with no lipofectamine (negative control).

FIG. 3 shows knockdown of AAVR mRNA with different LNA-based ASOs in Huh7 cells. The plot shows fold change in AAVR mRNA levels relative to untreated samples, as measured by qPCR 48 hours after treatment with 10, 50, 100 or 200 nM ASO. The dashed line indicates the level of mRNA expression for the untreated samples. On the horizonal axis, “siRNA” refers to siRNAVT011 targeting AAVR (positive control); “lipo” refers to RNAiMax Lipofectamine only (negative control); “scram” refers to non-targeting siRNA (IDT Cat #51-01-14-03) (negative control); “untreated”—refers to samples with no lipofectamine (negative control). For each ASO, nM concentration of ASO is indicated as a number in the name.

FIGS. 4A-4D show the time course of AAV receptor mRNA and protein expression after ASO treatment. FIGS. 4A and 4C show fold change in AAVR mRNA levels by qPCR relative to untreated samples, FIGS. 4B and 4D are images of Western Blot detection results for AAV receptor protein and beta-actin from cell lysates. FIGS. 4A and 4B show mRNA and protein panels for untreated samples and FIGS. 4C and 4D show mRNA and protein panels for the treated samples, with time points as indicated in hours. Error bars show standard deviation.

FIGS. 5A-B show bright field images of control and preconditioned samples 72 h post AAV9-GFP (Vector Biolabs Cat #7007) infection. FIG. 5A shows control untreated cells; FIG. 5B shows preconditioned cells dosed with ASOVTO02 (250 nM).

FIGS. 6A-B show GFP transgene expression in Huh7 cells. FIGS. 6A-B show representative fluorescent images of control and preconditioned samples 72 h post AAV9-GFP (Vector Biolabs Cat #7007) infection. FIG. 6A shows control untreated cells; FIG. 6B shows preconditioned cells dosed with LNA-1 (250 nM).

FIG. 7 shows AAV receptor protein quantification 7 days after dosing with AAVR-targeting ASO at 2, 7 or 20 mg/kg doses, 20 mg/kg of control ASO, or PBS. The data were normalized to PBS treated samples. Error bars represent standard deviation from the mean.

FIGS. 8A and 8B show AAV receptor protein levels in heart and muscle 7 days post dosing with AAVR ASO (ASOVT002) at 20 mg/kg or PBS treated animals. The data were normalized to PBS treated samples. Error bars represent standard deviation from the mean.

FIG. 9 shows AAV receptor protein quantification after 3-, 7- and 10-days post dosing with AAVR ASO (ASOVT002) at 20 mg/kg, or PBS treatment. The data were normalized to PBS treated samples. Error bars represent standard deviation from the mean.

FIG. 10 is a graphical representation of the in vivo study design to test effects of ASO-based AAVR knockdown on AAV distribution corresponding to the study in Example 6.

### DETAILED DESCRIPTION

Reference will now be made in detail to certain embodiments of the invention. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA. The disclosure provides a number of exemplary nucleotide sequences herein, and contemplates reverse complements of these nucleotide sequences, as well as RNA and/or DNA equivalents of any of these sequences. For example, an RNA equivalent of any of the DNA sequences disclosed herein would comprise uracils in place of thymines in the sequence, whereas a DNA equivalent of any of the RNA sequences disclosed herein would comprise thymines in place of uracils.

As used herein, “CRISPR” systems and “RNA-targeted endonucleases” or “Cas-nucleases” includes the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. In particular embodiments, the RNA-targeted endonuclease is a type II CRISPR Cas enzyme. Other examples of Cas nucleases include a Csm or Cmr complex of a type III CRISPR system or the Cas10, Csml, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., Nat. Rev. Microbiol., 9:467-477 (2011); Makarova et al., Nat. Rev. Microbiol., 13: 722-36 (2015); Shmakov et al., Molecular Cell, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 17, 18, 19, 20, 21, 22, 23, 24, or 25 contiguous nucleotides of a sequence selected from SEQ ID NOs: 1-35 (for SaCas9), and 100-225 (for SluCas9). In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

As used herein, the terms “about” or “approximately” means an acceptable error for a particular value as determined by one of ordinary skill in the art, which depends in part on how the value is measured or determined.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector also known as AAVMYO (see, e.g., Weinmann et al., 2020, Nature Communications, 11:5432), and Myo-AAV vectors described in Tabebordbar et al., 2021, Cell, 184:1-20 (see, e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. Structurally, AAVs are small (25 nm), single-DNA stranded non-enveloped viruses with an icosahedral capsid. As used herein, “AAV” can refer to naturally occurring or engineered AAV serotypes and recombinant AAVs (rAAVs) and variants that can differ in the composition and structure of their capsid protein have varying tropism, i.e., ability to transduce different cell types. When combined with active promoters, this tropism defines the site of gene expression.

As used herein “AAV-based gene therapy” refers to the administration of AAV vector(s) and use of any AAV or AAV (vector) system comprising a tissue-specific promoter in facilitating administration of gene therapy, which can include any known gene editing system in the art. A promoter as described herein can also be “cell specific,” meaning that the particular promoter selected for the AAV can direct expression of the selected transgene/nucleotide sequence of interest in a particular cell or cell type. In some embodiments, for example, the promoter is a muscle-specific promoter, including a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters. Any known promoters may be used in conjunction with the AAVs to administer the gene therapy to the intended target tissues or cells. As used herein, “non-liver AAV-based gene therapy” includes treating or preventing a disease or disorder using AAV-based gene therapy that is not a disease or disorder of the liver.

As used herein, “AAVR” or “AAV-R” or “AAV receptor” are used interchangeably to refer to AAV receptor protein. Synonyms for AAVR also include, FLJ44532, KIAA0319L, KIAA0319-like, KIAA1837, KIAA1837 dyslexia-associated protein, KIAA0319-like protein, and polycystic kidney disease 1-related. AAVR is a glycosylated membrane protein that is capable of recycling from the plasma membrane to the trans-Golgi network using the cellular endosomal network. AAVR is known to be the key receptor that mediates entry of a panel of AAV serotypes. See, e.g., Pillay, Nature, 530(7588): 108-112 (2016); Meyer et al., eLife 8:e44707. DOI: doi.org/10.7554/eLife.44707 (2019). AAVR knock out was found to render cells highly resistant to infection by AAV2; and where AAVR is overexpressed in cells, the cells were increasingly susceptible to AAV2 infection. In Pillay (2016), CRISPR/Cas9 genome engineering was used to generate isogenic AAVR knock-out cell lines in a panel of cell types representing human and murine tissues. AAVR knock-out cells were infected with a panel of various AAV serotypes including AAV1, 2, 3B, 5, 6, 8, and 9, where the knock-out cells seemed resistant to all AAV serotypes. Accordingly, multiple serotypes, including AAV1, AAV2, AAV3B, AAV5, AAV6, AAV8, and AAV9, require AAVR for transduction. In some embodiments, “AAV” refers to any AAV serotype and variant.

As used herein, “blocks AAV binding to an AAVR” or the like, means temporarily or permanently reducing, inhibiting, or blocking the ability of an AAV to bind to an AAVR. In some embodiments, “blocking” means downregulating gene expression of an AAVR such that less AAVR is expressed on a cell treated with a “blocking” agent (e.g., AAVR-specific siRNA or ASO) as compared to a control cell of the same cell type that is not treated with the “blocking” agent. In some embodiments, “blocking” means contacting the AAVR with an agent (e.g., an antibody or small molecule) that prohibits the binding of an AAV to the AAVR.

As used herein, “RNAi compound,” “RNAi molecule,” or “RNAi” are used interchangeably and refer to inhibitory RNA. RNAi refers to an antisense compound that acts to modulate a target nucleic acid and/or protein encoded by a target nucleic acid. RNAi compounds include but are not limited to small interfering RNA (siRNA), single-stranded RNA (ssRNA), microRNA, including microRNA mimics, double-stranded RNA (dsRNA), short hairpin RNA (shRNA), and expression cassettes encoding RNA capable of inducing RNA interference. For example, RNAi expression cassettes can be transcribed in cells to produce siRNA, separate sense and anti-sense strand linear siRNA, or small hairpin RNAs that can function as miRNAs. As used herein, referencing “RNAi” and “siRNA” refers to the terms as used in the broadest sense and encompasses, for example, any siRNA that has been modified (e.g., chemical modification, attachment of at least one receptor-binding ligand or moiety) so long as the molecule retains the ability to bind to target nucleic acids in target cells, thereby reducing the target gene's expression. RNAi molecules are readily designed and generated by techniques known in the art.

As used herein, “antisense oligonucleotides” or “ASOs” refer to short strands of DNA or RNA that bind to a complementary RNA sequence, thereby inhibiting its function. ASOs can effectively downregulate or upregulate the production of certain downstream proteins downstream by inhibiting specific RNA sequences, and can theoretically be used with both select loss of function and gain of function mutations.

As used herein, “liver targeting moiety” includes, but is not limited to, any ligand or conjugate that can be applied to an agent that blocks AAV binding to AAVR (e.g., siRNA, RNAi, or an anti-AAVR antibody) to enhance the agent's delivery and/or uptake by the liver, including any known liver-targeting conjugates, including, N-acetylgalactosamine (GalNAc) conjugates, and any other delivery system for liver or hepatic delivery of the agent (e.g., siRNA or RNAi). The selection of an appropriate ligand or conjugate for targeting siRNAs to particular body systems, organs, tissues or cells is considered to be within the ordinary skill of the art. For example, to target an siRNA to hepatocytes, cholesterol may be attached at one or more ends, including any combination of 5′- and 3′-ends, of an siRNA molecule. The resultant cholesterol-siRNA is delivered to hepatocytes in the liver, thereby providing a means to deliver siRNAs to this targeted location. Other ligands useful for targeting siRNAs to the liver include HBV surface antigen and low-density lipoprotein (LDL).

As used herein, “LNP” or “lipid nanoparticle” refers to a lipid-based delivery composition. LNPs are known in the art and refer to particles that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides, including siRNA or other RNAi, to subjects can be used herein.

As used herein, “antibody” is used in the broadest sense and encompasses various antibody structures, including but not limited to monoclonal antibodies, polyclonal antibodies, multispecific antibodies (e.g., bispecific antibodies), nanobodies, and antibody or antigen-binding fragments so long as they exhibit the desired antigen-binding activity. As used herein, “anti-AAVR antibody” refers to an antibody (as used in the broadest sense as set forth above) that blocks an interaction between AAVR and AAV.

The terms “composition” or “formulation” refer to a preparation which is in such form as to permit the biological activity of an active ingredient contained therein to be effective, and which contains no additional components which are unacceptably toxic to a subject to which the composition would be administered.

The terms “pharmaceutically acceptable carrier” or “pharmaceutically acceptable vehicle” refer to any diluent, adjuvant, excipient, or combinations thereof, in a pharmaceutical composition which allows, for example, facilitation of the administration of the active ingredient contained therein. Non-limiting examples of substances that can generally serve as pharmaceutically acceptable carriers include oils, glycols; polyols, such as glycerin, sorbitol, mannitol, and polyethylene glycol; esters; agar; buffering agents; water; isotonic saline solution; Ringer's solution; ethyl alcohol; pH buffer solution; and any other non-toxic compatible materials used in pharmaceutical preparations. Such carriers or vehicles should be non-toxic and should not substantially interfere with the efficacy of the active ingredient. Pharmaceutically acceptable carriers are well known and will be adapted by the person skilled in the art as a function of the nature, route, and mode of administration.

As used herein, “treatment” (and variations thereof such as “treat” or “treating”) refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. As used herein, “treatment” can include administrating a therapeutic or therapeutic regimen including optional adjuvant or pre-conditioning regimen to achieve a therapeutic or prophylactic benefit. As used herein, “treatment” also encompasses “ameliorating,” which refers to any beneficial effect on a phenotype or symptom, such as reducing its severity, slowing or delaying its development, arresting its development, or partially or completely reversing or eliminating it.

“Pre-conditioning,” “preconditioning,” or “conditioning” are used interchangeably herein and refer to the preparation of the subject in need of the non-liver AAV-based gene therapy for a suitable condition, which includes blocking AAV binding to AAV receptors in the liver prior to the subject receiving the AAV-based gene therapy.

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically. The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others. “Co-administration,” as used herein, means that a plurality of substances are administered sufficiently close together in time so that the agents act together. Co-administration encompasses administering substances together in a single formulation and administering substances in separate formulations close enough in time so that the agents act together.

As used herein, “subject” may be a mammal, such as a primate, ungulate (e.g., cow, pig, horse), cat, dog, domestic pet or domesticated mammal. In some cases, the mammal may be a rabbit, pig, horse, sheep, cow, cat or dog, or a human. In some embodiments, the subject is a human. In some embodiments, the subject is an adult human. In some embodiments, the subject is a juvenile human. In some embodiments, the subject is greater than about 18 years old, greater than about 25 years old, or greater than about 35 years old. In some embodiments, the subject is less than about 18 years old, less than about 16 years old, less than about 14 years old, less than about 12 years old, less than about 10 years old, less than about 8 years old, less than about 6 years old, less than about 5 years old, less than about 4 years old, less than about 3 years old, less than about 2 years old, less than about 1 year old, or less than about 6 months old.

As used herein, “knock down,” “knockdown,” or the like refers suppression of the expression of a gene product, such as, for example, suppression achieved by the use of antisense oligo deoxynucleotides and RNAi that specifically target the RNA product of the gene. Gene knockdown refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. “Knock down” includes partial and complete suppression.

### II. Compositions

Disclosed herein are compositions comprising agents to block and/or that are useful for blocking AAV binding to AAVR receptors. In some embodiments, the compositions comprise a delivery molecule that targets the liver (e.g., hepatocytes). In some embodiments, the composition comprises an agent that blocks AAV binding to an AAV receptor (AAVR) and a delivery molecule that delivers the agent to the liver.

In some embodiments, the compositions and agents are capable of temporarily blocking AAV binding to AAVR receptors in the liver, including for about 48 hours to 3 weeks. In some embodiments, the compositions and agents are capable of blocking AAV binding to AAVR receptors in the liver for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the compositions are capable of blocking AAV binding to AAVR receptors in the liver for about 7 days, about 8 days, about 9 days, about 10 days, about 11 days, about 12 days, about 13 days, about 14 days. In some embodiments, the compositions and agents are capable of blocking AAV binding to AAVR receptors in the liver for 1-7, 1-10, 1-28 days, 7-28 days, 14-28 days, 21-28 days, 1-21 days, 7-21 days, 14-21 days, 1-14 days, 7-14 days, or 1-7 days.

In some embodiments, the compositions and agents are capable of long-term blocking AAV binding to AAVR receptors in the liver, including for longer than about 3 weeks, longer than about 4 weeks, longer than about 5 weeks, longer than about 6 weeks, longer than about 7 weeks, longer than about 8 weeks, longer than about 9 weeks, and longer than about 10 weeks. In some embodiments, the compositions and agents are capable of long-term blocking AAV binding to AAVR receptors in the liver, including for longer than 10 weeks.

A. RNAi and siRNA

In some embodiments, RNAi is the agent used to block AAV binding to AAVR in the liver. RNA interference refers to sequence- or gene-specific suppression of gene expression (protein synthesis) mediated by RNAi/siRNA in an organism without generally suppressing other protein synthesis. RNAi induce RNA interference through interaction with the RNA interference pathway method of mammalian cells in order to degrade or inhibit the translation of messenger RNA (mRNA) transcripts of transgene in a sequence-specific manner. RNAi activity directed toward major receptor proteins can lead to decreased entry into or binding to those cells. RNAi includes the use of small interfering RNA (siRNA) to target particular sequences in cells. RNAi polynucleotides include siRNA, microRNA (miRNA), double-stranded RNA (dsRNA), short hairpin RNA (shRNA), and expression cassettes encoding RNA capable of inducing RNA interference.

In some embodiments, the agent that blocks AAV binding to an AAV receptor (AAVR) is small-interfering RNA (siRNA). Small interfering RNA (siRNA) are known for their ability to specifically interfere with protein expression in a target. siRNAs are designed to interact with a target ribonucleotide sequence, meaning they complement a target sequence sufficiently to bind to the target sequence. siRNAs generally contain 15-50 base pairs, preferably 21-25 base pairs, and are used to encode a sequence of a target gene or RNA expressed in a cell, with a nucleotide sequence identity (fully complementary) or a nearly double identity (partial complementarity). They have also been used to knock down AAVR expression in Huh-7 cells by treatment with siRNA specific for AAVR. In a study assessing the ability of ImmTOR to restore transduction of Huh-7 cells transfected with AAVR-specific siRNA, it was found that Huh-7 cells expressing approximately 20% of normal AAVR showed a 50% reduction in AAVAnc80-luciferase expression when treated with the siRNA. See Ilyinskii et al., Sci. Adv., 7 eabd0321 (2021). It has also been reported that systematically or locally delivered siRNA can induce a temporary gene expression knockdown effect by up to 90% from 48 hours to 3 weeks in animal experiments for eyes, brain, spinal cord, lungs, subcutaneous tissue, vagina, skin, isolated tumor, heart et al. See Kim, Korean J Anesthesiol. 59(6): 369-370 (2010). Accordingly, in some embodiments, siRNA specific for AAVR is used to block AAV binding to AAVR in the liver. In some embodiments, the agent that blocks AAV binding to an AAV receptor (AAVR) is a small-interfering RNA (siRNA). In some embodiments, the subject has not been administered a reagent that enhances targeting to the liver (e.g., ImmTOR) in conjunction with the AAV-based gene therapy.

In other contexts, siRNA can be considered to have limited use because of the transient nature of the suppression effect seen in cells where the siRNA has been administered. Additionally, siRNAs are known to be unstable in vivo with limited long-term effectiveness. In some embodiments herein, the composition comprises a siRNA and the disclosure contemplates utilization of this temporal trait that is often viewed as a detriment in different context.

In some embodiments, the administration of any of the RNAi molecules disclosed herein or a composition thereof is capable of temporarily blocking AAV binding to AAVR receptors, e.g., in the liver, including for about 48 hours to 3 weeks. In some embodiments, the RNAi molecules and/or compositions are capable of blocking AAV binding to AAVR receptors, e.g., in the liver for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the RNAi molecules and/or compositions are capable of blocking AAV binding to AAVR receptors, e.g., in the liver for about 7 days, about 8 days, about 9 days, about 10 days, about 11 days, about 12 days, about 13 days, about 14 days. In some embodiments, the RNAi molecules and/or compositions are capable of blocking AAV binding to AAVR receptors, e.g., in the liver for 1-7, 1-10, 1-28 days, 7-28 days, 14-28 days, 21-28 days, 1-21 days, 7-21 days, 14-21 days, 1-14 days, 7-14 days, or 1-7 days.

In some embodiments, any of the RNAi molecules disclosed herein targets an AAVR-encoding transcript. In some embodiments, the AAVR-encoding transcript comprises a sequence that is at least 80%, 85%, 90%, 91%, 92%, 93%, 94%, 95%, 96%, 97%, 98%, 99% or 100% identical to the sequence of SEQ ID NO: 300, or a reverse complement thereof. In some embodiments, the RNAi molecule targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 300, or a reverse complement thereof. In some embodiments, the RNAi molecule targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 300, or a reverse complement thereof

SEQ ID NO: 300:
GTTTCCGGCCGCCGTCGCTGTCCAGGGAGGCTGAGGCGAGAGGTAG
CTGTCCGGGTGGGGAGCCCGCACTACCTTCTTCCTCTTCCTCCTC
CTCCTCCGGGTGAGGGGAGCGAAGGTTGGGGGTCCCCGAGCCCAT
GGACCAGGAGGAGGCGGAGGCCGCCGAGAGCCGGGGCCCCGCTAT
GTGGCCCTGAGCCCCGTGTACTGGTTCTGCCTGTCTGGAGGGCCA
TGGAGAAGAGGCTGGGAGTCAAGCCAAATCCTGCTTCCTGGATTT
TATCAGGATATTATTGGCAGACATCTGCGAAGTGGTTGAGAAGCC
TGTACCTGTTTTATACTTGCTTTTGCTTCAGCGTTCTGTGGTTGT
CAACAGATGCCAGTGAGAGCAGGTGCCAGCAGGGGAAGACACAAT
TTGGAGTTGGCCTGAGATCTGGGGGAGAAAATCACCTCTGGCTTC
TTGAAGGAACCCCCTCTCTCCAGTCATGTTGGGCTGCCTGCTGCC
AGGACTCTGCCTGCCATGTCTTTTGGTGGCTAGAAGGGATGTGCA
TTCAGGCAGACTGCAGCAGGCCCCAGAGCTGCCGGGCTTTTAGGA
CACACTCCTCCAATTCCATGCTGGTGTTTTTAAAAAAATTCCAAA
CTGCAGATGATTTGGGCTTTCTACCTGAAGATGATGTACCACATC
TTCTGGGGCTAGGTTGGAACTGGGCATCTTGGAGGCAGAGCCCAC
CCAGAGCTGCACTCAGACCTGCTGTATCTTCCAGTGACCAGCAGA
GCTTAATCAGGAAGCTTCAGAAGAGAGGTAGTCCCAGTGACGTAG
TTACACCTATAGTGACACAGCATTCTAAAGTGAATGACTCCAACG
AATTAGGTGGTCTGACTACCAGTGGCTCTGCAGAGGTCCACAAGG
CGATTACAATTTCCAGTCCCCTAACCACAGACCTGACTGCAGAGC
TGTCTGGTGGGCCAAAGAATGTATCAGTGCAACCTGAAATATCAG
AGGGTCTTGCTACTACGCCCAGCACTCAACAAGTAAAAAGTTCTG
AGAAAACCCAGATTGCTGTCCCCCAGCCAGTGGCTCCCTCCTACA
GTTATGCTACCCCTACCCCCCAGGCCTCTTTCCAGAGCACCTCAG
CACCATACCCAGTTATAAAGGAACTGGTGGTATCTGCTGGAGAGA
GTGTCCAGATAACCCTGCCTAAGAATGAAGTTCAATTAAATGCAT
ATGTTCTCCAAGAACCACCTAAAGGAGAAACCTACACCTACGACT
GGCAGCTGATTACTCATCCTAGAGACTACAGTGGAGAAATGGAAG
GGAAACATTCCCAGATCCTCAAACTATCGAAGCTCACTCCAGGCC
TGTATGAATTCAAAGTGATTGTAGAGGGTCAAAATGCCCATGGGG
AAGGCTATGTGAACGTGACAGTCAAGCCAGAGCCCCGTAAGAATC
GGCCCCCCATTGCTATTGTGTCACCTCAGTTCCAGGAGATCTCTT
TGCCAACCACTTCTACAGTCATTGATGGCAGTCAAAGCACTGATG
ATGATAAAATCGTTCAGTACCATTGGGAAGAACTTAAGGGGCCTC
TAAGAGAAGAGAAGATTTCTGAAGATACAGCCATATTAAAACTAA
GTAAACTCGTCCCTGGGAACTACACTTTCAGCTTGACTGTAGTAG
ACTCTGATGGAGCTACCAACTCTACTACTGCAAACCTGACAGTGA
ACAAAGCTGTGGATTACCCCCCTGTGGCCAACGCAGGCCCCAACC
AAGTGATCACCCTGCCCCAAAACTCCATCACCCTCTTTGGGAACC
AGAGCACTGATGATCATGGCATCACCAGCTATGAGTGGTCACTCA
GCCCAAGCAGCAAAGGGAAAGTGGTGGAGATGCAGGGTGTTAGAA
CACCAACCTTACAGCTCTCTGCGATGCAAGAAGGAGACTACACTT
ACCAGCTCACAGTGACTGACACAATAGGACAGCAGGCCACTGCTC
AAGTGACTGTTATTGTGCAACCTGAAAACAATAAGCCTCCTCAGG
CAGATGCAGGCCCAGATAAAGAGCTGACCCTTCCTGTGGATAGCA
CAACCCTGGATGGCAGCAAGAGCTCAGATGATCAGAAAATTATCT
CATATCTCTGGGAAAAAACACAGGGACCTGATGGGGTGCAGCTCG
AGAATGCTAACAGCAGTGTTGCTACTGTGACTGGGCTGCAAGTGG
GGACCTATGTGTTCACCTTGACTGTCAAAGATGAGAGGAACCTGC
AAAGCCAGAGCTCTGTGAATGTCATTGTCAAAGAAGAAATAAACA
AACCACCTATAGCCAAGATAACTGGGAATGTGGTGATTACCCTAC
CCACGAGCACAGCAGAGCTGGATGGCTCTAAGTCCTCAGATGACA
AGGGAATAGTCAGCTACCTCTGGACTCGAGATGAGGGGAGCCCAG
CAGCAGGGGAGGTGTTAAATCACTCTGACCATCACCCTATCCTTT
TTCTTTCAAACCTGGTTGAGGGAACCTACACTTTTCACCTGAAAG
TGACCGATGCAAAGGGTGAGAGTGACACAGACCGGACCACTGTGG
AGGTGAAACCTGATCCCAGGAAAAACAACCTGGTGGAGATCATCT
TGGATATCAACGTCAGTCAGCTAACTGAGAGGCTGAAGGGGATGT
TCATCCGCCAGATTGGGGTCCTCCTGGGGGTGCTGGATTCCGACA
TCATTGTGCAAAAGATTCAGCCGTACACGGAGCAGAGCACCAAAA
TGGTATTTTTTGTTCAAAACGAGCCTCCCCACCAGATCTTCAAAG
GCCATGAGGTGGCAGCGATGCTCAAGAGTGAGCTGCGGAAGCAAA
AGGCAGACTTTTTGATATTCAGAGCCTTGGAAGTCAACACTGTCA
CATGTCAGCTGAACTGTTCCGACCATGGCCACTGTGACTCGTTCA
CCAAACGCTGTATCTGTGACCCTTTTTGGATGGAGAATTTCATCA
AGGTGCAGCTGAGGGATGGAGACAGCAACTGTGAGTGGAGCGTGT
TATATGTTATCATTGCTACCTTTGTCATTGTTGTTGCCTTGGGAA
TCCTGTCTTGGACTGTGATCTGTTGTTGTAAGAGGCAAAAAGGAA
AACCCAAGAGGAAAAGCAAGTACAAGATCCTGGATGCCACGGATC
AGGAAAGCCTGGAGCTGAAGCCAACCTCCCGAGCAGGCATCAAAC
AGAAAGGCCTTTTGCTAAGTAGCAGCCTGATGCACTCCGAGTCAG
AGCTGGACAGCGATGATGCCATCTTTACATGGCCAGACCGAGAGA
AGGGCAAACTCCTGCATGGTCAGAATGGCTCTGTACCCAACGGGC
AGACCCCTCTGAAGGCCAGGAGCCCGCGGGAGGAGATCCTGTAGC
CACCTGGTCTGTCTCCTCAGGGCAGGGCCCAGCACACTGCCCGGC
CAGTCCTCCTACCTCCCGAGTCTGCGGGCAGCTGCTGTCCCAGCA
TCTGCTGGTCATTTCGCCCTGACAGTCCCAACCAGAACCCCTGGG
ACTTGAATCCAGAGACGTCCTCCAGGAACCCCTCAACGAAGCTGT
GAATGAAGAGGTTTCCTCTTTAAACCTGTCTGGTGGGCCCCCAGA
TATCCTCACCTCAGGGCCTCCTTTTTTTGCAAACTCCTCCCCTCC
CCCGAGGGCAGACCCAGCCAGCTGCTAAGCTCTGCAGCTCCCCAG
TGGACAGTGTCATTGTGCCCAGAGTGCTGCAAGGTGAGGCCTGCT
GTGCTGCCCGCACACCTGAGTGCAAAACCAAGCACTGTGGGCATG
GTGTTTCCCTCTCTGGGGTAGAGTACGCCCTCTCGCTGGGCAAAG
AGGAAGTGGCACCCCTCCCCTCACCACAGATGCTGAGATGGTAGC
ATAGAAATGATGGCCGGGCGCGGTGGCTCACGCCTGTAATCCCAG
CACTTTGGGAGGCCGAGGCGGGCGGATCATGAGGTCAGGAGATCA
AGACCACCCTGGCTAACACGGTGAAACCCCATCTCTACTAAAAAT
AAAAAAAAAAATTAGCCGGGTTTGGTGGCGTATGCCTGTAATCCC
AGCTACTCGGGAGGCTGAGGCAGGAGAATTGCTTAAACCTGGGAG
GTGGAGGCTGCAGTGAGCCAAGATCGTGCCACTGCACTCCAGCCT
GAGTGACAGAGCAAGACTCCGTCAAAAAAAAAAAAAAAAAAAAAG
AAATGATATCTGGCCCCCCCTTAACACTGGAGCCCCACTCCCTTC
TCCCATCCGGCCCGAGATTAGGGAGGATTGACTGTGTCAGGGATG
GCGGGTGGCCTCTCTCGCTGCCAGGGCCCTTGTCAGAGCAGCCAG
GCTGGACAGACGGCCTCCCTCCTCTCCATCTGACCGGCACCTGCT
GCTTCGGGGCTTAGGCCACCGCTCCCTGTCCCCAGAGGAGATAGC
CCCAGATGGACTGGAATGTTGTGGCATGAGAGCGCATGTGTGCGA
TGGCCCCGCTGTGGTCCCCTCTCTGTCCCTCCATCTGTATGTGTT
CTGTGTCCCTTGCATGTGTGCGTGTTAGAGTGAGCGCGTATGCAT
CAACTCATTGGGCTCTTGGCTGCTCACAAGGCAAATTTGACTTGG
AAAGACTTTCATCTCCTTGGAACCAAGACTTCCTGAGTCCCCCTC
ACCCTGGCCCTGTTCCACCATGGTTATCTGGGTATTGGGGAATGG
AAACTTTGGGGGAGTGACTTTTTAAAGAGACACTTATAATTTCTA
CTACTGCACTACTGTCCATTGTGGGATGATTAAACATGGTATTTA
ACTGTG.

In some embodiments, the composition comprises an RNAi (e.g., siRNA) where the administration of the composition is capable of knocking down AAVR. In some embodiments, the RNAi (e.g., siRNA) comprises a ribonucleotide sequence at least 80% identical to a ribonucleotide sequence from the AAVR. Preferably, the RNAi (e.g., siRNA) molecule is at least 90%, 95%, 96%, 97%, 98%, 99% or 100% identical to the ribonucleotide sequence of the target. Most preferably, an RNAi (e.g., siRNA) will be 100% identical to the nucleotide sequence ofthe target. However, siRNA molecules with insertions, deletions or single point mutations relative to a target may also be effective. In some embodiments, the RNAi (e.g., siRNA) targets AAVR in the liver. In some embodiments, the RNAi is siRNA. Tools to assist siRNA design are readily available to the public and are known in the art.

In some embodiments, the composition comprises an RNAi molecule that is between 18-31 nucleotides in length. In some embodiments, the RNAi (e.g., siRNA) is between 19-27 nucleotides in length. In some embodiments, the RNAi (e.g., siRNA) is between 19-25 nucleotides in length. In some embodiments, the RNAi (e.g., siRNA) is between 19-23 nucleotides in length. In some embodiments, the RNAi (e.g., siRNA) is between 19-21 nucleotides in length. In some embodiments, the RNAi (e.g., siRNA) is no more than 21, 25, or 31 nucleotides in length. In some embodiments, the RNAi (e.g., siRNA) is 21 nucleotides in length. In some embodiments, the RNAi is siRNA and comprises the sequence of any of the sequences in Table 3 (SEQ ID NOs: 4300-9507). In some embodiments, the siRNA comprises no more than and no fewer than 19 contiguous nucleotides of any of the sequences in Table 3. In some embodiments, the siRNA comprises no more than and no fewer than 20 contiguous nucleotides of any of the sequences in Table 3.

SEQ ID NOs: 4300-9507 in Table 3 reflect exemplary sequences for the antisense 5′ to 3′ strand of siRNA.

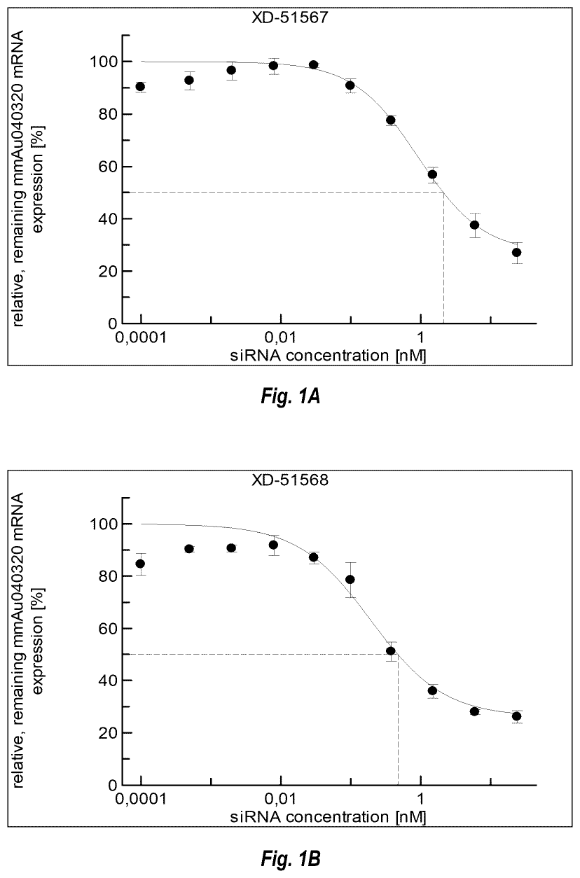

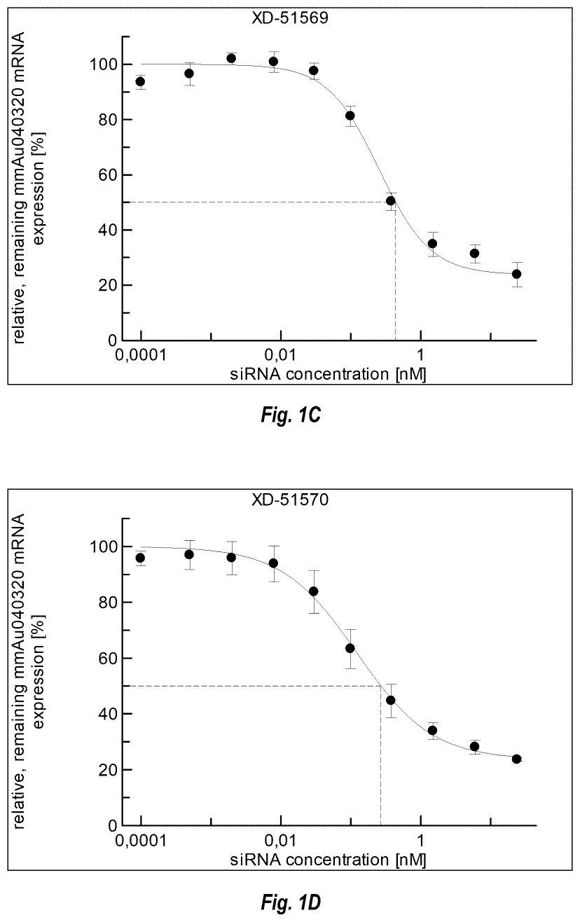

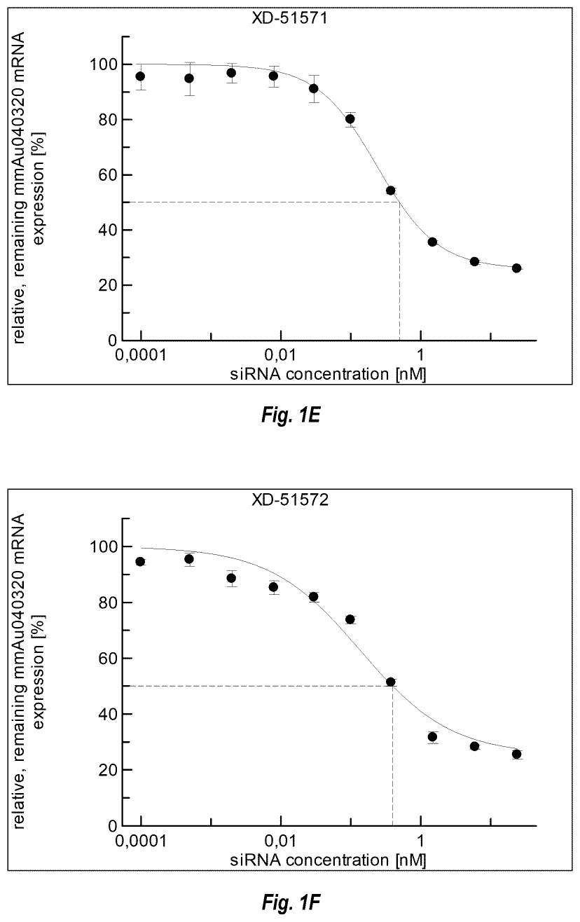

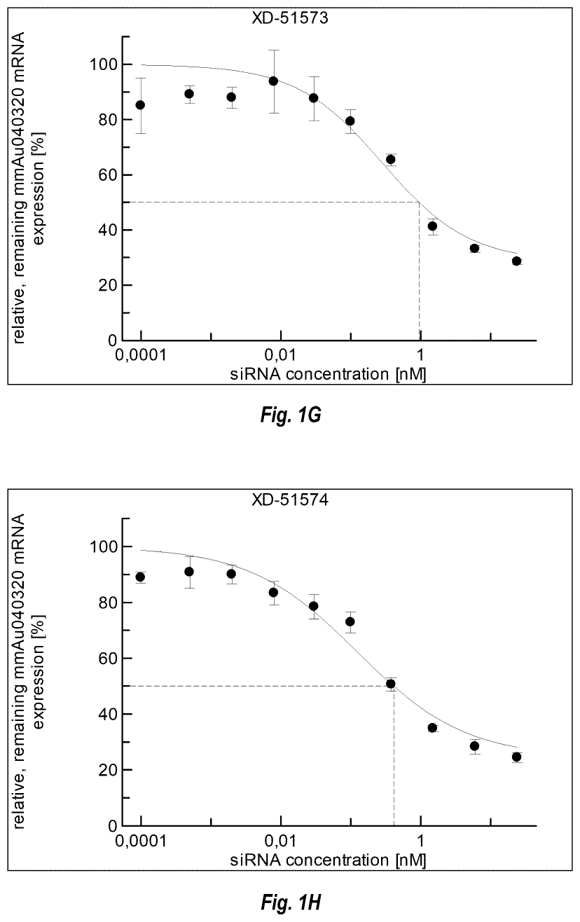

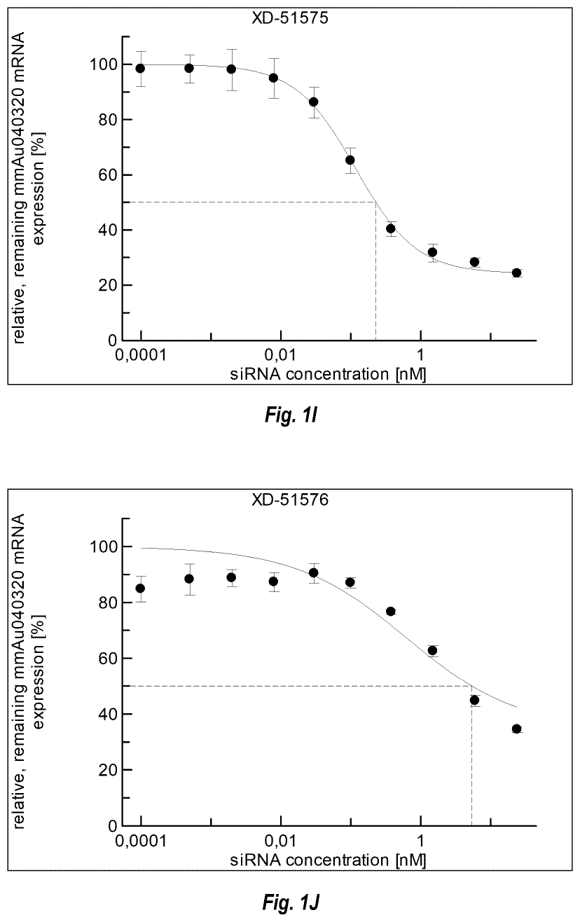

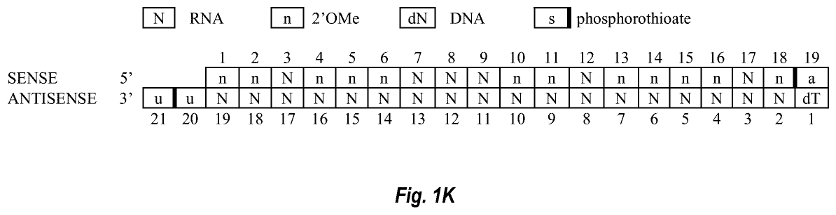

Sequences for 19-mer and 21-mer strands of siRNA with modifications that were used in the examples are set forth in Table A1, A2, and A3 below. In Table A2 (modified sequences), the “dT” denotes a DNA base instead ofan RNA base. Lower case letters in the sequences denote 2′OMe bases. Capital letters denote regular RNA bases. The “s” toward the 3′ end of the sense and antisense sequences denote the bond between the two bases in the siRNA sequence is a phosphorothioate bond. FIG. 1K shows a representative structure for the modified siRNA sequence.

TABLE A1
Exemplary siRNA sequences

| | | siRNA sense | | siRNA |
|---|---|---|---|---|
| | | strand | | antisense |
| | | core | | strand core |
| | SEQ | sequence | SEQ | sequence |
| | ID | (5′-3′) | ID | (5′-3′) |
| Duplex ID | NO: | 19 mer | NO: | 21 mer |
| XD-51567 | 9508 | GGUACUCCGC | 9509 | UUUUUACUUG |
| | | AAGUAAAAA | | CGGAGUACCA |
| | | | | G |
| XD-51568 | 9510 | CACUCAUCCU | 9511 | UUAGUCUGUA |
| | | ACAGACUAA | | GGAUGAGUGA |
| | | | | U |
| XD-51569 | 9512 | GCUUAACUGU | 9513 | UAGUCGACAA |
| | | UGUCGACUA | | CAGUUAAGCU |
| | | | | G |
| XD-51570 | 9514 | ACCUUGACUG | 9515 | UAUCUUUGAC |
| | | UCAAAGAUA | | AGUCAAGGUG |
| | | | | A |
| XD-51571 | 9516 | CCUACACGUU | 9517 | UUCAGGUGAA |
| | | UCACCUGAA | | ACGUGUAGGU |
| | | | | C |
| XD-51572 | 9518 | GGUGCUGGAU | 9519 | UAUGUCGGAA |
| | | UCCGACAUA | | UCCAGCACCC |
| | | | | C |
| XD-51573 | 9520 | CUGGAUUCCG | 9521 | UAAUGAUGUC |
| | | ACAUCAUUA | | GGAAUCCAGC |
| | | | | A |
| XD-51574 | 9522 | GCAAAAGAUU | 9523 | UUACGGCUGA |
| | | CAGCCGUAA | | AUCUUUUGCA |
| | | | | C |
| XD-51575 | 9524 | CAAAAGAUUC | 9525 | UGUACGGCUG |
| | | AGCCGUACA | | AAUCUUUUGC |
| | | | | A |
| XD-51576 | 9526 | CUGAUGCAUU | 9527 | UCGAUUCAGA |
| | | CUGAAUCGA | | AUGCAUCAGG |
| | | | | C |
| Unmodified | 9528 | GCGUUCUGUG | 9529 | GUUGACAACC |
| SIRNA | | GUUGUCAACA | | ACAGAACGCU |
| | | G | | G |
| Fluc | 9530 | CUUACGCUGA | 9531 | UCGAAGUACU |
| control | | GUACUUCGA | | CAGCGUAAGU |
| | | | | U |

TABLE A2
Modified siRNA sequences

| | | | | siRNA |
|---|---|---|---|---|
| | | siRNA sense | | antisense |
| | | strand | | strand |
| | | sequence | | sequence |
| | | (5′-3′) | | (5′-3′) |
| | SEQ | 19 mer | SEQ | 21 mer |
| | ID | with | ID | with |
| Duplex ID | NO: | modifications | NO: | modifications |
| XD-51567 | 9532 | ggUacuCCGc | 9533 | dTUUUUACUU |
| | | aAguaaAasa | | GCGgAGUACC |
| | | | | asg |
| XD-51568 | 9534 | caCucaUCCu | 9535 | dTUAGUCUGU |
| | | aCagacUasa | | AGGaUGAGUG |
| | | | | asu |
| XD-51569 | 9536 | gcUuaaCUGu | 9537 | dTAGUCGACA |
| | | uGucgaCusa | | ACAgUUAAGC |
| | | | | usg |
| XD-51570 | 9538 | acCuugACUg | 9539 | dTAUCUUUGA |
| | | uCaaagAusa | | CAGuCAAGGU |
| | | | | gsa |
| XD-51571 | 9540 | ccUacaCGUu | 9541 | dTUCAGGUGA |
| | | uCaccuGasa | | AACgUGUAGG |
| | | | | usc |
| XD-51572 | 9542 | ggUgcuGGAu | 9543 | dTAUGUCGGA |
| | | uCcgacAusa | | AUCCAGCACC |
| | | | | esc |
| XD-51573 | 9544 | cuGgauUCCg | 9545 | dTAAUGAUGU |
| | | aCaucaUusa | | CGGaAUCCAG |
| | | | | csa |
| XD-51574 | 9546 | gcAaaaGAUu | 9547 | dTUACGGCUG |
| | | cAgccgUasa | | AAUCUUUUGC |
| | | | | asc |
| XD-51575 | 9548 | caAaagAUUc | 9549 | dTGUACGGCU |
| | | aGccguAcsa | | GAAuCUUUUG |
| | | | | csa |
| XD-51576 | 9550 | cuGaugCAUu | 9551 | dTCGAUUCAG |
| | | cUgaauCgsa | | AAUgCAUCAG |
| | | | | gsc |
| unMod SiRNA | 9552 | GCGUUCUGUG | 9553 | GUUGACAACC |
| | | GUUGUCAACA | | ACAGAACGCU |
| | | G | | G |
| Fluc Control | 9554 | cuUacgCUGa | 9555 | dTCGAAGUAC |
| | | gUacuuCgsa | | UCAgCGUAAG |
| | | | | usu |

TABLE A3

| | |
|---|---|
| Core | Core |

| | sense | | antisense | | | | | | | | |
|---|---|---|---|---|---|---|---|---|---|---|---|
| | strand | | strand | Cross-reactivity | | | | | | | |
| SEQ | sequence | SEQ | sequence | 5 | 10 | 6 | 6 | 5 | 10 | 6 | 6 |
| ID | (5′-3′) | ID | (5′-3′) | 19 mer | | | | 21 mer | | | |
| NO: | 19 mer | NO: | 21 mer | Human | Mouse | Rhesus | Cyno | Human | Mouse | Rhesus | Cyno |
| 9508 | GGUACUCCGC | 9509 | UUUUUACUUG | 0 | 1 | 0 | 0 | 0 | 1 | 0 | 0 |
| | AAGUAAAAA | | CGGAGUACCA | | | | | | | | |
| | | | G | | | | | | | | |
| 9510 | CACUCAUCCU | 9511 | UUAGUCUGUA | 0 | 1 | 0 | 0 | 0 | 1 | 0 | 0 |
| | ACAGACUAA | | GGAUGAGUGA | | | | | | | | |
| | | | U | | | | | | | | |
| 9512 | GCUUAACUGU | 9513 | UAGUCGACAA | 0 | 1 | 0 | 0 | 0 | 1 | 0 | 0 |
| | UGUCGACUA | | CAGUUAAGCU | | | | | | | | |
| | | | G | | | | | | | | |
| 9514 | ACCUUGACUG | 9515 | UAUCUUUGAC | 1 | 1 | 1 | 1 | 1 | 1 | 1 | 1 |
| | UCAAAGAUA | | AGUCAAGGUG | | | | | | | | |
| | | | A | | | | | | | | |
| 9516 | CCUACACGUU | 9517 | UUCAGGUGAA | 0 | 1 | 1 | 1 | 0 | 1 | 1 | 1 |
| | UCACCUGAA | | ACGUGUAGGU | | | | | | | | |
| | | | C | | | | | | | | |
| 9518 | GGUGCUGGAU | 9519 | UAUGUCGGAA | 1 | 1 | 1 | 1 | 1 | 1 | 1 | 1 |
| | UCCGACAUA | | UCCAGCACCC | | | | | | | | |
| | | | C | | | | | | | | |
| 9520 | CUGGAUUCCG | 9521 | UAAUGAUGUC | 1 | 1 | 1 | 1 | 1 | 1 | 1 | 1 |
| | ACAUCAUUA | | GGAAUCCAGC | | | | | | | | |
| | | | A | | | | | | | | |
| 9522 | GCAAAAGAUU | 9523 | UUACGGCUGA | 1 | 1 | 1 | 1 | 1 | 1 | 1 | 1 |
| | CAGCCGUAA | | AUCUUUUGCA | | | | | | | | |
| | | | C | | | | | | | | |
| 9524 | CAAAAGAUUC | 9525 | UGUACGGCUG | 1 | 1 | 1 | 1 | 1 | 1 | 1 | 1 |
| | AGCCGUACA | | AAUCUUUUGC | | | | | | | | |
| | | | A | | | | | | | | |
| 9526 | CUGAUGCAUU | 9527 | UCGAUUCAGA | 0 | 1 | 0 | 0 | 0 | 1 | 0 | 0 |
| | CUGAAUCGA | | AUGCAUCAGG | | | | | | | | |
| | | | C | | | | | | | | |

| | siRNA strands with modifications | | | | Dose-response analysis | | |
|---|---|---|---|---|---|---|---|
| | | Sense | | Antisense | in Hepa1.6 cells | | |
| | SEQ | strand | SEQ | strand | | | Max. |
| Duplex | ID | sequence | ID | sequence | IC20 | IC50 | Inhib. |
| ID | NO: | (5′-3′) | NO: | (5′-3′) | [nM] | [nM] | [%] |
| XD-51567 | 9532 | ggUacuCCGc | 9533 | dTUUUUACUU | 0.326 | 2.1 | 73.1 |
| | | aAguaaAasa | | GCGgAGUACC | | | |
| | | | | asg | | | |
| XD-51568 | 9534 | caCucaUCCu | 9535 | dTUAGUCUGU | 0.059 | 0.47 | 73.9 |
| | | aCagacUasa | | AGGaUGAGUG | | | |
| | | | | asu | | | |
| XD-51569 | 9536 | gcUuaaCUGu | 9537 | dTAGUCGACA | 0.107 | 0.44 | 76.2 |
| | | uGucgaCusa | | ACAgUUAAGC | | | |
| | | | | usg | | | |
| XD-51570 | 9538 | acCuugACUg | 9539 | dTAUCUUUGA | 0.034 | 0.27 | 76.4 |
| | | uCaaagAusa | | CAGuCAAGGU | | | |
| | | | | gsa | | | |
| XD-51571 | 9540 | ccUacaCGUu | 9541 | dTUCAGGUGA | 0.089 | 0.49 | 74.1 |
| | | uCaccuGasa | | AACgUGUAGG | | | |
| | | | | usc | | | |
| XD-51572 | 9542 | ggUgcuGGAu | 9543 | dTAUGUCGGA | 0.031 | 0.4 | 74.6 |
| | | uCcgacAusa | | AUCcAGCACC | | | |
| | | | | esc | | | |
| XD-51573 | 9544 | cuGgauUCCg | 9545 | dTAAUGAUGU | 0.074 | 0.94 | 71.5 |
| | | aCaucaUusa | | CGGaAUCCAG | | | |
| | | | | csa | | | |
| XD-51574 | 9546 | gcAaaaGAUu | 9547 | dTUACGGCUG | 0.02 | 0.41 | 75.6 |
| | | cAgccgUasa | | AAUcUUUUGC | | | |
| | | | | asc | | | |
| XD-51575 | 9548 | caAaagAUUc | 9549 | dTGUACGGCU | 0.043 | 0.23 | 75.8 |
| | | aGccguAcsa | | GAAuCUUUUG | | | |
| | | | | csa | | | |
| XD-51576 | 9550 | cuGaugCAUu | 9551 | dTCGAUUCAG | 0.139 | 5.39 | 65.6 |
| | | cUgaauCgsa | | AAUgCAUCAG | | | |
| | | | | gsc | | | |

In some embodiments, the RNAi molecule is single-stranded. In some embodiments, the RNAi molecule is double-stranded. It should be noted that, any nucleotide lengths of any RNAi molecules recited in this application refer to a single strand of the RNAi molecule, even if that single strand is a member of a double-stranded RNAi molecule. For example, if an RNAi molecule is 21 nucleotides in length and is double stranded (without overhangs), the molecule would comprise a total of 42 nucleotides (21 nucleotides in each strand). In some embodiments, the RNAi molecule is double stranded and comprises blunt ends. In some embodiments, the RNAi molecule is double-stranded and comprises overhangs of one or more nucleotides. In some embodiments, the RNAi molecule is double stranded for only a portion of the molecule. For example, in some embodiments, a double-stranded RNAi molecule comprises overhangs on the sense and/or antisense strand of 1, 2, 3, 4, or 5 or more nucleotides. In some embodiments, a double-stranded RNAi molecule comprises overhangs on the sense and/or antisense strand of 1, 2 or 3 nucleotides.

In some embodiments, any of the RNAi molecules (e.g., siRNA molecules) disclosed herein comprises a nucleotide sequence that shares complementarity (e.g., 100% complementarity) with a target sequence in an AAVR RNA transcript. In some embodiments, the RNAi molecule comprises at least 17, 18, 19, 20, or 21 nucleotides that are complementary to a target sequence in an AAVR RNA transcript. In some embodiments, the RNAi molecule comprises at least 17, 18, 19, 20, or 21 nucleotides that are complementary to a target sequence in an AAVR RNA transcript, but wherein one or more nucleotides on the 3′ end of the RNAi molecule are not complementary to the target sequence in the AAVR RNA transcript. In some embodiments, the RNAi molecule comprises at least 17, 18, 19, 20, or 21 nucleotides that are complementary to a target sequence in an AAVR RNA transcript, but wherein one or more nucleotides on the 5′ end of the RNAi molecule are not complementary to the target sequence in the AAVR RNA transcript. In some embodiments, the RNAi molecule comprises at least 17, 18, 19, 20, or 21 nucleotides that are complementary to a target sequence in an AAVR RNA transcript, but wherein one or more nucleotides on the 3′ end and the 5′ end of the RNAi molecule are not complementary to the target sequence in the AAVR RNA transcript.

### B. Agent Delivery

In some embodiments, any of the agents that block AAV binding to an AAVR as disclosed herein are administered “naked”, i.e., without a molecule intended for a cell or tissue-specific delivery. For example, in some embodiments, the agent is administered “naked” in a pharmaceutically acceptable buffer, e.g., a buffered saline solution such as PBS.

In some embodiments, the compositions comprise an agent that blocks AAV binding to an AAV receptor (AAVR) and a delivery molecule that delivers the agent to the liver. In some embodiments, the compositions comprise a siRNA conjugated to a liver-targeting moiety. As used herein, “liver targeting moiety” includes but is not limited to any conjugate that can be applied to any of the agents that block AAV binding to an AAVR (e.g., siRNAs or RNAi) to enhance their delivery and/or uptake by the liver, including any known conjugates. Lipid moieties (e.g., lipid-conjugated siRNAs), such as cholesterol-conjugated siRNAs, and any other conjugates groups or moieties that are known in the art to effectively target the liver can also be used. In some embodiments, the delivery molecule comprises a lipid. In some embodiments, the compositions comprise lipid-conjugated siRNAs.

In some embodiments, the delivery molecule comprises at least one galactose or galactose derivative. In some embodiments, the compositions comprise siRNA conjugated to at least one galactose or galactose derivative. Galactose or galactose derivatives can target hepatocytes via their binding to the asialo glycoprotein receptor that is unique to and is highly expressed on the surface of hepatocytes (ASGPr). Binding of galactose moieties to ASGPr facilitates intracellular entry of the cell-specific target of the transferring polymer into the hepatocyte and the delivery polymer to the hepatocyte. Exemplary galactose or galactose derivatives include lactose, galactose, N-acetylgalactosamine (GalNAc), galactosamine, N-formylgalactosamine, N-acetylgalactosamine, N-propionylgalactosamine, Nn-butanoylgalactosamine, and N-isobutanoyl-galactosamine (Iobst, S T and Drickamer, K. JBC 1996, 271, 6686). In some embodiments, the delivery molecule comprises N-acetylgalactosamine (GalNAc). In some embodiments, the compositions comprise GalNAc-conjugated siRNAs.

As is also known in the art, the agent that blocks AAV binding to an AAVR can be delivered by non-viral tissue-specific delivery vehicles. In some embodiments, nanoparticles, liposomes, ribonucleoproteins, positively charged peptides, small molecule RNA-conjugates, aptamer-RNA chimeras, and RNA-fusion protein complexes are used to deliver the siRNA. In some embodiments, the delivery molecule comprises a lipid nanoparticle (LNP). In some embodiments, the composition comprises a LNP to deliver the agent to the liver.

A LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also be comprised of hydrophobic lipids, hydrophilic lipids, or both hydrophobic and hydrophilic lipids. Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20. The lipids can be combined in any number of molar ratios to produce a LNP. In addition, the polynucleotide(s) can be combined with lipid(s) in a wide range of molar ratios to produce a LNP.

### C. Anti-AAVR Antibodies

In some embodiments, the compositions comprise an anti-AAVR antibody to block AAV binding to an AAVR and otherwise reduce the interaction between AAVs and the receptor. It was previously found that AAV receptor (AAVR, also known as KIAA0319L) directly binds to AAV particles and is involved in AAV infection. In a study, it was found that anti-AAVR antibodies can block AAV2 infection. See Pillay, Nature, 530(7588): 108-112 (2016). Antibodies directed against AAVR were capable of potentially blocking AAV2 infection by more than 10-fold prior to infection, suggesting that blocking AAV access to AAVR on the cell surface substantially limits infection.

In some embodiments, the anti-AAVR antibody binds to an AAVR comprising a sequence that is at least 85%, 90%, 91%, 92%, 93%, 94%, 95%, 96%, 97%, 98%, 99% or 100% identical to SEQ ID NO: 301 and inhibits binding between the AAVR and an AAV. In some embodiments, the anti-AAVR antibody binds to any one or more of the following residues in AAVR: Arg406, Ser413, Ile419, Thr423, Ser425, Thr426, Val427, Asp429, Ser431, Gln432, Ser433, Thr434, Asp435, Asp436, Asp437, Lys438, Ile439, Tyr442, Glu458, Asp459, Ile462, and/or Lys464 of a protein comprising a sequence that is at least 85%, 90%, 91%, 92%, 93%, 94%, 95%, 96%, 97%, 98%, 99% or 100% identical to SEQ ID NO: 301. See, e.g., Meyer et al., 2019, eLife, 8:e44707. In some embodiments, the antibody binds to the AAVR and sterically blocks the interaction of the AAV to the AAVR. In some embodiments, the antibody binds to the AAVR and sterically blocks the interaction of the AAV for any one or more of the following residues in AAVR: Arg406, Ser413, Ile419, Thr423, Ser425, Thr426, Val427, Asp429, Ser431, Gln432, Ser433, Thr434, Asp435, Asp436, Asp437, Lys438, Ile439, Tyr442, Glu458, Asp459, Ile462, and/or Lys464 of a sequence that is at least 85%, 90%, 91%, 92%, 93%, 94%, 95%, 96%, 97%, 98%, 99% or 100% identical to SEQ ID NO: 301. In some embodiments, the compositions comprise anti-AAVR antibodies known in the art, including, for example ab105385 (Abcam).

SEQ ID NO: 301:
MEKRLGVKPNPASWILSGYYWQTSAKWLRSLYLFYTCFCFSVLWLS
TDASESRCQQGKTQFGVGLRSGGENHLWLLEGTPSLQSCWAACCQ
DSACHVFWWLEGMCIQADCSRPQSCRAFRTHSSNSMLVFLKKFQT
ADDLGFLPEDDVPHLLGLGWNWASWRQSPPRAALRPAVSSSDQQS
LIRKLQKRGSPSDVVTPIVTQHSKVNDSNELGGLTTSGSAEVHKA
ITISSPLTTDLTAELSGGPKNVSVQPEISEGLATTPSTQQVKSSE
KTQIAVPQPVAPSYSYATPTPQASFQSTSAPYPVIKELVVSAGES
VQITLPKNEVQLNAYVLQEPPKGETYTYDWQLITHPRDYSGEMEG
KHSQILKLSKLTPGLYEFKVIVEGQNAHGEGYVNVTVKPEPRKNR
PPIAIVSPQFQEISLPTTSTVIDGSQSTDDDKIVQYHWEELKGPL
REEKISEDTAILKLSKLVPGNYTFSLTVVDSDGATNSTTANLTVN
KAVDYPPVANAGPNQVITLPQNSITLFGNQSTDDHGITSYEWSLS
PSSKGKVVEMQGVRTPTLQLSAMQEGDYTYQLTVTDTIGQQATAQ
VTVIVQPENNKPPQADAGPDKELTLPVDSTTLDGSKSSDDQKIIS
YLWEKTQGPDGVQLENANSSVATVTGLQVGTYVFTLTVKDERNLQ
SQSSVNVIVKEEINKPPIAKITGNVVITLPTSTAELDGSKSSDDK
GIVSYLWTRDEGSPAAGEVLNHSDHHPILFLSNLVEGTYTFHLKV
TDAKGESDTDRTTVEVKPDPRKNNLVEIILDINVSQLTERLKGMF
IRQIGVLLGVLDSDIIVQKIQPYTEQSTKMVFFVQNEPPHQIFKG
HEVAAMLKSELRKQKADFLIFRALEVNTVTCQLNCSDHGHCDSFT
KRCICDPFWMENFIKVQLRDGDSNCEWSVLYVIIATFVIVVALGI
LSWTVICCCKRQKGKPKRKSKYKILDATDQESLELKPTSRAGIKQ
KGLLLSSSLMHSESELDSDDAIFTWPDREKGKLLHGQNGSVPNGQ
TPLKARSPREEIL

### D. Antisense Oligonucleotides

In some embodiments, the compositions comprise short single-stranded oligonucleotides (RNA or DNA) that are capable of binding to target sequences, e.g., inactivating or interfering with corresponding AAVR mRNA or DNA sequences, thereby down-regulating the expression of the target AAVR genes.

In some embodiments, the composition comprises an antisense oligonucleotide, also referred to herein as “ASO,” to block AAV binding to AAVR in the liver. The antisense oligonucleotide can be a single or double stranded DNA or RNA or chimeric mixtures or derivatives or modified versions thereof. As is known in the art, for antisense oligonucleotides to sufficiently inhibit their target sequence as efficiently as possible, there should be a degree of complementarity between the antisense oligonucleotides and the corresponding target sequence. Chemical modifications of ASOs are known in the art to increase their resistance to various nucleases, as well as their binding affinity to RNA targets. Phosphorothioate (PS) modification, in which a non-bridging oxygen is replaced by a sulfur atom in the phosphate backbone; 2′-O-methyl (2′-O-Me), 2′-O-methoxyethyl (MOE) modification; constrained ethyl (cEt) modification; and bicyclic nucleoside modifications such as 2′,4′-methylene bridged nucleic acids, commonly called locked nucleic acid (LNA) modification, are non-limiting examples. Accordingly, in some embodiments, the antisense oligonucleotide comprises a modified sequence. In some embodiments, the ASO contains MOE or LNA modifications.

In some embodiments, the antisense oligonucleotide is linked to ligands or conjugates known in the art and/or described herein or delivered by non-viral tissue-specific delivery vehicles, which may be used, e.g., to increase the cellular uptake of antisense oligonucleotides.

In some embodiments, the antisense oligonucleotide is administered without conjugation and without a non-viral tissue-specific delivery vehicle. In some embodiments, the antisense oligonucleotides are administered without a non-viral tissue-specific delivery vehicle and are administered in a composition comprising a pharmaceutically acceptable carrier.

In some embodiments, any of the ASOs disclosed herein are administered “naked”, i.e., without a molecule intended for a cell or tissue-specific delivery. For example, in some embodiments, the agent is administered “naked” in a pharmaceutically acceptable buffer, e.g., a buffered saline solution such as PBS.

In some embodiments, any of the ASOs disclosed herein is at least 14, 15, 16, 17, 18, 19, 20, 21, 22, 23, 24, or 25 nucleotides in length. In some embodiments, any of the ASOs disclosed herein is no more than 35, 34, 33, 32, 31, 30, 29, 28, 27, 26 or 25 nucleotides in length. In some embodiments, any of the ASOs disclosed herein is between 14-35, 14-30, 14-25, 14-20, 20-35, 20-30, 20-25, 25-35, or 25-30 nucleotides in length. In some embodiments, the ASO is less than 20 nucleotides in length. In some embodiments, the ASO is 14-18, 15-17, or 16 nucleotides in length.

Methods of generating antisense oligonucleotides are known in the art. In some embodiments, the antisense oligonucleotide targets an AAVR-encoding transcript. In some embodiments, the AAVR-encoding transcript comprises a sequence that is at least 80%, 85%, 90%, 91%, 92%, 93%, 94%, 95%, 96%, 97%, 98%, 99% or 100% identical to the sequence of SEQ ID NO: 300, or a reverse complement thereof. In some embodiments, the antisense oligonucleotide targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 300, or a reverse complement thereof. In some embodiments, the antisense oligonucleotide targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 300, or a reverse complement thereof.

In some embodiments, any of the ASOs disclosed herein comprises any of the sequences disclosed in Table B. In some embodiments, any of the ASOs disclosed herein comprises any of the sequences disclosed in the sequences of SEQ ID Nos: 9600-9625. In some embodiments, any of the ASOs disclosed herein comprises a sequence that is at least 80%, 85%, 90%, 91%, 92%, 93%, 94%, 95%, 96%, 97%, 98%, 99% or 100% identical to any of the sequences of SEQ ID Nos: 9600-9625. In some embodiments, the ASO comprises at least 14-20, 15-20, 16-20, 17-20, 18-20, or 19-20 consecutive nucleotides of any one or more SEQ ID Nos: 9600-9625 or of any sequence that is at least 80%, 85%, 90%, 91%, 92%, 93%, 94%, 95%, 96%, 97%, 98%, 99% or 100% identical to any of the sequences of SEQ ID Nos: 9600-9625. In some embodiments, the ASO comprises the nucleotide sequence of SEQ ID NO: 9600 or a nucleotide sequence that is at least 14-20, 15-20, 16-20, 17-20, 18-20, or 19-20 consecutive nucleotides of any one or more SEQ ID Nos: 9600. In some embodiments, the ASO comprises the nucleotide sequence of SEQ ID NO: 9601 or a nucleotide sequence that is at least 14-20, 15-20, 16-20, 17-20, 18-20, or 19-20 consecutive nucleotides of any one or more SEQ ID Nos: 9601. In some embodiments, the ASO comprises the nucleotide sequence of SEQ ID NO: 9602 or a nucleotide sequence that is at least 14-20, 15-20, 16-20, 17-20, 18-20, or 19-20 consecutive nucleotides of any one or more SEQ ID Nos: 9602. In some embodiments, the ASO comprises the nucleotide sequence of SEQ ID NO: 9603 or a nucleotide sequence that is at least 14-20, 15-20, 16-20, 17-20, 18-20, or 19-20 consecutive nucleotides of any one or more SEQ ID Nos: 9603. In some embodiments, the ASO comprises the nucleotide sequence of SEQ ID NO: 9604 or a nucleotide sequence that is at least 14-20, 15-20, 16-20, 17-20, 18-20, or 19-20 consecutive nucleotides of any one or more SEQ ID Nos: 9604. In some embodiments, the ASO comprises the nucleotide sequence of SEQ ID NO: 9605 or a nucleotide sequence that is at least 14-20, 15-20, 16-20, 17-20, 18-20, or 19-20 consecutive nucleotides of any one or more SEQ ID Nos: 9605. In some embodiments, the ASO comprises the nucleotide sequence of SEQ ID NO: 9606 or a nucleotide sequence that is at least 14-20, 15-20, 16-20, 17-20, 18-20, or 19-20 consecutive nucleotides of any one or more SEQ ID Nos: 9606. In some embodiments, the ASO comprises the nucleotide sequence of SEQ ID NO: 9607 or a nucleotide sequence that is at least 14-20, 15-20, 16-20, 17-20, 18-20, or 19-20 consecutive nucleotides of any one or more SEQ ID Nos: 9607. In some embodiments, the ASO comprises the nucleotide sequence of SEQ ID NO: 9608 or a nucleotide sequence that is at least 14-20, 15-20, 16-20, 17-20, 18-20, or 19-20 consecutive nucleotides of any one or more SEQ ID Nos: 9608. In some embodiments, the ASO comprises the nucleotide sequence of SEQ ID NO: 9609 or a nucleotide sequence that is at least 14-20, 15-20, 16-20, 17-20, 18-20, or 19-20 consecutive nucleotides of any one or more SEQ ID Nos: 9609. In some embodiments, the ASO comprises the nucleotide sequence of SEQ ID NO: 9610 or a nucleotide sequence that is at least 14-20, 15-20, 16-20, 17-20, 18-20, or 19-20 consecutive nucleotides of any one or more SEQ ID Nos: 9610. In some embodiments, the ASO comprises the nucleotide sequence of SEQ ID NO: 9611 or a nucleotide sequence that is at least 14-20, 15-20, 16-20, 17-20, 18-20, or 19-20 consecutive nucleotides of any one or more SEQ ID Nos: 9611. In some embodiments, the ASO comprises the nucleotide sequence of SEQ ID NO: 9612 or a nucleotide sequence that is at least 14-20, 15-20, 16-20, 17-20, 18-20, or 19-20 consecutive nucleotides of any one or more SEQ ID Nos: 9612. In some embodiments, the ASO comprises the nucleotide sequence of SEQ ID NO: 9613 or a nucleotide sequence that is at least 14-20, 15-20, 16-20, 17-20, 18-20, or 19-20 consecutive nucleotides of any one or more SEQ ID Nos: 9613. In some embodiments, the ASO comprises the nucleotide sequence of SEQ ID NO: 9614 or a nucleotide sequence that is at least 14-20, 15-20, 16-20, 17-20, 18-20, or 19-20 consecutive nucleotides of any one or more SEQ ID Nos: 9614. In some embodiments, the ASO comprises the nucleotide sequence of SEQ ID NO: 9615 or a nucleotide sequence that is at least 14-20, 15-20, 16-20, 17-20, 18-20, or 19-20 consecutive nucleotides of any one or more SEQ ID Nos: 9615. In some embodiments, the ASO comprises the nucleotide sequence of SEQ ID NO: 9616 or a nucleotide sequence that is at least 14-20, 15-20, 16-20, 17-20, 18-20, or 19-20 consecutive nucleotides of any one or more SEQ ID Nos: 9616. In some embodiments, the ASO comprises the nucleotide sequence of SEQ ID NO: 9617 or a nucleotide sequence that is at least 14-20, 15-20, 16-20, 17-20, 18-20, or 19-20 consecutive nucleotides of any one or more SEQ ID Nos: 9617. In some embodiments, the ASO comprises the nucleotide sequence of SEQ ID NO: 9618 or a nucleotide sequence that is at least 14-20, 15-20, 16-20, 17-20, 18-20, or 19-20 consecutive nucleotides of any one or more SEQ ID Nos: 9618. In some embodiments, the ASO comprises the nucleotide sequence of SEQ ID NO: 9619 or a nucleotide sequence that is at least 14-20, 15-20, 16-20, 17-20, 18-20, or 19-20 consecutive nucleotides of any one or more SEQ ID Nos: 9619. In some embodiments, the ASO comprises the nucleotide sequence of SEQ ID NO: 9620 or a nucleotide sequence that is at least 14-20, 15-20, 16-20, 17-20, 18-20, or 19-20 consecutive nucleotides of any one or more SEQ ID Nos: 9620. In some embodiments, the ASO comprises the nucleotide sequence of SEQ ID NO: 9621 or a nucleotide sequence that is at least 14-20, 15-20, 16-20, 17-20, 18-20, or 19-20 consecutive nucleotides of any one or more SEQ ID Nos: 9621. In some embodiments, the ASO comprises the nucleotide sequence of SEQ ID NO: 9622 or a nucleotide sequence that is at least 14-20, 15-20, 16-20, 17-20, 18-20, or 19-20 consecutive nucleotides of any one or more SEQ ID Nos: 9622. In some embodiments, the ASO comprises the nucleotide sequence of SEQ ID NO: 9623 or a nucleotide sequence that is at least 14-20, 15-20, 16-20, 17-20, 18-20, or 19-20 consecutive nucleotides of any one or more SEQ ID Nos: 9623. In some embodiments, the ASO comprises the nucleotide sequence of SEQ ID NO: 9624 or a nucleotide sequence that is at least 14-20, 15-20, 16-20, 17-20, 18-20, or 19-20 consecutive nucleotides of any one or more SEQ ID Nos: 9624. In some embodiments, the ASO comprises the nucleotide sequence of SEQ ID NO: 9625 or a nucleotide sequence that is at least 14-20, 15-20, 16-20, 17-20, 18-20, or 19-20 consecutive nucleotides of any one or more SEQ ID Nos: 9625. In some embodiments, the ASO comprises the nucleotide sequence of SEQ ID NO: 9626 or a nucleotide sequence that is at least 14-20, 15-20, 16-20, 17-20, 18-20, or 19-20 consecutive nucleotides of any one or more SEQ ID Nos: 9626. In some embodiments, the ASO is no more than 20, no more than 19, no more than 18, no more than 17, no more than 16, no more than 15 or no more than 15 nucleotides in length. In some embodiments, the ASO is no more than 16 nucleotides in length.

Sequences for the modified ASOs that were used in the examples are set forth in Table B below. In the ASO sequence modifications, the “*” indicates that the bond between the two bases is a phosphorothioate, while the “+” indicates that the nucleic acid is a locked nucleic acid (LNA). For the MOE-modified ASOs, “2MOEr” indicates that a specific base is a 2′-O-methoxy-ethyl Base (2′-MOE). A “5” before “2MOEr” indicates that it is the first (5′) base in the oligo, a “3” indicates that it is the last (3′) base in the oligo, and an “i” indicates that it is an internal base (middle of the oligo). The last letter is the identity of the base, namely A, T, C, or G; each base's designation is separated by slashes.

TABLE B

| CRO/ | SEQ | | |
|---|---|---|---|
| internal | ID | ASO Sequence | |
| label | NO: | Unmodified | ASO Sequence Modification |
| AAVR_ASO_1 | 9600 | CAGCTGCCAGTCGTAGGTGT | +C*+A*+G*+C*+T*G*C*C*A*G*T*C |
| | | | *G*T*A*+G*+G*+T*+G*+T |
| AAVR_ASO_2 | 9601 | TAGCCTTCCCCATGGGCATT | +T*+A*+G*+C*+C*T*T*C*C*C*C*A* |
| | | | T*G*G*+G*+C*+A*+T*+T |
| AAVR_3 | 9602 | ATCTCCTGGAACTGAGGTGA | +A*+T*+C*+T*+C*C*T*G*G*A*A*C |
| | | | *T*G*A*+G*+G*+T*+G*+A |
| AAVR_ASO_4 | 9603 | GATCACTTGGTTGGGGCCTG | +G*+A*+T*+C*+A*C*T*T*G*G*T*T* |
| | | | G*G*G*+G*+C*+C*+T*+G |
| AAVR_ASO_5/ | 9604 | CTGCATCTGCCTGAGGAGGC | +C*+T*+G*+C*+A*T*C*T*G*C*C*T* |
| IH_ASO5 | | | G*A*G*+G*+A*+G*+G*+C |
| AAVR_ASO_6/ | 9605 | CTCATCTTTGACAGTCAAGG | +C*+T*+C*+A*+T*C*T*T*T*G*A*C* |
| IH_ASO6 | | | A*G*T*+C*+A*+A*+G*+G |
| AAVR_7 | 9606 | ATCCCCTTCAGCCTCTCAGT | +A*+T*+C*+C*+C*C*T*T*C*A*G*C* |
| | | | C*T*C*+T*+C*+A*+G*+T |
| AAVR_8/ | 9607 | CCCCCAGGAGGACCCCAATC | +C*+C*+C*+C*+C*A*G*G*A*G*G*A |
| IH-ASO8 | | | *C*C*C*+C*+A*+A*+T*+C |
| LNA_1/ | 9608 | TCTTTGACAGTCAAGG | +T*+C*+T*T*T*G*A*C*A*G*T*C*A* |
| ASOVT002 | | | +A*+G*+G |
| LNA_2 | 9609 | TGTCGGAATCCAGCAC | +T*+G*+T*C*G*G*A*A*T*C*C*A*G |
| | | | *+C*+A*+C |
| LNA_3 | 9610 | ATGATGTCGGAATCCA | +A*+T*+G*A*T*G*T*C*G*G*A*A*T |
| | | | *+C*+C*+A |
| LNA_4 | 9611 | ACGGCTGAATCTTTTG | +A*+C*+G*G*C*T*G*A*A*T*C*T*T |
| | | | *+T*+T*+G |
| LNA_5 | 9612 | TACGGCTGAATCTTTT | +T*+A*+C*G*G*C*T*G*A*A*T*C*T |
| | | | *+T*+T*+T |
| LNA_IH5 | 9613 | GCATCTGCCTGAGGAG | +G*+C*+A*T*C*T*G*C*C*T*G*A*G |
| | | | *+G*+A*+G |
| LNA_IH6 | 9614 | CATCTTTGACAGTCAA | +C*+A*+T*C*T*T*T*G*A*C*A*G*T* |
| | | | +C*+A*+A |
| LNA_IH8 | 9615 | CCCAGGAGGACCCCAA | *+C*+A*+A |
| 18_MOE_1 | 9616 | ATCTTTGACAGTCAAGGT | /52MOErA//i2MOErT//i2MOErC//i2MO |
| | | | ErT/TTGACAGTCA/12MOErA//12MOEr |
| | | | G//12MOErG//32MOErT/ |
| 18_MOE_2 | 9617 | ATGTCGGAATCCAGCACC | /52MOErA//i2MOErT//i2MOErG//i2MO |
| | | | ErT/CGGAATCCAG/12MOErC//12MOE |
| | | | rA//12MOErC//32MOErC/ |
| 18_MOE_3 | 9618 | AATGATGTCGGAATCCAG | /52MOErA//i2MOErA//i2MOErT//i2MO |
| | | | ErG/ATGTCGGAAT/i2MOErC//i2MOE |
| | | | rC//i2MOErA//32MOErG/ |
| 18_MOE_4 | 9619 | TACGGCTGAATCTTTTGC | /52MOErT//i2MOErA//i2MOErC//i2MO |
| | | | ErG/GCTGAATCTT/12MOErT//i2MOEr |
| | | | T//12MOErG//32MOErC/ |
| 18_MOE_5 | 9620 | GTACGGCTGAATCTTTTG | /52MOErG//i2MOErT//i2MOErA//i2MO |
| | | | ErC/GGCTGAATCT/i2MOErT//i2MOEr |
| | | | T//12MOErT//32MOErG/ |
| 18_MOE_IH5 | 9621 | TGCATCTGCCTGAGGAGG | /52MOErT//i2MOErG//i2MOErC//i2MO |
| | | | ErA/TCTGCCTGAG/12MOErG//i2MOEr |
| | | | A//i2MOErG//32MOErG/ |
| 18_MOE_IH6 | 9622 | TCATCTTTGACAGTCAAG | /52MOErT//i2MOErC//i2MOErA//12MO |
| | | | ErT/CTTTGACAGT/12MOErC//i2MOEr |
| | | | A//12MOErA//32MOErG/ |
| 18_MOE_IH8 | 9623 | CCCCAGGAGGACCCCAAT | /52MOErC//i2MOErC//i2MOErC//i2MO |
| | | | ErC/AGGAGGACCC/i2MOErC//i2MOE |
| | | | rA//i2MOErA//32MOErT/ |
| LNA_Control | 9624 | GGCTACTACGCCGTCA | GGCTACTACGCCGTCA |
| MOE_Control | 9625 | TTAGTTTAATCACGCTCG | /52MOErT//i2MOErT//i2MOErA//12MO |
| | | | ErG/TTTAATCACG/i2MOErC//i2MOEr |
| | | | T//12MOErC//32MOErG/ |

E. Small Molecules and Other Agents that Block AAV Binding to AAVR

In some embodiments, the compositions comprise small molecules that block AAVR binding to AAVs and/or otherwise inhibit or reduce the interaction between AAVs and the receptor. It is contemplated that other suitable inhibitory agents can be produced using techniques known to those of ordinary skill in the art, including inhibitors of AAVR expression.

In some embodiments, the compositions comprise a soluble variant polypeptide, that blocks binding between an AAV particle and AAVR. In some embodiments, the soluble variant polypeptide is a variant of AAVR that has a portion of the protein that is sufficient for AAV to bind at a recognizable affinity, but which lacks a transmembrane domain (e.g., lacks the naturally present transmembrane domain of the corresponding wild type protein). For example, in some embodiments, the soluble AAVR polypeptide lacks the transmembrane domain, or the transmembrane domain and the cytoplasmic tail, of the corresponding wild type AAVR protein and is capable of binding to AAV, thereby blocking the AAV particle from binding to AAVR on the cell surface (e.g., in the liver). See, e.g., U.S. Ser. No. 10/633,662B2 (Pillay).

### F. AAV Compositions

In some embodiments, AAV vectors and/or compositions thereof are administered after administering any of the compositions above that block AAV binding to an AAVR and are delivered to the liver, wherein administration of any of the compositions that block AAV binding to AAVR increases the percentage of AAV delivered to a non-liver target. Accordingly, the present invention contemplates administering a single AAV vector or multiple AAV vectors that have a non-liver target and/or compositions thereof.

In some embodiments, the AAV is an AAV1, AAV2, AAV3, AAV4, AAV5, AAV6, AAV7, AAV8, AAVrh10, AAVrh74, AAV9, AAV9P, or Myo-AAV vector. In some embodiments, the one or more AAV vectors are recombinant or engineered AAV vectors. In some embodiments, the one or more AAV vectors comprise a tissue-specific (e.g., muscle-specific) promoter, e.g., which is operatively linked to a sequence encoding a guide RNA.

In some embodiments, any of the AAVs disclosed herein comprises a tRNA or a nucleotide sequence encoding a tRNA. In some embodiments, the tRNA is a suppressor tRNA. In some embodiments, the suppressor tRNA comprises an anticodon that hybridizes to a premature stop codon in a target gene (e.g., a mutant dystrophin gene) and that is capable of being aminoacylated with an amino acid. In some embodiments, any of the AAVs disclosed herein comprises a nucleotide sequence encoding any of the tRNA molecules described in one or more of US2020277607, US2022073933, US2020291401, US2022112489, WO2019090154, WO2019090169, WO2020150608, WO2021087401, WO2020069199, or WO2018161032 each of which applications is incorporated by reference herein in its entirety.

In some embodiments, the one or more AAV vectors include CRISPR-Cas components, any of which are known in the art. In some embodiments, the one or more AAV vectors comprise a nucleic acid encoding a Cas9 protein. Such embodiments include for example, AAV vectors comprising a nucleic acid encoding Staphylococcus aureus (SaCas9) and/or Staphylococcus lugdunensis (SluCas9) and further comprising a nucleic acid encoding one or more guide RNAs. In such embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9. In some embodiments, in addition to guide RNA and Cas9 sequences, the one or more vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA.

In some embodiments, the muscle-cell cell specific promoter is a variant of the CK8 promoter, called CK8e. In some embodiments, the size of the CK8e promoter is 436 bp. In some embodiments, the CK8e promoter comprises a nucleotide sequence that is at least 90%, 91%, 92%, 93%, 94%, 95%, 96%, 97%, 98%, 99% or 100% identical to the sequence of SEQ ID NO: 302:

| | |
|---|---|
| 1 | TGCCCATGTA AGGAGGCAAG GCCTGGGGAC |
| | ACCCGAGATG CCTGGTTATA ATTAACCCAG |
| 61 | ACATGTGGCT GCCCCCCCCC CCCCAACACC |
| | TGCTGCCTCT AAAAATAACC CTGCATGCCA |
| 121 | TGTTCCCGGC GAAGGGCCAG CTGTCCCCCG |
| | CCAGCTAGAC TCAGCACTTA GTTTAGGAAC |
| 181 | CAGTGAGCAA GTCAGCCCTT GGGGCAGCCC |
| | ATACAAGGCC ATGGGGCTGG GCAAGCTGCA |
| 241 | CGCCTGGGTC CGGGGTGGGC ACGGTGCCCG |
| | GGCAACGAGC TGAAAGCTCA TCTGCTCTCA |
| 361 | GGGGCCCCTC CCTGGGGACA GCCCCTCCTG |
| | GCTAGTCACA CCCTGTAGGC TCCTCTATAT |
| 361 | AACCCAGGGG CACAGGGGCT GCCCTCATTC |
| | TACCACCACC TCCACAGCAC AGACAGACAC |
| 421 | TCAGGAGCCA GCCAGC |

In some embodiments, the promoter for expression of any of the nucleic acids disclosed herein is a U6 promoter. In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 303:

GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACC.

In some embodiments, the promoter for expression of any of the nucleic acids disclosed herein is a H1 promoter. In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 304:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the promoter for expression of any of the nucleic acids disclosed herein is a 7SK2 promoter. In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 305:

CTGCAGTATTTAGCATGCCCCACCCATCTGCAAGGCATTCTGGATAGTGT
CAAAACAGCCGGAAATCAAGTCCGTTTATCTCAAACTTTAGCATTTTGGG
AATAAATGATATTTGCTATGCTGGTTAAATTAGATTTTAGTTAAATTTCC
TGCTGAAGCTCTAGTACGATAAGCAACTTGACCTAAGTGTAAAGTTGAGA
CTTCCTTCAGGTTTATATAGCTTGTGCGCCGCTTGGGTACCTC.

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. A discussion of modified guide RNAs can be found in WO2022/056000, which is incorporated herein in its entirety. In some embodiments, the guide RNAs are unmodified.

In some embodiments, the one or more vectors comprise multiple nucleic acids encoding more than one guide RNA. In some embodiments, the one or more vectors comprise two nucleic acids encoding two guide RNA sequences.

In some embodiments, the one or more vectors comprise a nucleic acid encoding a Cas9 protein (e.g., an SaCas9 protein or SluCas9 protein), a nucleic acid encoding a first guide RNA, and a nucleic acid encoding a second guide RNA. In some embodiments, the one or more vectors do not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the one or more vectors comprise a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the one or more vectors comprise a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9 or SluCas9 protein) is under the control of the CK8e promoter. In some embodiments, the first guide is under the control of the 7SK2 promoter, and the second guide is under the control of the Hlm promoter. In some embodiments, the first guide is under the control of the Hlm promoter, and the second guide is under the control of the 7SK2 promoter. In some embodiments, the first guide is under the control of the hU6c promoter, and the second guide is under the control of the Hlm promoter. In some embodiments, the first guide is under the control of the Hlm promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA.

In some embodiments, any of the vectors disclosed herein is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb from ITR to ITR in size, inclusive of both ITRs. In some embodiments, the vector is an AAV9 vector.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of a second sgRNA, a second sgRNA guide sequence, and a second sgRNA scaffold sequence. In some embodiments the promoter for expression of the nucleic acid encoding the first and/or second sgRNA is a hU6c promoter or a 7SK2 promoter. In some embodiments the promoter for expression of the nucleic acid encoding the second sgRNA is a Hlm promoter. In some embodiments, the promoter for SaCas9 is the CK8e promoter. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the non-liver target is the muscle. In such embodiments, the non-liver AAV-based gene therapy is used to treat DMD.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 306:

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 307 (designated herein as SaCas9-KKH or SACAS9KKH):

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRKLINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYKNDLIKINGELYRVIGVNNDLLNRIEVNMIDITY
REYLENMNDKRPPHIIKTIASKTQSIKKYSTDILGNLYEVKSKKHPQIIK
KG

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 308:

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 309 (designated herein as sRGN1):

MNQKFILGLDIGITSVGYGLIDYETKNIIDAGVRLFPEANVENNEGRRSK
RGSRRLKRRRIHRLDRVKHLLAEYDLLDLTNIPKSTNPYQTRVKGLNEKL
SKDELVIALLHIAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGKGSPFGWEGNIKKWFEQMMGHCTYFPEELR
SVKYSYSAELFNALNDLNNLVITRDEDAKLNYGEKFQIIENVFKQKKTPN
LKQIAIEIGVHETEIKGYRVNKSGTPEFTEFKLYHDLKSIVFDKSILENE
AILDQIAEILTIYQDEQSIKEELNKLPEILNEQDKAEIAKLIGYNGTHRL
SLKCIHLINEELWQTSRNQMEIFNYLNIKPNKVDLSEQNKIPKDMVNDFI
LSPVVKRTFIQSINVINKVIEKYGIPEDIIIELARENNSDDRKKFINNLQ
KKNEATRKRINEIIGQTGNQNAKRIVEKIRLHDQQEGKCLYSLKDIPLED
LLRNPNNYDIDHIIPRSVSFDDSMHNKVLVRREQNAKKNNQTPYQYLTSG
YADIKYSVFKQHVLNLAENKDRMTKKKREYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 310 (designated herein as sRGN2):

MNQKFILGLDIGITSVGYGLIDYETKNIIDAGVRLFPEANVENNEGRRSK
RGSRRLKRRRIHRLERVKSLLSEYKIISGLAPTNNQPYNIRVKGLTEQLT
KDELAVALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGQGSPFGWNGDLKKWYEMLMGHCTYFPQELRSVKY
AYSADLFNALNDLNNLIIQRDNSEKLEYHEKYHIIENVFKQKKKPTLKQI
AKEIGVNPEDIKGYRITKSGTPEFTEFKLYHDLKSVLFDQSILENEDVLD
QIAEILTIYQDKDSIKSKLTELDILLNEEDKENIAQLTGYNGTHRLSLKC
IRLVLEEQWYSSRNQMEIFTHLNIKPKKINLTAANKIPKAMIDEFILSPV
VKRTFIQSINVINKVIEKYGIPEDIIIELARENNSDDRKKFINNLQKKNE
ATRKRINEIIGQTGNQNAKRIVEKIRLHDQQEGKCLYSLESIALMDLLNN
PQNYEVDHIIPRSVAFDNSIHNKVLVKQIENSKKGNRTPYQYLNSSDAKL
SYNQFKQHILNLSKSKDRISKKKKDYLLEERDINKFEVQKEFINRNLVDT
RYATRELTSYLKAYFSANNMDVKVKTINGSFTNHLRKVWRFDKYRNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 311 (designated herein as sRGN3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 312 (designated herein as sRGN3.1):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 313 (designated herein as sRGN3.2):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 314 (designated herein as sRGN3.3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 315 (designated herein as sRGN4):

MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHNINVSSEDEDASNELSTKEQINRNNKLLKDK
YVCEVQLQRLKEGQIRGEKNRFKTTDILKEIDQLLKVQKDYHNLDIDFIN
QYKEIVETRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPEFTSFKLFHDLKKVVKDHAILDDIDLL
NQIAEILTIYQDKDSIVAELGQLEYLMSEADKQSISELTGYTGTHSLSLK
CMNMIIDELWHSSMNQMEVFTYLNMRPKKYELKGYQRIPTDMIDDAILSP
VVKRTFIQSINVINKVIEKYGIPEDIIIELARENNSDDRKKFINNLQKKN
EATRKRINEIIGQTGNQNAKRIVEKIRLHDQQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIETKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN

In some embodiments, the guide RNAs comprise as non-limiting examples the guide sequences disclosed in Tables 1A, 1B, and Table 2 below. For example, when the AAV vector comprises SaCas9, one or more spacer sequences is selected from any one of SEQ ID NOs: 1-35, 1000-1078, and 3000-3069; or when the AAV vector comprises SluCas9, one or more spacer sequences selected from any one of SEQ ID NOs: 100-225, 2000-2116, and 4000-4251 from the tables below. Additional exemplary AAV compositions, including varieties of RNP complexes (comprising one or more guide RNAs comprising and saCas9 or sluCas9, or a mutant Cas9 protein), are disclosed elsewhere in WO2022/056000, which is incorporated herein in its entirety.

TABLE 1A
Exemplary DMD guide sequences (human-hg38.p12)

| Sequence | | | | | |
|---|---|---|---|---|---|
| ID No. | | | | | |
| of Guide | | | | | |
| Sequence | EXON | CAS9 | strand | Guide sequence | pam |
| 1 | EXON43 | SACAS9 | + | GCAATGCTGCTGTCTTCTTGCT | ATGAAT |
| 2 | EXON43 | SACAS9 | − | AACAAAATGTACAAGGACCGAC | AAGGGT |
| 3 | EXON43 | SACAS9 | − | TGCAAAGTGCAACGCCTGTGGA | AAGGGT |
| 4 | EXON43 | SACAS9 | − | ATAGTCTACAACAAAGCTCAGG | TCGGAT |
| 5 | EXON43 | SACAS9 | − | CTGTTTTAAAATTTTTATATTA | CAGAAT |
| 6 | EXON44 | SACAS9 | + | ATTTAGCATGTTCCCAATTCTC | AGGAAT |
| 7 | EXON44 | SACAS9 | + | AATCGCCTGCAGGTAAAAGCAT | ATGGAT |
| 8 | EXON44 | SACAS9 | − | TCTCAGAAAGACACAAATTCCT | GAGAAT |
| 9 | EXON45 | SACAS9 | + | TCAGGCTTCCCAATTTTTCCTG | TAGAAT |
| 10 | EXON45 | SACAS9 | + | TAGAATACTGGCATCTGTTTTT | GAGGAT |
| 11 | EXON45 | SACAS9 | + | TGGCATCTGTTTTTGAGGATTG | CTGAAT |
| 12 | EXON45 | SACAS9 | + | TTGCCGCTGCCCAATGCCATCC | TGGAGT |
| 13 | EXON45 | SACAS9 | − | GAGGTAGGGCGACAGATCTAAT | AGGAAT |
| 14 | EXON45 | SACAS9 | − | TCTACAGGAAAAATTGGGAAGC | CTGAAT |
| 15 | EXON45 | SACAS9 | − | GCGGCAAACTGTTGTCAGAACA | TTGAAT |
| 16 | EXON45 | SACAS9 | − | TTTTGGTATCTTACAGGAACTC | CAGGAT |
| 17 | EXON50 | SACAS9 | − | ACTATTGGAGCCTGTAAGTATA | CTGGAT |
| 18 | EXON50 | SACAS9 | − | AGGAAGTTAGAAGATCTGAGCT | CTGAGT |
| 19 | EXON51 | SACAS9 | + | TAGTAACCACAGGTTGTGTCAC | CAGAGT |
| 20 | EXON51 | SACAS9 | + | GTTGTGTCACCAGAGTAACAGT | CTGAGT |
| 21 | EXON51 | SACAS9 | + | TCTGAGTAGGAGCTAAAATATT | TTGGGT |
| 22 | EXON51 | SACAS9 | − | GAGGGTGATGGTGGGTGACCTT | GAGGAT |
| 23 | EXON51 | SACAS9 | − | TATAAAATCACAGAGGGTGATG | GTGGGT |
| 24 | EXON51 | SACAS9 | − | TTGATCAAGTTATAAAATCACA | GAGGGT |
| 25 | EXON53 | SACAS9 | + | CCTTGGTTTCTGTGATTTTCTT | TTGGAT |
| 26 | EXON53 | SACAS9 | + | TCCTTAGCTTCCAGCCATTGTG | TTGAAT |
| 27 | EXON53 | SACAS9 | + | CTTGTACTTCATCCCACTGATT | CTGAAT |
| 28 | EXON53 | SACAS9 | + | ACTGATTCTGAATTCTTTCAAC | TAGAAT |
| 29 | EXON53 | SACAS9 | − | AGCCAAGCTTGAGTCATGGAAG | GAGGGT |
| 30 | EXON53 | SACAS9 | − | TTAGGACAGGCCAGAGCCAAGC | TTGAGT |
| 31 | EXON53 | SACAS9 | − | GCAACAGTTGAATGAAATGTTA | AAGGAT |
| 32 | EXON53 | SACAS9 | − | CCTTCAGAACCGGAGGCAACAG | TTGAAT |
| 33 | EXON53 | SACAS9 | − | AGTTGAAAGAATTCAGAATCAG | TGGGAT |
| 34 | EXON53 | SACAS9 | − | TTTTATTCTAGTTGAAAGAATT | CAGAAT |
| 35 | EXON53 | SACAS9 | − | TTTTTCCTTTTATTCTAGTTGA | AAGAAT |
| 100 | EXON43 | SLUCAS9 | + | ATATATGTGTTACCTACCCTTG | TCGG |
| 101 | EXON43 | SLUCAS9 | + | ACATTTTGTTAACTTTTTCCCA | TTGG |
| 102 | EXON43 | SLUCAS9 | + | CTTTTTCCCATTGGAAATCAAG | CTGG |
| 103 | EXON43 | SLUCAS9 | + | TTTTTCCCATTGGAAATCAAGC | TGGG |
| 104 | EXON43 | SLUCAS9 | + | TCCTGTAGCTTCACCCTTTCCA | CAGG |
| 105 | EXON43 | SLUCAS9 | − | AATGTACAAGGACCGACAAGGG | TAGG |
| 106 | EXON43 | SLUCAS9 | − | ACAAAATGTACAAGGACCGACA | AGGG |
| 107 | EXON43 | SLUCAS9 | − | AACAAAATGTACAAGGACCGAC | AAGG |
| 108 | EXON43 | SLUCAS9 | − | GGAAAAAGTTAACAAAATGTAC | AAGG |
| 109 | EXON43 | SLUCAS9 | − | TCTCTCCCAGCTTGATTTCCAA | TGGG |
| 110 | EXON43 | SLUCAS9 | − | CTCTCTCCCAGCTTGATTTCCA | ATGG |
| 111 | EXON43 | SLUCAS9 | − | GCCTGTGGAAAGGGTGAAGCTA | CAGG |
| 112 | EXON43 | SLUCAS9 | − | GCAAAGTGCAACGCCTGTGGAA | AGGG |
| 113 | EXON43 | SLUCAS9 | − | TGCAAAGTGCAACGCCTGTGGA | AAGG |
| 114 | EXON43 | SLUCAS9 | − | AGCATTGCAAAGTGCAACGCCT | GTGG |
| 115 | EXON43 | SLUCAS9 | − | ATAGTCTACAACAAAGCTCAGG | TCGG |
| 116 | EXON43 | SLUCAS9 | − | AAAGATAGTCTACAACAAAGCT | CAGG |
| 117 | EXON44 | SLUCAS9 | + | TATTTAGCATGTTCCCAATTCT | CAGG |
| 118 | EXON44 | SLUCAS9 | + | AACAGATCTGTCAAATCGCCTG | CAGG |
| 119 | EXON44 | SLUCAS9 | + | AATCGCCTGCAGGTAAAAGCAT | ATGG |
| 120 | EXON44 | SLUCAS9 | − | TGCTAAATACAAATGGTATCTT | AAGG |
| 121 | EXON44 | SLUCAS9 | − | ATTGGGAACATGCTAAATACAA | ATGG |
| 122 | EXON44 | SLUCAS9 | − | AAAGACACAAATTCCTGAGAAT | TGGG |
| 123 | EXON44 | SLUCAS9 | − | GAAAGACACAAATTCCTGAGAA | TTGG |
| 124 | EXON44 | SLUCAS9 | − | ATGATATAAAGATATTTAATCA | GTGG |
| 125 | EXON44 | SLUCAS9 | − | TTGACAGATCTGTTGAGAAATG | GCGG |
| 126 | EXON44 | SLUCAS9 | − | GATTTGACAGATCTGTTGAGAA | ATGG |
| 127 | EXON44 | SLUCAS9 | − | TTGATCCATATGCTTTTACCTG | CAGG |
| 128 | EXON45 | SLUCAS9 | + | AGACCTCCTGCCACCGCAGATT | CAGG |
| 129 | EXON45 | SLUCAS9 | + | TCCCAATTTTTCCTGTAGAATA | CTGG |
| 130 | EXON45 | SLUCAS9 | + | TAGAATACTGGCATCTGTTTTT | GAGG |
| 131 | EXON45 | SLUCAS9 | + | TTTGCCGCTGCCCAATGCCATC | CTGG |
| 132 | EXON45 | SLUCAS9 | + | GGAGTTCCTGTAAGATACCAAA | AAGG |
| 133 | EXON45 | SLUCAS9 | − | AGAGGTAGGGCGACAGATCTAA | TAGG |
| 134 | EXON45 | SLUCAS9 | − | CTGTCAGACAGAAAAAAGAGGT | AGGG |
| 135 | EXON45 | SLUCAS9 | − | GCTGTCAGACAGAAAAAAGAGG | TAGG |
| 136 | EXON45 | SLUCAS9 | − | AACAGCTGTCAGACAGAAAAAA | GAGG |
| 137 | EXON45 | SLUCAS9 | − | AAGCCTGAATCTGCGGTGGCAG | GAGG |
| 138 | EXON45 | SLUCAS9 | − | GGGAAGCCTGAATCTGCGGTGG | CAGG |
| 139 | EXON45 | SLUCAS9 | − | AATTGGGAAGCCTGAATCTGCG | GTGG |
| 140 | EXON45 | SLUCAS9 | − | AAAAATTGGGAAGCCTGAATCT | GCGG |
| 141 | EXON45 | SLUCAS9 | − | GCCAGTATTCTACAGGAAAAAT | TGGG |
| 142 | EXON45 | SLUCAS9 | − | TGCCAGTATTCTACAGGAAAAA | TTGG |
| 143 | EXON45 | SLUCAS9 | − | AAAAACAGATGCCAGTATTCTA | CAGG |
| 144 | EXON45 | SLUCAS9 | − | TGTCAGAACATTGAATGCAACT | GGGG |
| 145 | EXON45 | SLUCAS9 | − | TTGTCAGAACATTGAATGCAAC | TGGG |
| 146 | EXON45 | SLUCAS9 | − | GTTGTCAGAACATTGAATGCAA | CTGG |
| 147 | EXON45 | SLUCAS9 | − | AACTCCAGGATGGCATTGGGCA | GCGG |
| 148 | EXON45 | SLUCAS9 | − | TACAGGAACTCCAGGATGGCAT | TGGG |
| 149 | EXON45 | SLUCAS9 | − | TTACAGGAACTCCAGGATGGCA | TTGG |
| 150 | EXON45 | SLUCAS9 | − | GGTATCTTACAGGAACTCCAGG | ATGG |
| 151 | EXON45 | SLUCAS9 | − | TTTTGGTATCTTACAGGAACTC | CAGG |
| 152 | EXON45 | SLUCAS9 | − | GTTTTGCCTTTTTGGTATCTTA | CAGG |
| 153 | EXON45 | SLUCAS9 | − | TCTTTTCTCAAATAAAAAGACA | TGGG |
| 154 | EXON50 | SLUCAS9 | + | AGAATGGGATCCAGTATACTTA | CAGG |
| 155 | EXON50 | SLUCAS9 | + | AGTATACTTACAGGCTCCAATA | GTGG |
| 156 | EXON50 | SLUCAS9 | + | CAGGCTCCAATAGTGGTCAGTC | CAGG |
| 157 | EXON50 | SLUCAS9 | + | CAATAGTGGTCAGTCCAGGAGC | TAGG |
| 158 | EXON50 | SLUCAS9 | + | GTGGTCAGTCCAGGAGCTAGGT | CAGG |
| 159 | EXON50 | SLUCAS9 | + | TTGCCCTCAGCTCTTGAAGTAA | ACGG |
| 160 | EXON50 | SLUCAS9 | − | AGTATACTGGATCCCATTCTCT | TTGG |
| 161 | EXON50 | SLUCAS9 | − | ACTATTGGAGCCTGTAAGTATA | CTGG |
| 162 | EXON50 | SLUCAS9 | − | CTAGCTCCTGGACTGACCACTA | TTGG |
| 163 | EXON50 | SLUCAS9 | − | GCAAAGCAGCCTGACCTAGCTC | CTGG |
| 164 | EXON50 | SLUCAS9 | − | AAACCGTTTACTTCAAGAGCTG | AGGG |
| 165 | EXON50 | SLUCAS9 | − | TAAACCGTTTACTTCAAGAGCT | GAGG |
| 166 | EXON50 | SLUCAS9 | − | AGATCTGAGCTCTGAGTGGAAG | GCGG |
| 167 | EXON50 | SLUCAS9 | − | AGAAGATCTGAGCTCTGAGTGG | AAGG |
| 168 | EXON50 | SLUCAS9 | − | AGTTAGAAGATCTGAGCTCTGA | GTGG |
| 169 | EXON50 | SLUCAS9 | − | ATGTGTATGCTTTTCTGTTAAA | GAGG |
| 170 | EXON51 | SLUCAS9 | + | TGATCATCTCGTTGATATCCTC | AAGG |
| 171 | EXON51 | SLUCAS9 | + | TTGATCAAGCAGAGAAAGCCAG | TCGG |
| 172 | EXON51 | SLUCAS9 | + | AGTCGGTAAGTTCTGTCCAAGC | CCGG |
| 173 | EXON51 | SLUCAS9 | + | GCCCGGTTGAAATCTGCCAGAG | CAGG |
| 174 | EXON51 | SLUCAS9 | + | CAGAGCAGGTACCTCCAACATC | AAGG |
| 175 | EXON51 | SLUCAS9 | + | GGTACCTCCAACATCAAGGAAG | ATGG |
| 176 | EXON51 | SLUCAS9 | + | CAAGGAAGATGGCATTTCTAGT | TTGG |
| 177 | EXON51 | SLUCAS9 | + | AGATGGCATTTCTAGTTTGGAG | ATGG |
| 178 | EXON51 | SLUCAS9 | + | ATGGCAGTTTCCTTAGTAACCA | CAGG |
| 179 | EXON51 | SLUCAS9 | + | GTCACCAGAGTAACAGTCTGAG | TAGG |
| 180 | EXON51 | SLUCAS9 | + | TCTGAGTAGGAGCTAAAATATT | TTGG |
| 181 | EXON51 | SLUCAS9 | + | CTGAGTAGGAGCTAAAATATTT | TGGG |
| 182 | EXON51 | SLUCAS9 | + | AAATATTTTGGGTTTTTGCAAA | AAGG |
| 183 | EXON51 | SLUCAS9 | − | GTATGAGAAAAAATGATAAAAG | TTGG |
| 184 | EXON51 | SLUCAS9 | − | CAACGAGATGATCATCAAGCAG | AAGG |
| 185 | EXON51 | SLUCAS9 | − | GAGGGTGATGGTGGGTGACCTT | GAGG |
| 186 | EXON51 | SLUCAS9 | − | ATAAAATCACAGAGGGTGATGG | TGGG |
| 187 | EXON51 | SLUCAS9 | − | TATAAAATCACAGAGGGTGATG | GTGG |
| 188 | EXON51 | SLUCAS9 | − | AGTTATAAAATCACAGAGGGTG | ATGG |
| 189 | EXON51 | SLUCAS9 | − | TGATCAAGTTATAAAATCACAG | AGGG |
| 190 | EXON51 | SLUCAS9 | − | TTGATCAAGTTATAAAATCACA | GAGG |
| 191 | EXON51 | SLUCAS9 | − | GGGCTTGGACAGAACTTACCGA | CTGG |
| 192 | EXON51 | SLUCAS9 | − | CTCTGGCAGATTTCAACCGGGC | TTGG |
| 193 | EXON51 | SLUCAS9 | − | ACCTGCTCTGGCAGATTTCAAC | CGGG |
| 194 | EXON51 | SLUCAS9 | − | TACCTGCTCTGGCAGATTTCAA | CCGG |
| 195 | EXON51 | SLUCAS9 | − | CTTGATGTTGGAGGTACCTGCT | CTGG |
| 196 | EXON51 | SLUCAS9 | − | AATGCCATCTTCCTTGATGTTG | GAGG |
| 197 | EXON51 | SLUCAS9 | − | AGAAATGCCATCTTCCTTGATG | TTGG |
| 198 | EXON51 | SLUCAS9 | − | GGTGACACAACCTGTGGTTACT | AAGG |
| 199 | EXON51 | SLUCAS9 | − | TGTTACTCTGGTGACACAACCT | GTGG |
| 200 | EXON51 | SLUCAS9 | − | AGCTCCTACTCAGACTGTTACT | CTGG |
| 201 | EXON53 | SLUCAS9 | + | AAAGGTATCTTTGATACTAACC | TTGG |
| 202 | EXON53 | SLUCAS9 | + | CCTTGGTTTCTGTGATTTTCTT | TTGG |
| 203 | EXON53 | SLUCAS9 | + | CTTTTGGATTGCATCTACTGTA | TAGG |
| 204 | EXON53 | SLUCAS9 | + | TTTTGGATTGCATCTACTGTAT | AGGG |
| 205 | EXON53 | SLUCAS9 | + | ACCCTCCTTCCATGACTCAAGC | TTGG |
| 206 | EXON53 | SLUCAS9 | + | CTTCCATGACTCAAGCTTGGCT | CTGG |
| 207 | EXON53 | SLUCAS9 | + | ACATTTCATTCAACTGTTGCCT | CCGG |
| 208 | EXON53 | SLUCAS9 | + | TCAACTGTTGCCTCCGGTTCTG | AAGG |
| 209 | EXON53 | SLUCAS9 | + | TGAATTCTTTCAACTAGAATAA | AAGG |
| 210 | EXON53 | SLUCAS9 | − | CCAAAAGAAAATCACAGAAACC | AAGG |
| 211 | EXON53 | SLUCAS9 | − | GCCAAGCTTGAGTCATGGAAGG | AGGG |
| 212 | EXON53 | SLUCAS9 | − | AGCCAAGCTTGAGTCATGGAAG | GAGG |
| 213 | EXON53 | SLUCAS9 | − | CAGAGCCAAGCTTGAGTCATGG | AAGG |
| 214 | EXON53 | SLUCAS9 | − | AGGCCAGAGCCAAGCTTGAGTC | ATGG |
| 215 | EXON53 | SLUCAS9 | − | AGAAGCTGAGCAGGTCTTAGGA | CAGG |
| 216 | EXON53 | SLUCAS9 | − | AAGGAAGAAGCTGAGCAGGTCT | TAGG |
| 217 | EXON53 | SLUCAS9 | − | GGAAGCTAAGGAAGAAGCTGAG | CAGG |
| 218 | EXON53 | SLUCAS9 | − | TTCAACACAATGGCTGGAAGCT | AAGG |
| 219 | EXON53 | SLUCAS9 | − | GTTAAAGGATTCAACACAATGG | CTGG |
| 220 | EXON53 | SLUCAS9 | − | AAATGTTAAAGGATTCAACACA | ATGG |
| 221 | EXON53 | SLUCAS9 | − | GCAACAGTTGAATGAAATGTTA | AAGG |
| 222 | EXON53 | SLUCAS9 | − | TACAAGAACACCTTCAGAACCG | GAGG |
| 223 | EXON53 | SLUCAS9 | − | AAGTACAAGAACACCTTCAGAA | CCGG |
| 224 | EXON53 | SLUCAS9 | − | AGTTGAAAGAATTCAGAATCAG | TGGG |
| 225 | EXON53 | SLUCAS9 | − | TAGTTGAAAGAATTCAGAATCA | GTGG |

TABLE 1B
Exemplary DMD guide sequences (20-nucleotides and
21-nucleotides)

| Sequence | | | | |
|---|---|---|---|---|
| ID No. | | | | |
| of Guide | | | | |
| Sequence | EXON | CAS9 | Strand | Guide sequence |
| 3000 | EXON43 | SACAS9 | + | AATGCTGCTGTCTTCTTGCT |
| 3001 | EXON43 | SACAS9 | + | CAATGCTGCTGTCTTCTTGCT |
| 1 | EXON43 | SACAS9 | + | GCAATGCTGCTGTCTTCTTGCT |
| 3002 | EXON43 | SACAS9 | − | AACAAAATGTACAAGGACCG |
| 3003 | EXON43 | SACAS9 | − | AACAAAATGTACAAGGACCGA |
| 2 | EXON43 | SACAS9 | − | AACAAAATGTACAAGGACCGAC |
| 3004 | EXON43 | SACAS9 | − | TGCAAAGTGCAACGCCTGTG |
| 3005 | EXON43 | SACAS9 | − | TGCAAAGTGCAACGCCTGTGG |
| 3 | EXON43 | SACAS9 | − | TGCAAAGTGCAACGCCTGTGGA |
| 3006 | EXON43 | SACAS9 | − | ATAGTCTACAACAAAGCTCA |
| 3007 | EXON43 | SACAS9 | − | ATAGTCTACAACAAAGCTCAG |
| 4 | EXON43 | SACAS9 | − | ATAGTCTACAACAAAGCTCAGG |
| 3008 | EXON43 | SACAS9 | − | CTGTTTTAAAATTTTTATAT |
| 3009 | EXON43 | SACAS9 | − | CTGTTTTAAAATTTTTATATT |
| 5 | EXON43 | SACAS9 | − | CTGTTTTAAAATTTTTATATTA |
| 3010 | EXON44 | SACAS9 | + | TTAGCATGTTCCCAATTCTC |
| 3011 | EXON44 | SACAS9 | + | TTTAGCATGTTCCCAATTCTC |
| 6 | EXON44 | SACAS9 | + | ATTTAGCATGTTCCCAATTCTC |
| 3012 | EXON44 | SACAS9 | + | TCGCCTGCAGGTAAAAGCAT |
| 3013 | EXON44 | SACAS9 | + | ATCGCCTGCAGGTAAAAGCAT |
| 7 | EXON44 | SACAS9 | + | AATCGCCTGCAGGTAAAAGCAT |
| 3014 | EXON44 | SACAS9 | − | TCTCAGAAAGACACAAATTC |
| 3015 | EXON44 | SACAS9 | − | TCTCAGAAAGACACAAATTCC |
| 8 | EXON44 | SACAS9 | − | TCTCAGAAAGACACAAATTCCT |
| 3016 | EXON45 | SACAS9 | + | AGGCTTCCCAATTTTTCCTG |
| 3017 | EXON45 | SACAS9 | + | CAGGCTTCCCAATTTTTCCTG |
| 9 | EXON45 | SACAS9 | + | TCAGGCTTCCCAATTTTTCCTG |
| 3018 | EXON45 | SACAS9 | + | GAATACTGGCATCTGTTTTT |
| 3019 | EXON45 | SACAS9 | + | AGAATACTGGCATCTGTTTTT |
| 10 | EXON45 | SACAS9 | + | TAGAATACTGGCATCTGTTTTT |
| 3020 | EXON45 | SACAS9 | + | GCATCTGTTTTTGAGGATTG |
| 3021 | EXON45 | SACAS9 | + | GGCATCTGTTTTTGAGGATTG |
| 11 | EXON45 | SACAS9 | + | TGGCATCTGTTTTTGAGGATTG |
| 3022 | EXON45 | SACAS9 | + | GCCGCTGCCCAATGCCATCC |
| 3023 | EXON45 | SACAS9 | + | TGCCGCTGCCCAATGCCATCC |
| 12 | EXON45 | SACAS9 | + | TTGCCGCTGCCCAATGCCATCC |
| 3024 | EXON45 | SACAS9 | − | GAGGTAGGGCGACAGATCTA |
| 3025 | EXON45 | SACAS9 | − | GAGGTAGGGCGACAGATCTAA |
| 13 | EXON45 | SACAS9 | − | GAGGTAGGGCGACAGATCTAAT |
| 3026 | EXON45 | SACAS9 | − | TCTACAGGAAAAATTGGGAA |
| 3027 | EXON45 | SACAS9 | − | TCTACAGGAAAAATTGGGAAG |
| 14 | EXON45 | SACAS9 | − | TCTACAGGAAAAATTGGGAAGC |
| 3028 | EXON45 | SACAS9 | − | GCGGCAAACTGTTGTCAGAA |
| 3029 | EXON45 | SACAS9 | − | GCGGCAAACTGTTGTCAGAAC |
| 15 | EXON45 | SACAS9 | − | GCGGCAAACTGTTGTCAGAACA |
| 3030 | EXON45 | SACAS9 | − | TTTTGGTATCTTACAGGAAC |
| 3031 | EXON45 | SACAS9 | − | TTTTGGTATCTTACAGGAACT |
| 16 | EXON45 | SACAS9 | − | TTTTGGTATCTTACAGGAACTC |
| 3032 | EXON50 | SACAS9 | − | ACTATTGGAGCCTGTAAGTA |
| 3033 | EXON50 | SACAS9 | − | ACTATTGGAGCCTGTAAGTAT |
| 17 | EXON50 | SACAS9 | − | ACTATTGGAGCCTGTAAGTATA |
| 3034 | EXON50 | SACAS9 | − | AGGAAGTTAGAAGATCTGAG |
| 3035 | EXON50 | SACAS9 | − | AGGAAGTTAGAAGATCTGAGC |
| 18 | EXON50 | SACAS9 | − | AGGAAGTTAGAAGATCTGAGCT |
| 3036 | EXON51 | SACAS9 | + | GTAACCACAGGTTGTGTCAC |
| 3037 | EXON51 | SACAS9 | + | AGTAACCACAGGTTGTGTCAC |
| 19 | EXON51 | SACAS9 | + | TAGTAACCACAGGTTGTGTCAC |
| 3038 | EXON51 | SACAS9 | + | TGTGTCACCAGAGTAACAGT |
| 3039 | EXON51 | SACAS9 | + | TTGTGTCACCAGAGTAACAGT |
| 20 | EXON51 | SACAS9 | + | GTTGTGTCACCAGAGTAACAGT |
| 3040 | EXON51 | SACAS9 | + | TGAGTAGGAGCTAAAATATT |
| 3041 | EXON51 | SACAS9 | + | CTGAGTAGGAGCTAAAATATT |
| 21 | EXON51 | SACAS9 | + | TCTGAGTAGGAGCTAAAATATT |
| 3042 | EXON51 | SACAS9 | − | GAGGGTGATGGTGGGTGACC |
| 3043 | EXON51 | SACAS9 | − | GAGGGTGATGGTGGGTGACCT |
| 22 | EXON51 | SACAS9 | − | GAGGGTGATGGTGGGTGACCTT |
| 3044 | EXON51 | SACAS9 | − | TATAAAATCACAGAGGGTGA |
| 3045 | EXON51 | SACAS9 | − | TATAAAATCACAGAGGGTGAT |
| 23 | EXON51 | SACAS9 | − | TATAAAATCACAGAGGGTGATG |
| 3046 | EXON51 | SACAS9 | − | TTGATCAAGTTATAAAATCA |
| 3047 | EXON51 | SACAS9 | − | TTGATCAAGTTATAAAATCAC |
| 24 | EXON51 | SACAS9 | − | TTGATCAAGTTATAAAATCACA |
| 3048 | EXON53 | SACAS9 | + | TTGGTTTCTGTGATTTTCTT |
| 3049 | EXON53 | SACAS9 | + | CTTGGTTTCTGTGATTTTCTT |
| 25 | EXON53 | SACAS9 | + | CCTTGGTTTCTGTGATTTTCTT |
| 3050 | EXON53 | SACAS9 | + | CTTAGCTTCCAGCCATTGTG |
| 3051 | EXON53 | SACAS9 | + | CCTTAGCTTCCAGCCATTGTG |
| 26 | EXON53 | SACAS9 | + | TCCTTAGCTTCCAGCCATTGTG |
| 3052 | EXON53 | SACAS9 | + | TGTACTTCATCCCACTGATT |
| 3053 | EXON53 | SACAS9 | + | TTGTACTTCATCCCACTGATT |
| 27 | EXON53 | SACAS9 | + | CTTGTACTTCATCCCACTGATT |
| 3054 | EXON53 | SACAS9 | + | TGATTCTGAATTCTTTCAAC |
| 3055 | EXON53 | SACAS9 | + | CTGATTCTGAATTCTTTCAAC |
| 28 | EXON53 | SACAS9 | + | ACTGATTCTGAATTCTTTCAAC |
| 3056 | EXON53 | SACAS9 | − | AGCCAAGCTTGAGTCATGGA |
| 3057 | EXON53 | SACAS9 | − | AGCCAAGCTTGAGTCATGGAA |
| 29 | EXON53 | SACAS9 | − | AGCCAAGCTTGAGTCATGGAAG |
| 3058 | EXON53 | SACAS9 | − | TTAGGACAGGCCAGAGCCAA |
| 3059 | EXON53 | SACAS9 | − | TTAGGACAGGCCAGAGCCAAG |
| 30 | EXON53 | SACAS9 | − | TTAGGACAGGCCAGAGCCAAGC |
| 3060 | EXON53 | SACAS9 | − | GCAACAGTTGAATGAAATGT |
| 3061 | EXON53 | SACAS9 | − | GCAACAGTTGAATGAAATGTT |
| 31 | EXON53 | SACAS9 | − | GCAACAGTTGAATGAAATGTTA |
| 3062 | EXON53 | SACAS9 | − | CCTTCAGAACCGGAGGCAAC |
| 3063 | EXON53 | SACAS9 | − | CCTTCAGAACCGGAGGCAACA |
| 32 | EXON53 | SACAS9 | − | CCTTCAGAACCGGAGGCAACAG |
| 3064 | EXON53 | SACAS9 | − | AGTTGAAAGAATTCAGAATC |
| 3065 | EXON53 | SACAS9 | − | AGTTGAAAGAATTCAGAATCA |
| 33 | EXON53 | SACAS9 | − | AGTTGAAAGAATTCAGAATCAG |
| 3066 | EXON53 | SACAS9 | − | TTTTATTCTAGTTGAAAGAA |
| 3067 | EXON53 | SACAS9 | − | TTTTATTCTAGTTGAAAGAAT |
| 34 | EXON53 | SACAS9 | − | TTTTATTCTAGTTGAAAGAATT |
| 3068 | EXON53 | SACAS9 | − | TTTTTCCTTTTATTCTAGTT |
| 3069 | EXON53 | SACAS9 | − | TTTTTCCTTTTATTCTAGTTG |
| 35 | EXON53 | SACAS9 | − | TTTTTCCTTTTATTCTAGTTGA |
| 4000 | EXON43 | SLUCAS9 | + | ATATGTGTTACCTACCCTTG |
| 4001 | EXON43 | SLUCAS9 | + | TATATGTGTTACCTACCCTTG |
| 100 | EXON43 | SLUCAS9 | + | ATATATGTGTTACCTACCCTTG |
| 4002 | EXON43 | SLUCAS9 | + | ATTTTGTTAACTTTTTCCCA |
| 4003 | EXON43 | SLUCAS9 | + | CATTTTGTTAACTTTTTCCCA |
| 101 | EXON43 | SLUCAS9 | + | ACATTTTGTTAACTTTTTCCCA |
| 4004 | EXON43 | SLUCAS9 | + | TTTTCCCATTGGAAATCAAG |
| 4005 | EXON43 | SLUCAS9 | + | TTTTTCCCATTGGAAATCAAG |
| 102 | EXON43 | SLUCAS9 | + | CTTTTTCCCATTGGAAATCAAG |
| 4006 | EXON43 | SLUCAS9 | + | TTTCCCATTGGAAATCAAGC |
| 4007 | EXON43 | SLUCAS9 | + | TTTTCCCATTGGAAATCAAGC |
| 103 | EXON43 | SLUCAS9 | + | TTTTTCCCATTGGAAATCAAGC |
| 4008 | EXON43 | SLUCAS9 | + | CTGTAGCTTCACCCTTTCCA |
| 4009 | EXON43 | SLUCAS9 | + | CCTGTAGCTTCACCCTTTCCA |
| 104 | EXON43 | SLUCAS9 | + | TCCTGTAGCTTCACCCTTTCCA |
| 4010 | EXON43 | SLUCAS9 | − | AATGTACAAGGACCGACAAG |
| 4011 | EXON43 | SLUCAS9 | − | AATGTACAAGGACCGACAAGG |
| 105 | EXON43 | SLUCAS9 | − | AATGTACAAGGACCGACAAGGG |
| 4012 | EXON43 | SLUCAS9 | − | ACAAAATGTACAAGGACCGA |
| 4013 | EXON43 | SLUCAS9 | − | ACAAAATGTACAAGGACCGAC |
| 106 | EXON43 | SLUCAS9 | − | ACAAAATGTACAAGGACCGACA |
| 4014 | EXON43 | SLUCAS9 | − | AACAAAATGTACAAGGACCG |
| 4015 | EXON43 | SLUCAS9 | − | AACAAAATGTACAAGGACCGA |
| 107 | EXON43 | SLUCAS9 | − | AACAAAATGTACAAGGACCGAC |
| 4016 | EXON43 | SLUCAS9 | − | GGAAAAAGTTAACAAAATGT |
| 4017 | EXON43 | SLUCAS9 | − | GGAAAAAGTTAACAAAATGTA |
| 108 | EXON43 | SLUCAS9 | − | GGAAAAAGTTAACAAAATGTAC |
| 4018 | EXON43 | SLUCAS9 | − | TCTCTCCCAGCTTGATTTCC |
| 4019 | EXON43 | SLUCAS9 | − | TCTCTCCCAGCTTGATTTCCA |
| 109 | EXON43 | SLUCAS9 | − | TCTCTCCCAGCTTGATTTCCAA |
| 4020 | EXON43 | SLUCAS9 | − | CTCTCTCCCAGCTTGATTTC |
| 4021 | EXON43 | SLUCAS9 | − | CTCTCTCCCAGCTTGATTTCC |
| 110 | EXON43 | SLUCAS9 | − | CTCTCTCCCAGCTTGATTTCCA |
| 4022 | EXON43 | SLUCAS9 | − | GCCTGTGGAAAGGGTGAAGC |
| 4023 | EXON43 | SLUCAS9 | − | GCCTGTGGAAAGGGTGAAGCT |
| 111 | EXON43 | SLUCAS9 | − | GCCTGTGGAAAGGGTGAAGCTA |
| 4024 | EXON43 | SLUCAS9 | − | GCAAAGTGCAACGCCTGTGG |
| 4025 | EXON43 | SLUCAS9 | − | GCAAAGTGCAACGCCTGTGGA |
| 112 | EXON43 | SLUCAS9 | − | GCAAAGTGCAACGCCTGTGGAA |
| 4026 | EXON43 | SLUCAS9 | − | TGCAAAGTGCAACGCCTGTG |
| 4027 | EXON43 | SLUCAS9 | − | TGCAAAGTGCAACGCCTGTGG |
| 113 | EXON43 | SLUCAS9 | − | TGCAAAGTGCAACGCCTGTGGA |
| 4028 | EXON43 | SLUCAS9 | − | AGCATTGCAAAGTGCAACGC |
| 4029 | EXON43 | SLUCAS9 | − | AGCATTGCAAAGTGCAACGCC |
| 114 | EXON43 | SLUCAS9 | − | AGCATTGCAAAGTGCAACGCCT |
| 4030 | EXON43 | SLUCAS9 | − | ATAGTCTACAACAAAGCTCA |
| 4031 | EXON43 | SLUCAS9 | − | ATAGTCTACAACAAAGCTCAG |
| 115 | EXON43 | SLUCAS9 | − | ATAGTCTACAACAAAGCTCAGG |
| 4032 | EXON43 | SLUCAS9 | − | AAAGATAGTCTACAACAAAG |
| 4033 | EXON43 | SLUCAS9 | − | AAAGATAGTCTACAACAAAGC |
| 116 | EXON43 | SLUCAS9 | − | AAAGATAGTCTACAACAAAGCT |
| 4034 | EXON44 | SLUCAS9 | + | TTTAGCATGTTCCCAATTCT |
| 4035 | EXON44 | SLUCAS9 | + | ATTTAGCATGTTCCCAATTCT |
| 117 | EXON44 | SLUCAS9 | + | TATTTAGCATGTTCCCAATTCT |
| 4036 | EXON44 | SLUCAS9 | + | CAGATCTGTCAAATCGCCTG |
| 4037 | EXON44 | SLUCAS9 | + | ACAGATCTGTCAAATCGCCTG |
| 118 | EXON44 | SLUCAS9 | + | AACAGATCTGTCAAATCGCCTG |
| 4038 | EXON44 | SLUCAS9 | + | TCGCCTGCAGGTAAAAGCAT |
| 4039 | EXON44 | SLUCAS9 | + | ATCGCCTGCAGGTAAAAGCAT |
| 119 | EXON44 | SLUCAS9 | + | AATCGCCTGCAGGTAAAAGCAT |
| 4040 | EXON44 | SLUCAS9 | − | TGCTAAATACAAATGGTATC |
| 4041 | EXON44 | SLUCAS9 | − | TGCTAAATACAAATGGTATCT |
| 120 | EXON44 | SLUCAS9 | − | TGCTAAATACAAATGGTATCTT |
| 4042 | EXON44 | SLUCAS9 | − | ATTGGGAACATGCTAAATAC |
| 4043 | EXON44 | SLUCAS9 | − | ATTGGGAACATGCTAAATACA |
| 121 | EXON44 | SLUCAS9 | − | ATTGGGAACATGCTAAATACAA |
| 4044 | EXON44 | SLUCAS9 | − | AAAGACACAAATTCCTGAGA |
| 4045 | EXON44 | SLUCAS9 | − | AAAGACACAAATTCCTGAGAA |
| 122 | EXON44 | SLUCAS9 | − | AAAGACACAAATTCCTGAGAAT |
| 4046 | EXON44 | SLUCAS9 | − | GAAAGACACAAATTCCTGAG |
| 4047 | EXON44 | SLUCAS9 | − | GAAAGACACAAATTCCTGAGA |
| 123 | EXON44 | SLUCAS9 | − | GAAAGACACAAATTCCTGAGAA |
| 4048 | EXON44 | SLUCAS9 | − | ATGATATAAAGATATTTAAT |
| 4049 | EXON44 | SLUCAS9 | − | ATGATATAAAGATATTTAATC |
| 124 | EXON44 | SLUCAS9 | − | ATGATATAAAGATATTTAATCA |
| 4050 | EXON44 | SLUCAS9 | − | TTGACAGATCTGTTGAGAAA |
| 4051 | EXON44 | SLUCAS9 | − | TTGACAGATCTGTTGAGAAAT |
| 125 | EXON44 | SLUCAS9 | − | TTGACAGATCTGTTGAGAAATG |
| 4052 | EXON44 | SLUCAS9 | − | GATTTGACAGATCTGTTGAG |
| 4053 | EXON44 | SLUCAS9 | − | GATTTGACAGATCTGTTGAGA |
| 126 | EXON44 | SLUCAS9 | − | GATTTGACAGATCTGTTGAGAA |
| 4054 | EXON44 | SLUCAS9 | − | TTGATCCATATGCTTTTACC |
| 4055 | EXON44 | SLUCAS9 | − | TTGATCCATATGCTTTTACCT |
| 127 | EXON44 | SLUCAS9 | − | TTGATCCATATGCTTTTACCTG |
| 4056 | EXON45 | SLUCAS9 | + | ACCTCCTGCCACCGCAGATT |
| 4057 | EXON45 | SLUCAS9 | + | GACCTCCTGCCACCGCAGATT |
| 128 | EXON45 | SLUCAS9 | + | AGACCTCCTGCCACCGCAGATT |
| 4058 | EXON45 | SLUCAS9 | + | CCAATTTTTCCTGTAGAATA |
| 4059 | EXON45 | SLUCAS9 | + | CCCAATTTTTCCTGTAGAATA |
| 129 | EXON45 | SLUCAS9 | + | TCCCAATTTTTCCTGTAGAATA |
| 4060 | EXON45 | SLUCAS9 | + | GAATACTGGCATCTGTTTTT |
| 4061 | EXON45 | SLUCAS9 | + | AGAATACTGGCATCTGTTTTT |
| 130 | EXON45 | SLUCAS9 | + | TAGAATACTGGCATCTGTTTTT |
| 4062 | EXON45 | SLUCAS9 | + | TGCCGCTGCCCAATGCCATC |
| 4063 | EXON45 | SLUCAS9 | + | TTGCCGCTGCCCAATGCCATC |
| 131 | EXON45 | SLUCAS9 | + | TTTGCCGCTGCCCAATGCCATC |
| 4064 | EXON45 | SLUCAS9 | + | AGTTCCTGTAAGATACCAAA |
| 4065 | EXON45 | SLUCAS9 | + | GAGTTCCTGTAAGATACCAAA |
| 132 | EXON45 | SLUCAS9 | + | GGAGTTCCTGTAAGATACCAAA |
| 4066 | EXON45 | SLUCAS9 | − | AGAGGTAGGGCGACAGATCT |
| 4067 | EXON45 | SLUCAS9 | − | AGAGGTAGGGCGACAGATCTA |
| 133 | EXON45 | SLUCAS9 | − | AGAGGTAGGGCGACAGATCTAA |
| 4068 | EXON45 | SLUCAS9 | − | CTGTCAGACAGAAAAAAGAG |
| 4069 | EXON45 | SLUCAS9 | − | CTGTCAGACAGAAAAAAGAGG |
| 134 | EXON45 | SLUCAS9 | − | CTGTCAGACAGAAAAAAGAGGT |
| 4070 | EXON45 | SLUCAS9 | − | GCTGTCAGACAGAAAAAAGA |
| 4071 | EXON45 | SLUCAS9 | − | GCTGTCAGACAGAAAAAAGAG |
| 135 | EXON45 | SLUCAS9 | − | GCTGTCAGACAGAAAAAAGAGG |
| 4072 | EXON45 | SLUCAS9 | − | AACAGCTGTCAGACAGAAAA |
| 4073 | EXON45 | SLUCAS9 | − | AACAGCTGTCAGACAGAAAAA |
| 136 | EXON45 | SLUCAS9 | − | AACAGCTGTCAGACAGAAAAAA |
| 4074 | EXON45 | SLUCAS9 | − | AAGCCTGAATCTGCGGTGGC |
| 4075 | EXON45 | SLUCAS9 | − | AAGCCTGAATCTGCGGTGGCA |
| 137 | EXON45 | SLUCAS9 | − | AAGCCTGAATCTGCGGTGGCAG |
| 4076 | EXON45 | SLUCAS9 | − | GGGAAGCCTGAATCTGCGGT |
| 4077 | EXON45 | SLUCAS9 | − | GGGAAGCCTGAATCTGCGGTG |
| 138 | EXON45 | SLUCAS9 | − | GGGAAGCCTGAATCTGCGGTGG |
| 4078 | EXON45 | SLUCAS9 | − | AATTGGGAAGCCTGAATCTG |
| 4079 | EXON45 | SLUCAS9 | − | AATTGGGAAGCCTGAATCTGC |
| 139 | EXON45 | SLUCAS9 | − | AATTGGGAAGCCTGAATCTGCG |
| 4080 | EXON45 | SLUCAS9 | − | AAAAATTGGGAAGCCTGAAT |
| 4081 | EXON45 | SLUCAS9 | − | AAAAATTGGGAAGCCTGAATC |
| 140 | EXON45 | SLUCAS9 | − | AAAAATTGGGAAGCCTGAATCT |
| 4082 | EXON45 | SLUCAS9 | − | GCCAGTATTCTACAGGAAAA |
| 4083 | EXON45 | SLUCAS9 | | GCCAGTATTCTACAGGAAAAA |
| 141 | EXON45 | SLUCAS9 | − | GCCAGTATTCTACAGGAAAAAT |
| 4084 | EXON45 | SLUCAS9 | − | TGCCAGTATTCTACAGGAAA |
| 4085 | EXON45 | SLUCAS9 | − | TGCCAGTATTCTACAGGAAAA |
| 142 | EXON45 | SLUCAS9 | − | TGCCAGTATTCTACAGGAAAAA |
| 4086 | EXON45 | SLUCAS9 | − | AAAAACAGATGCCAGTATTC |
| 4087 | EXON45 | SLUCAS9 | − | AAAAACAGATGCCAGTATTCT |
| 143 | EXON45 | SLUCAS9 | − | AAAAACAGATGCCAGTATTCTA |
| 4088 | EXON45 | SLUCAS9 | − | TGTCAGAACATTGAATGCAA |
| 4089 | EXON45 | SLUCAS9 | − | TGTCAGAACATTGAATGCAAC |
| 144 | EXON45 | SLUCAS9 | − | TGTCAGAACATTGAATGCAACT |
| 4090 | EXON45 | SLUCAS9 | − | TTGTCAGAACATTGAATGCA |
| 4091 | EXON45 | SLUCAS9 | − | TTGTCAGAACATTGAATGCAA |
| 145 | EXON45 | SLUCAS9 | − | TTGTCAGAACATTGAATGCAAC |
| 4092 | EXON45 | SLUCAS9 | − | GTTGTCAGAACATTGAATGC |
| 4093 | EXON45 | SLUCAS9 | − | GTTGTCAGAACATTGAATGCA |
| 146 | EXON45 | SLUCAS9 | − | GTTGTCAGAACATTGAATGCAA |
| 4094 | EXON45 | SLUCAS9 | − | AACTCCAGGATGGCATTGGG |
| 4095 | EXON45 | SLUCAS9 | − | AACTCCAGGATGGCATTGGGC |
| 147 | EXON45 | SLUCAS9 | − | AACTCCAGGATGGCATTGGGCA |
| 4096 | EXON45 | SLUCAS9 | − | TACAGGAACTCCAGGATGGC |
| 4097 | EXON45 | SLUCAS9 | − | TACAGGAACTCCAGGATGGCA |
| 148 | EXON45 | SLUCAS9 | − | TACAGGAACTCCAGGATGGCAT |
| 4098 | EXON45 | SLUCAS9 | − | TTACAGGAACTCCAGGATGG |
| 4099 | EXON45 | SLUCAS9 | − | TTACAGGAACTCCAGGATGGC |
| 149 | EXON45 | SLUCAS9 | − | TTACAGGAACTCCAGGATGGCA |
| 4100 | EXON45 | SLUCAS9 | − | GGTATCTTACAGGAACTCCA |
| 4101 | EXON45 | SLUCAS9 | − | GGTATCTTACAGGAACTCCAG |
| 150 | EXON45 | SLUCAS9 | − | GGTATCTTACAGGAACTCCAGG |
| 4102 | EXON45 | SLUCAS9 | − | TTTTGGTATCTTACAGGAAC |
| 4103 | EXON45 | SLUCAS9 | − | TTTTGGTATCTTACAGGAACT |
| 151 | EXON45 | SLUCAS9 | − | TTTTGGTATCTTACAGGAACTC |
| 4104 | EXON45 | SLUCAS9 | − | GTTTTGCCTTTTTGGTATCT |
| 4105 | EXON45 | SLUCAS9 | − | GTTTTGCCTTTTTGGTATCTT |
| 152 | EXON45 | SLUCAS9 | − | GTTTTGCCTTTTTGGTATCTTA |
| 4106 | EXON45 | SLUCAS9 | − | TCTTTTCTCAAATAAAAAGA |
| 4107 | EXON45 | SLUCAS9 | − | TCTTTTCTCAAATAAAAAGAC |
| 153 | EXON45 | SLUCAS9 | − | TCTTTTCTCAAATAAAAAGACA |
| 4108 | EXON50 | SLUCAS9 | + | AATGGGATCCAGTATACTTA |
| 4109 | EXON50 | SLUCAS9 | + | GAATGGGATCCAGTATACTTA |
| 154 | EXON50 | SLUCAS9 | + | AGAATGGGATCCAGTATACTTA |
| 4110 | EXON50 | SLUCAS9 | + | TATACTTACAGGCTCCAATA |
| 4111 | EXON50 | SLUCAS9 | + | GTATACTTACAGGCTCCAATA |
| 155 | EXON50 | SLUCAS9 | + | AGTATACTTACAGGCTCCAATA |
| 4112 | EXON50 | SLUCAS9 | + | GGCTCCAATAGTGGTCAGTC |
| 4113 | EXON50 | SLUCAS9 | + | AGGCTCCAATAGTGGTCAGTC |
| 156 | EXON50 | SLUCAS9 | + | CAGGCTCCAATAGTGGTCAGTC |
| 4114 | EXON50 | SLUCAS9 | + | ATAGTGGTCAGTCCAGGAGC |
| 4115 | EXON50 | SLUCAS9 | + | AATAGTGGTCAGTCCAGGAGC |
| 157 | EXON50 | SLUCAS9 | + | CAATAGTGGTCAGTCCAGGAGC |
| 4116 | EXON50 | SLUCAS9 | + | GGTCAGTCCAGGAGCTAGGT |
| 4117 | EXON50 | SLUCAS9 | + | TGGTCAGTCCAGGAGCTAGGT |
| 158 | EXON50 | SLUCAS9 | + | GTGGTCAGTCCAGGAGCTAGGT |
| 4118 | EXON50 | SLUCAS9 | + | GCCCTCAGCTCTTGAAGTAA |
| 4119 | EXON50 | SLUCAS9 | + | TGCCCTCAGCTCTTGAAGTAA |
| 159 | EXON50 | SLUCAS9 | + | TTGCCCTCAGCTCTTGAAGTAA |
| 4120 | EXON50 | SLUCAS9 | − | AGTATACTGGATCCCATTCT |
| 4121 | EXON50 | SLUCAS9 | − | AGTATACTGGATCCCATTCTC |
| 160 | EXON50 | SLUCAS9 | − | AGTATACTGGATCCCATTCTCT |
| 4122 | EXON50 | SLUCAS9 | − | ACTATTGGAGCCTGTAAGTA |
| 4123 | EXON50 | SLUCAS9 | − | ACTATTGGAGCCTGTAAGTAT |
| 161 | EXON50 | SLUCAS9 | − | ACTATTGGAGCCTGTAAGTATA |
| 4124 | EXON50 | SLUCAS9 | − | CTAGCTCCTGGACTGACCAC |
| 4125 | EXON50 | SLUCAS9 | − | CTAGCTCCTGGACTGACCACT |
| 162 | EXON50 | SLUCAS9 | − | CTAGCTCCTGGACTGACCACTA |
| 4126 | EXON50 | SLUCAS9 | − | GCAAAGCAGCCTGACCTAGC |
| 4127 | EXON50 | SLUCAS9 | − | GCAAAGCAGCCTGACCTAGCT |
| 163 | EXON50 | SLUCAS9 | − | GCAAAGCAGCCTGACCTAGCTC |
| 4128 | EXON50 | SLUCAS9 | − | AAACCGTTTACTTCAAGAGC |
| 4129 | EXON50 | SLUCAS9 | − | AAACCGTTTACTTCAAGAGCT |
| 164 | EXON50 | SLUCAS9 | − | AAACCGTTTACTTCAAGAGCTG |
| 4130 | EXON50 | SLUCAS9 | − | TAAACCGTTTACTTCAAGAG |
| 4131 | EXON50 | SLUCAS9 | − | TAAACCGTTTACTTCAAGAGC |
| 165 | EXON50 | SLUCAS9 | − | TAAACCGTTTACTTCAAGAGCT |
| 4132 | EXON50 | SLUCAS9 | − | AGATCTGAGCTCTGAGTGGA |
| 4133 | EXON50 | SLUCAS9 | − | AGATCTGAGCTCTGAGTGGAA |
| 166 | EXON50 | SLUCAS9 | − | AGATCTGAGCTCTGAGTGGAAG |
| 4134 | EXON50 | SLUCAS9 | − | AGAAGATCTGAGCTCTGAGT |
| 4135 | EXON50 | SLUCAS9 | − | AGAAGATCTGAGCTCTGAGTG |
| 167 | EXON50 | SLUCAS9 | − | AGAAGATCTGAGCTCTGAGTGG |
| 4136 | EXON50 | SLUCAS9 | − | AGTTAGAAGATCTGAGCTCT |
| 4137 | EXON50 | SLUCAS9 | − | AGTTAGAAGATCTGAGCTCTG |
| 168 | EXON50 | SLUCAS9 | − | AGTTAGAAGATCTGAGCTCTGA |
| 4138 | EXON50 | SLUCAS9 | − | ATGTGTATGCTTTTCTGTTA |
| 4139 | EXON50 | SLUCAS9 | − | ATGTGTATGCTTTTCTGTTAA |
| 169 | EXON50 | SLUCAS9 | − | ATGTGTATGCTTTTCTGTTAAA |
| 4140 | EXON51 | SLUCAS9 | + | ATCATCTCGTTGATATCCTC |
| 4141 | EXON51 | SLUCAS9 | + | GATCATCTCGTTGATATCCTC |
| 170 | EXON51 | SLUCAS9 | + | TGATCATCTCGTTGATATCCTC |
| 4142 | EXON51 | SLUCAS9 | + | GATCAAGCAGAGAAAGCCAG |
| 4143 | EXON51 | SLUCAS9 | + | TGATCAAGCAGAGAAAGCCAG |
| 171 | EXON51 | SLUCAS9 | + | TTGATCAAGCAGAGAAAGCCAG |
| 4144 | EXON51 | SLUCAS9 | + | TCGGTAAGTTCTGTCCAAGC |
| 4145 | EXON51 | SLUCAS9 | + | GTCGGTAAGTTCTGTCCAAGC |
| 172 | EXON51 | SLUCAS9 | + | AGTCGGTAAGTTCTGTCCAAGC |
| 4146 | EXON51 | SLUCAS9 | + | CCGGTTGAAATCTGCCAGAG |
| 4147 | EXON51 | SLUCAS9 | + | CCCGGTTGAAATCTGCCAGAG |
| 173 | EXON51 | SLUCAS9 | + | GCCCGGTTGAAATCTGCCAGAG |
| 4148 | EXON51 | SLUCAS9 | + | GAGCAGGTACCTCCAACATC |
| 4149 | EXON51 | SLUCAS9 | + | AGAGCAGGTACCTCCAACATC |
| 174 | EXON51 | SLUCAS9 | + | CAGAGCAGGTACCTCCAACATC |
| 4150 | EXON51 | SLUCAS9 | + | TACCTCCAACATCAAGGAAG |
| 4151 | EXON51 | SLUCAS9 | + | GTACCTCCAACATCAAGGAAG |
| 175 | EXON51 | SLUCAS9 | + | GGTACCTCCAACATCAAGGAAG |
| 4152 | EXON51 | SLUCAS9 | + | AGGAAGATGGCATTTCTAGT |
| 4153 | EXON51 | SLUCAS9 | + | AAGGAAGATGGCATTTCTAGT |
| 176 | EXON51 | SLUCAS9 | + | CAAGGAAGATGGCATTTCTAGT |
| 4154 | EXON51 | SLUCAS9 | + | ATGGCATTTCTAGTTTGGAG |
| 4155 | EXON51 | SLUCAS9 | + | GATGGCATTTCTAGTTTGGAG |
| 177 | EXON51 | SLUCAS9 | + | AGATGGCATTTCTAGTTTGGAG |
| 4156 | EXON51 | SLUCAS9 | + | GGCAGTTTCCTTAGTAACCA |
| 4157 | EXON51 | SLUCAS9 | + | TGGCAGTTTCCTTAGTAACCA |
| 178 | EXON51 | SLUCAS9 | + | ATGGCAGTTTCCTTAGTAACCA |
| 4158 | EXON51 | SLUCAS9 | + | CACCAGAGTAACAGTCTGAG |
| 4159 | EXON51 | SLUCAS9 | + | TCACCAGAGTAACAGTCTGAG |
| 179 | EXON51 | SLUCAS9 | + | GTCACCAGAGTAACAGTCTGAG |
| 4160 | EXON51 | SLUCAS9 | + | TGAGTAGGAGCTAAAATATT |
| 4161 | EXON51 | SLUCAS9 | + | CTGAGTAGGAGCTAAAATATT |
| 180 | EXON51 | SLUCAS9 | + | TCTGAGTAGGAGCTAAAATATT |
| 4162 | EXON51 | SLUCAS9 | + | GAGTAGGAGCTAAAATATTT |
| 4163 | EXON51 | SLUCAS9 | + | TGAGTAGGAGCTAAAATATTT |
| 181 | EXON51 | SLUCAS9 | + | CTGAGTAGGAGCTAAAATATTT |
| 4164 | EXON51 | SLUCAS9 | + | ATATTTTGGGTTTTTGCAAA |
| 4165 | EXON51 | SLUCAS9 | + | AATATTTTGGGTTTTTGCAAA |
| 182 | EXON51 | SLUCAS9 | + | AAATATTTTGGGTTTTTGCAAA |
| 4166 | EXON51 | SLUCAS9 | − | GTATGAGAAAAAATGATAAA |
| 4167 | EXON51 | SLUCAS9 | − | GTATGAGAAAAAATGATAAAA |
| 183 | EXON51 | SLUCAS9 | − | GTATGAGAAAAAATGATAAAAG |
| 4168 | EXON51 | SLUCAS9 | − | CAACGAGATGATCATCAAGC |
| 4169 | EXON51 | SLUCAS9 | − | CAACGAGATGATCATCAAGCA |
| 184 | EXON51 | SLUCAS9 | − | CAACGAGATGATCATCAAGCAG |
| 4170 | EXON51 | SLUCAS9 | − | GAGGGTGATGGTGGGTGACC |
| 4171 | EXON51 | SLUCAS9 | − | GAGGGTGATGGTGGGTGACCT |
| 185 | EXON51 | SLUCAS9 | − | GAGGGTGATGGTGGGTGACCTT |
| 4172 | EXON51 | SLUCAS9 | − | ATAAAATCACAGAGGGTGAT |
| 4173 | EXON51 | SLUCAS9 | − | ATAAAATCACAGAGGGTGATG |
| 186 | EXON51 | SLUCAS9 | − | ATAAAATCACAGAGGGTGATGG |
| 4174 | EXON51 | SLUCAS9 | − | TATAAAATCACAGAGGGTGA |
| 4175 | EXON51 | SLUCAS9 | − | TATAAAATCACAGAGGGTGAT |
| 187 | EXON51 | SLUCAS9 | − | TATAAAATCACAGAGGGTGATG |
| 4176 | EXON51 | SLUCAS9 | − | AGTTATAAAATCACAGAGGG |
| 4177 | EXON51 | SLUCAS9 | − | AGTTATAAAATCACAGAGGGT |
| 188 | EXON51 | SLUCAS9 | − | AGTTATAAAATCACAGAGGGTG |
| 4178 | EXON51 | SLUCAS9 | − | TGATCAAGTTATAAAATCAC |
| 4179 | EXON51 | SLUCAS9 | − | TGATCAAGTTATAAAATCACA |
| 189 | EXON51 | SLUCAS9 | − | TGATCAAGTTATAAAATCACAG |
| 4180 | EXON51 | SLUCAS9 | − | TTGATCAAGTTATAAAATCA |
| 4181 | EXON51 | SLUCAS9 | − | TTGATCAAGTTATAAAATCAC |
| 190 | EXON51 | SLUCAS9 | − | TTGATCAAGTTATAAAATCACA |
| 4182 | EXON51 | SLUCAS9 | − | GGGCTTGGACAGAACTTACC |
| 4183 | EXON51 | SLUCAS9 | − | GGGCTTGGACAGAACTTACCG |
| 191 | EXON51 | SLUCAS9 | − | GGGCTTGGACAGAACTTACCGA |
| 4184 | EXON51 | SLUCAS9 | − | CTCTGGCAGATTTCAACCGG |
| 4185 | EXON51 | SLUCAS9 | − | CTCTGGCAGATTTCAACCGGG |
| 192 | EXON51 | SLUCAS9 | − | CTCTGGCAGATTTCAACCGGGC |
| 4186 | EXON51 | SLUCAS9 | − | ACCTGCTCTGGCAGATTTCA |
| 4187 | EXON51 | SLUCAS9 | − | ACCTGCTCTGGCAGATTTCAA |
| 193 | EXON51 | SLUCAS9 | − | ACCTGCTCTGGCAGATTTCAAC |
| 4188 | EXON51 | SLUCAS9 | − | TACCTGCTCTGGCAGATTTC |
| 4189 | EXON51 | SLUCAS9 | − | TACCTGCTCTGGCAGATTTCA |
| 194 | EXON51 | SLUCAS9 | − | TACCTGCTCTGGCAGATTTCAA |
| 4190 | EXON51 | SLUCAS9 | − | CTTGATGTTGGAGGTACCTG |
| 4191 | EXON51 | SLUCAS9 | − | CTTGATGTTGGAGGTACCTGC |
| 195 | EXON51 | SLUCAS9 | − | CTTGATGTTGGAGGTACCTGCT |
| 4192 | EXON51 | SLUCAS9 | − | AATGCCATCTTCCTTGATGT |
| 4193 | EXON51 | SLUCAS9 | − | AATGCCATCTTCCTTGATGTT |
| 196 | EXON51 | SLUCAS9 | − | AATGCCATCTTCCTTGATGTTG |
| 4194 | EXON51 | SLUCAS9 | − | AGAAATGCCATCTTCCTTGA |
| 4195 | EXON51 | SLUCAS9 | − | AGAAATGCCATCTTCCTTGAT |
| 197 | EXON51 | SLUCAS9 | − | AGAAATGCCATCTTCCTTGATG |
| 4196 | EXON51 | SLUCAS9 | − | GGTGACACAACCTGTGGTTA |
| 4197 | EXON51 | SLUCAS9 | − | GGTGACACAACCTGTGGTTAC |
| 198 | EXON51 | SLUCAS9 | − | GGTGACACAACCTGTGGTTACT |
| 4198 | EXON51 | SLUCAS9 | − | TGTTACTCTGGTGACACAAC |
| 4199 | EXON51 | SLUCAS9 | − | TGTTACTCTGGTGACACAACC |
| 199 | EXON51 | SLUCAS9 | − | TGTTACTCTGGTGACACAACCT |
| 4200 | EXON51 | SLUCAS9 | − | AGCTCCTACTCAGACTGTTA |
| 4201 | EXON51 | SLUCAS9 | − | AGCTCCTACTCAGACTGTTAC |
| 200 | EXON51 | SLUCAS9 | − | AGCTCCTACTCAGACTGTTACT |
| 4202 | EXON53 | SLUCAS9 | + | AGGTATCTTTGATACTAACC |
| 4203 | EXON53 | SLUCAS9 | + | AAGGTATCTTTGATACTAACC |
| 201 | EXON53 | SLUCAS9 | + | AAAGGTATCTTTGATACTAACC |
| 4204 | EXON53 | SLUCAS9 | + | TTGGTTTCTGTGATTTTCTT |
| 4205 | EXON53 | SLUCAS9 | + | CTTGGTTTCTGTGATTTTCTT |
| 202 | EXON53 | SLUCAS9 | + | CCTTGGTTTCTGTGATTTTCTT |
| 4206 | EXON53 | SLUCAS9 | + | TTTGGATTGCATCTACTGTA |
| 4207 | EXON53 | SLUCAS9 | + | TTTTGGATTGCATCTACTGTA |
| 203 | EXON53 | SLUCAS9 | + | CTTTTGGATTGCATCTACTGTA |
| 4208 | EXON53 | SLUCAS9 | + | TTGGATTGCATCTACTGTAT |
| 4209 | EXON53 | SLUCAS9 | + | TTTGGATTGCATCTACTGTAT |
| 204 | EXON53 | SLUCAS9 | + | TTTTGGATTGCATCTACTGTAT |
| 4210 | EXON53 | SLUCAS9 | + | CCTCCTTCCATGACTCAAGC |
| 4211 | EXON53 | SLUCAS9 | + | CCCTCCTTCCATGACTCAAGC |
| 205 | EXON53 | SLUCAS9 | + | ACCCTCCTTCCATGACTCAAGC |
| 4212 | EXON53 | SLUCAS9 | + | TCCATGACTCAAGCTTGGCT |
| 4213 | EXON53 | SLUCAS9 | + | TTCCATGACTCAAGCTTGGCT |
| 206 | EXON53 | SLUCAS9 | + | CTTCCATGACTCAAGCTTGGCT |
| 4214 | EXON53 | SLUCAS9 | + | ATTTCATTCAACTGTTGCCT |
| 4215 | EXON53 | SLUCAS9 | + | CATTTCATTCAACTGTTGCCT |
| 207 | EXON53 | SLUCAS9 | + | ACATTTCATTCAACTGTTGCCT |
| 4216 | EXON53 | SLUCAS9 | + | AACTGTTGCCTCCGGTTCTG |
| 4217 | EXON53 | SLUCAS9 | + | CAACTGTTGCCTCCGGTTCTG |
| 208 | EXON53 | SLUCAS9 | + | TCAACTGTTGCCTCCGGTTCTG |
| 4218 | EXON53 | SLUCAS9 | + | AATTCTTTCAACTAGAATAA |
| 4219 | EXON53 | SLUCAS9 | + | GAATTCTTTCAACTAGAATAA |
| 209 | EXON53 | SLUCAS9 | + | TGAATTCTTTCAACTAGAATAA |
| 4220 | EXON53 | SLUCAS9 | − | CCAAAAGAAAATCACAGAAA |
| 4221 | EXON53 | SLUCAS9 | − | CCAAAAGAAAATCACAGAAAC |
| 210 | EXON53 | SLUCAS9 | − | CCAAAAGAAAATCACAGAAACC |
| 4222 | EXON53 | SLUCAS9 | − | GCCAAGCTTGAGTCATGGAA |
| 4223 | EXON53 | SLUCAS9 | − | GCCAAGCTTGAGTCATGGAAG |
| 211 | EXON53 | SLUCAS9 | − | GCCAAGCTTGAGTCATGGAAGG |
| 4224 | EXON53 | SLUCAS9 | − | AGCCAAGCTTGAGTCATGGA |
| 4225 | EXON53 | SLUCAS9 | − | AGCCAAGCTTGAGTCATGGAA |
| 212 | EXON53 | SLUCAS9 | − | AGCCAAGCTTGAGTCATGGAAG |
| 4226 | EXON53 | SLUCAS9 | − | CAGAGCCAAGCTTGAGTCAT |
| 4227 | EXON53 | SLUCAS9 | − | CAGAGCCAAGCTTGAGTCATG |
| 213 | EXON53 | SLUCAS9 | − | CAGAGCCAAGCTTGAGTCATGG |
| 4228 | EXON53 | SLUCAS9 | − | AGGCCAGAGCCAAGCTTGAG |
| 4229 | EXON53 | SLUCAS9 | − | AGGCCAGAGCCAAGCTTGAGT |
| 214 | EXON53 | SLUCAS9 | − | AGGCCAGAGCCAAGCTTGAGTC |
| 4230 | EXON53 | SLUCAS9 | − | AGAAGCTGAGCAGGTCTTAG |
| 4231 | EXON53 | SLUCAS9 | − | AGAAGCTGAGCAGGTCTTAGG |
| 215 | EXON53 | SLUCAS9 | − | AGAAGCTGAGCAGGTCTTAGGA |
| 4232 | EXON53 | SLUCAS9 | − | AAGGAAGAAGCTGAGCAGGT |
| 4233 | EXON53 | SLUCAS9 | − | AAGGAAGAAGCTGAGCAGGTC |
| 216 | EXON53 | SLUCAS9 | − | AAGGAAGAAGCTGAGCAGGTCT |
| 4234 | EXON53 | SLUCAS9 | | GGAAGCTAAGGAAGAAGCTG |
| 4235 | EXON53 | SLUCAS9 | − | GGAAGCTAAGGAAGAAGCTGA |
| 217 | EXON53 | SLUCAS9 | − | GGAAGCTAAGGAAGAAGCTGAG |
| 4236 | EXON53 | SLUCAS9 | − | TTCAACACAATGGCTGGAAG |
| 4237 | EXON53 | SLUCAS9 | − | TTCAACACAATGGCTGGAAGC |
| 218 | EXON53 | SLUCAS9 | − | TTCAACACAATGGCTGGAAGCT |
| 4238 | EXON53 | SLUCAS9 | − | GTTAAAGGATTCAACACAAT |
| 4239 | EXON53 | SLUCAS9 | − | GTTAAAGGATTCAACACAATG |
| 219 | EXON53 | SLUCAS9 | − | GTTAAAGGATTCAACACAATGG |
| 4240 | EXON53 | SLUCAS9 | − | AAATGTTAAAGGATTCAACA |
| 4241 | EXON53 | SLUCAS9 | − | AAATGTTAAAGGATTCAACAC |
| 220 | EXON53 | SLUCAS9 | − | AAATGTTAAAGGATTCAACACA |
| 4242 | EXON53 | SLUCAS9 | − | GCAACAGTTGAATGAAATGT |
| 4243 | EXON53 | SLUCAS9 | − | GCAACAGTTGAATGAAATGTT |
| 221 | EXON53 | SLUCAS9 | − | GCAACAGTTGAATGAAATGTTA |
| 4244 | EXON53 | SLUCAS9 | − | TACAAGAACACCTTCAGAAC |
| 4245 | EXON53 | SLUCAS9 | − | TACAAGAACACCTTCAGAACC |
| 222 | EXON53 | SLUCAS9 | − | TACAAGAACACCTTCAGAACCG |
| 4246 | EXON53 | SLUCAS9 | − | AAGTACAAGAACACCTTCAG |
| 4247 | EXON53 | SLUCAS9 | − | AAGTACAAGAACACCTTCAGA |
| 223 | EXON53 | SLUCAS9 | − | AAGTACAAGAACACCTTCAGAA |
| 4248 | EXON53 | SLUCAS9 | − | AGTTGAAAGAATTCAGAATC |
| 4249 | EXON53 | SLUCAS9 | − | AGTTGAAAGAATTCAGAATCA |
| 224 | EXON53 | SLUCAS9 | − | AGTTGAAAGAATTCAGAATCAG |
| 4250 | EXON53 | SLUCAS9 | − | TAGTTGAAAGAATTCAGAAT |
| 4251 | EXON53 | SLUCAS9 | − | TAGTTGAAAGAATTCAGAATC |
| 225 | EXON53 | SLUCAS9 | − | TAGTTGAAAGAATTCAGAATCA |

TABLE 2
Additional DMD Guide Sequences (human-hg38.p12)

| | | | | Genomic | | | | | |
|---|---|---|---|---|---|---|---|---|---|
| | | | | coordinate | | | | | |
| | | | | chrX_stop | | | | | |
| | | | Genomic | (includes | | SEQ ID | | | |
| | | | coordinate | PAM | | NO Guide | | | Offtargets_ |
| EXON | CAS9 | ID | chrX_start | coordinates) | Strand | sequence | Guide sequence | PAM | grouped |
| 45 | SACAS9KKH | E45SaCas9KKH1 | 31968359 | 31968387 | + | 1000 | TGTTTGCAGACCTC | GCAGAT | 20 |
| | | | | | | | CTGCCACC | | |
| 45 | SACAS9KKH | E45SaCas9KKH2 | 31968373 | 31968401 | + | 1001 | CTGCCACCGCAGAT | CCCAAT | 9 |
| | | | | | | | TCAGGCTT | | |
| 45 | SACAS9KKH | E45SaCas9KKH3 | 31968387 | 31968415 | + | 9 | TCAGGCTTCCCAAT | TAGAAT | 37 |
| | | | | | | | TTTTCCTG | | |
| 45 | SACAS9KKH | E45SaCas9KKH4 | 31968409 | 31968437 | + | 10 | TAGAATACTGGCAT | GAGGAT | 63 |
| | | | | | | | CTGTTTTT | | |
| 45 | SACAS9KKH | E45SaCas9KKH5 | 31968417 | 31968445 | + | 11 | TGGCATCTGTTTTT | CTGAAT | 35 |
| | | | | | | | GAGGATTG | | |
| 45 | SACAS9KKH | E45SaCas9KKH6 | 31968432 | 31968460 | + | 1002 | AGGATTGCTGAATT | CCCAGT | 42 |
| | | | | | | | ATTTCTTC | | |
| 45 | SACAS9KKH | E45SaCas9KKH7 | 31968442 | 31968470 | + | 1003 | AATTATTTCTTCCC | TTCAAT | 68 |
| | | | | | | | CAGTTGCA | | |
| 45 | SACAS9KKH | E45SaCas9KKH8 | 31968456 | 31968484 | + | 1004 | CAGTTGCATTCAAT | AACAGT | 17 |
| | | | | | | | GTTCTGAC | | |
| 45 | SACAS9KKH | E45SaCas9KKH9 | 31968471 | 31968499 | + | 1005 | TTCTGACAACAGTT | CCCAAT | 6 |
| | | | | | | | TGCCGCTG | | |
| 45 | SACAS9KKH | E45SaCas9KKH10 | 31968484 | 31968512 | + | 12 | TTGCCGCTGCCCAA | TGGAGT | 5 |
| | | | | | | | TGCCATCC | | |
| 45 | SACAS9KKH | E45SaCas9KKH11 | 31968495 | 31968523 | + | 1006 | CAATGCCATCCTGG | TAAGAT | 27 |
| | | | | | | | AGTTCCTG | | |
| 45 | SACAS9KKH | E45SaCas9KKH12 | 31968517 | 31968545 | + | 1007 | TAAGATACCAAAAA | AAAAAT | 96 |
| | | | | | | | GGCAAAAC | | |
| 45 | SACAS9KKH | E45SaCas9KKH13 | 31968313 | 31968341 | − | 13 | GAGGTAGGGCGACA | AGGAAT | 10 |
| | | | | | | | GATCTAAT | | |
| 45 | SACAS9KKH | E45SaCas9KKH14 | 31968319 | 31968347 | − | 1008 | AAAAAAGAGGTAGG | TCTAAT | 75 |
| | | | | | | | GCGACAGA | | |
| 45 | SACAS9KKH | E45SaCas9KKH15 | 31968324 | 31968352 | − | 1009 | GACAGAAAAAAGAG | ACAGAT | 51 |
| | | | | | | | GTAGGGCG | | |
| 45 | SACAS9KKH | E45SaCas9KKH16 | 31968336 | 31968364 | − | 1010 | AAACAGCTGTCAGA | AGAGGT | 123 |
| | | | | | | | CAGAAAAA | | |
| 45 | SACAS9KKH | E45SaCas9KKH17 | 31968368 | 31968396 | − | 1011 | GAAGCCTGAATCTG | GGAGGT | 19 |
| | | | | | | | CGGTGGCA | | |
| 45 | SACAS9KKH | E45SaCas9KKH18 | 31968378 | 31968406 | − | 1012 | GAAAAATTGGGAAG | TGCGGT | 29 |
| | | | | | | | CCTGAATC | | |
| 45 | SACAS9KKH | E45SaCas9KKH19 | 31968385 | 31968413 | − | 14 | TCTACAGGAAAAAT | CTGAAT | 64 |
| | | | | | | | TGGGAAGC | | |
| 45 | SACAS9KKH | E45SaCas9KKH20 | 31968399 | 31968427 | − | 1013 | ACAGATGCCAGTAT | AAAAAT | 22 |
| | | | | | | | TCTACAGG | | |
| 45 | SACAS9KKH | E45SaCas9KKH21 | 31968415 | 31968443 | − | 1014 | TCAGCAATCCTCAA | GCCAGT | 50 |
| | | | | | | | AAACAGAT | | |
| 45 | SACAS9KKH | E45SaCas9KKH22 | 31968421 | 31968449 | − | 1015 | AATAATTCAGCAAT | ACAGAT | 99 |
| | | | | | | | CCTCAAAA | | |
| 45 | SACAS9KKH | E45SaCas9KKH23 | 31968435 | 31968463 | − | 1016 | GCAACTGGGGAAGA | AGCAAT | 43 |
| | | | | | | | AATAATTC | | |
| 45 | SACAS9KKH | E45SaCas9KKH24 | 31968443 | 31968471 | − | 1017 | CATTGAATGCAACT | AATAAT | 54 |
| | | | | | | | GGGGAAGA | | |
| 45 | SACAS9KKH | E45SaCas9KKH25 | 31968446 | 31968474 | − | 1018 | GAACATTGAATGCA | AGAAAT | 23 |
| | | | | | | | ACTGGGGA | | |
| 45 | SACAS9KKH | E45SaCas9KKH26 | 31968463 | 31968491 | − | 15 | GCGGCAAACTGTTG | TTGAAT | 5 |
| | | | | | | | TCAGAACA | | |
| 45 | SACAS9KKH | E45SaCas9KKH27 | 31968502 | 31968530 | − | 16 | TTTTGGTATCTTAC | CAGGAT | 52 |
| | | | | | | | AGGAACTC | | |
| 45 | SLUCAS9KH | E45SLCas9KH1 | 31968332 | 31968358 | + | 2000 | CCCTACCTCTTTTT | ACAG | 23 |
| | | | | | | | TCTGTCTG | | |
| 45 | SLUCAS9KH | E45SLCas9KH2 | 31968342 | 31968368 | + | 2001 | TTTTTCTGTCTGAC | GCAG | 22 |
| | | | | | | | AGCTGTTT | | |
| 45 | SLUCAS9KH | E45SLCas9KH3 | 31968359 | 31968385 | + | 2002 | TGTTTGCAGACCTC | GCAG | 9 |
| | | | | | | | CTGCCACC | | |
| 45 | SLUCAS9KH | E45SLCas9KH4 | 31968365 | 31968391 | + | 2003 | CAGACCTCCTGCCA | TCAG | 9 |
| | | | | | | | CCGCAGAT | | |
| 45 | SLUCAS9KH | E45SLCas9KH5 | 31968366 | 31968392 | + | 128 | AGACCTCCTGCCAC | CAGG | 10 |
| | | | | | | | CGCAGATT | | |
| 45 | SLUCAS9KH | E45SLCas9KH6 | 31968386 | 31968412 | + | 2004 | TTCAGGCTTCCCAA | GTAG | 11 |
| | | | | | | | TTTTTCCT | | |
| 45 | SLUCAS9KH | E45SLCas9KH7 | 31968394 | 31968420 | + | 129 | TCCCAATTTTTCCT | CTGG | 19 |
| | | | | | | | GTAGAATA | | |
| 45 | SLUCAS9KH | E45SLCas9KH8 | 31968408 | 31968434 | + | 2005 | GTAGAATACTGGCA | TGAG | 16 |
| | | | | | | | TCTGTTTT | | |
| 45 | SLUCAS9KH | E45SLCas9KH9 | 31968409 | 31968435 | + | 130 | TAGAATACTGGCAT | GAGG | 19 |
| | | | | | | | CTGTTTTT | | |
| 45 | SLUCAS9KH | E45SLCas9KH10 | 31968433 | 31968459 | + | 2006 | GGATTGCTGAATTA | CCAG | 18 |
| | | | | | | | TTTCTTCC | | |
| 45 | SLUCAS9KH | E45SLCas9KH11 | 31968457 | 31968483 | + | 2007 | AGTTGCATTCAATG | ACAG | 10 |
| | | | | | | | TTCTGACA | | |
| 45 | SLUCAS9KH | E45SLCas9KH12 | 31968483 | 31968509 | + | 131 | TTTGCCGCTGCCCA | CTGG | 2 |
| | | | | | | | ATGCCATC | | |
| 45 | SLUCAS9KH | E45SLCas9KH13 | 31968485 | 31968511 | + | 2008 | TGCCGCTGCCCAAT | GGAG | 7 |
| | | | | | | | GCCATCCT | | |
| 45 | SLUCAS9KH | E45SLCas9KH14 | 31968495 | 31968521 | + | 2009 | CAATGCCATCCTGG | TAAG | 14 |
| | | | | | | | AGTTCCTG | | |
| 45 | SLUCAS9KH | E45SLCas9KH15 | 31968506 | 31968532 | + | 2010 | TGGAGTTCCTGTAA | AAAG | 10 |
| | | | | | | | GATACCAA | | |
| 45 | SLUCAS9KH | E45SLCas9KH16 | 31968507 | 31968533 | + | 132 | GGAGTTCCTGTAAG | AAGG | 9 |
| | | | | | | | ATACCAAA | | |
| 45 | SLUCAS9KH | E45SLCas9KH17 | 31968316 | 31968342 | − | 133 | AGAGGTAGGGCGAC | TAGG | 6 |
| | | | | | | | AGATCTAA | | |
| 45 | SLUCAS9KH | E45SLCas9KH18 | 31968317 | 31968343 | − | 2011 | AAGAGGTAGGGCGA | ATAG | 5 |
| | | | | | | | CAGATCTA | | |
| 45 | SLUCAS9KH | E45SLCas9KH19 | 31968326 | 31968352 | − | 2012 | GACAGAAAAAAGAG | ACAG | 31 |
| | | | | | | | GTAGGGCG | | |
| 45 | SLUCAS9KH | E45SLCas9KH20 | 31968332 | 31968358 | − | 134 | CTGTCAGACAGAAA | AGGG | 28 |
| | | | | | | | AAAGAGGT | | |
| 45 | SLUCAS9KH | E45SLCas9KH21 | 31968333 | 31968359 | − | 135 | GCTGTCAGACAGAA | TAGG | 43 |
| | | | | | | | AAAAGAGG | | |
| 45 | SLUCAS9KH | E45SLCas9KH22 | 31968334 | 31968360 | − | 2013 | AGCTGTCAGACAGA | GTAG | 67 |
| | | | | | | | AAAAAGAG | | |
| 45 | SLUCAS9KH | E45SLCas9KH23 | 31968337 | 31968363 | − | 136 | AACAGCTGTCAGAC | GAGG | 55 |
| | | | | | | | AGAAAAAA | | |
| 45 | SLUCAS9KH | E45SLCas9KH24 | 31968338 | 31968364 | − | 2014 | AAACAGCTGTCAGA | AGAG | 37 |
| | | | | | | | CAGAAAAA | | |
| 45 | SLUCAS9KH | E45SLCas9KH25 | 31968340 | 31968366 | − | 2015 | GCAAACAGCTGTCA | AAAG | 27 |
| | | | | | | | GACAGAAA | | |
| 45 | SLUCAS9KH | E45SLCas9KH26 | 31968347 | 31968373 | − | 2016 | GAGGTCTGCAAACA | ACAG | 23 |
| | | | | | | | GCTGTCAG | | |
| 45 | SLUCAS9KH | E45SLCas9KH27 | 31968351 | 31968377 | − | 2017 | GCAGGAGGTCTGCA | TCAG | 16 |
| | | | | | | | AACAGCTG | | |
| 45 | SLUCAS9KH | E45SLCas9KH28 | 31968358 | 31968384 | − | 2018 | TGCGGTGGCAGGAG | ACAG | 14 |
| | | | | | | | GTCTGCAA | | |
| 45 | SLUCAS9KH | E45SLCas9KH29 | 31968369 | 31968395 | − | 137 | AAGCCTGAATCTGC | GAGG | 11 |
| | | | | | | | GGTGGCAG | | |
| 45 | SLUCAS9KH | E45SLCas9KH30 | 31968370 | 31968396 | − | 2019 | GAAGCCTGAATCTG | GGAG | 15 |
| | | | | | | | CGGTGGCA | | |
| 45 | SLUCAS9KH | E45SLCas9KH31 | 31968372 | 31968398 | − | 138 | GGGAAGCCTGAATC | CAGG | 8 |
| | | | | | | | TGCGGTGG | | |
| 45 | SLUCAS9KH | E45SLCas9KH32 | 31968373 | 31968399 | − | 2020 | TGGGAAGCCTGAAT | GCAG | 6 |
| | | | | | | | CTGCGGTG | | |
| 45 | SLUCAS9KH | E45SLCas9KH33 | 31968376 | 31968402 | − | 139 | AATTGGGAAGCCTG | GTGG | 7 |
| | | | | | | | AATCTGCG | | |
| 45 | SLUCAS9KH | E45SLCas9KH34 | 31968379 | 31968405 | − | 140 | AAAAATTGGGAAGC | GCGG | 26 |
| | | | | | | | CTGAATCT | | |
| 45 | SLUCAS9KH | E45SLCas9KH35 | 31968392 | 31968418 | − | 2021 | AGTATTCTACAGGA | GAAG | 28 |
| | | | | | | | AAAATTGG | | |
| 45 | SLUCAS9KH | E45SLCas9KH36 | 31968395 | 31968421 | − | 141 | GCCAGTATTCTACA | TGGG | 16 |
| | | | | | | | GGAAAAAT | | |
| 45 | SLUCAS9KH | E45SLCas9KH37 | 31968396 | 31968422 | − | 142 | TGCCAGTATTCTAC | TTGG | 17 |
| | | | | | | | AGGAAAAA | | |
| 45 | SLUCAS9KH | E45SLCas9KH38 | 31968405 | 31968431 | − | 143 | AAAAACAGATGCCA | CAGG | 21 |
| | | | | | | | GTATTCTA | | |
| 45 | SLUCAS9KH | E45SLCas9KH39 | 31968406 | 31968432 | − | 2022 | CAAAAACAGATGCC | ACAG | 28 |
| | | | | | | | AGTATTCT | | |
| 45 | SLUCAS9KH | E45SLCas9KH40 | 31968416 | 31968442 | − | 2023 | CAGCAATCCTCAAA | CCAG | 23 |
| | | | | | | | AACAGATG | | |
| 45 | SLUCAS9KH | E45SLCas9KH41 | 31968423 | 31968449 | − | 2024 | AATAATTCAGCAAT | ACAG | 31 |
| | | | | | | | CCTCAAAA | | |
| 45 | SLUCAS9KH | E45SLCas9KH42 | 31968439 | 31968465 | − | 2025 | ATGCAACTGGGGAA | TCAG | 33 |
| | | | | | | | GAAATAAT | | |
| 45 | SLUCAS9KH | E45SLCas9KH43 | 31968450 | 31968476 | − | 2026 | CAGAACATTGAATG | GAAG | 11 |
| | | | | | | | CAACTGGG | | |
| 45 | SLUCAS9KH | E45SLCas9KH44 | 31968453 | 31968479 | − | 144 | TGTCAGAACATTGA | GGGG | 12 |
| | | | | | | | ATGCAACT | | |
| 45 | SLUCAS9KH | E45SLCas9KH45 | 31968454 | 31968480 | − | 145 | TTGTCAGAACATTG | TGGG | 30 |
| | | | | | | | AATGCAAC | | |
| 45 | SLUCAS9KH | E45SLCas9KH46 | 31968455 | 31968481 | − | 146 | GTTGTCAGAACATT | CTGG | 12 |
| | | | | | | | GAATGCAA | | |
| 45 | SLUCAS9KH | E45SLCas9KH47 | 31968473 | 31968499 | − | 2027 | ATTGGGCAGCGGCA | TCAG | 3 |
| | | | | | | | AACTGTTG | | |
| 45 | SLUCAS9KH | E45SLCas9KH48 | 31968487 | 31968513 | − | 147 | AACTCCAGGATGGC | GCGG | 9 |
| | | | | | | | ATTGGGCA | | |
| 45 | SLUCAS9KH | E45SLCas9KH49 | 31968490 | 31968516 | − | 2028 | AGGAACTCCAGGAT | GCAG | 16 |
| | | | | | | | GGCATTGG | | |
| 45 | SLUCAS9KH | E45SLCas9KH50 | 31968493 | 31968519 | − | 148 | TACAGGAACTCCAG | TGGG | 10 |
| | | | | | | | GATGGCAT | | |
| 45 | SLUCAS9KH | E45SLCas9KH51 | 31968494 | 31968520 | − | 149 | TTACAGGAACTCCA | TTGG | 13 |
| | | | | | | | GGATGGCA | | |
| 45 | SLUCAS9KH | E45SLCas9KH52 | 31968500 | 31968526 | − | 150 | GGTATCTTACAGGA | ATGG | 7 |
| | | | | | | | ACTCCAGG | | |
| 45 | SLUCAS9KH | E45SLCas9KH53 | 31968504 | 31968530 | − | 151 | TTTTGGTATCTTAC | CAGG | 35 |
| | | | | | | | AGGAACTC | | |
| 45 | SLUCAS9KH | E45SLCas9KH54 | 31968505 | 31968531 | − | 2029 | TTTTTGGTATCTTA | CCAG | 21 |
| | | | | | | | CAGGAACT | | |
| 45 | SLUCAS9KH | E45SLCas9KH55 | 31968513 | 31968539 | − | 152 | GTTTTGCCTTTTTG | CAGG | 32 |
| | | | | | | | GTATCTTA | | |
| 45 | SLUCAS9KH | E45SLCas9KH56 | 31968514 | 31968540 | − | 2030 | TGTTTTGCCTTTTT | ACAG | 58 |
| | | | | | | | GGTATCTT | | |
| 51 | SACAS9KKH | E51SaCas9KKH1 | 31773943 | 31773971 | + | 1019 | TCATTTTTTCTCAT | CTTGAT | 91 |
| | | | | | | | ACCTTCTG | | |
| 51 | SACAS9KKH | E51SaCas9KKH2 | 31773946 | 31773974 | + | 1020 | TTTTTTCTCATACC | GATGAT | 132 |
| | | | | | | | TTCTGCTT | | |
| 51 | SACAS9KKH | E51SaCas9KKH3 | 31773958 | 31773986 | + | 1021 | CCTTCTGCTTGATG | GTTGAT | 23 |
| | | | | | | | ATCATCTC | | |
| 51 | SACAS9KKH | E51SaCas9KKH4 | 31773969 | 31773997 | + | 1022 | ATGATCATCTCGTT | CAAGGT | 15 |
| | | | | | | | GATATCCT | | |
| 51 | SACAS9KKH | E51SaCas9KKH5 | 31773992 | 31774020 | + | 1023 | AAGGTCACCCACCA | TGTGAT | 33 |
| | | | | | | | TCACCCTC | | |
| 51 | SACAS9KKH | E51SaCas9KKH6 | 31774005 | 31774033 | + | 1024 | ATCACCCTCTGTGA | CTTGAT | 45 |
| | | | | | | | TTTTATAA | | |
| 51 | SACAS9KKH | E51SaCas9KKH7 | 31774023 | 31774051 | + | 1025 | ATAACTTGATCAAG | GCCAGT | 101 |
| | | | | | | | CAGAGAAA | | |
| 51 | SACAS9KKH | E51SaCas9KKH8 | 31774027 | 31774055 | + | 1026 | CTTGATCAAGCAGA | GTCGGT | 55 |
| | | | | | | | GAAAGCCA | | |
| 51 | SACAS9KKH | E51SaCas9KKH9 | 31774031 | 31774059 | + | 1027 | ATCAAGCAGAGAAA | GTAAGT | 16 |
| | | | | | | | GCCAGTCG | | |
| 51 | SACAS9KKH | E51SaCas9KKH10 | 31774047 | 31774075 | + | 1028 | CAGTCGGTAAGTTC | CCCGGT | 7 |
| | | | | | | | TGTCCAAG | | |
| 51 | SACAS9KKH | E51SaCas9KKH11 | 31774053 | 31774081 | + | 1029 | GTAAGTTCTGTCCA | TGAAAT | 4 |
| | | | | | | | AGCCCGGT | | |
| 51 | SACAS9KKH | E51SaCas9KKH12 | 31774067 | 31774095 | + | 1030 | AGCCCGGTTGAAAT | GCAGGT | 7 |
| | | | | | | | CTGCCAGA | | |
| 51 | SACAS9KKH | E51SaCas9KKH13 | 31774088 | 31774116 | + | 1031 | AGCAGGTACCTCCA | GAAGAT | 23 |
| | | | | | | | ACATCAAG | | |
| 51 | SACAS9KKH | E51SaCas9KKH14 | 31774100 | 31774128 | + | 1032 | CAACATCAAGGAAG | TCTAGT | 54 |
| | | | | | | | ATGGCATT | | |
| 51 | SACAS9KKH | E51SaCas9KKH15 | 31774108 | 31774136 | + | 1033 | AGGAAGATGGCATT | GGAGAT | 62 |
| | | | | | | | TCTAGTTT | | |
| 51 | SACAS9KKH | E51SaCas9KKH16 | 31774114 | 31774142 | + | 1034 | ATGGCATTTCTAGT | GGCAGT | 56 |
| | | | | | | | TTGGAGAT | | |
| 51 | SACAS9KKH | E51SaCas9KKH17 | 31774123 | 31774151 | + | 1035 | CTAGTTTGGAGATG | CTTAGT | 32 |
| | | | | | | | GCAGTTTC | | |
| 51 | SACAS9KKH | E51SaCas9KKH18 | 31774133 | 31774161 | + | 1036 | GATGGCAGTTTCCT | ACAGGT | 24 |
| | | | | | | | TAGTAACC | | |
| 51 | SACAS9KKH | E51SaCas9KKH19 | 31774147 | 31774175 | + | 19 | TAGTAACCACAGGT | CAGAGT | 18 |
| | | | | | | | TGTGTCAC | | |
| 51 | SACAS9KKH | E51SaCas9KKH20 | 31774153 | 31774181 | + | 1037 | CCACAGGTTGTGTC | AACAGT | 20 |
| | | | | | | | ACCAGAGT | | |
| 51 | SACAS9KKH | E51SaCas9KKH21 | 31774159 | 31774187 | + | 20 | GTTGTGTCACCAGA | CTGAGT | 21 |
| | | | | | | | GTAACAGT | | |
| 51 | SACAS9KKH | E51SaCas9KKH22 | 31774171 | 31774199 | + | 1038 | GAGTAACAGTCTGA | TAAAAT | 18 |
| | | | | | | | GTAGGAGC | | |
| 51 | SACAS9KKH | E51SaCas9KKH23 | 31774180 | 31774208 | + | 21 | TCTGAGTAGGAGCT | TTGGGT | 38 |
| | | | | | | | AAAATATT | | |
| 51 | SACAS9KKH | E51SaCas9KKH24 | 31773936 | 31773964 | − | 1039 | AGAAGGTATGAGAA | AAAAGT | 236 |
| | | | | | | | AAAATGAT | | |
| 51 | SACAS9KKH | E51SaCas9KKH25 | 31773942 | 31773970 | − | 1040 | TCAAGCAGAAGGTA | AATGAT | 99 |
| | | | | | | | TGAGAAAA | | |
| 51 | SACAS9KKH | E51SaCas9KKH26 | 31773945 | 31773973 | − | 1041 | TCATCAAGCAGAAG | AAAAAT | 43 |
| | | | | | | | GTATGAGA | | |
| 51 | SACAS9KKH | E51SaCas9KKH27 | 31773957 | 31773985 | − | 1042 | TCAACGAGATGATC | GAAGGT | 10 |
| | | | | | | | ATCAAGCA | | |
| 51 | SACAS9KKH | E51SaCas9KKH28 | 31773972 | 31774000 | − | 1043 | GTGACCTTGAGGAT | GATGAT | 7 |
| | | | | | | | ATCAACGA | | |
| 51 | SACAS9KKH | E51SaCas9KKH29 | 31773975 | 31774003 | − | 1044 | TGGGTGACCTTGAG | CGAGAT | 68 |
| | | | | | | | GATATCAA | | |
| 51 | SACAS9KKH | E51SaCas9KKH30 | 31773986 | 31774014 | − | 22 | GAGGGTGATGGTGG | GAGGAT | 46 |
| | | | | | | | GTGACCTT | | |
| 51 | SACAS9KKH | E51SaCas9KKH31 | 31773998 | 31774026 | − | 23 | TATAAAATCACAGA | GTGGGT | 42 |
| | | | | | | | GGGTGATG | | |
| 51 | SACAS9KKH | E51SaCas9KKH32 | 31774002 | 31774030 | − | 1045 | AAGTTATAAAATCA | GATGGT | 77 |
| | | | | | | | CAGAGGGT | | |
| 51 | SACAS9KKH | E51SaCas9KKH33 | 31774005 | 31774033 | − | 1046 | ATCAAGTTATAAAA | GGTGAT | 90 |
| | | | | | | | TCACAGAG | | |
| 51 | SACAS9KKH | E51SaCas9KKH34 | 31774008 | 31774036 | − | 24 | TTGATCAAGTTATA | GAGGGT | 86 |
| | | | | | | | AAATCACA | | |
| 51 | SACAS9KKH | E51SaCas9KKH35 | 31774018 | 31774046 | − | 1047 | CTTTCTCTGCTTGA | TAAAAT | 45 |
| | | | | | | | TCAAGTTA | | |
| 51 | SACAS9KKH | E51SaCas9KKH36 | 31774026 | 31774054 | − | 1048 | CCGACTGGCTTTCT | TCAAGT | 18 |
| | | | | | | | CTGCTTGA | | |
| 51 | SACAS9KKH | E51SaCas9KKH37 | 31774031 | 31774059 | − | 1049 | ACTTACCGACTGGC | CTTGAT | 11 |
| | | | | | | | TTTCTCTG | | |
| 51 | SACAS9KKH | E51SaCas9KKH38 | 31774079 | 31774107 | − | 1050 | GATGTTGGAGGTAC | GCAGAT | 22 |
| | | | | | | | CTGCTCTG | | |
| 51 | SACAS9KKH | E51SaCas9KKH39 | 31774095 | 31774123 | − | 1051 | AAATGCCATCTTCC | GGAGGT | 57 |
| | | | | | | | TTGATGTT | | |
| 51 | SACAS9KKH | E51SaCas9KKH40 | 31774104 | 31774132 | − | 1052 | CCAAACTAGAAATG | CTTGAT | 46 |
| | | | | | | | CCATCTTC | | |
| 51 | SACAS9KKH | E51SaCas9KKH41 | 31774119 | 31774147 | − | 1053 | AGGAAACTGCCATC | AGAAAT | 63 |
| | | | | | | | TCCAAACT | | |
| 51 | SACAS9KKH | E51SaCas9KKH42 | 31774152 | 31774180 | − | 1054 | CTGTTACTCTGGTG | TGTGGT | 27 |
| | | | | | | | ACACAACC | | |
| 51 | SACAS9KKH | E51SaCas9KKH43 | 31774167 | 31774195 | − | 1055 | TAGCTCCTACTCAG | TCTGGT | 13 |
| | | | | | | | ACTGTTAC | | |
| 51 | SLUCAS9KH | E51SLCas9KH1 | 31773969 | 31773995 | + | 2031 | ATGATCATCTCGTT | CAAG | 6 |
| | | | | | | | GATATCCT | | |
| 51 | SLUCAS9KH | E51SLCas9KH2 | 31773970 | 31773996 | + | 170 | TGATCATCTCGTTG | AAGG | 6 |
| | | | | | | | ATATCCTC | | |
| 51 | SLUCAS9KH | E51SLCas9KH3 | 31774011 | 31774037 | + | 2032 | CTCTGTGATTTTAT | CAAG | 18 |
| | | | | | | | AACTTGAT | | |
| 51 | SLUCAS9KH | E51SLCas9KH4 | 31774014 | 31774040 | + | 2033 | TGTGATTTTATAAC | GCAG | 29 |
| | | | | | | | TTGATCAA | | |
| 51 | SLUCAS9KH | E51SLCas9KH5 | 31774016 | 31774042 | + | 2034 | TGATTTTATAACTT | AGAG | 15 |
| | | | | | | | GATCAAGC | | |
| 51 | SLUCAS9KH | E51SLCas9KH6 | 31774020 | 31774046 | + | 2035 | TTTATAACTTGATC | AAAG | 7 |
| | | | | | | | AAGCAGAG | | |
| 51 | SLUCAS9KH | E51SLCas9KH7 | 31774024 | 31774050 | + | 2036 | TAACTTGATCAAGC | CCAG | 24 |
| | | | | | | | AGAGAAAG | | |
| 51 | SLUCAS9KH | E51SLCas9KH8 | 31774028 | 31774054 | + | 171 | TTGATCAAGCAGAG | TCGG | 23 |
| | | | | | | | AAAGCCAG | | |
| 51 | SLUCAS9KH | E51SLCas9KH9 | 31774032 | 31774058 | + | 2037 | TCAAGCAGAGAAAG | TAAG | 7 |
| | | | | | | | CCAGTCGG | | |
| 51 | SLUCAS9KH | E51SLCas9KH10 | 31774043 | 31774069 | + | 2038 | AAGCCAGTCGGTAA | CAAG | 7 |
| | | | | | | | GTTCTGTC | | |
| 51 | SLUCAS9KH | E51SLCas9KH11 | 31774048 | 31774074 | + | 172 | AGTCGGTAAGTTCT | CCGG | 2 |
| | | | | | | | GTCCAAGC | | |
| 51 | SLUCAS9KH | E51SLCas9KH12 | 31774062 | 31774088 | + | 2039 | GTCCAAGCCCGGTT | CCAG | 1 |
| | | | | | | | GAAATCTG | | |
| 51 | SLUCAS9KH | E51SLCas9KH13 | 31774064 | 31774090 | + | 2040 | CCAAGCCCGGTTGA | AGAG | 2 |
| | | | | | | | AATCTGCC | | |
| 51 | SLUCAS9KH | E51SLCas9KH14 | 31774067 | 31774093 | + | 2041 | AGCCCGGTTGAAAT | GCAG | 4 |
| | | | | | | | CTGCCAGA | | |
| 51 | SLUCAS9KH | E51SLCas9KH15 | 31774068 | 31774094 | + | 173 | GCCCGGTTGAAATC | CAGG | 5 |
| | | | | | | | TGCCAGAG | | |
| 51 | SLUCAS9KH | E51SLCas9KH16 | 31774084 | 31774110 | + | 2042 | CCAGAGCAGGTACC | CAAG | 14 |
| | | | | | | | TCCAACAT | | |
| 51 | SLUCAS9KH | E51SLCas9KH17 | 31774085 | 31774111 | + | 174 | CAGAGCAGGTACCT | AAGG | 13 |
| | | | | | | | CCAACATC | | |
| 51 | SLUCAS9KH | E51SLCas9KH18 | 31774088 | 31774114 | + | 2043 | AGCAGGTACCTCCA | GAAG | 12 |
| | | | | | | | ACATCAAG | | |
| 51 | SLUCAS9KH | E51SLCas9KH19 | 31774092 | 31774118 | + | 175 | GGTACCTCCAACAT | ATGG | 6 |
| | | | | | | | CAAGGAAG | | |
| 51 | SLUCAS9KH | E51SLCas9KH20 | 31774101 | 31774127 | + | 2044 | AACATCAAGGAAGA | CTAG | 32 |
| | | | | | | | TGGCATTT | | |
| 51 | SLUCAS9KH | E51SLCas9KH21 | 31774106 | 31774132 | + | 176 | CAAGGAAGATGGCA | TTGG | 21 |
| | | | | | | | TTTCTAGT | | |
| 51 | SLUCAS9KH | E51SLCas9KH22 | 31774108 | 31774134 | + | 2045 | AGGAAGATGGCATT | GGAG | 50 |
| | | | | | | | TCTAGTTT | | |
| 51 | SLUCAS9KH | E51SLCas9KH23 | 31774112 | 31774138 | + | 177 | AGATGGCATTTCTA | ATGG | 10 |
| | | | | | | | GTTTGGAG | | |
| 51 | SLUCAS9KH | E51SLCas9KH24 | 31774115 | 31774141 | + | 2046 | TGGCATTTCTAGTT | GCAG | 22 |
| | | | | | | | TGGAGATG | | |
| 51 | SLUCAS9KH | E51SLCas9KH25 | 31774124 | 31774150 | + | 2047 | TAGTTTGGAGATGG | TTAG | 20 |
| | | | | | | | CAGTTTCC | | |
| 51 | SLUCAS9KH | E51SLCas9KH26 | 31774133 | 31774159 | + | 2048 | GATGGCAGTTTCCT | ACAG | 11 |
| | | | | | | | TAGTAACC | | |
| 51 | SLUCAS9KH | E51SLCas9KH27 | 31774134 | 31774160 | + | 178 | ATGGCAGTTTCCTT | CAGG | 14 |
| | | | | | | | AGTAACCA | | |
| 51 | SLUCAS9KH | E51SLCas9KH28 | 31774146 | 31774172 | + | 2049 | TTAGTAACCACAGG | CCAG | 7 |
| | | | | | | | TTGTGTCA | | |
| 51 | SLUCAS9KH | E51SLCas9KH29 | 31774148 | 31774174 | + | 2050 | AGTAACCACAGGTT | AGAG | 7 |
| | | | | | | | GTGTCACC | | |
| 51 | SLUCAS9KH | E51SLCas9KH30 | 31774154 | 31774180 | + | 2051 | CACAGGTTGTGTCA | ACAG | 14 |
| | | | | | | | CCAGAGTA | | |
| 51 | SLUCAS9KH | E51SLCas9KH31 | 31774160 | 31774186 | + | 2052 | TTGTGTCACCAGAG | TGAG | 9 |
| | | | | | | | TAACAGTC | | |
| 51 | SLUCAS9KH | E51SLCas9KH32 | 31774163 | 31774189 | + | 2053 | TGTCACCAGAGTAA | GTAG | 9 |
| | | | | | | | CAGTCTGA | | |
| 51 | SLUCAS9KH | E51SLCas9KH33 | 31774164 | 31774190 | + | 179 | GTCACCAGAGTAAC | TAGG | 14 |
| | | | | | | | AGTCTGAG | | |
| 51 | SLUCAS9KH | E51SLCas9KH34 | 31774166 | 31774192 | + | 2054 | CACCAGAGTAACAG | GGAG | 11 |
| | | | | | | | TCTGAGTA | | |
| 51 | SLUCAS9KH | E51SLCas9KH35 | 31774180 | 31774206 | + | 180 | TCTGAGTAGGAGCT | TTGG | 11 |
| | | | | | | | AAAATATT | | |
| 51 | SLUCAS9KH | E51SLCas9KH36 | 31774181 | 31774207 | + | 181 | CTGAGTAGGAGCTA | TGGG | 21 |
| | | | | | | | AAATATTT | | |
| 51 | SLUCAS9KH | E51SLCas9KH37 | 31774194 | 31774220 | + | 2055 | AAAATATTTTGGGT | AAAG | 56 |
| | | | | | | | TTTTGCAA | | |
| 51 | SLUCAS9KH | E51SLCas9KH38 | 31774195 | 31774221 | + | 182 | AAATATTTTGGGTT | AAGG | 61 |
| | | | | | | | TTTGCAAA | | |
| 51 | SLUCAS9KH | E51SLCas9KH39 | 31773927 | 31773953 | − | 2056 | GAAAAAATGATAAA | GAAG | 77 |
| | | | | | | | AGTTGGCA | | |
| 51 | SLUCAS9KH | E51SLCas9KH40 | 31773930 | 31773956 | − | 2057 | TGAGAAAAAATGAT | GCAG | 124 |
| | | | | | | | AAAAGTTG | | |
| 51 | SLUCAS9KH | E51SLCas9KH41 | 31773933 | 31773959 | − | 183 | GTATGAGAAAAAAT | TTGG | 99 |
| | | | | | | | GATAAAAG | | |
| 51 | SLUCAS9KH | E51SLCas9KH42 | 31773937 | 31773963 | − | 2058 | GAAGGTATGAGAAA | AAAG | 64 |
| | | | | | | | AAATGATA | | |
| 51 | SLUCAS9KH | E51SLCas9KH43 | 31773952 | 31773978 | − | 2059 | GATGATCATCAAGC | TGAG | 11 |
| | | | | | | | AGAAGGTA | | |
| 51 | SLUCAS9KH | E51SLCas9KH44 | 31773958 | 31773984 | − | 184 | CAACGAGATGATCA | AAGG | 7 |
| | | | | | | | TCAAGCAG | | |
| 51 | SLUCAS9KH | E51SLCas9KH45 | 31773959 | 31773985 | − | 2060 | TCAACGAGATGATC | GAAG | 3 |
| | | | | | | | ATCAAGCA | | |
| 51 | SLUCAS9KH | E51SLCas9KH46 | 31773962 | 31773988 | − | 2061 | ATATCAACGAGATG | GCAG | 7 |
| | | | | | | | ATCATCAA | | |
| 51 | SLUCAS9KH | E51SLCas9KH47 | 31773965 | 31773991 | − | 2062 | AGGATATCAACGAG | CAAG | 7 |
| | | | | | | | ATGATCAT | | |
| 51 | SLUCAS9KH | E51SLCas9KH48 | 31773977 | 31774003 | − | 2063 | TGGGTGACCTTGAG | CGAG | 17 |
| | | | | | | | GATATCAA | | |
| 51 | SLUCAS9KH | E51SLCas9KH49 | 31773988 | 31774014 | − | 185 | GAGGGTGATGGTGG | GAGG | 22 |
| | | | | | | | GTGACCTT | | |
| 51 | SLUCAS9KH | E51SLCas9KH50 | 31773989 | 31774015 | − | 2064 | AGAGGGTGATGGTG | TGAG | 48 |
| | | | | | | | GGTGACCT | | |
| 51 | SLUCAS9KH | E51SLCas9KH51 | 31773999 | 31774025 | − | 186 | ATAAAATCACAGAG | TGGG | 27 |
| | | | | | | | GGTGATGG | | |
| 51 | SLUCAS9KH | E51SLCas9KH52 | 31774000 | 31774026 | − | 187 | TATAAAATCACAGA | GTGG | 23 |
| | | | | | | | GGGTGATG | | |
| 51 | SLUCAS9KH | E51SLCas9KH53 | 31774003 | 31774029 | − | 188 | AGTTATAAAATCAC | ATGG | 20 |
| | | | | | | | AGAGGGTG | | |
| 51 | SLUCAS9KH | E51SLCas9KH54 | 31774009 | 31774035 | − | 189 | TGATCAAGTTATAA | AGGG | 29 |
| | | | | | | | AATCACAG | | |
| 51 | SLUCAS9KH | E51SLCas9KH55 | 31774010 | 31774036 | − | 190 | TTGATCAAGTTATA | GAGG | 30 |
| | | | | | | | AAATCACA | | |
| 51 | SLUCAS9KH | E51SLCas9KH56 | 31774011 | 31774037 | − | 2065 | CTTGATCAAGTTAT | AGAG | 14 |
| | | | | | | | AAAATCAC | | |
| 51 | SLUCAS9KH | E51SLCas9KH57 | 31774013 | 31774039 | − | 2066 | TGCTTGATCAAGTT | ACAG | 11 |
| | | | | | | | ATAAAATC | | |
| 51 | SLUCAS9KH | E51SLCas9KH58 | 31774027 | 31774053 | − | 2067 | CGACTGGCTTTCTC | CAAG | 8 |
| | | | | | | | TGCTTGAT | | |
| 51 | SLUCAS9KH | E51SLCas9KH59 | 31774046 | 31774072 | − | 191 | GGGCTTGGACAGAA | CTGG | 2 |
| | | | | | | | CTTACCGA | | |
| 51 | SLUCAS9KH | E51SLCas9KH60 | 31774060 | 31774086 | − | 2068 | GGCAGATTTCAACC | ACAG | 5 |
| | | | | | | | GGGCTTGG | | |
| 51 | SLUCAS9KH | E51SLCas9KH61 | 31774064 | 31774090 | − | 192 | CTCTGGCAGATTTC | TTGG | 1 |
| | | | | | | | AACCGGGC | | |
| 51 | SLUCAS9KH | E51SLCas9KH62 | 31774069 | 31774095 | − | 193 | ACCTGCTCTGGCAG | CGGG | 10 |
| | | | | | | | ATTTCAAC | | |
| 51 | SLUCAS9KH | E51SLCas9KH63 | 31774070 | 31774096 | − | 194 | TACCTGCTCTGGCA | CCGG | 10 |
| | | | | | | | GATTTCAA | | |
| 51 | SLUCAS9KH | E51SLCas9KH64 | 31774081 | 31774107 | − | 2069 | GATGTTGGAGGTAC | GCAG | 7 |
| | | | | | | | CTGCTCTG | | |
| 51 | SLUCAS9KH | E51SLCas9KH65 | 31774084 | 31774110 | − | 195 | CTTGATGTTGGAGG | CTGG | 7 |
| | | | | | | | TACCTGCT | | |
| 51 | SLUCAS9KH | E51SLCas9KH66 | 31774096 | 31774122 | − | 196 | AATGCCATCTTCCT | GAGG | 22 |
| | | | | | | | TGATGTTG | | |
| 51 | SLUCAS9KH | E51SLCas9KH67 | 31774097 | 31774123 | − | 2070 | AAATGCCATCTTCC | GGAG | 19 |
| | | | | | | | TTGATGTT | | |
| 51 | SLUCAS9KH | E51SLCas9KH68 | 31774099 | 31774125 | − | 197 | AGAAATGCCATCTT | TTGG | 26 |
| | | | | | | | CCTTGATG | | |
| 51 | SLUCAS9KH | E51SLCas9KH69 | 31774123 | 31774149 | − | 2071 | TAAGGAAACTGCCA | CTAG | 41 |
| | | | | | | | TCTCCAAA | | |
| 51 | SLUCAS9KH | E51SLCas9KH70 | 31774144 | 31774170 | − | 198 | GGTGACACAACCTG | AAGG | 6 |
| | | | | | | | TGGTTACT | | |
| 51 | SLUCAS9KH | E51SLCas9KH71 | 31774145 | 31774171 | − | 2072 | TGGTGACACAACCT | TAAG | 10 |
| | | | | | | | GTGGTTAC | | |
| 51 | SLUCAS9KH | E51SLCas9KH72 | 31774153 | 31774179 | − | 199 | TGTTACTCTGGTGA | GTGG | 22 |
| | | | | | | | CACAACCT | | |
| 51 | SLUCAS9KH | E51SLCas9KH73 | 31774168 | 31774194 | − | 200 | AGCTCCTACTCAGA | CTGG | 2 |
| | | | | | | | CTGTTACT | | |
| 51 | SLUCAS9KH | E51SLCas9KH74 | 31774181 | 31774207 | − | 2073 | CCCAAAATATTTTA | TCAG | 10 |
| | | | | | | | GCTCCTAC | | |
| 51 | SLUCAS9KH | E51SLCas9KH75 | 31774192 | 31774218 | − | 2074 | TTTTGCAAAAACCC | TTAG | 43 |
| | | | | | | | AAAATATT | | |
| 53 | SACAS9KKH | E53SaCas9KKH1 | 31679352 | 31679380 | + | 1056 | AAAAGGTATCTTTG | CTTGGT | 45 |
| | | | | | | | ATACTAAC | | |
| 53 | SACAS9KKH | E53SaCas9KKH2 | 31679361 | 31679389 | + | 1057 | CTTTGATACTAACC | TGTGAT | 20 |
| | | | | | | | TTGGTTTC | | |
| 53 | SACAS9KKH | E53SaCas9KKH3 | 31679373 | 31679401 | + | 25 | CCTTGGTTTCTGTG | TTGGAT | 122 |
| | | | | | | | ATTTTCTT | | |
| 53 | SACAS9KKH | E53SaCas9KKH4 | 31679477 | 31679505 | + | 26 | TCCTTAGCTTCCAG | TTGAAT | 42 |
| | | | | | | | CCATTGTG | | |
| 53 | SACAS9KKH | E53SaCas9KKH5 | 31679510 | 31679538 | + | 1058 | AACATTTCATTCAA | TCCGGT | 46 |
| | | | | | | | CTGTTGCC | | |
| 53 | SACAS9KKH | E53SaCas9KKH6 | 31679519 | 31679547 | + | 1059 | TTCAACTGTTGCCT | GAAGGT | 9 |
| | | | | | | | CCGGTTCT | | |
| 53 | SACAS9KKH | E53SaCas9KKH7 | 31679543 | 31679571 | + | 1060 | AGGTGTTCTTGTAC | ACTGAT | 16 |
| | | | | | | | TTCATCCC | | |
| 53 | SACAS9KKH | E53SaCas9KKH8 | 31679550 | 31679578 | + | 27 | CTTGTACTTCATCC | CTGAAT | 58 |
| | | | | | | | CACTGATT | | |
| 53 | SACAS9KKH | E53SaCas9KKH9 | 31679565 | 31679593 | + | 28 | ACTGATTCTGAATT | TAGAAT | 78 |
| | | | | | | | CTTTCAAC | | |
| 53 | SACAS9KKH | E53SaCas9KKH10 | 31679577 | 31679605 | + | 1061 | TTCTTTCAACTAGA | AAAAAT | 86 |
| | | | | | | | ATAAAAGG | | |
| 53 | SACAS9KKH | E53SaCas9KKH11 | 31679581 | 31679609 | + | 1062 | TTCAACTAGAATAA | ATAAAT | 420 |
| | | | | | | | AAGGAAAA | | |
| 53 | SACAS9KKH | E53SaCas9KKH12 | 31679588 | 31679616 | + | 1063 | AGAATAAAAGGAAA | TATAGT | 5266 |
| | | | | | | | AATAAATA | | |
| 53 | SACAS9KKH | E53SaCas9KKH13 | 31679346 | 31679374 | − | 1064 | GTTAGTATCAAAGA | TAAAAT | 31 |
| | | | | | | | TACCTTTT | | |
| 53 | SACAS9KKH | E53SaCas9KKH14 | 31679359 | 31679387 | − | 1065 | CACAGAAACCAAGG | AAAGAT | 33 |
| | | | | | | | TTAGTATC | | |
| 53 | SACAS9KKH | E53SaCas9KKH15 | 31679368 | 31679396 | − | 1066 | AAAGAAAATCACAG | GTTAGT | 456 |
| | | | | | | | AAACCAAG | | |
| 53 | SACAS9KKH | E53SaCas9KKH16 | 31679372 | 31679400 | − | 1067 | TCCAAAAGAAAATC | CAAGGT | 246 |
| | | | | | | | ACAGAAAC | | |
| 53 | SACAS9KKH | E53SaCas9KKH17 | 31679387 | 31679415 | − | 1068 | ATACAGTAGATGCA | GAAAAT | 49 |
| | | | | | | | ATCCAAAA | | |
| 53 | SACAS9KKH | E53SaCas9KKH18 | 31679399 | 31679427 | − | 1069 | AGGAGGGTCCCTAT | TGCAAT | 13 |
| | | | | | | | ACAGTAGA | | |
| 53 | SACAS9KKH | E53SaCas9KKH19 | 31679404 | 31679432 | − | 1070 | ATGGAAGGAGGGTC | GTAGAT | 20 |
| | | | | | | | CCTATACA | | |
| 53 | SACAS9KKH | E53SaCas9KKH20 | 31679408 | 31679436 | − | 1071 | AGTCATGGAAGGAG | TACAGT | 27 |
| | | | | | | | GGTCCCTA | | |
| 53 | SACAS9KKH | E53SaCas9KKH21 | 31679419 | 31679447 | − | 29 | AGCCAAGCTTGAGT | GAGGGT | 36 |
| | | | | | | | CATGGAAG | | |
| 53 | SACAS9KKH | E53SaCas9KKH22 | 31679433 | 31679461 | − | 30 | TTAGGACAGGCCAG | TTGAGT | 30 |
| | | | | | | | AGCCAAGC | | |
| 53 | SACAS9KKH | E53SaCas9KKH23 | 31679462 | 31679490 | − | 1072 | TGGAAGCTAAGGAA | GCAGGT | 103 |
| | | | | | | | GAAGCTGA | | |
| 53 | SACAS9KKH | E53SaCas9KKH24 | 31679493 | 31679521 | − | 1073 | AATGAAATGTTAAA | CACAAT | 200 |
| | | | | | | | GGATTCAA | | |
| 53 | SACAS9KKH | E53SaCas9KKH25 | 31679503 | 31679531 | − | 31 | GCAACAGTTGAATG | AAGGAT | 155 |
| | | | | | | | AAATGTTA | | |
| 53 | SACAS9KKH | E53SaCas9KKH26 | 31679513 | 31679541 | − | 1074 | AGAACCGGAGGCAA | TGAAAT | 11 |
| | | | | | | | CAGTTGAA | | |
| 53 | SACAS9KKH | E53SaCas9KKH27 | 31679518 | 31679546 | − | 32 | CCTTCAGAACCGGA | TTGAAT | 8 |
| | | | | | | | GGCAACAG | | |
| 53 | SACAS9KKH | E53SaCas9KKH28 | 31679523 | 31679551 | − | 1075 | GAACACCTTCAGAA | AACAGT | 10 |
| | | | | | | | CCGGAGGC | | |
| 53 | SACAS9KKH | E53SaCas9KKH29 | 31679555 | 31679583 | − | 1076 | AAAGAATTCAGAAT | TGAAGT | 106 |
| | | | | | | | CAGTGGGA | | |
| 53 | SACAS9KKH | E53SaCas9KKH30 | 31679560 | 31679588 | − | 33 | AGTTGAAAGAATTC | TGGGAT | 110 |
| | | | | | | | AGAATCAG | | |
| 53 | SACAS9KKH | E53SaCas9KKH31 | 31679565 | 31679593 | − | 1077 | ATTCTAGTTGAAAG | ATCAGT | 134 |
| | | | | | | | AATTCAGA | | |
| 53 | SACAS9KKH | E53SaCas9KKH32 | 31679569 | 31679597 | − | 34 | TTTTATTCTAGTTG | CAGAAT | 399 |
| | | | | | | | AAAGAATT | | |
| 53 | SACAS9KKH | E53SaCas9KKH33 | 31679576 | 31679604 | − | 35 | TTTTTCCTTTTATT | AAGAAT | 475 |
| | | | | | | | CTAGTTGA | | |
| 53 | SACAS9KKH | E53SaCas9KKH34 | 31679585 | 31679613 | − | 1078 | ATATATTTATTTTT | TCTAGT | 2133 |
| | | | | | | | CCTTTTAT | | |
| 53 | SLUCAS9KH | E53SLCas9KH1 | 31679353 | 31679379 | + | 201 | AAAGGTATCTTTGA | TTGG | 13 |
| | | | | | | | TACTAACC | | |
| 53 | SLUCAS9KH | E53SLCas9KH2 | 31679373 | 31679399 | + | 202 | CCTTGGTTTCTGTG | TTGG | 51 |
| | | | | | | | ATTTTCTT | | |
| 53 | SLUCAS9KH | E53SLCas9KH3 | 31679391 | 31679417 | + | 2075 | TCTTTTGGATTGCA | ATAG | 18 |
| | | | | | | | TCTACTGT | | |
| 53 | SLUCAS9KH | E53SLCas9KH4 | 31679392 | 31679418 | + | 203 | CTTTTGGATTGCAT | TAGG | 12 |
| | | | | | | | CTACTGTA | | |
| 53 | SLUCAS9KH | E53SLCas9KH5 | 31679393 | 31679419 | + | 204 | TTTTGGATTGCATC | AGGG | 15 |
| | | | | | | | TACTGTAT | | |
| 53 | SLUCAS9KH | E53SLCas9KH6 | 31679414 | 31679440 | + | 2076 | TAGGGACCCTCCTT | CAAG | 11 |
| | | | | | | | CCATGACT | | |
| 53 | SLUCAS9KH | E53SLCas9KH7 | 31679419 | 31679445 | + | 205 | ACCCTCCTTCCATG | TTGG | 11 |
| | | | | | | | ACTCAAGC | | |
| 53 | SLUCAS9KH | E53SLCas9KH8 | 31679425 | 31679451 | + | 206 | CTTCCATGACTCAA | CTGG | 10 |
| | | | | | | | GCTTGGCT | | |
| 53 | SLUCAS9KH | E53SLCas9KH9 | 31679436 | 31679462 | + | 2077 | CAAGCTTGGCTCTG | TAAG | 16 |
| | | | | | | | GCCTGTCC | | |
| 53 | SLUCAS9KH | E53SLCas9KH10 | 31679446 | 31679472 | + | 2078 | TCTGGCCTGTCCTA | TCAG | 18 |
| | | | | | | | AGACCTGC | | |
| 53 | SLUCAS9KH | E53SLCas9KH11 | 31679458 | 31679484 | + | 2079 | TAAGACCTGCTCAG | TTAG | 16 |
| | | | | | | | CTTCTTCC | | |
| 53 | SLUCAS9KH | E53SLCas9KH12 | 31679465 | 31679491 | + | 2080 | TGCTCAGCTTCTTC | CCAG | 24 |
| | | | | | | | CTTAGCTT | | |
| 53 | SLUCAS9KH | E53SLCas9KH13 | 31679511 | 31679537 | + | 207 | ACATTTCATTCAAC | CCGG | 15 |
| | | | | | | | TGTTGCCT | | |
| 53 | SLUCAS9KH | E53SLCas9KH14 | 31679519 | 31679545 | + | 2081 | TTCAACTGTTGCCT | GAAG | 6 |
| | | | | | | | CCGGTTCT | | |
| 53 | SLUCAS9KH | E53SLCas9KH15 | 31679520 | 31679546 | + | 208 | TCAACTGTTGCCTC | AAGG | 4 |
| | | | | | | | CGGTTCTG | | |
| 53 | SLUCAS9KH | E53SLCas9KH16 | 31679564 | 31679590 | + | 2082 | CACTGATTCTGAAT | CTAG | 32 |
| | | | | | | | TCTTTCAA | | |
| 53 | SLUCAS9KH | E53SLCas9KH17 | 31679572 | 31679598 | + | 2083 | CTGAATTCTTTCAA | AAAG | 71 |
| | | | | | | | CTAGAATA | | |
| 53 | SLUCAS9KH | E53SLCas9KH18 | 31679573 | 31679599 | + | 209 | TGAATTCTTTCAAC | AAGG | 48 |
| | | | | | | | TAGAATAA | | |
| 53 | SLUCAS9KH | E53SLCas9KH19 | 31679589 | 31679615 | + | 2084 | GAATAAAAGGAAAA | ATAG | 574 |
| | | | | | | | ATAAATAT | | |
| 53 | SLUCAS9KH | E53SLCas9KH20 | 31679592 | 31679618 | + | 2085 | TAAAAGGAAAAATA | GTAG | 765 |
| | | | | | | | AATATATA | | |
| 53 | SLUCAS9KH | E53SLCas9KH21 | 31679361 | 31679387 | − | 2086 | CACAGAAACCAAGG | AAAG | 6 |
| | | | | | | | TTAGTATC | | |
| 53 | SLUCAS9KH | E53SLCas9KH22 | 31679369 | 31679395 | − | 2087 | AAGAAAATCACAGA | TTAG | 88 |
| | | | | | | | AACCAAGG | | |
| 53 | SLUCAS9KH | E53SLCas9KH23 | 31679373 | 31679399 | − | 210 | CCAAAAGAAAATCA | AAGG | 52 |
| | | | | | | | CAGAAACC | | |
| 53 | SLUCAS9KH | E53SLCas9KH24 | 31679374 | 31679400 | − | 2088 | TCCAAAAGAAAATC | CAAG | 75 |
| | | | | | | | ACAGAAAC | | |
| 53 | SLUCAS9KH | E53SLCas9KH25 | 31679382 | 31679408 | − | 2089 | AGATGCAATCCAAA | ACAG | 89 |
| | | | | | | | AGAAAATC | | |
| 53 | SLUCAS9KH | E53SLCas9KH26 | 31679392 | 31679418 | − | 2090 | CCTATACAGTAGAT | AAAG | 4 |
| | | | | | | | GCAATCCA | | |
| 53 | SLUCAS9KH | E53SLCas9KH27 | 31679406 | 31679432 | − | 2091 | ATGGAAGGAGGGTC | GTAG | 8 |
| | | | | | | | CCTATACA | | |
| 53 | SLUCAS9KH | E53SLCas9KH28 | 31679409 | 31679435 | − | 2092 | GTCATGGAAGGAGG | ACAG | 12 |
| | | | | | | | GTCCCTAT | | |
| 53 | SLUCAS9KH | E53SLCas9KH29 | 31679420 | 31679446 | − | 211 | GCCAAGCTTGAGTC | AGGG | 10 |
| | | | | | | | ATGGAAGG | | |
| 53 | SLUCAS9KH | E53SLCas9KH30 | 31679421 | 31679447 | − | 212 | AGCCAAGCTTGAGT | GAGG | 21 |
| | | | | | | | CATGGAAG | | |
| 53 | SLUCAS9KH | E53SLCas9KH31 | 31679422 | 31679448 | − | 2093 | GAGCCAAGCTTGAG | GGAG | 14 |
| | | | | | | | TCATGGAA | | |
| 53 | SLUCAS9KH | E53SLCas9KH32 | 31679424 | 31679450 | − | 213 | CAGAGCCAAGCTTG | AAGG | 18 |
| | | | | | | | AGTCATGG | | |
| 53 | SLUCAS9KH | E53SLCas9KH33 | 31679425 | 31679451 | − | 2094 | CCAGAGCCAAGCTT | GAAG | 18 |
| | | | | | | | GAGTCATG | | |
| 53 | SLUCAS9KH | E53SLCas9KH34 | 31679428 | 31679454 | − | 214 | AGGCCAGAGCCAAG | ATGG | 23 |
| | | | | | | | CTTGAGTC | | |
| 53 | SLUCAS9KH | E53SLCas9KH35 | 31679434 | 31679460 | − | 2095 | TAGGACAGGCCAGA | TGAG | 17 |
| | | | | | | | GCCAAGCT | | |
| 53 | SLUCAS9KH | E53SLCas9KH36 | 31679440 | 31679466 | − | 2096 | AGGTCTTAGGACAG | CAAG | 23 |
| | | | | | | | GCCAGAGC | | |
| 53 | SLUCAS9KH | E53SLCas9KH37 | 31679445 | 31679471 | − | 2097 | TGAGCAGGTCTTAG | AGAG | 27 |
| | | | | | | | GACAGGCC | | |
| 53 | SLUCAS9KH | E53SLCas9KH38 | 31679447 | 31679473 | − | 2098 | GCTGAGCAGGTCTT | CCAG | 35 |
| | | | | | | | AGGACAGG | | |
| 53 | SLUCAS9KH | E53SLCas9KH39 | 31679451 | 31679477 | − | 215 | AGAAGCTGAGCAGG | CAGG | 44 |
| | | | | | | | TCTTAGGA | | |
| 53 | SLUCAS9KH | E53SLCas9KH40 | 31679452 | 31679478 | − | 2099 | AAGAAGCTGAGCAG | ACAG | 16 |
| | | | | | | | GTCTTAGG | | |
| 53 | SLUCAS9KH | E53SLCas9KH41 | 31679456 | 31679482 | − | 216 | AAGGAAGAAGCTGA | TAGG | 53 |
| | | | | | | | GCAGGTCT | | |
| 53 | SLUCAS9KH | E53SLCas9KH42 | 31679457 | 31679483 | − | 2100 | TAAGGAAGAAGCTG | TTAG | 25 |
| | | | | | | | AGCAGGTC | | |
| 53 | SLUCAS9KH | E53SLCas9KH43 | 31679463 | 31679489 | − | 217 | GGAAGCTAAGGAAG | CAGG | 70 |
| | | | | | | | AAGCTGAG | | |
| 53 | SLUCAS9KH | E53SLCas9KH44 | 31679464 | 31679490 | − | 2101 | TGGAAGCTAAGGAA | GCAG | 52 |
| | | | | | | | GAAGCTGA | | |
| 53 | SLUCAS9KH | E53SLCas9KH45 | 31679467 | 31679493 | − | 2102 | GGCTGGAAGCTAAG | TGAG | 41 |
| | | | | | | | GAAGAAGC | | |
| 53 | SLUCAS9KH | E53SLCas9KH46 | 31679472 | 31679498 | − | 2103 | ACAATGGCTGGAAG | GAAG | 27 |
| | | | | | | | CTAAGGAA | | |
| 53 | SLUCAS9KH | E53SLCas9KH47 | 31679475 | 31679501 | − | 2104 | AACACAATGGCTGG | GAAG | 18 |
| | | | | | | | AAGCTAAG | | |
| 53 | SLUCAS9KH | E53SLCas9KH48 | 31679478 | 31679504 | − | 218 | TTCAACACAATGGC | AAGG | 87 |
| | | | | | | | TGGAAGCT | | |
| 53 | SLUCAS9KH | E53SLCas9KH49 | 31679479 | 31679505 | − | 2105 | ATTCAACACAATGG | TAAG | 24 |
| | | | | | | | CTGGAAGC | | |
| 53 | SLUCAS9KH | E53SLCas9KH50 | 31679484 | 31679510 | − | 2106 | AAAGGATTCAACAC | GAAG | 12 |
| | | | | | | | AATGGCTG | | |
| 53 | SLUCAS9KH | E53SLCas9KH51 | 31679487 | 31679513 | − | 219 | GTTAAAGGATTCAA | CTGG | 12 |
| | | | | | | | CACAATGG | | |
| 53 | SLUCAS9KH | E53SLCas9KH52 | 31679491 | 31679517 | − | 220 | AAATGTTAAAGGAT | ATGG | 37 |
| | | | | | | | TCAACACA | | |
| 53 | SLUCAS9KH | E53SLCas9KH53 | 31679505 | 31679531 | − | 221 | GCAACAGTTGAATG | AAGG | 28 |
| | | | | | | | AAATGTTA | | |
| 53 | SLUCAS9KH | E53SLCas9KH54 | 31679506 | 31679532 | − | 2107 | GGCAACAGTTGAAT | AAAG | 25 |
| | | | | | | | GAAATGTT | | |
| 53 | SLUCAS9KH | E53SLCas9KH55 | 31679524 | 31679550 | − | 2108 | AACACCTTCAGAAC | ACAG | 7 |
| | | | | | | | CGGAGGCA | | |
| 53 | SLUCAS9KH | E53SLCas9KH56 | 31679530 | 31679556 | − | 222 | TACAAGAACACCTT | GAGG | 10 |
| | | | | | | | CAGAACCG | | |
| 53 | SLUCAS9KH | E53SLCas9KH57 | 31679531 | 31679557 | − | 2109 | GTACAAGAACACCT | GGAG | 10 |
| | | | | | | | TCAGAACC | | |
| 53 | SLUCAS9KH | E53SLCas9KH58 | 31679533 | 31679559 | − | 223 | AAGTACAAGAACAC | CCGG | 17 |
| | | | | | | | CTTCAGAA | | |
| 53 | SLUCAS9KH | E53SLCas9KH59 | 31679539 | 31679565 | − | 2110 | GGGATGAAGTACAA | TCAG | 18 |
| | | | | | | | GAACACCT | | |
| 53 | SLUCAS9KH | E53SLCas9KH60 | 31679550 | 31679576 | − | 2111 | TCAGAATCAGTGGG | CAAG | 27 |
| | | | | | | | ATGAAGTA | | |
| 53 | SLUCAS9KH | E53SLCas9KH61 | 31679556 | 31679582 | − | 2112 | AAGAATTCAGAATC | GAAG | 27 |
| | | | | | | | AGTGGGAT | | |
| 53 | SLUCAS9KH | E53SLCas9KH62 | 31679562 | 31679588 | − | 224 | AGTTGAAAGAATTC | TGGG | 95 |
| | | | | | | | AGAATCAG | | |
| 53 | SLUCAS9KH | E53SLCas9KH63 | 31679563 | 31679589 | − | 225 | TAGTTGAAAGAATT | GTGG | 33 |
| | | | | | | | CAGAATCA | | |
| 53 | SLUCAS9KH | E53SLCas9KH64 | 31679566 | 31679592 | − | 2113 | TTCTAGTTGAAAGA | TCAG | 43 |
| | | | | | | | ATTCAGAA | | |
| 53 | SLUCAS9KH | E53SLCas9KH65 | 31679572 | 31679598 | − | 2114 | CTTTTATTCTAGTT | TCAG | 61 |
| | | | | | | | GAAAGAAT | | |
| 53 | SLUCAS9KH | E53SLCas9KH66 | 31679579 | 31679605 | − | 2115 | ATTTTTCCTTTTAT | AAAG | 122 |
| | | | | | | | TCTAGTTG | | |
| 53 | SLUCAS9KH | E53SLCas9KH67 | 31679586 | 31679612 | − | 2116 | TATATTTATTTTTC | CTAG | 978 |
| | | | | | | | CTTTTATT | | |

In some embodiments, the AAV vectors and/or compositions thereof comprise a single nucleic acid molecule comprising: i) a nucleic acid encoding Staphylococcus aureus Cas9 (SaCas9) or Staphylococcus lugdunensis (SluCas9) and at least one, at least two, or at least three guide RNAs; or ii) a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or iii) a nucleic acid encoding Staphylococcus aureus Cas9 (SaCas9) or Staphylococcus lugdunensis (SluCas9) and one to three guide RNAs.

In some embodiments, the AAV vectors and/or compositions thereof comprises at least two nucleic acid molecules comprising a first nucleic acid encoding Staphylococcus aureus Cas9 (SaCas9) or Staphylococcus lugdunensis (SluCas9); and a second nucleic acid that does not encode a SaCas9 or SluCas9 and encodes any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, the AAV vectors and/or compositions thereof comprises at least two nucleic acid molecules comprising a first nucleic acid encoding Staphylococcus aureus Cas9 (SaCas9) or Staphylococcus lugdunensis (SluCas9) and i) at least one, at least two, or at least three guide RNAs; or ii) from one to n guide RNAs, wherein n is no more than the maximum number of guide RNAs that can be expressed from said nucleic acid; or iii) one to three guide RNAs; and a second nucleic acid that does not encode a SaCas9 or SluCas9, optionally wherein the second nucleic acid comprises any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, the AAV vectors and/or compositions thereof comprises at least two nucleic acid molecules comprising a first nucleic acid encoding Staphylococcus aureus Cas9 (SaCas9) or Staphylococcus lugdunensis (SluCas9) and at least one, at least two, or at least three guide RNAs; and a second nucleic acid that does not encode a SaCas9 or SluCas9 and encodes from one to six guide RNAs.

In some embodiments, the AAV vectors and/or compositions thereof comprises at least two nucleic acid molecules comprising a first nucleic acid encoding Staphylococcus aureus Cas9 (SaCas9) or Staphylococcus lugdunensis (SluCas9) and at least two guide RNAs, wherein at least one guide RNA binds upstream of sequence to be excised and at least one guide RNA binds downstream of sequence to be excised; and a second nucleic acid that does not encode a SaCas9 or SluCas9 and encodes at least one additional copy of the guide RNAs encoded in the first nucleic acid. In some embodiments, the guide RNA excises a portion of a DMD gene, optionally an exon, intron, or exon/intron junction.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and a first and a second guide RNA that function to excise a portion of a DMD gene; and a second nucleic acid encoding at least 2 or at least 3 copies of the first guide RNA and at least 2 or at least 3 copies of the second guide RNA.

In some embodiments, a composition is provided comprising, consisting of, or consisting essentially of one or more nucleic acid molecules encoding an endonuclease and a pair of guide RNAs, wherein each guide RNA targets a different sequence in a DMD gene, wherein the endonuclease and pair of guide RNAs are capable of excising a target sequence in DNA that is between 5-250 nucleotides in length. In some embodiments, the endonuclease is a class 2, type II Cas endonuclease. In some embodiments, the class 2, type II Cas endonuclease is SpCas9, SaCas9, or SluCas9. In some embodiments, the endonuclease is not a class 2, type V Cas endonuclease. In some embodiments, the excised target sequence comprises a splice acceptor site or a splice donor site. In some embodiments, the excised target sequence comprises a premature stop codon in the DMD gene. In some embodiments, the excised target sequence does not comprise an entire exon of the DMD gene. In some embodiments, any of the methods and/or ribonucleoprotein complexes disclosed herein do not destroy/specifically alter the sequence of a splice acceptor site, splice donor site, or premature stop codon site.

### III. Methods

Disclosed herein are methods comprising (a) administering to a subject an agent that blocks AAV binding to an AAV receptor (AAVR) in the liver, and then (b) administering an AAV vector to the subject.

Disclosed herein are methods comprising increasing the percentage of AAV delivered to a non-liver target in a subject, comprising (a) a pre-conditioning step comprising administering to the subject a composition comprising an agent that blocks AAV binding to an AAV receptor (AAVR) in the liver, and then (b) administering an AAV vector targeting a non-liver tissue. Including such a composition, for example, in a conditioning or pre-conditioning regimen could reduce liver infectivity, protect liver health, and increase potency to target tissues.

The methods disclosed herein may be used to improve the tropism of an AAV for a non-liver target in a subject, comprising administering to the subject a composition comprises an agent that blocks AAV binding to an AAV receptor (AAVR) and a delivery molecule that delivers the agent to the liver, and then administering an AAV vector, wherein the AAV vector is not intended to target liver.

The methods disclosed herein may also be used to decrease tropism of AAV to the liver in a subject comprising administering to the subject a composition comprises an agent that blocks AAV binding to an AAV receptor (AAVR) comprising a small or large molecule and a delivery molecule that delivers the agent to the liver, and then administering an AAV vector, wherein the AAV vector is not intended for the liver. In some embodiments, administration of the composition comprising the agent that blocks AAV binding to an AAV receptor increases the percentage of AAV delivered to the non-liver target.

In some embodiments, the methods comprise administering an agent that blocks AAV binding to an AAV receptor (AAVR) and a delivery molecule that delivers the agent to the liver and induces long term blocking of AAV binding to AAVR, e.g., longer than about 3 weeks. In some embodiments, the blocking of AAV binding to AAV receptors in the liver is not temporary. In some embodiments, the blocking of AAV binding to AAV receptors in the liver is temporary.

In some embodiments, administering to a subject in need thereof a composition for blocking AAV binding to AAV receptors in the liver occurs prior to administering the non-liver AAV-based gene therapy, for example at least about 3 weeks, at least about 4 weeks, at least about 5 weeks, at least about 6 weeks, at least about 7 weeks, at least about 8 weeks, at least about 9 weeks, or at least about 10 weeks prior to administering the AAV. In particular embodiments, the agent that blocks binding to AAV receptors in the liver (e.g., any of the siRNAs or ASOs disclosed herein) is administered to the subject 1-2, 1-3, 2-5, 4-7, 6-9, 8-11, 10-13 or 12-15 days prior to administering any of the AAVs disclosed herein. In some embodiments, the composition that blocks AAV binding to an AAV receptor (AAVR) comprises an siRNA. In some embodiments, the composition that blocks AAV binding to an AAV receptor (AAVR) comprises an ASO. In some embodiments, the composition that blocks AAV binding to an AAV receptor (AAVR) comprises an anti-AAV antibody that blocks AAV binding to AAV receptors. In some embodiments, the composition that blocks AAV binding to an AAV receptor (AAVR) comprises a non-RNAi and non-antibody inhibitor. In some embodiments, the compositions comprise at least one small molecule inhibitor or anti-sense oligonucleotide.

As set forth in detail above, the compositions for blocking AAV binding to AAV receptors in the liver include but are not limited to compositions comprising RNAi specific for AAVR and compositions comprising antisense oligonucleotides (ASOs) targeting an AAVR-encoding transcript.

In some embodiments, systemically or locally delivered siRNA induces a temporary gene expression knockdown effect by up to 90% from 48 hours to 3 weeks in animal experiments for eyes, brain, spinal cord, lungs, subcutaneous tissue, vagina, skin, isolated tumor, heart et al. See Kim, Korean J Anesthesiol. 59(6): 369-370 (2010). Small interfering RNA targeted to the liver, including by way of conjugated siRNA and siRNA-LNP targeted delivery, can temporarily block AAV binding to AAVRs in the liver. After the siRNA targeted to the liver is administered and temporary blocking of the AAVR is induced, non-liver AAV-based gene therapy can be administered.

In some embodiments, systemically or locally delivered ASOs are delivered to induce a temporary gene expression knockdown effect of about 50%, about 60%, about 70%, or about 80% from about 24 hours to about 48 hours to greater than or about 10 days in accordance with the Examples and Figures presented herein. After the ASO is administered and temporary knockdown of the AAVR is induced, non-liver AAV-based gene therapy can be administered.

The non-liver AAV-based gene therapy is enhanced because the gene therapy can be more effectively routed to the intended target by way of the decreased AAV tropism to the liver. Because AAVR have been shown to contribute significantly to AAV vector transduction efficiency and tropism and have been shown to bind directly to AAV particles and to be rate limiting for viral transduction, blocking the AAVR lessens the tropism of AAV to those cells (e.g., hepatocytes). By decreasing AAV tropism to the liver, the non-liver AAV-based gene therapy is enhanced or improved because of the lowered risk of off-target liver toxicity. Accordingly in some embodiments, administering to the subject administering an agent that blocks AAV binding to an AAV receptor (AAVR) temporarily blocks AAV binding to AAV receptors in the liver. In some embodiments, a subject who is a) treated with an agent that blocks AAV binding to an AAV receptor (AAVR) and b) subsequently treated with an AAV, displays a reduction of at least 10%, 20%, 30%, 40%, 50%, 60%, 70%, 80%, 90%, 95%, or 99% in levels of AAV within the liver as compared to a subject who is treated with the AAV but not treated with the agent.

In some embodiments, the method includes administering an siRNA or ASO that is capable of temporarily blocking AAV binding to AAVR receptors in the liver, including for about 48 hours to 3 weeks. In some embodiments, the siRNA or ASO blocks AAV binding to AAVR receptors in the liver for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the siRNA or ASO blocks AAV binding to AAVR receptors in the liver for about 7 days, about 8 days, about 9 days, about 10 days, about 11 days, about 12 days, about 13 days, about 14 days. In some embodiments, the compositions are capable of blocking AAV binding to AAVR receptors in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the siRNA or ASO knocks down AAVR and comprises a ribonucleotide sequence at least 80% identical to a ribonucleotide sequence from the AAVR. Preferably, the siRNA or ASO molecule is at least 90%, 95%, 96%, 97%, 98%, 99% or 100% identical to the ribonucleotide sequence of the target. Most preferably, an siRNA or ASO will be 100% identical to the nucleotide sequence of a target agent or virus. However, siRNA or ASO molecules with insertions, deletions or single point mutations relative to a target may also be effective. Tools to assist siRNA design or ASO design are readily available to the public and are known in the art.

In some embodiments, the subject is administered an amount of an siRNA that knocks down the levels of AAVR in the liver by at least 10%, 20%, 30%, 40%, 50%, 60%, 70%, 80%, 90%, 95%, or 99% as compared to a control subject not administered the siRNA. In some embodiments, the subject is administered an amount of the siRNA that knocks down the levels of AAVR in the liver by 80-100%, 80-95%, 10-90%, 10-70%, 10-50%, 10-30%, 30-90%, 30-70%, 30-50%, 50-90%, 50-70%, or 70-90% as compared to a control subject not administered the siRNA.

In some embodiments, the subject is administered an amount of the siRNA that knocks down the levels of AAVR in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the subject is administered an amount of an ASO that knocks down the levels of AAVR in the liver by at least 10%, 20%, 30%, 40%, 50%, 60%, 70%, 80%, 90%, 95%, or 99% as compared to a control subject not administered the ASO. In some embodiments, the subject is administered an amount of an ASO that knocks down the levels of AAVR in the liver by 80-100%, 80-95%, 10-90%, 10-70%, 10-50%, 10-30%, 30-90%, 30-70%, 30-50%, 50-90%, 50-70%, or 70-90% as compared to a control subject not administered the ASO.

In some embodiments, the subject is administered an amount of the ASO that knocks down the levels of AAVR in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours. In some embodiments, the subject is administered an amount of the ASO that knocks down the levels of AAVR in the liver for 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the method includes administering an agent that blocks AAV binding to an AAV receptor (AAVR), e.g., siRNA or ASO, that is conjugated to a liver-targeting moiety. In some embodiments, the method enhances their delivery and/or uptake by the liver. In some embodiments, the method includes administering siRNA or ASO conjugated to a lipid, such as cholesterol. In some embodiments, the method includes administering siRNA or ASO conjugated to at least one galactose or galactose derivative, including but not limited to lactose, galactose, N-acetylgalactosamine (GalNAc), galactosamine, N-formylgalactosamine, N-acetylgalactosamine, N-propionylgalactosamine, Nn-butanoylgalactosamine, and N-isobutanoyl-galactosamine (Iobst, S T and Drickamer, K. JBC 1996, 271, 6686). In some embodiments, the method includes administering GalNAc-conjugated siRNAs.

In some embodiments, the method includes administering a composition comprising an agent that blocks AAV binding to an AAV receptor (AAVR) delivered by non-viral tissue-specific delivery vehicles including but not limited to nanoparticles, liposomes, ribonucleoproteins, positively charged peptides, small molecule RNA-conjugates, aptamer-RNA chimeras, and RNA-fusion protein complexes. In some embodiments, the agent is delivered to a cell or a patient by a lipid nanoparticle (LNP). In some embodiments, the method includes administering a composition comprising a siRNA encapsulated in a LNP. In some embodiments, the agent is delivered to a cell or a patient without being conjugated and/or without a non-viral tissue-specific delivery vehicle. In some embodiments, the method includes administering a composition comprising an ASO and a pharmaceutically acceptable carrier, for example, phosphate-buffered saline (PBS).

Exemplary modes of administration of the composition for blocking AAV binding to AAV receptors in the liver, e.g., a conjugated siRNA or siRNA-LNP delivery system or ASO, include oral administration, parenteral administration, administration by injection (e.g., intravenous, subcutaneous, intramuscular, intrathecal (IT), intracerebroventricular (ICV), etc.), and any other suitable mode of administration. In some embodiments, administration of the agent for blocking AAV binding to AAV receptors in the liver (e.g., siRNA or ASO) and compositions thereof include intraocular administration, such as intravitreal, intraretinal, subretinal, subtenon, peri- and retro-orbital, trans-comeal and trans-scleral administration. In some embodiments, the agent may be administered to a patient by intravenous injection, subcutaneous injection, oral delivery, liposome delivery or intranasal delivery. The agent may then accumulate in a target body system, organ, tissue or cell type of the patient.

In some embodiments, other drugs that facilitate increased uptake of an agent (e.g., siRNA) in the liver may also be co-administered with the agent (e.g., siRNA) conjugated to a liver-targeting moiety. In some embodiments, the method comprises co-administering a cholesterol-conjugated agent (e.g., siRNA) with a statin drug to block AAV binding to AAVR in the liver. In some embodiments, a statin drug can be co-administered with the cholesterol-agent to enhance uptake of cholesterol-conjugated agent in the liver. See US20150361432A1 for a general discussion regarding co-administering statin drugs with cholesterol-siRNA to increase the expression of LDL receptors on the surface of liver hepatocytes. As a consequence of the increase in LDL receptor expression, the level of cholesterol is lowered in plasma. Without wishing to be bound by theory, by administering a statin drug, the level of competing cholesterol in plasma is reduced and the level of LDL receptors for binding cholesterol-agent in the liver are increased, allowing for more efficient uptake of cholesterol labeled agent by hepatocytes. The statin can be administered before, with or after the administration of the cholesterol-agent.

In some embodiments, the method includes administering an AAV vector, including any of the AAV vectors described herein. In some embodiments, the AAV vector targets a non-liver tissue. In some embodiments, the method comprises administering to a subject the composition comprising an agent that blocks AAV binding to an AAV receptor (AAVR) as part of the pre-conditioning treatment prior to receiving or administering the AAV vector or AAV-based gene therapy directed to the non-liver target tissue. In some embodiments, the composition for blocking AAV binding to AAV receptors in the liver is administered to a subject at least once before the administration of the AAV Vector or AAV-based gene therapy directed to the non-liver target tissue. In some embodiments, the composition for blocking AAV binding to AAV receptors in the liver is administered to a subject at least once before the AAV-based gene therapy as part of a pre-conditioning regimen that may include administering other agents.

In particular embodiments, the subject is not administered an agent that blocks AAV binding to an AAV vector at the same time as being administered an AAV.

In some embodiments, the disclosure provides for administering to a subject the composition comprising an agent that blocks AAV binding to an AAV receptor (AAVR) (e.g., siRNA or ASO) about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the AAV vector. In some embodiments, the disclosure provides for administering to a subject the composition comprising an agent that blocks AAV binding to an AAV receptor (AAVR) occurs about 2-21, 10-21, 14-21, 2-17, 10-17, 1-14 days, 2-14 days, 3-14 days, 7-14 days, 1-10 days, 2-10 days, 3-10 days, 7-10 days, 1-7 days, 2-7 days, 3-7 days, 1-4 days, or 1-3 days, or about 24 hours to about 48 hours prior to administering the AAV vector. In some embodiments, administering the composition comprising an agent that blocks AAV binding to an AAV receptor (AAVR) occurs about 1 day, about 2 days, about 3 days, about 4 days, about 5 days, about 6 days, about 7 days, about 8 days, about 9 days, about 10 days, about 11 days, about 12 days, about 13 days, or about 14 days prior to administering the AAV vector. In some embodiments, administering the composition comprising an agent that blocks AAV binding to an AAV receptor (AAVR) occurs about 24 hours to about 48 hours prior to administering the AAV vector. In some embodiments, the subject is administered more than one dose (e.g., 2, 3 or 4) of the agent that blocks AAV binding to the AAVR prior to being administered the AAV.

In some embodiments, administering the composition comprising an agent that blocks AAV binding to an AAV receptor (AAVR) (e.g., siRNA or ASO) immediately precedes administering the AAV vector targeting a non-liver tissue. In some embodiments, the composition comprising an agent that blocks AAV binding to an AAV receptor (AAVR) and the AAV vector are co-administered.

The non-liver AAV-based gene therapy includes treating or preventing a disease or disorder, such as a genetic disease or disorder, in a subject in need thereof that is not a disease or disorder of the liver. In some embodiments, the AAV vector is intended for the brain; central nervous system; spinal cord; eye; retina; bone; cardiac muscle, skeletal muscle, and/or smooth muscle; lung; pancreas; heart; and/or kidney. In some embodiments, the AAV vector is intended for cardiac muscle, skeletal muscle, and/or smooth muscle.

In some embodiments, the method results in an increased percentage of AAV delivered to the non-liver target. In some embodiments, the method results in at least a 10%, 30%, 50%, 75%, 100%, 125%, 150%, 200%, 250%, 300%, 350%, 400%, 450%, 500%, 600%, 700%, 800%, 900%, or 1000% increase of AAV in brain, central nervous system, spinal cord, eye, retina, bone, cardiac muscle, skeletal muscle, and/or smooth muscle, lung, pancreas, heart, and/or kidney of the subject receiving the agent that blocks AAV binding to an AAV receptor (AAVR) in the liver and subsequently an AAV as compared to the AAV in the corresponding tissue of a control subject that received the AAV but did not receive the agent that blocks AAV binding to an AAV receptor (AAVR) in the liver. In some embodiments, the method results in a 10-50%, 50-100%, 100-250%, 250-500%, 500-750%, 750-1000%, or 1000-2000% increase of AAV in brain, central nervous system, spinal cord, eye, retina, bone, cardiac muscle, skeletal muscle, smooth muscle, lung, pancreas, heart, and/or kidney of the subject receiving the agent that blocks AAV binding to an AAV receptor (AAVR) in the liver and subsequently an AAV as compared to the AAV in the corresponding tissue of a control subject that received the AAV but did not receive the agent that blocks AAV binding to an AAV receptor (AAVR) in the liver. In some embodiments, the method results in at least a 10%, 30%, 50%, 75%, 100%, 125%, 150%, 200%, 250%, 300%, 350%, 400%, 450%, 500%, 600%, 700%, 800%, 900%, or 1000% increase of AAV in skeletal muscle of the subject receiving the agent that blocks AAV binding to an AAV receptor (AAVR) in the liver and subsequently an AAV, as compared to the AAV in the muscle of a control subject that received the AAV but did not receive the agent. In some embodiments, the method results in a 10-50%, 50-100%, 100-250%, 250-500%, 500-750%, 750-1000%, or 1000-2000% increase of AAV in skeletal muscle of the subject receiving the agent that blocks AAV binding to an AAV receptor (AAVR) in the liver and subsequently an AAV, as compared to the AAV in the muscle of a control subject that received the AAV but did not receive the agent.

In some embodiments, the non-liver AAV-based gene therapy is used to treat a genetic disease or disorder, where the disorder is a muscle disease or disorder. The muscle disease or disorder may be selected from, for example, Duchenne Muscular Dystrophy (DMD), Becker muscular dystrophy (BMD), Emery-Dreifuss dystrophy, myotonic dystrophy, limb-girdle muscular dystrophy, oculopharyngeal muscular dystrophy, congenital dystrophy, familial periodic paralysis. In some embodiments, the muscle disease or disorder may be mitochondrial oxidative phosphorylation disorder, or a glycogen storage disease (e.g., von Gierke's disease, Pompe's disease, Forbes-Cori disease, Andersen's disease, McArdle's disease, Hers' disease, Tarui's disease, or Fanconi-Bickel syndrome.) In particular embodiments, the non-liver AAV-based gene therapy is used to treat DMD. In some embodiments, the non-liver AAV-based gene therapy is used to treat myotonic dystrophy.

In some embodiments, the method includes administering a single AAV vector or multiple AAV vectors that have a non-liver target. In some embodiments, the method comprises administering an AAV1, AAV2, AAV3, AAV4, AAV5, AAV6, AAV7, AAV8, AAVrh10, AAVrh74, AAV9, AAV9P, or Myo-AAV vector. In some embodiments, the methods include administering AAV vectors that are recombinant or engineered AAV vectors. In some embodiments, the AAV vector comprises a tissue-specific (e.g., muscle-specific) promoter, e.g., which is operatively linked to a sequence encoding a guide RNA. In preferred embodiments, the AAV vector is less than 5 kb from ITR to ITR in size, inclusive of both ITRs. In particular embodiments, the AAV vector is less than 4.9 kb from ITR to ITR in size, inclusive of both ITRs. In further embodiments, the AAV vector is less than 4.85 kb from ITR to ITR in size, inclusive of both ITRs. In further embodiments, the AAV vector is less than 4.8 kb from ITR to ITR in size, inclusive of both ITRs. In further embodiments, the AAV vector is less than 4.75 kb from ITR to ITR in size, inclusive of both ITRs. In further embodiments, the AAV vector is less than 4.7 kb from ITR to ITR in size, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb from ITR to ITR in size, inclusive of both ITRs. In some embodiments, the vector is an AAV9 vector.

In some embodiments, the AAV vectors and AAV based gene therapy involve administering CRISPR-Cas components, any of which are known in the art. In some embodiments, the method includes administering one or more AAV vectors comprising a nucleic acid encoding a Cas9 protein. Such embodiments include for example, one or more AAV vectors comprising a nucleic acid encoding Staphylococcus aureus (SaCas9) and/or Staphylococcus lugdunensis (SluCas9) and further comprising a nucleic acid encoding one or more guide RNAs. In such embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9. In some embodiments, in addition to guide RNA and Cas9 sequences, the vectors further comprise nucleic acids that do not encode guide RNAs. In some embodiments, the vector comprises one or more nucleotide sequence(s) encoding a crRNA, a trRNA, or a crRNA and trRNA. A discussion of different AAV compositions useful in the present methods, including exemplary guide RNAs, promoters, and particular spacer sequences are disclosed in WO2022/056000 and elsewhere herein.

In particular embodiments, the non-liver target is the muscle and the method comprises administering an AAV vector to the subject subsequent to administering to a subject an agent that blocks AAV binding to an AAV receptor (AAVR) in the liver. In particular embodiments, the method comprises administering an AAV vector targeting the muscle subsequent to a pre-conditioning step comprising administering to the subject a composition comprising an agent that blocks AAV binding to an AAV receptor (AAVR) in the liver, wherein the pre-conditioning step increases the percentage of AAV delivered to the non-liver target. In particular embodiments, the non-liver AAV-based gene therapy is used to treat DMD. In such embodiments, the guide RNAs comprise as non-limiting examples the guide sequences disclosed in Tables 1A, 1B, and Table 2. For example, when the AAV vector comprises SaCas9, one or more spacer sequences is selected from any one of SEQ ID NOs: 1-35, 1000-1078, and 3000-3069; or when the AAV vector comprises SluCas9a, one or more spacer sequences selected from any one of SEQ ID NOs: 100-225, 2000-2116, and 4000-4251.

In some embodiments, the methods include administering AAV vectors that further comprise molecules for enhancing tropism for the target host cells or tissue. Uptake of AAVs by vascular endothelial and other target cell types can further be enhanced by using AAVs that further comprise one or more molecules for enhancing tropism of the vector for particular target host cells. In some embodiments, the one or more molecules for enhancing tropism are proteins. In some embodiments, the one or more molecules for enhancing tropism of the viral vector are peptides. In some embodiments, the one or more peptides target the viral vector to proteins upregulated in cells associated with the particular genetic disease or disorder to be treated. Such peptides and proteins are known in the art for enhancing tropism toward target host cells and may be incorporated into the AAVs through any of various methodologies known in the art.

Exemplary modes of administration of the non-liver AAV-based gene therapy include oral, rectal, transmucosal, topical, transdermal, inhalation, parenteral (e.g., intravenous, subcutaneous, intradermal, intramuscular, and intra-articular, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, the virus may be administered locally, for example in a depot or sustained-release formulation.

In some embodiments, the subject is a human subject. In some embodiments, the subject is being treated for a genetic disease or a disorder that is not a disease or disorder of the liver. In some embodiments, the subject is being treated for a muscle disease or disorder. In some embodiments, the subject has been or is being treated with a non-liver AAV-based gene therapy.

### EXAMPLES

The following examples are provided to illustrate certain disclosed embodiments and are not to be construed as limiting the scope of this disclosure in any way.

### 1. Example 1: Study of the Effect of Anti-AAVR siRNA on AAV Infectivity in Cell Models

To evaluate the effects of anti-AAVR siRNA and ASO administration on AAV infectivity in liver cells, liver cell models were transfected with either a siRNA targeting AAVR (anti-AAVR siRNA), an ASO, or a control. Cells were lysed for mRNA and protein extraction followed by qRT-PCR (mRNA) analysis and Western Blot analysis (protein) to assess the degree of AAVR mRNA knockdown.

### a) Dose Response (CRO):

Hepa1-6 cells were seeded at a density of 20 k cells per well in 96-well tissue culture plates. Cells were immediately transfected with siRNAs targeting mmAu040320 (mouse AAVR gene) at 10 different doses using Lipofectamine 2000 (Invitrogen 11668027). A Quantigene 2.0 branched DNA (bDNA) probe set was designed for the target mRNAs. Relative mmAu040320/mmGAPDH ratios were normalized to the respective mean ratio in mock treated cells and cells were transfected with a control siRNA targeting.

FIGS. 1A-1J show the effects of siRNA concentration on relative mRNA expression and shows 10-point dose response curves for each of the 10 siRNA sequences evaluated, set forth in Tables A1-A3. The top performing mmAu040320-targeting siRNAs were used in subsequent studies.

b) Knockdown of AAVR in Mouse Myoblasts with ASOs and siRNA:

C2C12 (mouse myoblast) cells were seeded at 14.4 k per well in a 96-well tissue culture plate. Next-day cells were treated with 50 nM ASO or 10 nM siRNA formulated with RNAiMax following manufacturer's protocol. After 48 hours, cells were lysed using a Cells-to-CT kit, following manufacturer's protocol. mRNA levels were measured by TaqMan assay, in a multiplexed reaction, using beta actin as a housekeeping control gene for normalization across samples. qPCR was run on Quantstudio 6 flex using mastermix from a Cells-to-CT 1-step TaqMan Kit (Invitrogen A25603). 20 ul reactions were conducted in technical triplicates for each RNA sample according to the user manual. 2 ul of cell lysate was added to each reaction, and Taqman probes (mouse): AAVR: FAM Mm00460200_m1 and Act-B: VIC Mm02619580_g1 were used. Delta Delta Ct analysis was conducted to find fold change of the samples in relation to the untreated control samples.

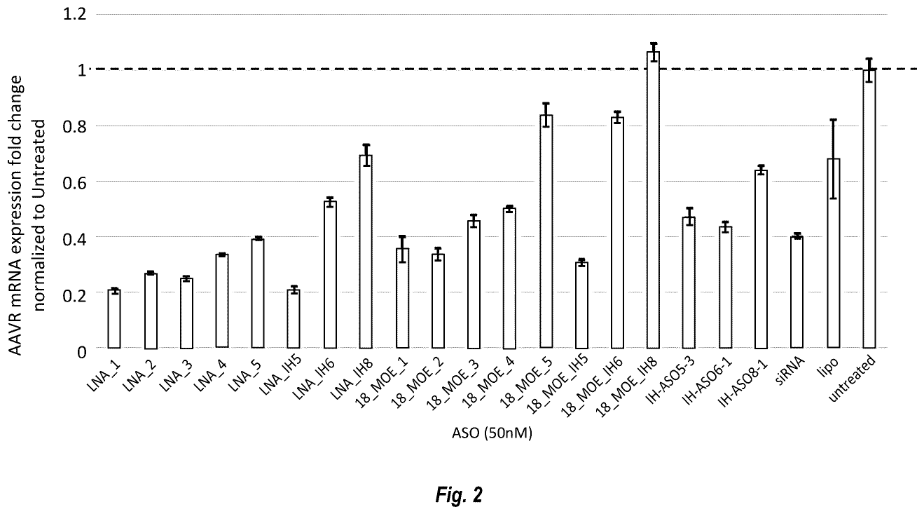

FIG. 2 shows the amount of change normalized to untreated cells and B-Actin in the C2C12 (mouse myoblast) cells.

c) Knockdown of AAVR mRNA in Liver Cell Lines with siRNA and ASO

Huh7 cells were seeded at 15 k per well in a 96-well tissue culture plate and grown overnight in growth media DMEM supplemented with 10% FBS and PenStrap. The next-day cells were dosed with 10-200 nM for ASOs or siRNA controls (siRNAVT011 targeting AAVR positive control; and non-targeting siRNA as negative control). The ASO was mixed with Opti-MEM (GIBCO 31985062) and combined with a mixture of Lipofectamine RNAiMAx Transfection reagent (Invitrogen 13778150) and incubated for 15 min at room temperature, following manufacturer's protocol (www.thermofisher.com/order/catalog/product/13778150). Untreated cells were left until media change. Cells were lysed after 48 hrs for mRNA analysis. Cells were lysed using lysis buffer from Cells-to-CT 1-step TaqMan Kit (Invitrogen A25603). qPCR was run on Quantstudio 6 flex using mastermix from Cells-to-CT 1-step TaqMan Kit (Invitrogen A25603). 20 ul reactions were conducted in technical duplicates for each RNA sample according to the user manual. 2 ul of lysate was added to each reaction and Taqman probes (human): AAVR: FAM Hs00967343_m1 and Act-B: VIC Hs01060665_g1 were used. Delta Delta Ct analysis was conducted to find fold change in relation to the untreated control samples.

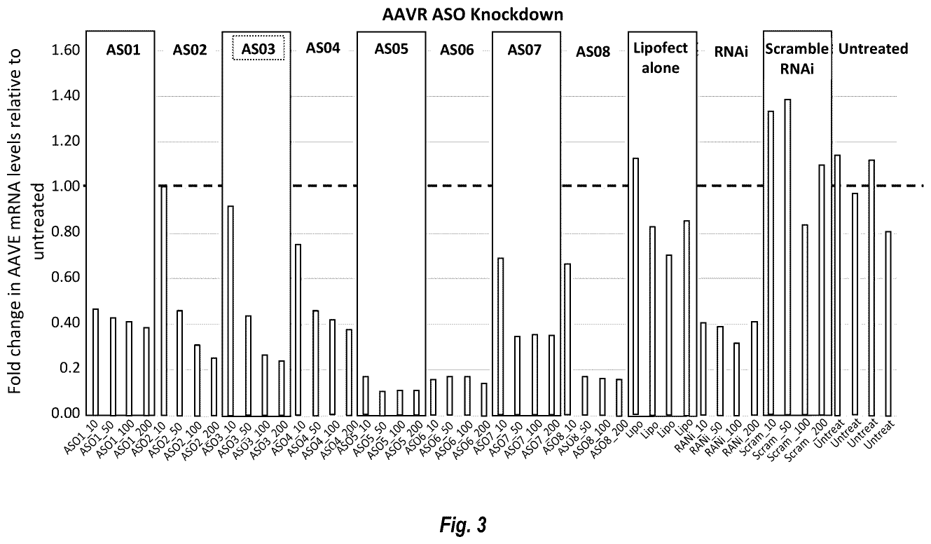

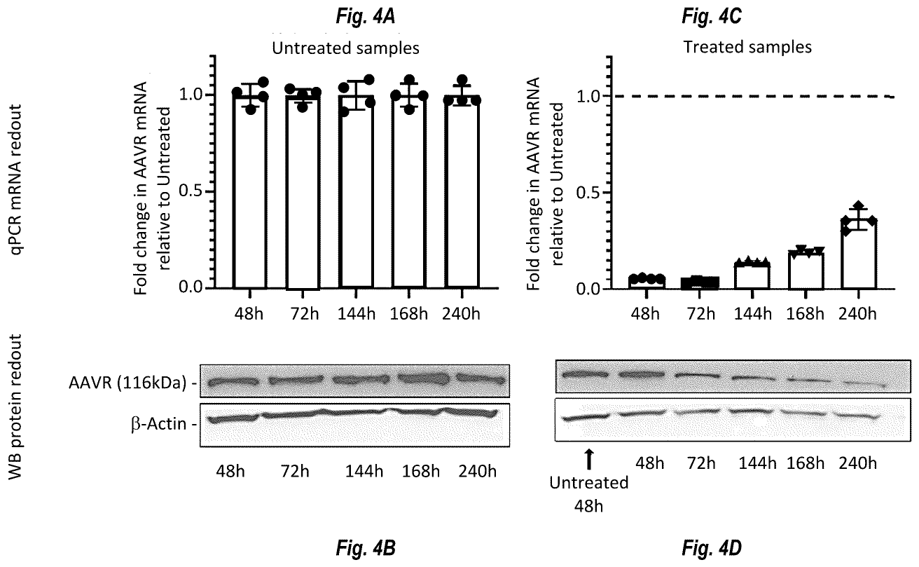

FIG. 3 shows the effect of AAVR knockdown by 7 different ASOs as compared to untreated Huh7 cells and negative and positive controls.

d) ASO Effect on AAVR mRNA and Protein in Liver Cell Lines

Cell treatment and harvesting: Samples were collected from 12-well plates. Cells were seeded at 15 k per well and grown overnight at 37° C. with 5% CO2. Cells were transfected with ASO (50 nM) or siRNA (10 nM) using RNAiMax Lipofectamine (Invitrogen 13778150), according to manufacturer procedure. Media was changed at 48 h and 168 h post dosing.

Samples were harvested at each time point. Cells were washed with DPBS and incubated with TrypLE™ Express Enzyme for 5 min in 37° C. with 5% CO2. Once detached, TrypLE was neutralized with DMEM and cells were transferred to a tube and pelleted for 5 min at 1000 rpm. Media was removed and cells were resuspended in 1 mL DPBS. 100 uL was moved to a new tube. Both new and original tubes were pelleted and DPBS was removed. A 100 uL tube was used for assessing mRNA, and a 900 uL tube was used for assessing protein, as further described below. Cell pellets were stored at −80° C. until processing.

Western blotting (protein): Pellets were resuspended in ice cold extraction buffer: RIPA buffer supplemented with cOmplete™, Mini Protease Inhibitor Cocktail (Roche). Protein extracts were pre-cleared (4 C at 14,000 G for 15 min). The protein levels were measured by Pierce BCA assay (ThermoFisher, 23227) following manufacturer's protocol (assets.thermofisher.com/TFS-Assets/LSG/manuals/MAN0011430_Pierce_BCA_Protein_Asy_UG.pdf). All samples were diluted with extraction buffer to 0.33 mg/mL or the lowest sample concentration.

4-12% Bis-Tris plus gels (Invitrogen #NW04120BOX) were loaded with 10 ug protein extract mixed with loading buffer. Gels were run in MOPS buffer at +4 C and the proteins were transferred to the nitrocellulose (Trans-Blot Transfer Pack, 1704158, Bio-Rad) membrane using transfer set and dry transfer apparatus (high molecular weight settings on Trans-Blot Turbo Transfer System, Bio-Rad). Transfer was checked using Ponceau stain following manufacturer's protocol (Thermos Scientific, J63139), membranes were cut between 50-75 kDA markers and blocked in 5% milk overnight at 4° C.

Membranes were washed with TBST and incubated with primary antibodies for either AAVR (1:2000; 21016-1-AP, Proteintech) or B-actin detection (1:1000; 13E5, Cell Signaling), for 4 hours at room temperature. Next samples were washed with TBST and incubated with HRP-conjugated secondary antibody (1:200,000; 31460, Thermofisher) for 1 hour at room temperature, washed three times with TBST and visualized with ultra-sensitive enhanced chemiluminescent substrate (SuperSignal™ West Femto Maximum Sensitivity Substrate, 34095, Thermofisher).

mRNA qPCR: Cells were lysed using lysis buffer from Cells-to-CT 1-step TaqMan Kit (Invitrogen A25603). qPCR was run on Quantstudio 6 flex using mastermix from Cells-to-CT 1-step TaqMan Kit (Invitrogen A25603). 20 ul reactions were conducted in technical triplicates for each RNA sample according to the user manual. 2 ul of lysate was added to each reaction and Taqman probes (human): AAVR: FAM Hs00967343_m1 and Act-B: VIC Hs01060665_g1 were used. Delta Delta Ct analysis was conducted to find fold change in relation to untreated control samples.

### 2. Example 2: In Vitro Proof of Concept Study

The effects of AAVR knockdown on AAV transduction efficiency were evaluated in vitro. Huh7 cells were preconditioned with siRNA or ASO or left untreated. After 48 hours, cells were infected with AAV9 carrying a pCMV-GFP transgene. Relative transduction efficiency between treated or untreated cells was evaluated by fluorescence microscopy 72 hours after infection.

Cells were seeded in 96 well plates and transfected with AAVR-targeting siRNA or ASO using RNAiMax Lipofectamine (Invitrogen 13778150) according to manufacturer procedure. The dose for treatment varied between 2.5-250 nM per modality of choice. Media was changed 48 h post dosing and cells were spinfected with AAV9-GFP at 2E3 viral particles per well at 1000×g for 1.5 hours at 4 C. AAV-containing media was removed, cells were washed once with 200 uL/well of room temperature media to remove unbound virus. 150 uL/well of fresh media was added and cells were placed back to incubator and grown further at 37° C. with 5% CO2. The next day media was changed and cells were imaged 72 h post infection using wide field and FITC settings at 4×, 10× 40× magnification.

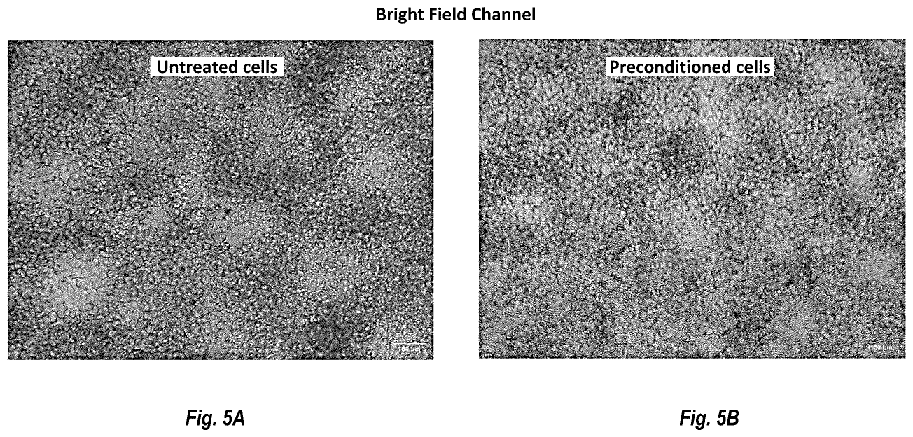

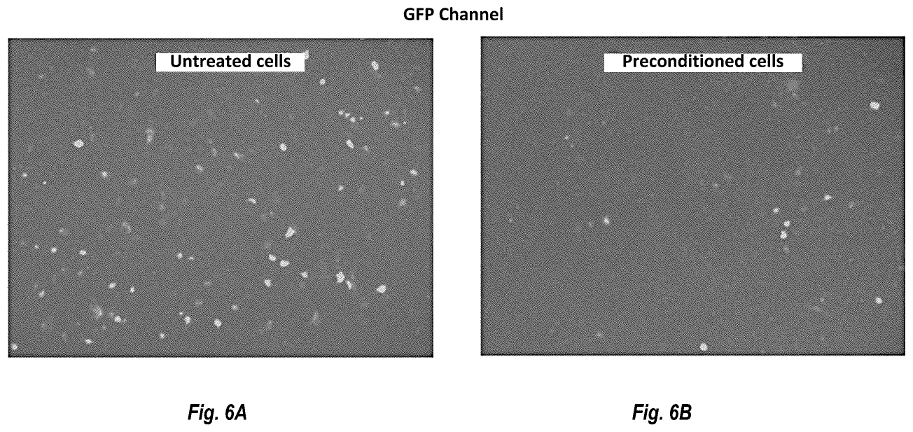

As shown in FIGS. 5A-B and 6A-B, AAVR-targeting ASO preconditioning reduced AAV9-delievered pCMV-GFP transgene expression in Huh7 cells (FIGS. 5B and 6B).

### 3. Example 3: Study of the Effect of Liver Targeted Anti-AAVR siRNA on AAV Biodistribution in Mice

To evaluate the effects of anti-AAVR siRNA administration on AAV infectivity in a mouse model, wild-type mice will be pre-conditioned with either a siRNA targeting AAVR (anti-AAVR siRNA) or a control non-targeting siRNA formulated as liver-targeted LNP or directly conjugated to a liver targeting moiety, like GalNAc. After sufficient time has passed to achieve AAVR knockdown, AAV9 encoding green fluorescent protein (GFP) driven by a ubiquitous promoter (CMV or similar) will be administered systemically.

After allowing time for robust GFP expression, mice will be sacrificed, and a panel of tissues will be harvested for DNA extraction and histology analysis to characterize AAV biodistribution and quantify vector genomes (VG) per cell in those tissues. GFP expression and vector genome quantification in the liver as compared to various muscle and CNS tissues and other organs will be assessed. The timing and dose of siRNA and AAV administration will be guided by pilot dose-ranging studies.

Tissues from anti-AAVR and control siRNA treated mice will be compared for GFP distribution as well as vector genome number per cell.

### 4. Example 4: In Vivo Dose Range Study

An in vivo dose range study was conducted to evaluate efficacy of ASOVT002 (ASO) in 6-8 weeks old Wildtype (WT) mice for dose selection.

A single dose of ASOVT002 was administrated via intraperitoneal injection at the dose of 2, 7, and 20 mg/kg, and the mice were euthanized 7-day post injection for sample analysis. The level of AAV receptor (AAVR) protein in liver was assessed to determine initial ASO dosing.

Protein extraction (liver): The pre-portioned frozen tissues were transferred to BeadRupture tubes with ceramic beads (19-040E, BeadRupture Elite, Omni International) and submerged in RIPA buffer (ThermoFisher Scientific, 89901) or 10% SDS extraction buffer (1 mM EDTA pH8, 100 mM NaCl, 62.5 mM Tris pH 6.5, 10% SDS, 10% Glycerol, and water) supplemented with HALT Protease Inhibitor, 200 uL for 10-20 mg of frozen sample; and immediately placed into BeadRuptor homogenizer arms and homogenized for 20 s at 6 m/s, for 3 cycles (total). Lysates were spun down for 5 min at 13,000 RCF and supernatant was transferred to fresh tubes and spun down again for 15 min at 15,000 RCF to remove leftover impurities. Protein concentration was checked with Pierce BCA assay (ThermoFisher Scientific, 23227) following manufacturer's protocol and diluted to 5 mg/mL each. Samples were prepared for Jess Abby & Wes Separation module (“Jess”), following manufacturer's protocol from the SM-W004-1 kit. Primary antibody (ab105385, Abcam) was used for AAVR detection at 1:300 dilution and housekeeping gene (GAPDH, PA1987 or FASN, 3180S, from Fisher Scientific) detection at 1:100 dilution; a matching secondary antibody from manufacturer was used. Samples were run using 24-well cartridges using kits for Jess. The data were acquired using the capillary gel electrophoresis system ProteinSimple “Jess” and analyzed using Compass for Simple Western software.

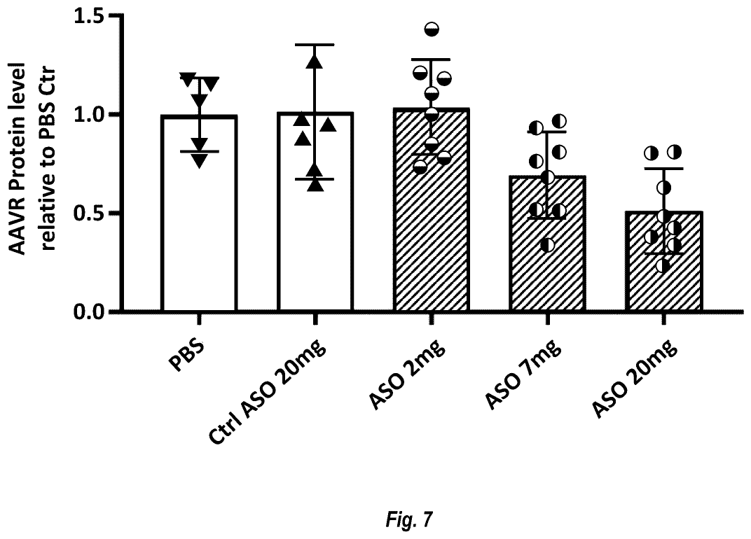

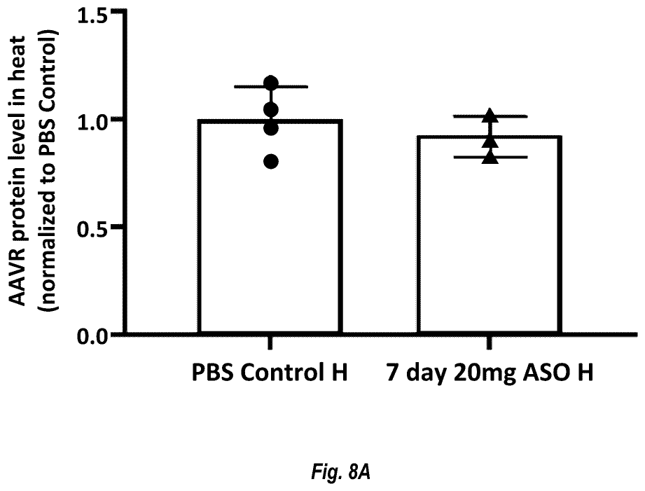

As shown in FIG. 7, AAV receptor protein was quantified 7 days after dosing with AAVR-targeting ASO at 2, 7 or 20 mg/kg doses, 20 mg/kg of control ASO, or PBS. The 20 mg dose of ASO VT002 induced about 50% knockdown of AAVR protein 7 days post treatment.

Protein extraction (heart and muscle): The pre-portioned frozen tissues were transferred to BeadRupture tubes with ceramic beads (19-040E, BeadRupture Elite, Omni International) and submerged in 10% SDS extraction buffer (1 mM EDTA pH8, 100 mM NaCl, 62.5 mM Tris pH 6.5, 10% SDS, 10% Glycerol, and water) supplemented with HALT Protease Inhibitor, 200 uL for 10-20 mg of frozen sample; and immediately placed into BeadRuptor homogenizer arms and homogenized for 20 s at 6 m/s, for 3 cycles (total). Lysates were spun down for 5 min at 13,000 RCF and supernatant was transferred to fresh tubes and spiun down again for 15 min at 15,000 RCF to remove leftover impurities. Protein concentration was checked with Pierce BCA assay (ThermoFisher Scientific, 23227) following manufacturer's protocol and diluted to 5 mg/mL each. Samples were prepared for Jess Abby & Wes Separation module (“Jess”), following manufacturer's protocol from SM-W004-1 kit. Primary antibody (ab105385, Abcam) was used for AAVR detection at 1:300 dilution and housekeeping gene (GAPDH, PA1987 Fisher Scientific) detection at 1:50 dilution; a matching secondary antibody from manufacturer was used. Samples were run using 24-well cartridges using kits for Jess. The data were acquired using the capillary gel electrophoresis system ProteinSimple “Jess” and analyzed using Compass for Simple Western software.

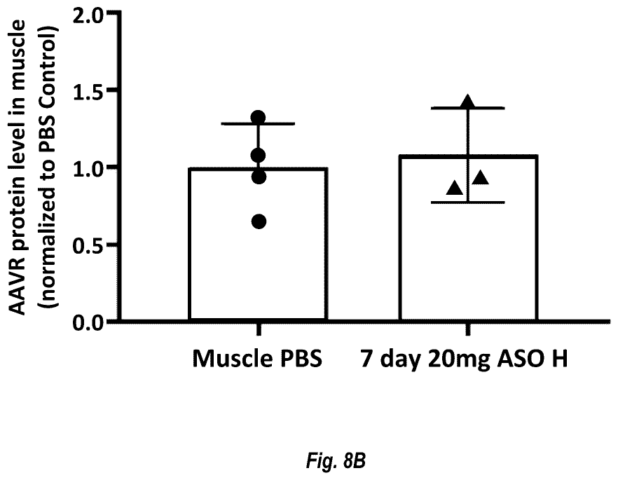

As shown in FIG. 8, AAV receptor protein was quantified 7 days after dosing with AAVR-targeting ASO at 20 mg/kg dose or PBS (control) treated animal. AAVR-targeting ASO treatment did not influence AAVR protein expression in heart and muscle tissues.

### 5. Example 5: In Vivo Kinetic Study

An in vivo study was conducted to determine AAVR protein knockdown kinetics. The kinetics of ASOVT002 was evaluated in 6-8 weeks old Wildtype (WT) mice for timepoint selection. A single dose of ASOVT002 was administrated via intraperitoneal injection at a dose of 20 mg/kg, and the mice were euthanized at 3-, 7-, or 10-day post injection for sample analysis. The level of AAV receptor (AAVR) protein in liver at different timepoints was assessed via the capillary gel electrophoresis system Jess.

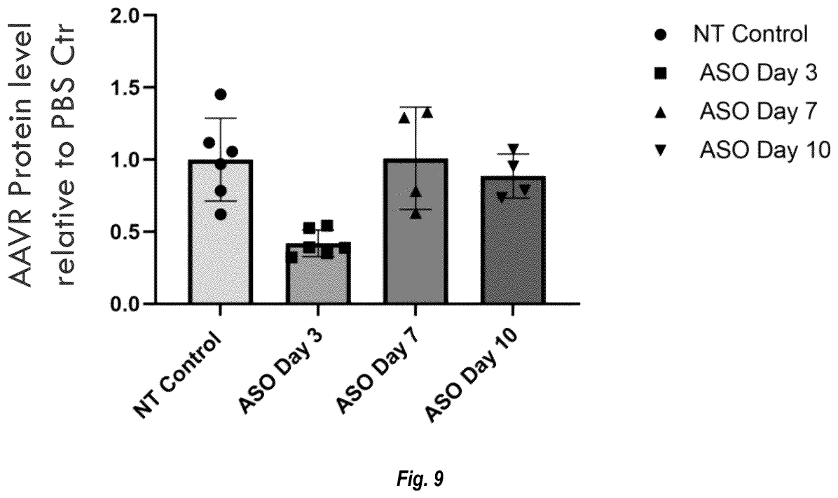

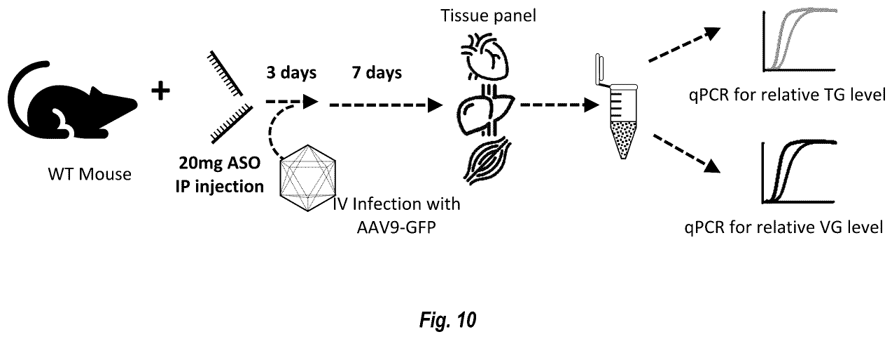

Protein extraction (liver): The pre-portioned frozen tissues were transferred to BeadRupture tubes with ceramic beads (19-040E, BeadRupture Elite, Omni International) and submerged in 10% SDS extraction buffer (1 mM EDTA pH8, 100 mM NaCl, 62.5 mM Tris pH 6.5, 10% SDS, 10% Glycerol, and water) supplemented with HALT Protease Inhibitor, 200 uL for 10-20 mg of frozen sample; and immediately placed into BeadRuptor homogenizer arms and homogenized for 20 s at 6 m/s, for 3 cycles (total). Lysates were spun down for 5 min at 13,000 RCF. The supernatant was transferred to a fresh tube and spun down again for 15 min at 15,000 RCF to remove leftover impurities. Next, protein concentration was checked with Pierce BCA assay (ThermoFisher Scientific, 23227) according to the manufacturer's protocol and diluted to 5 mg/mL each. Next, samples were prepared for Jess Abby & Wes Separation module (“Jess”), according to manufacturer protocol from SM-W004-1 kit. Primary antibody (ab105385, Abcam) was used for AAVR detection at 1:300 dilution and housekeeping gene (FASN, 3180S, from Fisher Scientific) detection at 1:50 dilution; with a matching secondary antibody from manufacturer. Samples were run using 24-well cartridges using kits for Jess. The data were acquired using the capillary gel electrophoresis system ProteinSimple “Jess” and analyzed using Compass for Simple Western software. As shown in FIG. 9, AAV receptor protein was quantified after 3, 7, and 10 days post-dosing with AAVR ASO at 20 mg/kg or PBS (control) treatment.

### 6. Example 6: Week-Long Conditioning Study

This study was designed to evaluate the AAV9-CMV-EGFP expression after the knockdown of AAVR in liver by ASO. At day 0, the ASOVT002 was intraperitoneally injected to 6-8 week-old wildtype (WT) mice at a dose of 20 mg/kg, followed by an intravenous injection of AAV9-CMV-EGFP, at the dose of 2×10¹² (vg/kg) at day 3 and euthanasia at day 10 of the study (7-days post AAV9-CMV-EGFP dosing). Tissues were collected for RNA and DNA. AAV9-CMV-EGFP vector genome and transgene expression was assessed by qPCR and qRT-PCR, where AAVR protein and mRNA expression in liver, skeletal muscle and heart was assessed by Jess and qRT-PCR, respectively.

TABLE 3
Additional siRNA Sequences

| SEQ ID | siRNA sequence antisense_ | SEQ ID | siRNA sequence antisense_ |
|---|---|---|---|
| NO | 5′ to 3′_rna | NO | 5′ to 3′ rna |
| 4300 | CAGUUAAAUACCAUGUUUAAU | 6904 | UUAGCAUUCUCGAGCUGCACC |
| 4301 | AGUUAAAUACCAUGUUUAAUC | 6905 | UAGCAUUCUCGAGCUGCACCC |
| 4302 | GUUAAAUACCAUGUUUAAUCA | 6906 | AGCAUUCUCGAGCUGCACCCC |
| 4303 | UUAAAUACCAUGUUUAAUCAU | 6907 | GCAUUCUCGAGCUGCACCCCA |
| 4304 | UAAAUACCAUGUUUAAUCAUC | 6908 | CAUUCUCGAGCUGCACCCCAU |
| 4305 | AAAUACCAUGUUUAAUCAUCC | 6909 | AUUCUCGAGCUGCACCCCAUC |
| 4306 | AAUACCAUGUUUAAUCAUCCC | 6910 | UUCUCGAGCUGCACCCCAUCA |
| 4307 | AUACCAUGUUUAAUCAUCCCA | 6911 | UCUCGAGCUGCACCCCAUCAG |
| 4308 | UACCAUGUUUAAUCAUCCCAC | 6912 | CUCGAGCUGCACCCCAUCAGG |
| 4309 | ACCAUGUUUAAUCAUCCCACA | 6913 | UCGAGCUGCACCCCAUCAGGU |
| 4310 | CCAUGUUUAAUCAUCCCACAA | 6914 | CGAGCUGCACCCCAUCAGGUC |
| 4311 | CAUGUUUAAUCAUCCCACAAU | 6915 | GAGCUGCACCCCAUCAGGUCC |
| 4312 | AUGUUUAAUCAUCCCACAAUG | 6916 | AGCUGCACCCCAUCAGGUCCC |
| 4313 | UGUUUAAUCAUCCCACAAUGG | 6917 | GCUGCACCCCAUCAGGUCCCU |
| 4314 | GUUUAAUCAUCCCACAAUGGA | 6918 | CUGCACCCCAUCAGGUCCCUG |
| 4315 | UUUAAUCAUCCCACAAUGGAC | 6919 | UGCACCCCAUCAGGUCCCUGU |
| 4316 | UUAAUCAUCCCACAAUGGACA | 6920 | GCACCCCAUCAGGUCCCUGUG |
| 4317 | UAAUCAUCCCACAAUGGACAG | 6921 | CACCCCAUCAGGUCCCUGUGU |
| 4318 | AAUCAUCCCACAAUGGACAGU | 6922 | ACCCCAUCAGGUCCCUGUGUU |
| 4319 | AUCAUCCCACAAUGGACAGUA | 6923 | CCCCAUCAGGUCCCUGUGUUU |
| 4320 | UCAUCCCACAAUGGACAGUAG | 6924 | CCCAUCAGGUCCCUGUGUUUU |
| 4321 | CAUCCCACAAUGGACAGUAGU | 6925 | CCAUCAGGUCCCUGUGUUUUU |
| 4322 | AUCCCACAAUGGACAGUAGUG | 6926 | CAUCAGGUCCCUGUGUUUUUU |
| 4323 | UCCCACAAUGGACAGUAGUGC | 6927 | AUCAGGUCCCUGUGUUUUUUC |
| 4324 | CCCACAAUGGACAGUAGUGCA | 6928 | UCAGGUCCCUGUGUUUUUUCC |
| 4325 | CCACAAUGGACAGUAGUGCAG | 6929 | CAGGUCCCUGUGUUUUUUCCC |
| 4326 | CACAAUGGACAGUAGUGCAGU | 6930 | AGGUCCCUGUGUUUUUUCCCA |
| 4327 | ACAAUGGACAGUAGUGCAGUA | 6931 | GGUCCCUGUGUUUUUUCCCAG |
| 4328 | CAAUGGACAGUAGUGCAGUAG | 6932 | GUCCCUGUGUUUUUUCCCAGA |
| 4329 | AAUGGACAGUAGUGCAGUAGU | 6933 | UCCCUGUGUUUUUUCCCAGAG |
| 4330 | AUGGACAGUAGUGCAGUAGUA | 6934 | CCCUGUGUUUUUUCCCAGAGA |
| 4331 | UGGACAGUAGUGCAGUAGUAG | 6935 | CCUGUGUUUUUUCCCAGAGAU |
| 4332 | GGACAGUAGUGCAGUAGUAGA | 6936 | CUGUGUUUUUUCCCAGAGAUA |
| 4333 | GACAGUAGUGCAGUAGUAGAA | 6937 | UGUGUUUUUUCCCAGAGAUAU |
| 4334 | ACAGUAGUGCAGUAGUAGAAA | 6938 | GUGUUUUUUCCCAGAGAUAUG |
| 4335 | CAGUAGUGCAGUAGUAGAAAU | 6939 | UGUUUUUUCCCAGAGAUAUGA |
| 4336 | AGUAGUGCAGUAGUAGAAAUU | 6940 | GUUUUUUCCCAGAGAUAUGAG |
| 4337 | GUAGUGCAGUAGUAGAAAUUA | 6941 | UUUUUUCCCAGAGAUAUGAGA |
| 4338 | UAGUGCAGUAGUAGAAAUUAU | 6942 | UUUUUCCCAGAGAUAUGAGAU |
| 4339 | AGUGCAGUAGUAGAAAUUAUA | 6943 | UUUUCCCAGAGAUAUGAGAUA |
| 4340 | GUGCAGUAGUAGAAAUUAUAA | 6944 | UUUCCCAGAGAUAUGAGAUAA |
| 4341 | UGCAGUAGUAGAAAUUAUAAG | 6945 | UUCCCAGAGAUAUGAGAUAAU |
| 4342 | GCAGUAGUAGAAAUUAUAAGU | 6946 | UCCCAGAGAUAUGAGAUAAUU |
| 4343 | CAGUAGUAGAAAUUAUAAGUG | 6947 | CCCAGAGAUAUGAGAUAAUUU |
| 4344 | AGUAGUAGAAAUUAUAAGUGU | 6948 | CCAGAGAUAUGAGAUAAUUUU |
| 4345 | GUAGUAGAAAUUAUAAGUGUC | 6949 | CAGAGAUAUGAGAUAAUUUUC |
| 4346 | UAGUAGAAAUUAUAAGUGUCU | 6950 | AGAGAUAUGAGAUAAUUUUCU |
| 4347 | AGUAGAAAUUAUAAGUGUCUC | 6951 | GAGAUAUGAGAUAAUUUUCUG |
| 4348 | GUAGAAAUUAUAAGUGUCUCU | 6952 | AGAUAUGAGAUAAUUUUCUGA |
| 4349 | UAGAAAUUAUAAGUGUCUCUU | 6953 | GAUAUGAGAUAAUUUUCUGAU |
| 4350 | AGAAAUUAUAAGUGUCUCUUU | 6954 | AUAUGAGAUAAUUUUCUGAUC |
| 4351 | GAAAUUAUAAGUGUCUCUUUA | 6955 | UAUGAGAUAAUUUUCUGAUCA |
| 4352 | AAAUUAUAAGUGUCUCUUUAA | 6956 | AUGAGAUAAUUUUCUGAUCAU |
| 4353 | AAUUAUAAGUGUCUCUUUAAA | 6957 | UGAGAUAAUUUUCUGAUCAUC |
| 4354 | AUUAUAAGUGUCUCUUUAAAA | 6958 | GAGAUAAUUUUCUGAUCAUCU |
| 4355 | UUAUAAGUGUCUCUUUAAAAA | 6959 | AGAUAAUUUUCUGAUCAUCUG |
| 4356 | UAUAAGUGUCUCUUUAAAAAG | 6960 | GAUAAUUUUCUGAUCAUCUGA |
| 4357 | AUAAGUGUCUCUUUAAAAAGU | 6961 | AUAAUUUUCUGAUCAUCUGAG |
| 4358 | UAAGUGUCUCUUUAAAAAGUC | 6962 | UAAUUUUCUGAUCAUCUGAGC |
| 4359 | AAGUGUCUCUUUAAAAAGUCA | 6963 | AAUUUUCUGAUCAUCUGAGCU |
| 4360 | AGUGUCUCUUUAAAAAGUCAC | 6964 | AUUUUCUGAUCAUCUGAGCUC |
| 4361 | GUGUCUCUUUAAAAAGUCACU | 6965 | UUUUCUGAUCAUCUGAGCUCU |
| 4362 | UGUCUCUUUAAAAAGUCACUC | 6966 | UUUCUGAUCAUCUGAGCUCUU |
| 4363 | GUCUCUUUAAAAAGUCACUCC | 6967 | UUCUGAUCAUCUGAGCUCUUG |
| 4364 | UCUCUUUAAAAAGUCACUCCC | 6968 | UCUGAUCAUCUGAGCUCUUGC |
| 4365 | CUCUUUAAAAAGUCACUCCCC | 6969 | CUGAUCAUCUGAGCUCUUGCU |
| 4366 | UCUUUAAAAAGUCACUCCCCC | 6970 | UGAUCAUCUGAGCUCUUGCUG |
| 4367 | CUUUAAAAAGUCACUCCCCCA | 6971 | GAUCAUCUGAGCUCUUGCUGC |
| 4368 | UUUAAAAAGUCACUCCCCCAA | 6972 | AUCAUCUGAGCUCUUGCUGCC |
| 4369 | UUAAAAAGUCACUCCCCCAAA | 6973 | UCAUCUGAGCUCUUGCUGCCA |
| 4370 | UAAAAAGUCACUCCCCCAAAG | 6974 | CAUCUGAGCUCUUGCUGCCAU |
| 4371 | AAAAAGUCACUCCCCCAAAGU | 6975 | AUCUGAGCUCUUGCUGCCAUC |
| 4372 | AAAAGUCACUCCCCCAAAGUU | 6976 | UCUGAGCUCUUGCUGCCAUCC |
| 4373 | AAAGUCACUCCCCCAAAGUUU | 6977 | CUGAGCUCUUGCUGCCAUCCA |
| 4374 | AAGUCACUCCCCCAAAGUUUC | 6978 | UGAGCUCUUGCUGCCAUCCAG |
| 4375 | AGUCACUCCCCCAAAGUUUCC | 6979 | GAGCUCUUGCUGCCAUCCAGG |
| 4376 | GUCACUCCCCCAAAGUUUCCA | 6980 | AGCUCUUGCUGCCAUCCAGGG |
| 4377 | UCACUCCCCCAAAGUUUCCAU | 6981 | GCUCUUGCUGCCAUCCAGGGU |
| 4378 | CACUCCCCCAAAGUUUCCAUU | 6982 | CUCUUGCUGCCAUCCAGGGUU |
| 4379 | ACUCCCCCAAAGUUUCCAUUC | 6983 | UCUUGCUGCCAUCCAGGGUUG |
| 4380 | CUCCCCCAAAGUUUCCAUUCC | 6984 | CUUGCUGCCAUCCAGGGUUGU |
| 4381 | UCCCCCAAAGUUUCCAUUCCC | 6985 | UUGCUGCCAUCCAGGGUUGUG |
| 4382 | CCCCCAAAGUUUCCAUUCCCC | 6986 | UGCUGCCAUCCAGGGUUGUGC |
| 4383 | CCCCAAAGUUUCCAUUCCCCA | 6987 | GCUGCCAUCCAGGGUUGUGCU |
| 4384 | CCCAAAGUUUCCAUUCCCCAA | 6988 | CUGCCAUCCAGGGUUGUGCUA |
| 4385 | CCAAAGUUUCCAUUCCCCAAU | 6989 | UGCCAUCCAGGGUUGUGCUAU |
| 4386 | CAAAGUUUCCAUUCCCCAAUA | 6990 | GCCAUCCAGGGUUGUGCUAUC |
| 4387 | AAAGUUUCCAUUCCCCAAUAC | 6991 | CCAUCCAGGGUUGUGCUAUCC |
| 4388 | AAGUUUCCAUUCCCCAAUACC | 6992 | CAUCCAGGGUUGUGCUAUCCA |
| 4389 | AGUUUCCAUUCCCCAAUACCC | 6993 | AUCCAGGGUUGUGCUAUCCAC |
| 4390 | GUUUCCAUUCCCCAAUACCCA | 6994 | UCCAGGGUUGUGCUAUCCACA |
| 4391 | UUUCCAUUCCCCAAUACCCAG | 6995 | CCAGGGUUGUGCUAUCCACAG |
| 4392 | UUCCAUUCCCCAAUACCCAGA | 6996 | CAGGGUUGUGCUAUCCACAGG |
| 4393 | UCCAUUCCCCAAUACCCAGAU | 6997 | AGGGUUGUGCUAUCCACAGGA |
| 4394 | CCAUUCCCCAAUACCCAGAUA | 6998 | GGGUUGUGCUAUCCACAGGAA |
| 4395 | CAUUCCCCAAUACCCAGAUAA | 6999 | GGUUGUGCUAUCCACAGGAAG |
| 4396 | AUUCCCCAAUACCCAGAUAAC | 7000 | GUUGUGCUAUCCACAGGAAGG |
| 4397 | UUCCCCAAUACCCAGAUAACC | 7001 | UUGUGCUAUCCACAGGAAGGG |
| 4398 | UCCCCAAUACCCAGAUAACCA | 7002 | UGUGCUAUCCACAGGAAGGGU |
| 4399 | CCCCAAUACCCAGAUAACCAU | 7003 | GUGCUAUCCACAGGAAGGGUC |
| 4400 | CCCAAUACCCAGAUAACCAUG | 7004 | UGCUAUCCACAGGAAGGGUCA |
| 4401 | CCAAUACCCAGAUAACCAUGG | 7005 | GCUAUCCACAGGAAGGGUCAG |
| 4402 | CAAUACCCAGAUAACCAUGGU | 7006 | CUAUCCACAGGAAGGGUCAGC |
| 4403 | AAUACCCAGAUAACCAUGGUG | 7007 | UAUCCACAGGAAGGGUCAGCU |
| 4404 | AUACCCAGAUAACCAUGGUGG | 7008 | AUCCACAGGAAGGGUCAGCUC |
| 4405 | UACCCAGAUAACCAUGGUGGA | 7009 | UCCACAGGAAGGGUCAGCUCU |
| 4406 | ACCCAGAUAACCAUGGUGGAA | 7010 | CCACAGGAAGGGUCAGCUCUU |
| 4407 | CCCAGAUAACCAUGGUGGAAC | 7011 | CACAGGAAGGGUCAGCUCUUU |
| 4408 | CCAGAUAACCAUGGUGGAACA | 7012 | ACAGGAAGGGUCAGCUCUUUA |
| 4409 | CAGAUAACCAUGGUGGAACAG | 7013 | CAGGAAGGGUCAGCUCUUUAU |
| 4410 | AGAUAACCAUGGUGGAACAGG | 7014 | AGGAAGGGUCAGCUCUUUAUC |
| 4411 | GAUAACCAUGGUGGAACAGGG | 7015 | GGAAGGGUCAGCUCUUUAUCU |
| 4412 | AUAACCAUGGUGGAACAGGGC | 7016 | GAAGGGUCAGCUCUUUAUCUG |
| 4413 | UAACCAUGGUGGAACAGGGCC | 7017 | AAGGGUCAGCUCUUUAUCUGG |
| 4414 | AACCAUGGUGGAACAGGGCCA | 7018 | AGGGUCAGCUCUUUAUCUGGG |
| 4415 | ACCAUGGUGGAACAGGGCCAG | 7019 | GGGUCAGCUCUUUAUCUGGGC |
| 4416 | CCAUGGUGGAACAGGGCCAGG | 7020 | GGUCAGCUCUUUAUCUGGGCC |
| 4417 | CAUGGUGGAACAGGGCCAGGG | 7021 | GUCAGCUCUUUAUCUGGGCCU |
| 4418 | AUGGUGGAACAGGGCCAGGGU | 7022 | UCAGCUCUUUAUCUGGGCCUG |
| 4419 | UGGUGGAACAGGGCCAGGGUG | 7023 | CAGCUCUUUAUCUGGGCCUGC |
| 4420 | GGUGGAACAGGGCCAGGGUGA | 7024 | AGCUCUUUAUCUGGGCCUGCA |
| 4421 | GUGGAACAGGGCCAGGGUGAG | 7025 | GCUCUUUAUCUGGGCCUGCAU |
| 4422 | UGGAACAGGGCCAGGGUGAGG | 7026 | CUCUUUAUCUGGGCCUGCAUC |
| 4423 | GGAACAGGGCCAGGGUGAGGG | 7027 | UCUUUAUCUGGGCCUGCAUCU |
| 4424 | GAACAGGGCCAGGGUGAGGGG | 7028 | CUUUAUCUGGGCCUGCAUCUG |
| 4425 | AACAGGGCCAGGGUGAGGGGG | 7029 | UUUAUCUGGGCCUGCAUCUGC |
| 4426 | ACAGGGCCAGGGUGAGGGGGA | 7030 | UUAUCUGGGCCUGCAUCUGCC |
| 4427 | CAGGGCCAGGGUGAGGGGGAC | 7031 | UAUCUGGGCCUGCAUCUGCCU |
| 4428 | AGGGCCAGGGUGAGGGGGACU | 7032 | AUCUGGGCCUGCAUCUGCCUG |
| 4429 | GGGCCAGGGUGAGGGGGACUC | 7033 | UCUGGGCCUGCAUCUGCCUGA |
| 4430 | GGCCAGGGUGAGGGGGACUCA | 7034 | CUGGGCCUGCAUCUGCCUGAG |
| 4431 | GCCAGGGUGAGGGGGACUCAG | 7035 | UGGGCCUGCAUCUGCCUGAGG |
| 4432 | CCAGGGUGAGGGGGACUCAGG | 7036 | GGGCCUGCAUCUGCCUGAGGA |
| 4433 | CAGGGUGAGGGGGACUCAGGA | 7037 | GGCCUGCAUCUGCCUGAGGAG |
| 4434 | AGGGUGAGGGGGACUCAGGAA | 7038 | GCCUGCAUCUGCCUGAGGAGG |
| 4435 | GGGUGAGGGGGACUCAGGAAG | 7039 | CCUGCAUCUGCCUGAGGAGGC |
| 4436 | GGUGAGGGGGACUCAGGAAGU | 7040 | CUGCAUCUGCCUGAGGAGGCU |
| 4437 | GUGAGGGGGACUCAGGAAGUC | 7041 | UGCAUCUGCCUGAGGAGGCUU |
| 4438 | UGAGGGGGACUCAGGAAGUCU | 7042 | GCAUCUGCCUGAGGAGGCUUA |
| 4439 | GAGGGGGACUCAGGAAGUCUU | 7043 | CAUCUGCCUGAGGAGGCUUAU |
| 4440 | AGGGGGACUCAGGAAGUCUUG | 7044 | AUCUGCCUGAGGAGGCUUAUU |
| 4441 | GGGGGACUCAGGAAGUCUUGG | 7045 | UCUGCCUGAGGAGGCUUAUUG |
| 4442 | GGGGACUCAGGAAGUCUUGGU | 7046 | CUGCCUGAGGAGGCUUAUUGU |
| 4443 | GGGACUCAGGAAGUCUUGGUU | 7047 | UGCCUGAGGAGGCUUAUUGUU |
| 4444 | GGACUCAGGAAGUCUUGGUUC | 7048 | GCCUGAGGAGGCUUAUUGUUU |
| 4445 | GACUCAGGAAGUCUUGGUUCC | 7049 | CCUGAGGAGGCUUAUUGUUUU |
| 4446 | ACUCAGGAAGUCUUGGUUCCA | 7050 | CUGAGGAGGCUUAUUGUUUUC |
| 4447 | CUCAGGAAGUCUUGGUUCCAA | 7051 | UGAGGAGGCUUAUUGUUUUCA |
| 4448 | UCAGGAAGUCUUGGUUCCAAG | 7052 | GAGGAGGCUUAUUGUUUUCAG |
| 4449 | CAGGAAGUCUUGGUUCCAAGG | 7053 | AGGAGGCUUAUUGUUUUCAGG |
| 4450 | AGGAAGUCUUGGUUCCAAGGA | 7054 | GGAGGCUUAUUGUUUUCAGGU |
| 4451 | GGAAGUCUUGGUUCCAAGGAG | 7055 | GAGGCUUAUUGUUUUCAGGUU |
| 4452 | GAAGUCUUGGUUCCAAGGAGA | 7056 | AGGCUUAUUGUUUUCAGGUUG |
| 4453 | AAGUCUUGGUUCCAAGGAGAU | 7057 | GGCUUAUUGUUUUCAGGUUGC |
| 4454 | AGUCUUGGUUCCAAGGAGAUG | 7058 | GCUUAUUGUUUUCAGGUUGCA |
| 4455 | GUCUUGGUUCCAAGGAGAUGA | 7059 | CUUAUUGUUUUCAGGUUGCAC |
| 4456 | UCUUGGUUCCAAGGAGAUGAA | 7060 | UUAUUGUUUUCAGGUUGCACA |
| 4457 | CUUGGUUCCAAGGAGAUGAAA | 7061 | UAUUGUUUUCAGGUUGCACAA |
| 4458 | UUGGUUCCAAGGAGAUGAAAG | 7062 | AUUGUUUUCAGGUUGCACAAU |
| 4459 | UGGUUCCAAGGAGAUGAAAGU | 7063 | UUGUUUUCAGGUUGCACAAUA |
| 4460 | GGUUCCAAGGAGAUGAAAGUC | 7064 | UGUUUUCAGGUUGCACAAUAA |
| 4461 | GUUCCAAGGAGAUGAAAGUCU | 7065 | GUUUUCAGGUUGCACAAUAAC |
| 4462 | UUCCAAGGAGAUGAAAGUCUU | 7066 | UUUUCAGGUUGCACAAUAACA |
| 4463 | UCCAAGGAGAUGAAAGUCUUU | 7067 | UUUCAGGUUGCACAAUAACAG |
| 4464 | CCAAGGAGAUGAAAGUCUUUC | 7068 | UUCAGGUUGCACAAUAACAGU |
| 4465 | CAAGGAGAUGAAAGUCUUUCC | 7069 | UCAGGUUGCACAAUAACAGUC |
| 4466 | AAGGAGAUGAAAGUCUUUCCA | 7070 | CAGGUUGCACAAUAACAGUCA |
| 4467 | AGGAGAUGAAAGUCUUUCCAA | 7071 | AGGUUGCACAAUAACAGUCAC |
| 4468 | GGAGAUGAAAGUCUUUCCAAG | 7072 | GGUUGCACAAUAACAGUCACU |
| 4469 | GAGAUGAAAGUCUUUCCAAGU | 7073 | GUUGCACAAUAACAGUCACUU |
| 4470 | AGAUGAAAGUCUUUCCAAGUC | 7074 | UUGCACAAUAACAGUCACUUG |
| 4471 | GAUGAAAGUCUUUCCAAGUCA | 7075 | UGCACAAUAACAGUCACUUGA |
| 4472 | AUGAAAGUCUUUCCAAGUCAA | 7076 | GCACAAUAACAGUCACUUGAG |
| 4473 | UGAAAGUCUUUCCAAGUCAAA | 7077 | CACAAUAACAGUCACUUGAGC |
| 4474 | GAAAGUCUUUCCAAGUCAAAU | 7078 | ACAAUAACAGUCACUUGAGCA |
| 4475 | AAAGUCUUUCCAAGUCAAAUU | 7079 | CAAUAACAGUCACUUGAGCAG |
| 4476 | AAGUCUUUCCAAGUCAAAUUU | 7080 | AAUAACAGUCACUUGAGCAGU |
| 4477 | AGUCUUUCCAAGUCAAAUUUG | 7081 | AUAACAGUCACUUGAGCAGUG |
| 4478 | GUCUUUCCAAGUCAAAUUUGC | 7082 | UAACAGUCACUUGAGCAGUGG |
| 4479 | UCUUUCCAAGUCAAAUUUGCC | 7083 | AACAGUCACUUGAGCAGUGGC |
| 4480 | CUUUCCAAGUCAAAUUUGCCU | 7084 | ACAGUCACUUGAGCAGUGGCC |
| 4481 | UUUCCAAGUCAAAUUUGCCUU | 7085 | CAGUCACUUGAGCAGUGGCCU |
| 4482 | UUCCAAGUCAAAUUUGCCUUG | 7086 | AGUCACUUGAGCAGUGGCCUG |
| 4483 | UCCAAGUCAAAUUUGCCUUGU | 7087 | GUCACUUGAGCAGUGGCCUGC |
| 4484 | CCAAGUCAAAUUUGCCUUGUG | 7088 | UCACUUGAGCAGUGGCCUGCU |
| 4485 | CAAGUCAAAUUUGCCUUGUGA | 7089 | CACUUGAGCAGUGGCCUGCUG |
| 4486 | AAGUCAAAUUUGCCUUGUGAG | 7090 | ACUUGAGCAGUGGCCUGCUGU |
| 4487 | AGUCAAAUUUGCCUUGUGAGC | 7091 | CUUGAGCAGUGGCCUGCUGUC |
| 4488 | GUCAAAUUUGCCUUGUGAGCA | 7092 | UUGAGCAGUGGCCUGCUGUCC |
| 4489 | UCAAAUUUGCCUUGUGAGCAG | 7093 | UGAGCAGUGGCCUGCUGUCCU |
| 4490 | CAAAUUUGCCUUGUGAGCAGC | 7094 | GAGCAGUGGCCUGCUGUCCUA |
| 4491 | AAAUUUGCCUUGUGAGCAGCC | 7095 | AGCAGUGGCCUGCUGUCCUAU |
| 4492 | AAUUUGCCUUGUGAGCAGCCA | 7096 | GCAGUGGCCUGCUGUCCUAUU |
| 4493 | AUUUGCCUUGUGAGCAGCCAA | 7097 | CAGUGGCCUGCUGUCCUAUUG |
| 4494 | UUUGCCUUGUGAGCAGCCAAG | 7098 | AGUGGCCUGCUGUCCUAUUGU |
| 4495 | UUGCCUUGUGAGCAGCCAAGA | 7099 | GUGGCCUGCUGUCCUAUUGUG |
| 4496 | UGCCUUGUGAGCAGCCAAGAG | 7100 | UGGCCUGCUGUCCUAUUGUGU |
| 4497 | GCCUUGUGAGCAGCCAAGAGC | 7101 | GGCCUGCUGUCCUAUUGUGUC |
| 4498 | CCUUGUGAGCAGCCAAGAGCC | 7102 | GCCUGCUGUCCUAUUGUGUCA |
| 4499 | CUUGUGAGCAGCCAAGAGCCC | 7103 | CCUGCUGUCCUAUUGUGUCAG |
| 4500 | UUGUGAGCAGCCAAGAGCCCA | 7104 | CUGCUGUCCUAUUGUGUCAGU |
| 4501 | UGUGAGCAGCCAAGAGCCCAA | 7105 | UGCUGUCCUAUUGUGUCAGUC |
| 4502 | GUGAGCAGCCAAGAGCCCAAU | 7106 | GCUGUCCUAUUGUGUCAGUCA |
| 4503 | UGAGCAGCCAAGAGCCCAAUG | 7107 | CUGUCCUAUUGUGUCAGUCAC |
| 4504 | GAGCAGCCAAGAGCCCAAUGA | 7108 | UGUCCUAUUGUGUCAGUCACU |
| 4505 | AGCAGCCAAGAGCCCAAUGAG | 7109 | GUCCUAUUGUGUCAGUCACUG |
| 4506 | GCAGCCAAGAGCCCAAUGAGU | 7110 | UCCUAUUGUGUCAGUCACUGU |
| 4507 | CAGCCAAGAGCCCAAUGAGUU | 7111 | CCUAUUGUGUCAGUCACUGUG |
| 4508 | AGCCAAGAGCCCAAUGAGUUG | 7112 | CUAUUGUGUCAGUCACUGUGA |
| 4509 | GCCAAGAGCCCAAUGAGUUGA | 7113 | UAUUGUGUCAGUCACUGUGAG |
| 4510 | CCAAGAGCCCAAUGAGUUGAU | 7114 | AUUGUGUCAGUCACUGUGAGC |
| 4511 | CAAGAGCCCAAUGAGUUGAUG | 7115 | UUGUGUCAGUCACUGUGAGCU |
| 4512 | AAGAGCCCAAUGAGUUGAUGC | 7116 | UGUGUCAGUCACUGUGAGCUG |
| 4513 | AGAGCCCAAUGAGUUGAUGCA | 7117 | GUGUCAGUCACUGUGAGCUGG |
| 4514 | GAGCCCAAUGAGUUGAUGCAU | 7118 | UGUCAGUCACUGUGAGCUGGU |
| 4515 | AGCCCAAUGAGUUGAUGCAUA | 7119 | GUCAGUCACUGUGAGCUGGUA |
| 4516 | GCCCAAUGAGUUGAUGCAUAC | 7120 | UCAGUCACUGUGAGCUGGUAA |
| 4517 | CCCAAUGAGUUGAUGCAUACG | 7121 | CAGUCACUGUGAGCUGGUAAG |
| 4518 | CCAAUGAGUUGAUGCAUACGC | 7122 | AGUCACUGUGAGCUGGUAAGU |
| 4519 | CAAUGAGUUGAUGCAUACGCG | 7123 | GUCACUGUGAGCUGGUAAGUG |
| 4520 | AAUGAGUUGAUGCAUACGCGC | 7124 | UCACUGUGAGCUGGUAAGUGU |
| 4521 | AUGAGUUGAUGCAUACGCGCU | 7125 | CACUGUGAGCUGGUAAGUGUA |
| 4522 | UGAGUUGAUGCAUACGCGCUC | 7126 | ACUGUGAGCUGGUAAGUGUAG |
| 4523 | GAGUUGAUGCAUACGCGCUCA | 7127 | CUGUGAGCUGGUAAGUGUAGU |
| 4524 | AGUUGAUGCAUACGCGCUCAC | 7128 | UGUGAGCUGGUAAGUGUAGUC |
| 4525 | GUUGAUGCAUACGCGCUCACU | 7129 | GUGAGCUGGUAAGUGUAGUCU |
| 4526 | UUGAUGCAUACGCGCUCACUC | 7130 | UGAGCUGGUAAGUGUAGUCUC |
| 4527 | UGAUGCAUACGCGCUCACUCU | 7131 | GAGCUGGUAAGUGUAGUCUCC |
| 4528 | GAUGCAUACGCGCUCACUCUA | 7132 | AGCUGGUAAGUGUAGUCUCCU |
| 4529 | AUGCAUACGCGCUCACUCUAA | 7133 | GCUGGUAAGUGUAGUCUCCUU |
| 4530 | UGCAUACGCGCUCACUCUAAC | 7134 | CUGGUAAGUGUAGUCUCCUUC |
| 4531 | GCAUACGCGCUCACUCUAACA | 7135 | UGGUAAGUGUAGUCUCCUUCU |
| 4532 | CAUACGCGCUCACUCUAACAC | 7136 | GGUAAGUGUAGUCUCCUUCUU |
| 4533 | AUACGCGCUCACUCUAACACG | 7137 | GUAAGUGUAGUCUCCUUCUUG |
| 4534 | UACGCGCUCACUCUAACACGC | 7138 | UAAGUGUAGUCUCCUUCUUGC |
| 4535 | ACGCGCUCACUCUAACACGCA | 7139 | AAGUGUAGUCUCCUUCUUGCA |
| 4536 | CGCGCUCACUCUAACACGCAC | 7140 | AGUGUAGUCUCCUUCUUGCAU |
| 4537 | GCGCUCACUCUAACACGCACA | 7141 | GUGUAGUCUCCUUCUUGCAUC |
| 4538 | CGCUCACUCUAACACGCACAC | 7142 | UGUAGUCUCCUUCUUGCAUCG |
| 4539 | GCUCACUCUAACACGCACACA | 7143 | GUAGUCUCCUUCUUGCAUCGC |
| 4540 | CUCACUCUAACACGCACACAU | 7144 | UAGUCUCCUUCUUGCAUCGCA |
| 4541 | UCACUCUAACACGCACACAUG | 7145 | AGUCUCCUUCUUGCAUCGCAG |
| 4542 | CACUCUAACACGCACACAUGC | 7146 | GUCUCCUUCUUGCAUCGCAGA |
| 4543 | ACUCUAACACGCACACAUGCA | 7147 | UCUCCUUCUUGCAUCGCAGAG |
| 4544 | CUCUAACACGCACACAUGCAA | 7148 | CUCCUUCUUGCAUCGCAGAGA |
| 4545 | UCUAACACGCACACAUGCAAG | 7149 | UCCUUCUUGCAUCGCAGAGAG |
| 4546 | CUAACACGCACACAUGCAAGG | 7150 | CCUUCUUGCAUCGCAGAGAGC |
| 4547 | UAACACGCACACAUGCAAGGG | 7151 | CUUCUUGCAUCGCAGAGAGCU |
| 4548 | AACACGCACACAUGCAAGGGA | 7152 | UUCUUGCAUCGCAGAGAGCUG |
| 4549 | ACACGCACACAUGCAAGGGAC | 7153 | UCUUGCAUCGCAGAGAGCUGU |
| 4550 | CACGCACACAUGCAAGGGACA | 7154 | CUUGCAUCGCAGAGAGCUGUA |
| 4551 | ACGCACACAUGCAAGGGACAC | 7155 | UUGCAUCGCAGAGAGCUGUAA |
| 4552 | CGCACACAUGCAAGGGACACA | 7156 | UGCAUCGCAGAGAGCUGUAAG |
| 4553 | GCACACAUGCAAGGGACACAG | 7157 | GCAUCGCAGAGAGCUGUAAGG |
| 4554 | CACACAUGCAAGGGACACAGA | 7158 | CAUCGCAGAGAGCUGUAAGGU |
| 4555 | ACACAUGCAAGGGACACAGAA | 7159 | AUCGCAGAGAGCUGUAAGGUU |
| 4556 | CACAUGCAAGGGACACAGAAC | 7160 | UCGCAGAGAGCUGUAAGGUUG |
| 4557 | ACAUGCAAGGGACACAGAACA | 7161 | CGCAGAGAGCUGUAAGGUUGG |
| 4558 | CAUGCAAGGGACACAGAACAC | 7162 | GCAGAGAGCUGUAAGGUUGGU |
| 4559 | AUGCAAGGGACACAGAACACA | 7163 | CAGAGAGCUGUAAGGUUGGUG |
| 4560 | UGCAAGGGACACAGAACACAU | 7164 | AGAGAGCUGUAAGGUUGGUGU |
| 4561 | GCAAGGGACACAGAACACAUA | 7165 | GAGAGCUGUAAGGUUGGUGUU |
| 4562 | CAAGGGACACAGAACACAUAC | 7166 | AGAGCUGUAAGGUUGGUGUUC |
| 4563 | AAGGGACACAGAACACAUACA | 7167 | GAGCUGUAAGGUUGGUGUUCU |
| 4564 | AGGGACACAGAACACAUACAG | 7168 | AGCUGUAAGGUUGGUGUUCUA |
| 4565 | GGGACACAGAACACAUACAGA | 7169 | GCUGUAAGGUUGGUGUUCUAA |
| 4566 | GGACACAGAACACAUACAGAU | 7170 | CUGUAAGGUUGGUGUUCUAAC |
| 4567 | GACACAGAACACAUACAGAUG | 7171 | UGUAAGGUUGGUGUUCUAACA |
| 4568 | ACACAGAACACAUACAGAUGG | 7172 | GUAAGGUUGGUGUUCUAACAC |
| 4569 | CACAGAACACAUACAGAUGGA | 7173 | UAAGGUUGGUGUUCUAACACC |
| 4570 | ACAGAACACAUACAGAUGGAG | 7174 | AAGGUUGGUGUUCUAACACCC |
| 4571 | CAGAACACAUACAGAUGGAGG | 7175 | AGGUUGGUGUUCUAACACCCU |
| 4572 | AGAACACAUACAGAUGGAGGG | 7176 | GGUUGGUGUUCUAACACCCUG |
| 4573 | GAACACAUACAGAUGGAGGGA | 7177 | GUUGGUGUUCUAACACCCUGC |
| 4574 | AACACAUACAGAUGGAGGGAC | 7178 | UUGGUGUUCUAACACCCUGCA |
| 4575 | ACACAUACAGAUGGAGGGACA | 7179 | UGGUGUUCUAACACCCUGCAU |
| 4576 | CACAUACAGAUGGAGGGACAG | 7180 | GGUGUUCUAACACCCUGCAUC |
| 4577 | ACAUACAGAUGGAGGGACAGA | 7181 | GUGUUCUAACACCCUGCAUCU |
| 4578 | CAUACAGAUGGAGGGACAGAG | 7182 | UGUUCUAACACCCUGCAUCUC |
| 4579 | AUACAGAUGGAGGGACAGAGA | 7183 | GUUCUAACACCCUGCAUCUCC |
| 4580 | UACAGAUGGAGGGACAGAGAG | 7184 | UUCUAACACCCUGCAUCUCCA |
| 4581 | ACAGAUGGAGGGACAGAGAGG | 7185 | UCUAACACCCUGCAUCUCCAC |
| 4582 | CAGAUGGAGGGACAGAGAGGG | 7186 | CUAACACCCUGCAUCUCCACC |
| 4583 | AGAUGGAGGGACAGAGAGGGG | 7187 | UAACACCCUGCAUCUCCACCA |
| 4584 | GAUGGAGGGACAGAGAGGGGA | 7188 | AACACCCUGCAUCUCCACCAC |
| 4585 | AUGGAGGGACAGAGAGGGGAC | 7189 | ACACCCUGCAUCUCCACCACU |
| 4586 | UGGAGGGACAGAGAGGGGACC | 7190 | CACCCUGCAUCUCCACCACUU |
| 4587 | GGAGGGACAGAGAGGGGACCA | 7191 | ACCCUGCAUCUCCACCACUUU |
| 4588 | GAGGGACAGAGAGGGGACCAC | 7192 | CCCUGCAUCUCCACCACUUUC |
| 4589 | AGGGACAGAGAGGGGACCACA | 7193 | CCUGCAUCUCCACCACUUUCC |
| 4590 | GGGACAGAGAGGGGACCACAG | 7194 | CUGCAUCUCCACCACUUUCCC |
| 4591 | GGACAGAGAGGGGACCACAGC | 7195 | UGCAUCUCCACCACUUUCCCU |
| 4592 | GACAGAGAGGGGACCACAGCG | 7196 | GCAUCUCCACCACUUUCCCUU |
| 4593 | ACAGAGAGGGGACCACAGCGG | 7197 | CAUCUCCACCACUUUCCCUUU |
| 4594 | CAGAGAGGGGACCACAGCGGG | 7198 | AUCUCCACCACUUUCCCUUUG |
| 4595 | AGAGAGGGGACCACAGCGGGG | 7199 | UCUCCACCACUUUCCCUUUGC |
| 4596 | GAGAGGGGACCACAGCGGGGC | 7200 | CUCCACCACUUUCCCUUUGCU |
| 4597 | AGAGGGGACCACAGCGGGGCC | 7201 | UCCACCACUUUCCCUUUGCUG |
| 4598 | GAGGGGACCACAGCGGGGCCA | 7202 | CCACCACUUUCCCUUUGCUGC |
| 4599 | AGGGGACCACAGCGGGGCCAU | 7203 | CACCACUUUCCCUUUGCUGCU |
| 4600 | GGGGACCACAGCGGGGCCAUC | 7204 | ACCACUUUCCCUUUGCUGCUU |
| 4601 | GGGACCACAGCGGGGCCAUCG | 7205 | CCACUUUCCCUUUGCUGCUUG |
| 4602 | GGACCACAGCGGGGCCAUCGC | 7206 | CACUUUCCCUUUGCUGCUUGG |
| 4603 | GACCACAGCGGGGCCAUCGCA | 7207 | ACUUUCCCUUUGCUGCUUGGG |
| 4604 | ACCACAGCGGGGCCAUCGCAC | 7208 | CUUUCCCUUUGCUGCUUGGGC |
| 4605 | CCACAGCGGGGCCAUCGCACA | 7209 | UUUCCCUUUGCUGCUUGGGCU |
| 4606 | CACAGCGGGGCCAUCGCACAC | 7210 | UUCCCUUUGCUGCUUGGGCUG |
| 4607 | ACAGCGGGGCCAUCGCACACA | 7211 | UCCCUUUGCUGCUUGGGCUGA |
| 4608 | CAGCGGGGCCAUCGCACACAU | 7212 | CCCUUUGCUGCUUGGGCUGAG |
| 4609 | AGCGGGGCCAUCGCACACAUG | 7213 | CCUUUGCUGCUUGGGCUGAGU |
| 4610 | GCGGGGCCAUCGCACACAUGC | 7214 | CUUUGCUGCUUGGGCUGAGUG |
| 4611 | CGGGGCCAUCGCACACAUGCG | 7215 | UUUGCUGCUUGGGCUGAGUGA |
| 4612 | GGGGCCAUCGCACACAUGCGC | 7216 | UUGCUGCUUGGGCUGAGUGAC |
| 4613 | GGGCCAUCGCACACAUGCGCU | 7217 | UGCUGCUUGGGCUGAGUGACC |
| 4614 | GGCCAUCGCACACAUGCGCUC | 7218 | GCUGCUUGGGCUGAGUGACCA |
| 4615 | GCCAUCGCACACAUGCGCUCU | 7219 | CUGCUUGGGCUGAGUGACCAC |
| 4616 | CCAUCGCACACAUGCGCUCUC | 7220 | UGCUUGGGCUGAGUGACCACU |
| 4617 | CAUCGCACACAUGCGCUCUCA | 7221 | GCUUGGGCUGAGUGACCACUC |
| 4618 | AUCGCACACAUGCGCUCUCAU | 7222 | CUUGGGCUGAGUGACCACUCA |
| 4619 | UCGCACACAUGCGCUCUCAUG | 7223 | UUGGGCUGAGUGACCACUCAU |
| 4620 | CGCACACAUGCGCUCUCAUGC | 7224 | UGGGCUGAGUGACCACUCAUA |
| 4621 | GCACACAUGCGCUCUCAUGCC | 7225 | GGGCUGAGUGACCACUCAUAG |
| 4622 | CACACAUGCGCUCUCAUGCCA | 7226 | GGCUGAGUGACCACUCAUAGC |
| 4623 | ACACAUGCGCUCUCAUGCCAC | 7227 | GCUGAGUGACCACUCAUAGCU |
| 4624 | CACAUGCGCUCUCAUGCCACA | 7228 | CUGAGUGACCACUCAUAGCUG |
| 4625 | ACAUGCGCUCUCAUGCCACAA | 7229 | UGAGUGACCACUCAUAGCUGG |
| 4626 | CAUGCGCUCUCAUGCCACAAC | 7230 | GAGUGACCACUCAUAGCUGGU |
| 4627 | AUGCGCUCUCAUGCCACAACA | 7231 | AGUGACCACUCAUAGCUGGUG |
| 4628 | UGCGCUCUCAUGCCACAACAU | 7232 | GUGACCACUCAUAGCUGGUGA |
| 4629 | GCGCUCUCAUGCCACAACAUU | 7233 | UGACCACUCAUAGCUGGUGAU |
| 4630 | CGCUCUCAUGCCACAACAUUC | 7234 | GACCACUCAUAGCUGGUGAUG |
| 4631 | GCUCUCAUGCCACAACAUUCC | 7235 | ACCACUCAUAGCUGGUGAUGC |
| 4632 | CUCUCAUGCCACAACAUUCCA | 7236 | CCACUCAUAGCUGGUGAUGCC |
| 4633 | UCUCAUGCCACAACAUUCCAG | 7237 | CACUCAUAGCUGGUGAUGCCA |
| 4634 | CUCAUGCCACAACAUUCCAGU | 7238 | ACUCAUAGCUGGUGAUGCCAU |
| 4635 | UCAUGCCACAACAUUCCAGUC | 7239 | CUCAUAGCUGGUGAUGCCAUG |
| 4636 | CAUGCCACAACAUUCCAGUCC | 7240 | UCAUAGCUGGUGAUGCCAUGA |
| 4637 | AUGCCACAACAUUCCAGUCCA | 7241 | CAUAGCUGGUGAUGCCAUGAU |
| 4638 | UGCCACAACAUUCCAGUCCAU | 7242 | AUAGCUGGUGAUGCCAUGAUC |
| 4639 | GCCACAACAUUCCAGUCCAUC | 7243 | UAGCUGGUGAUGCCAUGAUCA |
| 4640 | CCACAACAUUCCAGUCCAUCU | 7244 | AGCUGGUGAUGCCAUGAUCAU |
| 4641 | CACAACAUUCCAGUCCAUCUG | 7245 | GCUGGUGAUGCCAUGAUCAUC |
| 4642 | ACAACAUUCCAGUCCAUCUGG | 7246 | CUGGUGAUGCCAUGAUCAUCA |
| 4643 | CAACAUUCCAGUCCAUCUGGG | 7247 | UGGUGAUGCCAUGAUCAUCAG |
| 4644 | AACAUUCCAGUCCAUCUGGGG | 7248 | GGUGAUGCCAUGAUCAUCAGU |
| 4645 | ACAUUCCAGUCCAUCUGGGGC | 7249 | GUGAUGCCAUGAUCAUCAGUG |
| 4646 | CAUUCCAGUCCAUCUGGGGCU | 7250 | UGAUGCCAUGAUCAUCAGUGC |
| 4647 | AUUCCAGUCCAUCUGGGGCUA | 7251 | GAUGCCAUGAUCAUCAGUGCU |
| 4648 | UUCCAGUCCAUCUGGGGCUAU | 7252 | AUGCCAUGAUCAUCAGUGCUC |
| 4649 | UCCAGUCCAUCUGGGGCUAUC | 7253 | UGCCAUGAUCAUCAGUGCUCU |
| 4650 | CCAGUCCAUCUGGGGCUAUCU | 7254 | GCCAUGAUCAUCAGUGCUCUG |
| 4651 | CAGUCCAUCUGGGGCUAUCUC | 7255 | CCAUGAUCAUCAGUGCUCUGG |
| 4652 | AGUCCAUCUGGGGCUAUCUCC | 7256 | CAUGAUCAUCAGUGCUCUGGU |
| 4653 | GUCCAUCUGGGGCUAUCUCCU | 7257 | AUGAUCAUCAGUGCUCUGGUU |
| 4654 | UCCAUCUGGGGCUAUCUCCUC | 7258 | UGAUCAUCAGUGCUCUGGUUC |
| 4655 | CCAUCUGGGGCUAUCUCCUCU | 7259 | GAUCAUCAGUGCUCUGGUUCC |
| 4656 | CAUCUGGGGCUAUCUCCUCUG | 7260 | AUCAUCAGUGCUCUGGUUCCC |
| 4657 | AUCUGGGGCUAUCUCCUCUGG | 7261 | UCAUCAGUGCUCUGGUUCCCA |
| 4658 | UCUGGGGCUAUCUCCUCUGGG | 7262 | CAUCAGUGCUCUGGUUCCCAA |
| 4659 | CUGGGGCUAUCUCCUCUGGGG | 7263 | AUCAGUGCUCUGGUUCCCAAA |
| 4660 | UGGGGCUAUCUCCUCUGGGGA | 7264 | UCAGUGCUCUGGUUCCCAAAG |
| 4661 | GGGGCUAUCUCCUCUGGGGAC | 7265 | CAGUGCUCUGGUUCCCAAAGA |
| 4662 | GGGCUAUCUCCUCUGGGGACA | 7266 | AGUGCUCUGGUUCCCAAAGAG |
| 4663 | GGCUAUCUCCUCUGGGGACAG | 7267 | GUGCUCUGGUUCCCAAAGAGG |
| 4664 | GCUAUCUCCUCUGGGGACAGG | 7268 | UGCUCUGGUUCCCAAAGAGGG |
| 4665 | CUAUCUCCUCUGGGGACAGGG | 7269 | GCUCUGGUUCCCAAAGAGGGU |
| 4666 | UAUCUCCUCUGGGGACAGGGA | 7270 | CUCUGGUUCCCAAAGAGGGUG |
| 4667 | AUCUCCUCUGGGGACAGGGAG | 7271 | UCUGGUUCCCAAAGAGGGUGA |
| 4668 | UCUCCUCUGGGGACAGGGAGC | 7272 | CUGGUUCCCAAAGAGGGUGAU |
| 4669 | CUCCUCUGGGGACAGGGAGCG | 7273 | UGGUUCCCAAAGAGGGUGAUG |
| 4670 | UCCUCUGGGGACAGGGAGCGG | 7274 | GGUUCCCAAAGAGGGUGAUGG |
| 4671 | CCUCUGGGGACAGGGAGCGGU | 7275 | GUUCCCAAAGAGGGUGAUGGA |
| 4672 | CUCUGGGGACAGGGAGCGGUG | 7276 | UUCCCAAAGAGGGUGAUGGAG |
| 4673 | UCUGGGGACAGGGAGCGGUGG | 7277 | UCCCAAAGAGGGUGAUGGAGU |
| 4674 | CUGGGGACAGGGAGCGGUGGC | 7278 | CCCAAAGAGGGUGAUGGAGUU |
| 4675 | UGGGGACAGGGAGCGGUGGCC | 7279 | CCAAAGAGGGUGAUGGAGUUU |
| 4676 | GGGGACAGGGAGCGGUGGCCU | 7280 | CAAAGAGGGUGAUGGAGUUUU |
| 4677 | GGGACAGGGAGCGGUGGCCUA | 7281 | AAAGAGGGUGAUGGAGUUUUG |
| 4678 | GGACAGGGAGCGGUGGCCUAA | 7282 | AAGAGGGUGAUGGAGUUUUGG |
| 4679 | GACAGGGAGCGGUGGCCUAAG | 7283 | AGAGGGUGAUGGAGUUUUGGG |
| 4680 | ACAGGGAGCGGUGGCCUAAGC | 7284 | GAGGGUGAUGGAGUUUUGGGG |
| 4681 | CAGGGAGCGGUGGCCUAAGCC | 7285 | AGGGUGAUGGAGUUUUGGGGC |
| 4682 | AGGGAGCGGUGGCCUAAGCCC | 7286 | GGGUGAUGGAGUUUUGGGGCA |
| 4683 | GGGAGCGGUGGCCUAAGCCCC | 7287 | GGUGAUGGAGUUUUGGGGCAG |
| 4684 | GGAGCGGUGGCCUAAGCCCCG | 7288 | GUGAUGGAGUUUUGGGGCAGG |
| 4685 | GAGCGGUGGCCUAAGCCCCGA | 7289 | UGAUGGAGUUUUGGGGCAGGG |
| 4686 | AGCGGUGGCCUAAGCCCCGAA | 7290 | GAUGGAGUUUUGGGGCAGGGU |
| 4687 | GCGGUGGCCUAAGCCCCGAAG | 7291 | AUGGAGUUUUGGGGCAGGGUG |
| 4688 | CGGUGGCCUAAGCCCCGAAGC | 7292 | UGGAGUUUUGGGGCAGGGUGA |
| 4689 | GGUGGCCUAAGCCCCGAAGCA | 7293 | GGAGUUUUGGGGCAGGGUGAU |
| 4690 | GUGGCCUAAGCCCCGAAGCAG | 7294 | GAGUUUUGGGGCAGGGUGAUC |
| 4691 | UGGCCUAAGCCCCGAAGCAGC | 7295 | AGUUUUGGGGCAGGGUGAUCA |
| 4692 | GGCCUAAGCCCCGAAGCAGCA | 7296 | GUUUUGGGGCAGGGUGAUCAC |
| 4693 | GCCUAAGCCCCGAAGCAGCAG | 7297 | UUUUGGGGCAGGGUGAUCACU |
| 4694 | CCUAAGCCCCGAAGCAGCAGG | 7298 | UUUGGGGCAGGGUGAUCACUU |
| 4695 | CUAAGCCCCGAAGCAGCAGGU | 7299 | UUGGGGCAGGGUGAUCACUUG |
| 4696 | UAAGCCCCGAAGCAGCAGGUG | 7300 | UGGGGCAGGGUGAUCACUUGG |
| 4697 | AAGCCCCGAAGCAGCAGGUGC | 7301 | GGGGCAGGGUGAUCACUUGGU |
| 4698 | AGCCCCGAAGCAGCAGGUGCC | 7302 | GGGCAGGGUGAUCACUUGGUU |
| 4699 | GCCCCGAAGCAGCAGGUGCCG | 7303 | GGCAGGGUGAUCACUUGGUUG |
| 4700 | CCCCGAAGCAGCAGGUGCCGG | 7304 | GCAGGGUGAUCACUUGGUUGG |
| 4701 | CCCGAAGCAGCAGGUGCCGGU | 7305 | CAGGGUGAUCACUUGGUUGGG |
| 4702 | CCGAAGCAGCAGGUGCCGGUC | 7306 | AGGGUGAUCACUUGGUUGGGG |
| 4703 | CGAAGCAGCAGGUGCCGGUCA | 7307 | GGGUGAUCACUUGGUUGGGGC |
| 4704 | GAAGCAGCAGGUGCCGGUCAG | 7308 | GGUGAUCACUUGGUUGGGGCC |
| 4705 | AAGCAGCAGGUGCCGGUCAGA | 7309 | GUGAUCACUUGGUUGGGGCCU |
| 4706 | AGCAGCAGGUGCCGGUCAGAU | 7310 | UGAUCACUUGGUUGGGGCCUG |
| 4707 | GCAGCAGGUGCCGGUCAGAUG | 7311 | GAUCACUUGGUUGGGGCCUGC |
| 4708 | CAGCAGGUGCCGGUCAGAUGG | 7312 | AUCACUUGGUUGGGGCCUGCG |
| 4709 | AGCAGGUGCCGGUCAGAUGGA | 7313 | UCACUUGGUUGGGGCCUGCGU |
| 4710 | GCAGGUGCCGGUCAGAUGGAG | 7314 | CACUUGGUUGGGGCCUGCGUU |
| 4711 | CAGGUGCCGGUCAGAUGGAGA | 7315 | ACUUGGUUGGGGCCUGCGUUG |
| 4712 | AGGUGCCGGUCAGAUGGAGAG | 7316 | CUUGGUUGGGGCCUGCGUUGG |
| 4713 | GGUGCCGGUCAGAUGGAGAGG | 7317 | UUGGUUGGGGCCUGCGUUGGC |
| 4714 | GUGCCGGUCAGAUGGAGAGGA | 7318 | UGGUUGGGGCCUGCGUUGGCC |
| 4715 | UGCCGGUCAGAUGGAGAGGAG | 7319 | GGUUGGGGCCUGCGUUGGCCA |
| 4716 | GCCGGUCAGAUGGAGAGGAGG | 7320 | GUUGGGGCCUGCGUUGGCCAC |
| 4717 | CCGGUCAGAUGGAGAGGAGGG | 7321 | UUGGGGCCUGCGUUGGCCACA |
| 4718 | CGGUCAGAUGGAGAGGAGGGA | 7322 | UGGGGCCUGCGUUGGCCACAG |
| 4719 | GGUCAGAUGGAGAGGAGGGAG | 7323 | GGGGCCUGCGUUGGCCACAGG |
| 4720 | GUCAGAUGGAGAGGAGGGAGG | 7324 | GGGCCUGCGUUGGCCACAGGG |
| 4721 | UCAGAUGGAGAGGAGGGAGGC | 7325 | GGCCUGCGUUGGCCACAGGGG |
| 4722 | CAGAUGGAGAGGAGGGAGGCC | 7326 | GCCUGCGUUGGCCACAGGGGG |
| 4723 | AGAUGGAGAGGAGGGAGGCCG | 7327 | CCUGCGUUGGCCACAGGGGGG |
| 4724 | GAUGGAGAGGAGGGAGGCCGU | 7328 | CUGCGUUGGCCACAGGGGGGU |
| 4725 | AUGGAGAGGAGGGAGGCCGUC | 7329 | UGCGUUGGCCACAGGGGGGUA |
| 4726 | UGGAGAGGAGGGAGGCCGUCU | 7330 | GCGUUGGCCACAGGGGGGUAA |
| 4727 | GGAGAGGAGGGAGGCCGUCUG | 7331 | CGUUGGCCACAGGGGGGUAAU |
| 4728 | GAGAGGAGGGAGGCCGUCUGU | 7332 | GUUGGCCACAGGGGGGUAAUC |
| 4729 | AGAGGAGGGAGGCCGUCUGUC | 7333 | UUGGCCACAGGGGGGUAAUCC |
| 4730 | GAGGAGGGAGGCCGUCUGUCC | 7334 | UGGCCACAGGGGGGUAAUCCA |
| 4731 | AGGAGGGAGGCCGUCUGUCCA | 7335 | GGCCACAGGGGGGUAAUCCAC |
| 4732 | GGAGGGAGGCCGUCUGUCCAG | 7336 | GCCACAGGGGGGUAAUCCACA |
| 4733 | GAGGGAGGCCGUCUGUCCAGC | 7337 | CCACAGGGGGGUAAUCCACAG |
| 4734 | AGGGAGGCCGUCUGUCCAGCC | 7338 | CACAGGGGGGUAAUCCACAGC |
| 4735 | GGGAGGCCGUCUGUCCAGCCU | 7339 | ACAGGGGGGUAAUCCACAGCU |
| 4736 | GGAGGCCGUCUGUCCAGCCUG | 7340 | CAGGGGGGUAAUCCACAGCUU |
| 4737 | GAGGCCGUCUGUCCAGCCUGG | 7341 | AGGGGGGUAAUCCACAGCUUU |
| 4738 | AGGCCGUCUGUCCAGCCUGGC | 7342 | GGGGGGUAAUCCACAGCUUUG |
| 4739 | GGCCGUCUGUCCAGCCUGGCU | 7343 | GGGGGUAAUCCACAGCUUUGU |
| 4740 | GCCGUCUGUCCAGCCUGGCUG | 7344 | GGGGUAAUCCACAGCUUUGUU |
| 4741 | CCGUCUGUCCAGCCUGGCUGC | 7345 | GGGUAAUCCACAGCUUUGUUC |
| 4742 | CGUCUGUCCAGCCUGGCUGCU | 7346 | GGUAAUCCACAGCUUUGUUCA |
| 4743 | GUCUGUCCAGCCUGGCUGCUC | 7347 | GUAAUCCACAGCUUUGUUCAC |
| 4744 | UCUGUCCAGCCUGGCUGCUCU | 7348 | UAAUCCACAGCUUUGUUCACU |
| 4745 | CUGUCCAGCCUGGCUGCUCUG | 7349 | AAUCCACAGCUUUGUUCACUG |
| 4746 | UGUCCAGCCUGGCUGCUCUGA | 7350 | AUCCACAGCUUUGUUCACUGU |
| 4747 | GUCCAGCCUGGCUGCUCUGAC | 7351 | UCCACAGCUUUGUUCACUGUC |
| 4748 | UCCAGCCUGGCUGCUCUGACA | 7352 | CCACAGCUUUGUUCACUGUCA |
| 4749 | CCAGCCUGGCUGCUCUGACAA | 7353 | CACAGCUUUGUUCACUGUCAG |
| 4750 | CAGCCUGGCUGCUCUGACAAG | 7354 | ACAGCUUUGUUCACUGUCAGG |
| 4751 | AGCCUGGCUGCUCUGACAAGG | 7355 | CAGCUUUGUUCACUGUCAGGU |
| 4752 | GCCUGGCUGCUCUGACAAGGG | 7356 | AGCUUUGUUCACUGUCAGGUU |
| 4753 | CCUGGCUGCUCUGACAAGGGC | 7357 | GCUUUGUUCACUGUCAGGUUU |
| 4754 | CUGGCUGCUCUGACAAGGGCC | 7358 | CUUUGUUCACUGUCAGGUUUG |
| 4755 | UGGCUGCUCUGACAAGGGCCC | 7359 | UUUGUUCACUGUCAGGUUUGC |
| 4756 | GGCUGCUCUGACAAGGGCCCU | 7360 | UUGUUCACUGUCAGGUUUGCA |
| 4757 | GCUGCUCUGACAAGGGCCCUG | 7361 | UGUUCACUGUCAGGUUUGCAG |
| 4758 | CUGCUCUGACAAGGGCCCUGG | 7362 | GUUCACUGUCAGGUUUGCAGU |
| 4759 | UGCUCUGACAAGGGCCCUGGC | 7363 | UUCACUGUCAGGUUUGCAGUA |
| 4760 | GCUCUGACAAGGGCCCUGGCA | 7364 | UCACUGUCAGGUUUGCAGUAG |
| 4761 | CUCUGACAAGGGCCCUGGCAG | 7365 | CACUGUCAGGUUUGCAGUAGU |
| 4762 | UCUGACAAGGGCCCUGGCAGC | 7366 | ACUGUCAGGUUUGCAGUAGUA |
| 4763 | CUGACAAGGGCCCUGGCAGCG | 7367 | CUGUCAGGUUUGCAGUAGUAG |
| 4764 | UGACAAGGGCCCUGGCAGCGA | 7368 | UGUCAGGUUUGCAGUAGUAGA |
| 4765 | GACAAGGGCCCUGGCAGCGAG | 7369 | GUCAGGUUUGCAGUAGUAGAG |
| 4766 | ACAAGGGCCCUGGCAGCGAGA | 7370 | UCAGGUUUGCAGUAGUAGAGU |
| 4767 | CAAGGGCCCUGGCAGCGAGAG | 7371 | CAGGUUUGCAGUAGUAGAGUU |
| 4768 | AAGGGCCCUGGCAGCGAGAGA | 7372 | AGGUUUGCAGUAGUAGAGUUG |
| 4769 | AGGGCCCUGGCAGCGAGAGAG | 7373 | GGUUUGCAGUAGUAGAGUUGG |
| 4770 | GGGCCCUGGCAGCGAGAGAGG | 7374 | GUUUGCAGUAGUAGAGUUGGU |
| 4771 | GGCCCUGGCAGCGAGAGAGGC | 7375 | UUUGCAGUAGUAGAGUUGGUA |
| 4772 | GCCCUGGCAGCGAGAGAGGCC | 7376 | UUGCAGUAGUAGAGUUGGUAG |
| 4773 | CCCUGGCAGCGAGAGAGGCCA | 7377 | UGCAGUAGUAGAGUUGGUAGC |
| 4774 | CCUGGCAGCGAGAGAGGCCAC | 7378 | GCAGUAGUAGAGUUGGUAGCU |
| 4775 | CUGGCAGCGAGAGAGGCCACC | 7379 | CAGUAGUAGAGUUGGUAGCUC |
| 4776 | UGGCAGCGAGAGAGGCCACCC | 7380 | AGUAGUAGAGUUGGUAGCUCC |
| 4777 | GGCAGCGAGAGAGGCCACCCG | 7381 | GUAGUAGAGUUGGUAGCUCCA |
| 4778 | GCAGCGAGAGAGGCCACCCGC | 7382 | UAGUAGAGUUGGUAGCUCCAU |
| 4779 | CAGCGAGAGAGGCCACCCGCC | 7383 | AGUAGAGUUGGUAGCUCCAUC |
| 4780 | AGCGAGAGAGGCCACCCGCCA | 7384 | GUAGAGUUGGUAGCUCCAUCA |
| 4781 | GCGAGAGAGGCCACCCGCCAU | 7385 | UAGAGUUGGUAGCUCCAUCAG |
| 4782 | CGAGAGAGGCCACCCGCCAUC | 7386 | AGAGUUGGUAGCUCCAUCAGA |
| 4783 | GAGAGAGGCCACCCGCCAUCC | 7387 | GAGUUGGUAGCUCCAUCAGAG |
| 4784 | AGAGAGGCCACCCGCCAUCCC | 7388 | AGUUGGUAGCUCCAUCAGAGU |
| 4785 | GAGAGGCCACCCGCCAUCCCU | 7389 | GUUGGUAGCUCCAUCAGAGUC |
| 4786 | AGAGGCCACCCGCCAUCCCUG | 7390 | UUGGUAGCUCCAUCAGAGUCU |
| 4787 | GAGGCCACCCGCCAUCCCUGA | 7391 | UGGUAGCUCCAUCAGAGUCUA |
| 4788 | AGGCCACCCGCCAUCCCUGAC | 7392 | GGUAGCUCCAUCAGAGUCUAC |
| 4789 | GGCCACCCGCCAUCCCUGACA | 7393 | GUAGCUCCAUCAGAGUCUACU |
| 4790 | GCCACCCGCCAUCCCUGACAC | 7394 | UAGCUCCAUCAGAGUCUACUA |
| 4791 | CCACCCGCCAUCCCUGACACA | 7395 | AGCUCCAUCAGAGUCUACUAC |
| 4792 | CACCCGCCAUCCCUGACACAG | 7396 | GCUCCAUCAGAGUCUACUACA |
| 4793 | ACCCGCCAUCCCUGACACAGU | 7397 | CUCCAUCAGAGUCUACUACAG |
| 4794 | CCCGCCAUCCCUGACACAGUC | 7398 | UCCAUCAGAGUCUACUACAGU |
| 4795 | CCGCCAUCCCUGACACAGUCA | 7399 | CCAUCAGAGUCUACUACAGUC |
| 4796 | CGCCAUCCCUGACACAGUCAA | 7400 | CAUCAGAGUCUACUACAGUCA |
| 4797 | GCCAUCCCUGACACAGUCAAU | 7401 | AUCAGAGUCUACUACAGUCAA |
| 4798 | CCAUCCCUGACACAGUCAAUC | 7402 | UCAGAGUCUACUACAGUCAAG |
| 4799 | CAUCCCUGACACAGUCAAUCC | 7403 | CAGAGUCUACUACAGUCAAGC |
| 4800 | AUCCCUGACACAGUCAAUCCU | 7404 | AGAGUCUACUACAGUCAAGCU |
| 4801 | UCCCUGACACAGUCAAUCCUC | 7405 | GAGUCUACUACAGUCAAGCUG |
| 4802 | CCCUGACACAGUCAAUCCUCC | 7406 | AGUCUACUACAGUCAAGCUGA |
| 4803 | CCUGACACAGUCAAUCCUCCC | 7407 | GUCUACUACAGUCAAGCUGAA |
| 4804 | CUGACACAGUCAAUCCUCCCU | 7408 | UCUACUACAGUCAAGCUGAAA |
| 4805 | UGACACAGUCAAUCCUCCCUA | 7409 | CUACUACAGUCAAGCUGAAAG |
| 4806 | GACACAGUCAAUCCUCCCUAA | 7410 | UACUACAGUCAAGCUGAAAGU |
| 4807 | ACACAGUCAAUCCUCCCUAAU | 7411 | ACUACAGUCAAGCUGAAAGUG |
| 4808 | CACAGUCAAUCCUCCCUAAUC | 7412 | CUACAGUCAAGCUGAAAGUGU |
| 4809 | ACAGUCAAUCCUCCCUAAUCU | 7413 | UACAGUCAAGCUGAAAGUGUA |
| 4810 | CAGUCAAUCCUCCCUAAUCUC | 7414 | ACAGUCAAGCUGAAAGUGUAG |
| 4811 | AGUCAAUCCUCCCUAAUCUCG | 7415 | CAGUCAAGCUGAAAGUGUAGU |
| 4812 | GUCAAUCCUCCCUAAUCUCGG | 7416 | AGUCAAGCUGAAAGUGUAGUU |
| 4813 | UCAAUCCUCCCUAAUCUCGGG | 7417 | GUCAAGCUGAAAGUGUAGUUC |
| 4814 | CAAUCCUCCCUAAUCUCGGGC | 7418 | UCAAGCUGAAAGUGUAGUUCC |
| 4815 | AAUCCUCCCUAAUCUCGGGCC | 7419 | CAAGCUGAAAGUGUAGUUCCC |
| 4816 | AUCCUCCCUAAUCUCGGGCCG | 7420 | AAGCUGAAAGUGUAGUUCCCA |
| 4817 | UCCUCCCUAAUCUCGGGCCGG | 7421 | AGCUGAAAGUGUAGUUCCCAG |
| 4818 | CCUCCCUAAUCUCGGGCCGGA | 7422 | GCUGAAAGUGUAGUUCCCAGG |
| 4819 | CUCCCUAAUCUCGGGCCGGAU | 7423 | CUGAAAGUGUAGUUCCCAGGG |
| 4820 | UCCCUAAUCUCGGGCCGGAUG | 7424 | UGAAAGUGUAGUUCCCAGGGA |
| 4821 | CCCUAAUCUCGGGCCGGAUGG | 7425 | GAAAGUGUAGUUCCCAGGGAC |
| 4822 | CCUAAUCUCGGGCCGGAUGGG | 7426 | AAAGUGUAGUUCCCAGGGACG |
| 4823 | CUAAUCUCGGGCCGGAUGGGA | 7427 | AAGUGUAGUUCCCAGGGACGA |
| 4824 | UAAUCUCGGGCCGGAUGGGAG | 7428 | AGUGUAGUUCCCAGGGACGAG |
| 4825 | AAUCUCGGGCCGGAUGGGAGA | 7429 | GUGUAGUUCCCAGGGACGAGU |
| 4826 | AUCUCGGGCCGGAUGGGAGAA | 7430 | UGUAGUUCCCAGGGACGAGUU |
| 4827 | UCUCGGGCCGGAUGGGAGAAG | 7431 | GUAGUUCCCAGGGACGAGUUU |
| 4828 | CUCGGGCCGGAUGGGAGAAGG | 7432 | UAGUUCCCAGGGACGAGUUUA |
| 4829 | UCGGGCCGGAUGGGAGAAGGG | 7433 | AGUUCCCAGGGACGAGUUUAC |
| 4830 | CGGGCCGGAUGGGAGAAGGGA | 7434 | GUUCCCAGGGACGAGUUUACU |
| 4831 | GGGCCGGAUGGGAGAAGGGAG | 7435 | UUCCCAGGGACGAGUUUACUU |
| 4832 | GGCCGGAUGGGAGAAGGGAGU | 7436 | UCCCAGGGACGAGUUUACUUA |
| 4833 | GCCGGAUGGGAGAAGGGAGUG | 7437 | CCCAGGGACGAGUUUACUUAG |
| 4834 | CCGGAUGGGAGAAGGGAGUGG | 7438 | CCAGGGACGAGUUUACUUAGU |
| 4835 | CGGAUGGGAGAAGGGAGUGGG | 7439 | CAGGGACGAGUUUACUUAGUU |
| 4836 | GGAUGGGAGAAGGGAGUGGGG | 7440 | AGGGACGAGUUUACUUAGUUU |
| 4837 | GAUGGGAGAAGGGAGUGGGGC | 7441 | GGGACGAGUUUACUUAGUUUU |
| 4838 | AUGGGAGAAGGGAGUGGGGCU | 7442 | GGACGAGUUUACUUAGUUUUA |
| 4839 | UGGGAGAAGGGAGUGGGGCUC | 7443 | GACGAGUUUACUUAGUUUUAA |
| 4840 | GGGAGAAGGGAGUGGGGCUCC | 7444 | ACGAGUUUACUUAGUUUUAAU |
| 4841 | GGAGAAGGGAGUGGGGCUCCA | 7445 | CGAGUUUACUUAGUUUUAAUA |
| 4842 | GAGAAGGGAGUGGGGCUCCAG | 7446 | GAGUUUACUUAGUUUUAAUAU |
| 4843 | AGAAGGGAGUGGGGCUCCAGU | 7447 | AGUUUACUUAGUUUUAAUAUG |
| 4844 | GAAGGGAGUGGGGCUCCAGUG | 7448 | GUUUACUUAGUUUUAAUAUGG |
| 4845 | AAGGGAGUGGGGCUCCAGUGU | 7449 | UUUACUUAGUUUUAAUAUGGC |
| 4846 | AGGGAGUGGGGCUCCAGUGUU | 7450 | UUACUUAGUUUUAAUAUGGCU |
| 4847 | GGGAGUGGGGCUCCAGUGUUA | 7451 | UACUUAGUUUUAAUAUGGCUG |
| 4848 | GGAGUGGGGCUCCAGUGUUAA | 7452 | ACUUAGUUUUAAUAUGGCUGU |
| 4849 | GAGUGGGGCUCCAGUGUUAAG | 7453 | CUUAGUUUUAAUAUGGCUGUA |
| 4850 | AGUGGGGCUCCAGUGUUAAGG | 7454 | UUAGUUUUAAUAUGGCUGUAU |
| 4851 | GUGGGGCUCCAGUGUUAAGGG | 7455 | UAGUUUUAAUAUGGCUGUAUC |
| 4852 | UGGGGCUCCAGUGUUAAGGGG | 7456 | AGUUUUAAUAUGGCUGUAUCU |
| 4853 | GGGGCUCCAGUGUUAAGGGGG | 7457 | GUUUUAAUAUGGCUGUAUCUU |
| 4854 | GGGCUCCAGUGUUAAGGGGGG | 7458 | UUUUAAUAUGGCUGUAUCUUC |
| 4855 | GGCUCCAGUGUUAAGGGGGGG | 7459 | UUUAAUAUGGCUGUAUCUUCA |
| 4856 | GCUCCAGUGUUAAGGGGGGGC | 7460 | UUAAUAUGGCUGUAUCUUCAG |
| 4857 | CUCCAGUGUUAAGGGGGGGCC | 7461 | UAAUAUGGCUGUAUCUUCAGA |
| 4858 | UCCAGUGUUAAGGGGGGGCCA | 7462 | AAUAUGGCUGUAUCUUCAGAA |
| 4859 | CCAGUGUUAAGGGGGGGCCAG | 7463 | AUAUGGCUGUAUCUUCAGAAA |
| 4860 | CAGUGUUAAGGGGGGGCCAGA | 7464 | UAUGGCUGUAUCUUCAGAAAU |
| 4861 | AGUGUUAAGGGGGGGCCAGAU | 7465 | AUGGCUGUAUCUUCAGAAAUC |
| 4862 | GUGUUAAGGGGGGGCCAGAUA | 7466 | UGGCUGUAUCUUCAGAAAUCU |
| 4863 | UGUUAAGGGGGGGCCAGAUAU | 7467 | GGCUGUAUCUUCAGAAAUCUU |
| 4864 | GUUAAGGGGGGGCCAGAUAUC | 7468 | GCUGUAUCUUCAGAAAUCUUC |
| 4865 | UUAAGGGGGGGCCAGAUAUCA | 7469 | CUGUAUCUUCAGAAAUCUUCU |
| 4866 | UAAGGGGGGGCCAGAUAUCAU | 7470 | UGUAUCUUCAGAAAUCUUCUC |
| 4867 | AAGGGGGGGCCAGAUAUCAUU | 7471 | GUAUCUUCAGAAAUCUUCUCU |
| 4868 | AGGGGGGGCCAGAUAUCAUUU | 7472 | UAUCUUCAGAAAUCUUCUCUU |
| 4869 | GGGGGGGCCAGAUAUCAUUUC | 7473 | AUCUUCAGAAAUCUUCUCUUC |
| 4870 | GGGGGGCCAGAUAUCAUUUCU | 7474 | UCUUCAGAAAUCUUCUCUUCU |
| 4871 | GGGGGCCAGAUAUCAUUUCUU | 7475 | CUUCAGAAAUCUUCUCUUCUC |
| 4872 | GGGGCCAGAUAUCAUUUCUUU | 7476 | UUCAGAAAUCUUCUCUUCUCU |
| 4873 | GGGCCAGAUAUCAUUUCUUUU | 7477 | UCAGAAAUCUUCUCUUCUCUU |
| 4874 | GGCCAGAUAUCAUUUCUUUUU | 7478 | CAGAAAUCUUCUCUUCUCUUA |
| 4875 | GCCAGAUAUCAUUUCUUUUUU | 7479 | AGAAAUCUUCUCUUCUCUUAG |
| 4876 | CCAGAUAUCAUUUCUUUUUUU | 7480 | GAAAUCUUCUCUUCUCUUAGA |
| 4877 | CAGAUAUCAUUUCUUUUUUUU | 7481 | AAAUCUUCUCUUCUCUUAGAG |
| 4878 | AGAUAUCAUUUCUUUUUUUUU | 7482 | AAUCUUCUCUUCUCUUAGAGG |
| 4879 | GAUAUCAUUUCUUUUUUUUUU | 7483 | AUCUUCUCUUCUCUUAGAGGC |
| 4880 | AUAUCAUUUCUUUUUUUUUUU | 7484 | UCUUCUCUUCUCUUAGAGGCC |
| 4881 | UAUCAUUUCUUUUUUUUUUUU | 7485 | CUUCUCUUCUCUUAGAGGCCC |
| 4882 | AUCAUUUCUUUUUUUUUUUUU | 7486 | UUCUCUUCUCUUAGAGGCCCC |
| 4883 | UCAUUUCUUUUUUUUUUUUUU | 7487 | UCUCUUCUCUUAGAGGCCCCU |
| 4884 | CAUUUCUUUUUUUUUUUUUUU | 7488 | CUCUUCUCUUAGAGGCCCCUU |
| 4885 | AUUUCUUUUUUUUUUUUUUUU | 7489 | UCUUCUCUUAGAGGCCCCUUA |
| 4886 | UUUCUUUUUUUUUUUUUUUUU | 7490 | CUUCUCUUAGAGGCCCCUUAA |
| 4887 | UUCUUUUUUUUUUUUUUUUUU | 7491 | UUCUCUUAGAGGCCCCUUAAG |
| 4888 | UCUUUUUUUUUUUUUUUUUUU | 7492 | UCUCUUAGAGGCCCCUUAAGU |
| 4889 | CUUUUUUUUUUUUUUUUUUUU | 7493 | CUCUUAGAGGCCCCUUAAGUU |
| 4890 | UUUUUUUUUUUUUUUUUUUUU | 7494 | UCUUAGAGGCCCCUUAAGUUC |
| 4891 | UUUUUUUUUUUUUUUUUUUUG | 7495 | CUUAGAGGCCCCUUAAGUUCU |
| 4892 | UUUUUUUUUUUUUUUUUUUGA | 7496 | UUAGAGGCCCCUUAAGUUCUU |
| 4893 | UUUUUUUUUUUUUUUUUUGAC | 7497 | UAGAGGCCCCUUAAGUUCUUC |
| 4894 | UUUUUUUUUUUUUUUUUGACG | 7498 | AGAGGCCCCUUAAGUUCUUCC |
| 4895 | UUUUUUUUUUUUUUUUGACGG | 7499 | GAGGCCCCUUAAGUUCUUCCC |
| 4896 | UUUUUUUUUUUUUUUGACGGA | 7500 | AGGCCCCUUAAGUUCUUCCCA |
| 4897 | UUUUUUUUUUUUUUGACGGAG | 7501 | GGCCCCUUAAGUUCUUCCCAA |
| 4898 | UUUUUUUUUUUUUGACGGAGU | 7502 | GCCCCUUAAGUUCUUCCCAAU |
| 4899 | UUUUUUUUUUUUGACGGAGUC | 7503 | CCCCUUAAGUUCUUCCCAAUG |
| 4900 | UUUUUUUUUUUGACGGAGUCU | 7504 | CCCUUAAGUUCUUCCCAAUGG |
| 4901 | UUUUUUUUUUGACGGAGUCUU | 7505 | CCUUAAGUUCUUCCCAAUGGU |
| 4902 | UUUUUUUUUGACGGAGUCUUG | 7506 | CUUAAGUUCUUCCCAAUGGUA |
| 4903 | UUUUUUUUGACGGAGUCUUGC | 7507 | UUAAGUUCUUCCCAAUGGUAC |
| 4904 | UUUUUUUGACGGAGUCUUGCU | 7508 | UAAGUUCUUCCCAAUGGUACU |
| 4905 | UUUUUUGACGGAGUCUUGCUC | 7509 | AAGUUCUUCCCAAUGGUACUG |
| 4906 | UUUUUGACGGAGUCUUGCUCU | 7510 | AGUUCUUCCCAAUGGUACUGA |
| 4907 | UUUUGACGGAGUCUUGCUCUG | 7511 | GUUCUUCCCAAUGGUACUGAA |
| 4908 | UUUGACGGAGUCUUGCUCUGU | 7512 | UUCUUCCCAAUGGUACUGAAC |
| 4909 | UUGACGGAGUCUUGCUCUGUC | 7513 | UCUUCCCAAUGGUACUGAACG |
| 4910 | UGACGGAGUCUUGCUCUGUCA | 7514 | CUUCCCAAUGGUACUGAACGA |
| 4911 | GACGGAGUCUUGCUCUGUCAC | 7515 | UUCCCAAUGGUACUGAACGAU |
| 4912 | ACGGAGUCUUGCUCUGUCACU | 7516 | UCCCAAUGGUACUGAACGAUU |
| 4913 | CGGAGUCUUGCUCUGUCACUC | 7517 | CCCAAUGGUACUGAACGAUUU |
| 4914 | GGAGUCUUGCUCUGUCACUCA | 7518 | CCAAUGGUACUGAACGAUUUU |
| 4915 | GAGUCUUGCUCUGUCACUCAG | 7519 | CAAUGGUACUGAACGAUUUUA |
| 4916 | AGUCUUGCUCUGUCACUCAGG | 7520 | AAUGGUACUGAACGAUUUUAU |
| 4917 | GUCUUGCUCUGUCACUCAGGC | 7521 | AUGGUACUGAACGAUUUUAUC |
| 4918 | UCUUGCUCUGUCACUCAGGCU | 7522 | UGGUACUGAACGAUUUUAUCA |
| 4919 | CUUGCUCUGUCACUCAGGCUG | 7523 | GGUACUGAACGAUUUUAUCAU |
| 4920 | UUGCUCUGUCACUCAGGCUGG | 7524 | GUACUGAACGAUUUUAUCAUC |
| 4921 | UGCUCUGUCACUCAGGCUGGA | 7525 | UACUGAACGAUUUUAUCAUCA |
| 4922 | GCUCUGUCACUCAGGCUGGAG | 7526 | ACUGAACGAUUUUAUCAUCAU |
| 4923 | CUCUGUCACUCAGGCUGGAGU | 7527 | CUGAACGAUUUUAUCAUCAUC |
| 4924 | UCUGUCACUCAGGCUGGAGUG | 7528 | UGAACGAUUUUAUCAUCAUCA |
| 4925 | CUGUCACUCAGGCUGGAGUGC | 7529 | GAACGAUUUUAUCAUCAUCAG |
| 4926 | UGUCACUCAGGCUGGAGUGCA | 7530 | AACGAUUUUAUCAUCAUCAGU |
| 4927 | GUCACUCAGGCUGGAGUGCAG | 7531 | ACGAUUUUAUCAUCAUCAGUG |
| 4928 | UCACUCAGGCUGGAGUGCAGU | 7532 | CGAUUUUAUCAUCAUCAGUGC |
| 4929 | CACUCAGGCUGGAGUGCAGUG | 7533 | GAUUUUAUCAUCAUCAGUGCU |
| 4930 | ACUCAGGCUGGAGUGCAGUGG | 7534 | AUUUUAUCAUCAUCAGUGCUU |
| 4931 | CUCAGGCUGGAGUGCAGUGGC | 7535 | UUUUAUCAUCAUCAGUGCUUU |
| 4932 | UCAGGCUGGAGUGCAGUGGCA | 7536 | UUUAUCAUCAUCAGUGCUUUG |
| 4933 | CAGGCUGGAGUGCAGUGGCAC | 7537 | UUAUCAUCAUCAGUGCUUUGA |
| 4934 | AGGCUGGAGUGCAGUGGCACG | 7538 | UAUCAUCAUCAGUGCUUUGAC |
| 4935 | GGCUGGAGUGCAGUGGCACGA | 7539 | AUCAUCAUCAGUGCUUUGACU |
| 4936 | GCUGGAGUGCAGUGGCACGAU | 7540 | UCAUCAUCAGUGCUUUGACUG |
| 4937 | CUGGAGUGCAGUGGCACGAUC | 7541 | CAUCAUCAGUGCUUUGACUGC |
| 4938 | UGGAGUGCAGUGGCACGAUCU | 7542 | AUCAUCAGUGCUUUGACUGCC |
| 4939 | GGAGUGCAGUGGCACGAUCUU | 7543 | UCAUCAGUGCUUUGACUGCCA |
| 4940 | GAGUGCAGUGGCACGAUCUUG | 7544 | CAUCAGUGCUUUGACUGCCAU |
| 4941 | AGUGCAGUGGCACGAUCUUGG | 7545 | AUCAGUGCUUUGACUGCCAUC |
| 4942 | GUGCAGUGGCACGAUCUUGGC | 7546 | UCAGUGCUUUGACUGCCAUCA |
| 4943 | UGCAGUGGCACGAUCUUGGCU | 7547 | CAGUGCUUUGACUGCCAUCAA |
| 4944 | GCAGUGGCACGAUCUUGGCUC | 7548 | AGUGCUUUGACUGCCAUCAAU |
| 4945 | CAGUGGCACGAUCUUGGCUCA | 7549 | GUGCUUUGACUGCCAUCAAUG |
| 4946 | AGUGGCACGAUCUUGGCUCAC | 7550 | UGCUUUGACUGCCAUCAAUGA |
| 4947 | GUGGCACGAUCUUGGCUCACU | 7551 | GCUUUGACUGCCAUCAAUGAC |
| 4948 | UGGCACGAUCUUGGCUCACUG | 7552 | CUUUGACUGCCAUCAAUGACU |
| 4949 | GGCACGAUCUUGGCUCACUGC | 7553 | UUUGACUGCCAUCAAUGACUG |
| 4950 | GCACGAUCUUGGCUCACUGCA | 7554 | UUGACUGCCAUCAAUGACUGU |
| 4951 | CACGAUCUUGGCUCACUGCAG | 7555 | UGACUGCCAUCAAUGACUGUA |
| 4952 | ACGAUCUUGGCUCACUGCAGC | 7556 | GACUGCCAUCAAUGACUGUAG |
| 4953 | CGAUCUUGGCUCACUGCAGCC | 7557 | ACUGCCAUCAAUGACUGUAGA |
| 4954 | GAUCUUGGCUCACUGCAGCCU | 7558 | CUGCCAUCAAUGACUGUAGAA |
| 4955 | AUCUUGGCUCACUGCAGCCUC | 7559 | UGCCAUCAAUGACUGUAGAAG |
| 4956 | UCUUGGCUCACUGCAGCCUCC | 7560 | GCCAUCAAUGACUGUAGAAGU |
| 4957 | CUUGGCUCACUGCAGCCUCCA | 7561 | CCAUCAAUGACUGUAGAAGUG |
| 4958 | UUGGCUCACUGCAGCCUCCAC | 7562 | CAUCAAUGACUGUAGAAGUGG |
| 4959 | UGGCUCACUGCAGCCUCCACC | 7563 | AUCAAUGACUGUAGAAGUGGU |
| 4960 | GGCUCACUGCAGCCUCCACCU | 7564 | UCAAUGACUGUAGAAGUGGUU |
| 4961 | GCUCACUGCAGCCUCCACCUC | 7565 | CAAUGACUGUAGAAGUGGUUG |
| 4962 | CUCACUGCAGCCUCCACCUCC | 7566 | AAUGACUGUAGAAGUGGUUGG |
| 4963 | UCACUGCAGCCUCCACCUCCC | 7567 | AUGACUGUAGAAGUGGUUGGC |
| 4964 | CACUGCAGCCUCCACCUCCCA | 7568 | UGACUGUAGAAGUGGUUGGCA |
| 4965 | ACUGCAGCCUCCACCUCCCAG | 7569 | GACUGUAGAAGUGGUUGGCAA |
| 4966 | CUGCAGCCUCCACCUCCCAGG | 7570 | ACUGUAGAAGUGGUUGGCAAA |
| 4967 | UGCAGCCUCCACCUCCCAGGU | 7571 | CUGUAGAAGUGGUUGGCAAAG |
| 4968 | GCAGCCUCCACCUCCCAGGUU | 7572 | UGUAGAAGUGGUUGGCAAAGA |
| 4969 | CAGCCUCCACCUCCCAGGUUU | 7573 | GUAGAAGUGGUUGGCAAAGAG |
| 4970 | AGCCUCCACCUCCCAGGUUUA | 7574 | UAGAAGUGGUUGGCAAAGAGA |
| 4971 | GCCUCCACCUCCCAGGUUUAA | 7575 | AGAAGUGGUUGGCAAAGAGAU |
| 4972 | CCUCCACCUCCCAGGUUUAAG | 7576 | GAAGUGGUUGGCAAAGAGAUC |
| 4973 | CUCCACCUCCCAGGUUUAAGC | 7577 | AAGUGGUUGGCAAAGAGAUCU |
| 4974 | UCCACCUCCCAGGUUUAAGCA | 7578 | AGUGGUUGGCAAAGAGAUCUC |
| 4975 | CCACCUCCCAGGUUUAAGCAA | 7579 | GUGGUUGGCAAAGAGAUCUCC |
| 4976 | CACCUCCCAGGUUUAAGCAAU | 7580 | UGGUUGGCAAAGAGAUCUCCU |
| 4977 | ACCUCCCAGGUUUAAGCAAUU | 7581 | GGUUGGCAAAGAGAUCUCCUG |
| 4978 | CCUCCCAGGUUUAAGCAAUUC | 7582 | GUUGGCAAAGAGAUCUCCUGG |
| 4979 | CUCCCAGGUUUAAGCAAUUCU | 7583 | UUGGCAAAGAGAUCUCCUGGA |
| 4980 | UCCCAGGUUUAAGCAAUUCUC | 7584 | UGGCAAAGAGAUCUCCUGGAA |
| 4981 | CCCAGGUUUAAGCAAUUCUCC | 7585 | GGCAAAGAGAUCUCCUGGAAC |
| 4982 | CCAGGUUUAAGCAAUUCUCCU | 7586 | GCAAAGAGAUCUCCUGGAACU |
| 4983 | CAGGUUUAAGCAAUUCUCCUG | 7587 | CAAAGAGAUCUCCUGGAACUG |
| 4984 | AGGUUUAAGCAAUUCUCCUGC | 7588 | AAAGAGAUCUCCUGGAACUGA |
| 4985 | GGUUUAAGCAAUUCUCCUGCC | 7589 | AAGAGAUCUCCUGGAACUGAG |
| 4986 | GUUUAAGCAAUUCUCCUGCCU | 7590 | AGAGAUCUCCUGGAACUGAGG |
| 4987 | UUUAAGCAAUUCUCCUGCCUC | 7591 | GAGAUCUCCUGGAACUGAGGU |
| 4988 | UUAAGCAAUUCUCCUGCCUCA | 7592 | AGAUCUCCUGGAACUGAGGUG |
| 4989 | UAAGCAAUUCUCCUGCCUCAG | 7593 | GAUCUCCUGGAACUGAGGUGA |
| 4990 | AAGCAAUUCUCCUGCCUCAGC | 7594 | AUCUCCUGGAACUGAGGUGAC |
| 4991 | AGCAAUUCUCCUGCCUCAGCC | 7595 | UCUCCUGGAACUGAGGUGACA |
| 4992 | GCAAUUCUCCUGCCUCAGCCU | 7596 | CUCCUGGAACUGAGGUGACAC |
| 4993 | CAAUUCUCCUGCCUCAGCCUC | 7597 | UCCUGGAACUGAGGUGACACA |
| 4994 | AAUUCUCCUGCCUCAGCCUCC | 7598 | CCUGGAACUGAGGUGACACAA |
| 4995 | AUUCUCCUGCCUCAGCCUCCC | 7599 | CUGGAACUGAGGUGACACAAU |
| 4996 | UUCUCCUGCCUCAGCCUCCCG | 7600 | UGGAACUGAGGUGACACAAUA |
| 4997 | UCUCCUGCCUCAGCCUCCCGA | 7601 | GGAACUGAGGUGACACAAUAG |
| 4998 | CUCCUGCCUCAGCCUCCCGAG | 7602 | GAACUGAGGUGACACAAUAGC |
| 4999 | UCCUGCCUCAGCCUCCCGAGU | 7603 | AACUGAGGUGACACAAUAGCA |
| 5000 | CCUGCCUCAGCCUCCCGAGUA | 7604 | ACUGAGGUGACACAAUAGCAA |
| 5001 | CUGCCUCAGCCUCCCGAGUAG | 7605 | CUGAGGUGACACAAUAGCAAU |
| 5002 | UGCCUCAGCCUCCCGAGUAGC | 7606 | UGAGGUGACACAAUAGCAAUG |
| 5003 | GCCUCAGCCUCCCGAGUAGCU | 7607 | GAGGUGACACAAUAGCAAUGG |
| 5004 | CCUCAGCCUCCCGAGUAGCUG | 7608 | AGGUGACACAAUAGCAAUGGG |
| 5005 | CUCAGCCUCCCGAGUAGCUGG | 7609 | GGUGACACAAUAGCAAUGGGG |
| 5006 | UCAGCCUCCCGAGUAGCUGGG | 7610 | GUGACACAAUAGCAAUGGGGG |
| 5007 | CAGCCUCCCGAGUAGCUGGGA | 7611 | UGACACAAUAGCAAUGGGGGG |
| 5008 | AGCCUCCCGAGUAGCUGGGAU | 7612 | GACACAAUAGCAAUGGGGGGC |
| 5009 | GCCUCCCGAGUAGCUGGGAUU | 7613 | ACACAAUAGCAAUGGGGGGCC |
| 5010 | CCUCCCGAGUAGCUGGGAUUA | 7614 | CACAAUAGCAAUGGGGGGCCG |
| 5011 | CUCCCGAGUAGCUGGGAUUAC | 7615 | ACAAUAGCAAUGGGGGGCCGA |
| 5012 | UCCCGAGUAGCUGGGAUUACA | 7616 | CAAUAGCAAUGGGGGGCCGAU |
| 5013 | CCCGAGUAGCUGGGAUUACAG | 7617 | AAUAGCAAUGGGGGGCCGAUU |
| 5014 | CCGAGUAGCUGGGAUUACAGG | 7618 | AUAGCAAUGGGGGGCCGAUUC |
| 5015 | CGAGUAGCUGGGAUUACAGGC | 7619 | UAGCAAUGGGGGGCCGAUUCU |
| 5016 | GAGUAGCUGGGAUUACAGGCA | 7620 | AGCAAUGGGGGGCCGAUUCUU |
| 5017 | AGUAGCUGGGAUUACAGGCAU | 7621 | GCAAUGGGGGGCCGAUUCUUA |
| 5018 | GUAGCUGGGAUUACAGGCAUA | 7622 | CAAUGGGGGGCCGAUUCUUAC |
| 5019 | UAGCUGGGAUUACAGGCAUAC | 7623 | AAUGGGGGGCCGAUUCUUACG |
| 5020 | AGCUGGGAUUACAGGCAUACG | 7624 | AUGGGGGGCCGAUUCUUACGG |
| 5021 | GCUGGGAUUACAGGCAUACGC | 7625 | UGGGGGGCCGAUUCUUACGGG |
| 5022 | CUGGGAUUACAGGCAUACGCC | 7626 | GGGGGGCCGAUUCUUACGGGG |
| 5023 | UGGGAUUACAGGCAUACGCCA | 7627 | GGGGGCCGAUUCUUACGGGGC |
| 5024 | GGGAUUACAGGCAUACGCCAC | 7628 | GGGGCCGAUUCUUACGGGGCU |
| 5025 | GGAUUACAGGCAUACGCCACC | 7629 | GGGCCGAUUCUUACGGGGCUC |
| 5026 | GAUUACAGGCAUACGCCACCA | 7630 | GGCCGAUUCUUACGGGGCUCU |
| 5027 | AUUACAGGCAUACGCCACCAA | 7631 | GCCGAUUCUUACGGGGCUCUG |
| 5028 | UUACAGGCAUACGCCACCAAA | 7632 | CCGAUUCUUACGGGGCUCUGG |
| 5029 | UACAGGCAUACGCCACCAAAC | 7633 | CGAUUCUUACGGGGCUCUGGC |
| 5030 | ACAGGCAUACGCCACCAAACC | 7634 | GAUUCUUACGGGGCUCUGGCU |
| 5031 | CAGGCAUACGCCACCAAACCC | 7635 | AUUCUUACGGGGCUCUGGCUU |
| 5032 | AGGCAUACGCCACCAAACCCG | 7636 | UUCUUACGGGGCUCUGGCUUG |
| 5033 | GGCAUACGCCACCAAACCCGG | 7637 | UCUUACGGGGCUCUGGCUUGA |
| 5034 | GCAUACGCCACCAAACCCGGC | 7638 | CUUACGGGGCUCUGGCUUGAC |
| 5035 | CAUACGCCACCAAACCCGGCU | 7639 | UUACGGGGCUCUGGCUUGACU |
| 5036 | AUACGCCACCAAACCCGGCUA | 7640 | UACGGGGCUCUGGCUUGACUG |
| 5037 | UACGCCACCAAACCCGGCUAA | 7641 | ACGGGGCUCUGGCUUGACUGU |
| 5038 | ACGCCACCAAACCCGGCUAAU | 7642 | CGGGGCUCUGGCUUGACUGUC |
| 5039 | CGCCACCAAACCCGGCUAAUU | 7643 | GGGGCUCUGGCUUGACUGUCA |
| 5040 | GCCACCAAACCCGGCUAAUUU | 7644 | GGGCUCUGGCUUGACUGUCAC |
| 5041 | CCACCAAACCCGGCUAAUUUU | 7645 | GGCUCUGGCUUGACUGUCACG |
| 5042 | CACCAAACCCGGCUAAUUUUU | 7646 | GCUCUGGCUUGACUGUCACGU |
| 5043 | ACCAAACCCGGCUAAUUUUUU | 7647 | CUCUGGCUUGACUGUCACGUU |
| 5044 | CCAAACCCGGCUAAUUUUUUU | 7648 | UCUGGCUUGACUGUCACGUUC |
| 5045 | CAAACCCGGCUAAUUUUUUUU | 7649 | CUGGCUUGACUGUCACGUUCA |
| 5046 | AAACCCGGCUAAUUUUUUUUU | 7650 | UGGCUUGACUGUCACGUUCAC |
| 5047 | AACCCGGCUAAUUUUUUUUUU | 7651 | GGCUUGACUGUCACGUUCACA |
| 5048 | ACCCGGCUAAUUUUUUUUUUU | 7652 | GCUUGACUGUCACGUUCACAU |
| 5049 | CCCGGCUAAUUUUUUUUUUUA | 7653 | CUUGACUGUCACGUUCACAUA |
| 5050 | CCGGCUAAUUUUUUUUUUUAU | 7654 | UUGACUGUCACGUUCACAUAG |
| 5051 | CGGCUAAUUUUUUUUUUUAUU | 7655 | UGACUGUCACGUUCACAUAGC |
| 5052 | GGCUAAUUUUUUUUUUUAUUU | 7656 | GACUGUCACGUUCACAUAGCC |
| 5053 | GCUAAUUUUUUUUUUUAUUUU | 7657 | ACUGUCACGUUCACAUAGCCU |
| 5054 | CUAAUUUUUUUUUUUAUUUUU | 7658 | CUGUCACGUUCACAUAGCCUU |
| 5055 | UAAUUUUUUUUUUUAUUUUUA | 7659 | UGUCACGUUCACAUAGCCUUC |
| 5056 | AAUUUUUUUUUUUAUUUUUAG | 7660 | GUCACGUUCACAUAGCCUUCC |
| 5057 | AUUUUUUUUUUUAUUUUUAGU | 7661 | UCACGUUCACAUAGCCUUCCC |
| 5058 | UUUUUUUUUUUAUUUUUAGUA | 7662 | CACGUUCACAUAGCCUUCCCC |
| 5059 | UUUUUUUUUUAUUUUUAGUAG | 7663 | ACGUUCACAUAGCCUUCCCCA |
| 5060 | UUUUUUUUUAUUUUUAGUAGA | 7664 | CGUUCACAUAGCCUUCCCCAU |
| 5061 | UUUUUUUUAUUUUUAGUAGAG | 7665 | GUUCACAUAGCCUUCCCCAUG |
| 5062 | UUUUUUUAUUUUUAGUAGAGA | 7666 | UUCACAUAGCCUUCCCCAUGG |
| 5063 | UUUUUUAUUUUUAGUAGAGAU | 7667 | UCACAUAGCCUUCCCCAUGGG |
| 5064 | UUUUUAUUUUUAGUAGAGAUG | 7668 | CACAUAGCCUUCCCCAUGGGC |
| 5065 | UUUUAUUUUUAGUAGAGAUGG | 7669 | ACAUAGCCUUCCCCAUGGGCA |
| 5066 | UUUAUUUUUAGUAGAGAUGGG | 7670 | CAUAGCCUUCCCCAUGGGCAU |
| 5067 | UUAUUUUUAGUAGAGAUGGGG | 7671 | AUAGCCUUCCCCAUGGGCAUU |
| 5068 | UAUUUUUAGUAGAGAUGGGGU | 7672 | UAGCCUUCCCCAUGGGCAUUU |
| 5069 | AUUUUUAGUAGAGAUGGGGUU | 7673 | AGCCUUCCCCAUGGGCAUUUU |
| 5070 | UUUUUAGUAGAGAUGGGGUUU | 7674 | GCCUUCCCCAUGGGCAUUUUG |
| 5071 | UUUUAGUAGAGAUGGGGUUUC | 7675 | CCUUCCCCAUGGGCAUUUUGA |
| 5072 | UUUAGUAGAGAUGGGGUUUCA | 7676 | CUUCCCCAUGGGCAUUUUGAC |
| 5073 | UUAGUAGAGAUGGGGUUUCAC | 7677 | UUCCCCAUGGGCAUUUUGACC |
| 5074 | UAGUAGAGAUGGGGUUUCACC | 7678 | UCCCCAUGGGCAUUUUGACCC |
| 5075 | AGUAGAGAUGGGGUUUCACCG | 7679 | CCCCAUGGGCAUUUUGACCCU |
| 5076 | GUAGAGAUGGGGUUUCACCGU | 7680 | CCCAUGGGCAUUUUGACCCUC |
| 5077 | UAGAGAUGGGGUUUCACCGUG | 7681 | CCAUGGGCAUUUUGACCCUCU |
| 5078 | AGAGAUGGGGUUUCACCGUGU | 7682 | CAUGGGCAUUUUGACCCUCUA |
| 5079 | GAGAUGGGGUUUCACCGUGUU | 7683 | AUGGGCAUUUUGACCCUCUAC |
| 5080 | AGAUGGGGUUUCACCGUGUUA | 7684 | UGGGCAUUUUGACCCUCUACA |
| 5081 | GAUGGGGUUUCACCGUGUUAG | 7685 | GGGCAUUUUGACCCUCUACAA |
| 5082 | AUGGGGUUUCACCGUGUUAGC | 7686 | GGCAUUUUGACCCUCUACAAU |
| 5083 | UGGGGUUUCACCGUGUUAGCC | 7687 | GCAUUUUGACCCUCUACAAUC |
| 5084 | GGGGUUUCACCGUGUUAGCCA | 7688 | CAUUUUGACCCUCUACAAUCA |
| 5085 | GGGUUUCACCGUGUUAGCCAG | 7689 | AUUUUGACCCUCUACAAUCAC |
| 5086 | GGUUUCACCGUGUUAGCCAGG | 7690 | UUUUGACCCUCUACAAUCACU |
| 5087 | GUUUCACCGUGUUAGCCAGGG | 7691 | UUUGACCCUCUACAAUCACUU |
| 5088 | UUUCACCGUGUUAGCCAGGGU | 7692 | UUGACCCUCUACAAUCACUUU |
| 5089 | UUCACCGUGUUAGCCAGGGUG | 7693 | UGACCCUCUACAAUCACUUUG |
| 5090 | UCACCGUGUUAGCCAGGGUGG | 7694 | GACCCUCUACAAUCACUUUGA |
| 5091 | CACCGUGUUAGCCAGGGUGGU | 7695 | ACCCUCUACAAUCACUUUGAA |
| 5092 | ACCGUGUUAGCCAGGGUGGUC | 7696 | CCCUCUACAAUCACUUUGAAU |
| 5093 | CCGUGUUAGCCAGGGUGGUCU | 7697 | CCUCUACAAUCACUUUGAAUU |
| 5094 | CGUGUUAGCCAGGGUGGUCUU | 7698 | CUCUACAAUCACUUUGAAUUC |
| 5095 | GUGUUAGCCAGGGUGGUCUUG | 7699 | UCUACAAUCACUUUGAAUUCA |
| 5096 | UGUUAGCCAGGGUGGUCUUGA | 7700 | CUACAAUCACUUUGAAUUCAU |
| 5097 | GUUAGCCAGGGUGGUCUUGAU | 7701 | UACAAUCACUUUGAAUUCAUA |
| 5098 | UUAGCCAGGGUGGUCUUGAUC | 7702 | ACAAUCACUUUGAAUUCAUAC |
| 5099 | UAGCCAGGGUGGUCUUGAUCU | 7703 | CAAUCACUUUGAAUUCAUACA |
| 5100 | AGCCAGGGUGGUCUUGAUCUC | 7704 | AAUCACUUUGAAUUCAUACAG |
| 5101 | GCCAGGGUGGUCUUGAUCUCC | 7705 | AUCACUUUGAAUUCAUACAGG |
| 5102 | CCAGGGUGGUCUUGAUCUCCU | 7706 | UCACUUUGAAUUCAUACAGGC |
| 5103 | CAGGGUGGUCUUGAUCUCCUG | 7707 | CACUUUGAAUUCAUACAGGCC |
| 5104 | AGGGUGGUCUUGAUCUCCUGA | 7708 | ACUUUGAAUUCAUACAGGCCU |
| 5105 | GGGUGGUCUUGAUCUCCUGAC | 7709 | CUUUGAAUUCAUACAGGCCUG |
| 5106 | GGUGGUCUUGAUCUCCUGACC | 7710 | UUUGAAUUCAUACAGGCCUGG |
| 5107 | GUGGUCUUGAUCUCCUGACCU | 7711 | UUGAAUUCAUACAGGCCUGGA |
| 5108 | UGGUCUUGAUCUCCUGACCUC | 7712 | UGAAUUCAUACAGGCCUGGAG |
| 5109 | GGUCUUGAUCUCCUGACCUCA | 7713 | GAAUUCAUACAGGCCUGGAGU |
| 5110 | GUCUUGAUCUCCUGACCUCAU | 7714 | AAUUCAUACAGGCCUGGAGUG |
| 5111 | UCUUGAUCUCCUGACCUCAUG | 7715 | AUUCAUACAGGCCUGGAGUGA |
| 5112 | CUUGAUCUCCUGACCUCAUGA | 7716 | UUCAUACAGGCCUGGAGUGAG |
| 5113 | UUGAUCUCCUGACCUCAUGAU | 7717 | UCAUACAGGCCUGGAGUGAGC |
| 5114 | UGAUCUCCUGACCUCAUGAUC | 7718 | CAUACAGGCCUGGAGUGAGCU |
| 5115 | GAUCUCCUGACCUCAUGAUCC | 7719 | AUACAGGCCUGGAGUGAGCUU |
| 5116 | AUCUCCUGACCUCAUGAUCCG | 7720 | UACAGGCCUGGAGUGAGCUUC |
| 5117 | UCUCCUGACCUCAUGAUCCGC | 7721 | ACAGGCCUGGAGUGAGCUUCG |
| 5118 | CUCCUGACCUCAUGAUCCGCC | 7722 | CAGGCCUGGAGUGAGCUUCGA |
| 5119 | UCCUGACCUCAUGAUCCGCCC | 7723 | AGGCCUGGAGUGAGCUUCGAU |
| 5120 | CCUGACCUCAUGAUCCGCCCG | 7724 | GGCCUGGAGUGAGCUUCGAUA |
| 5121 | CUGACCUCAUGAUCCGCCCGC | 7725 | GCCUGGAGUGAGCUUCGAUAG |
| 5122 | UGACCUCAUGAUCCGCCCGCC | 7726 | CCUGGAGUGAGCUUCGAUAGU |
| 5123 | GACCUCAUGAUCCGCCCGCCU | 7727 | CUGGAGUGAGCUUCGAUAGUU |
| 5124 | ACCUCAUGAUCCGCCCGCCUC | 7728 | UGGAGUGAGCUUCGAUAGUUU |
| 5125 | CCUCAUGAUCCGCCCGCCUCG | 7729 | GGAGUGAGCUUCGAUAGUUUG |
| 5126 | CUCAUGAUCCGCCCGCCUCGG | 7730 | GAGUGAGCUUCGAUAGUUUGA |
| 5127 | UCAUGAUCCGCCCGCCUCGGC | 7731 | AGUGAGCUUCGAUAGUUUGAG |
| 5128 | CAUGAUCCGCCCGCCUCGGCC | 7732 | GUGAGCUUCGAUAGUUUGAGG |
| 5129 | AUGAUCCGCCCGCCUCGGCCU | 7733 | UGAGCUUCGAUAGUUUGAGGA |
| 5130 | UGAUCCGCCCGCCUCGGCCUC | 7734 | GAGCUUCGAUAGUUUGAGGAU |
| 5131 | GAUCCGCCCGCCUCGGCCUCC | 7735 | AGCUUCGAUAGUUUGAGGAUC |
| 5132 | AUCCGCCCGCCUCGGCCUCCC | 7736 | GCUUCGAUAGUUUGAGGAUCU |
| 5133 | UCCGCCCGCCUCGGCCUCCCA | 7737 | CUUCGAUAGUUUGAGGAUCUG |
| 5134 | CCGCCCGCCUCGGCCUCCCAA | 7738 | UUCGAUAGUUUGAGGAUCUGG |
| 5135 | CGCCCGCCUCGGCCUCCCAAA | 7739 | UCGAUAGUUUGAGGAUCUGGG |
| 5136 | GCCCGCCUCGGCCUCCCAAAG | 7740 | CGAUAGUUUGAGGAUCUGGGA |
| 5137 | CCCGCCUCGGCCUCCCAAAGU | 7741 | GAUAGUUUGAGGAUCUGGGAA |
| 5138 | CCGCCUCGGCCUCCCAAAGUG | 7742 | AUAGUUUGAGGAUCUGGGAAU |
| 5139 | CGCCUCGGCCUCCCAAAGUGC | 7743 | UAGUUUGAGGAUCUGGGAAUG |
| 5140 | GCCUCGGCCUCCCAAAGUGCU | 7744 | AGUUUGAGGAUCUGGGAAUGU |
| 5141 | CCUCGGCCUCCCAAAGUGCUG | 7745 | GUUUGAGGAUCUGGGAAUGUU |
| 5142 | CUCGGCCUCCCAAAGUGCUGG | 7746 | UUUGAGGAUCUGGGAAUGUUU |
| 5143 | UCGGCCUCCCAAAGUGCUGGG | 7747 | UUGAGGAUCUGGGAAUGUUUC |
| 5144 | CGGCCUCCCAAAGUGCUGGGA | 7748 | UGAGGAUCUGGGAAUGUUUCC |
| 5145 | GGCCUCCCAAAGUGCUGGGAU | 7749 | GAGGAUCUGGGAAUGUUUCCC |
| 5146 | GCCUCCCAAAGUGCUGGGAUU | 7750 | AGGAUCUGGGAAUGUUUCCCU |
| 5147 | CCUCCCAAAGUGCUGGGAUUA | 7751 | GGAUCUGGGAAUGUUUCCCUU |
| 5148 | CUCCCAAAGUGCUGGGAUUAC | 7752 | GAUCUGGGAAUGUUUCCCUUC |
| 5149 | UCCCAAAGUGCUGGGAUUACA | 7753 | AUCUGGGAAUGUUUCCCUUCC |
| 5150 | CCCAAAGUGCUGGGAUUACAG | 7754 | UCUGGGAAUGUUUCCCUUCCA |
| 5151 | CCAAAGUGCUGGGAUUACAGG | 7755 | CUGGGAAUGUUUCCCUUCCAU |
| 5152 | CAAAGUGCUGGGAUUACAGGC | 7756 | UGGGAAUGUUUCCCUUCCAUU |
| 5153 | AAAGUGCUGGGAUUACAGGCG | 7757 | GGGAAUGUUUCCCUUCCAUUU |
| 5154 | AAGUGCUGGGAUUACAGGCGU | 7758 | GGAAUGUUUCCCUUCCAUUUC |
| 5155 | AGUGCUGGGAUUACAGGCGUG | 7759 | GAAUGUUUCCCUUCCAUUUCU |
| 5156 | GUGCUGGGAUUACAGGCGUGA | 7760 | AAUGUUUCCCUUCCAUUUCUC |
| 5157 | UGCUGGGAUUACAGGCGUGAG | 7761 | AUGUUUCCCUUCCAUUUCUCC |
| 5158 | GCUGGGAUUACAGGCGUGAGC | 7762 | UGUUUCCCUUCCAUUUCUCCA |
| 5159 | CUGGGAUUACAGGCGUGAGCC | 7763 | GUUUCCCUUCCAUUUCUCCAC |
| 5160 | UGGGAUUACAGGCGUGAGCCA | 7764 | UUUCCCUUCCAUUUCUCCACU |
| 5161 | GGGAUUACAGGCGUGAGCCAC | 7765 | UUCCCUUCCAUUUCUCCACUG |
| 5162 | GGAUUACAGGCGUGAGCCACC | 7766 | UCCCUUCCAUUUCUCCACUGU |
| 5163 | GAUUACAGGCGUGAGCCACCG | 7767 | CCCUUCCAUUUCUCCACUGUA |
| 5164 | AUUACAGGCGUGAGCCACCGC | 7768 | CCUUCCAUUUCUCCACUGUAG |
| 5165 | UUACAGGCGUGAGCCACCGCG | 7769 | CUUCCAUUUCUCCACUGUAGU |
| 5166 | UACAGGCGUGAGCCACCGCGC | 7770 | UUCCAUUUCUCCACUGUAGUC |
| 5167 | ACAGGCGUGAGCCACCGCGCC | 7771 | UCCAUUUCUCCACUGUAGUCU |
| 5168 | CAGGCGUGAGCCACCGCGCCC | 7772 | CCAUUUCUCCACUGUAGUCUC |
| 5169 | AGGCGUGAGCCACCGCGCCCG | 7773 | CAUUUCUCCACUGUAGUCUCU |
| 5170 | GGCGUGAGCCACCGCGCCCGG | 7774 | AUUUCUCCACUGUAGUCUCUA |
| 5171 | GCGUGAGCCACCGCGCCCGGC | 7775 | UUUCUCCACUGUAGUCUCUAG |
| 5172 | CGUGAGCCACCGCGCCCGGCC | 7776 | UUCUCCACUGUAGUCUCUAGG |
| 5173 | GUGAGCCACCGCGCCCGGCCA | 7777 | UCUCCACUGUAGUCUCUAGGA |
| 5174 | UGAGCCACCGCGCCCGGCCAU | 7778 | CUCCACUGUAGUCUCUAGGAU |
| 5175 | GAGCCACCGCGCCCGGCCAUC | 7779 | UCCACUGUAGUCUCUAGGAUG |
| 5176 | AGCCACCGCGCCCGGCCAUCA | 7780 | CCACUGUAGUCUCUAGGAUGA |
| 5177 | GCCACCGCGCCCGGCCAUCAU | 7781 | CACUGUAGUCUCUAGGAUGAG |
| 5178 | CCACCGCGCCCGGCCAUCAUU | 7782 | ACUGUAGUCUCUAGGAUGAGU |
| 5179 | CACCGCGCCCGGCCAUCAUUU | 7783 | CUGUAGUCUCUAGGAUGAGUA |
| 5180 | ACCGCGCCCGGCCAUCAUUUC | 7784 | UGUAGUCUCUAGGAUGAGUAA |
| 5181 | CCGCGCCCGGCCAUCAUUUCU | 7785 | GUAGUCUCUAGGAUGAGUAAU |
| 5182 | CGCGCCCGGCCAUCAUUUCUA | 7786 | UAGUCUCUAGGAUGAGUAAUC |
| 5183 | GCGCCCGGCCAUCAUUUCUAU | 7787 | AGUCUCUAGGAUGAGUAAUCA |
| 5184 | CGCCCGGCCAUCAUUUCUAUG | 7788 | GUCUCUAGGAUGAGUAAUCAG |
| 5185 | GCCCGGCCAUCAUUUCUAUGC | 7789 | UCUCUAGGAUGAGUAAUCAGC |
| 5186 | CCCGGCCAUCAUUUCUAUGCU | 7790 | CUCUAGGAUGAGUAAUCAGCU |
| 5187 | CCGGCCAUCAUUUCUAUGCUA | 7791 | UCUAGGAUGAGUAAUCAGCUG |
| 5188 | CGGCCAUCAUUUCUAUGCUAC | 7792 | CUAGGAUGAGUAAUCAGCUGC |
| 5189 | GGCCAUCAUUUCUAUGCUACC | 7793 | UAGGAUGAGUAAUCAGCUGCC |
| 5190 | GCCAUCAUUUCUAUGCUACCA | 7794 | AGGAUGAGUAAUCAGCUGCCA |
| 5191 | CCAUCAUUUCUAUGCUACCAU | 7795 | GGAUGAGUAAUCAGCUGCCAG |
| 5192 | CAUCAUUUCUAUGCUACCAUC | 7796 | GAUGAGUAAUCAGCUGCCAGU |
| 5193 | AUCAUUUCUAUGCUACCAUCU | 7797 | AUGAGUAAUCAGCUGCCAGUC |
| 5194 | UCAUUUCUAUGCUACCAUCUC | 7798 | UGAGUAAUCAGCUGCCAGUCG |
| 5195 | CAUUUCUAUGCUACCAUCUCA | 7799 | GAGUAAUCAGCUGCCAGUCGU |
| 5196 | AUUUCUAUGCUACCAUCUCAG | 7800 | AGUAAUCAGCUGCCAGUCGUA |
| 5197 | UUUCUAUGCUACCAUCUCAGC | 7801 | GUAAUCAGCUGCCAGUCGUAG |
| 5198 | UUCUAUGCUACCAUCUCAGCA | 7802 | UAAUCAGCUGCCAGUCGUAGG |
| 5199 | UCUAUGCUACCAUCUCAGCAU | 7803 | AAUCAGCUGCCAGUCGUAGGU |
| 5200 | CUAUGCUACCAUCUCAGCAUC | 7804 | AUCAGCUGCCAGUCGUAGGUG |
| 5201 | UAUGCUACCAUCUCAGCAUCU | 7805 | UCAGCUGCCAGUCGUAGGUGU |
| 5202 | AUGCUACCAUCUCAGCAUCUG | 7806 | CAGCUGCCAGUCGUAGGUGUA |
| 5203 | UGCUACCAUCUCAGCAUCUGU | 7807 | AGCUGCCAGUCGUAGGUGUAG |
| 5204 | GCUACCAUCUCAGCAUCUGUG | 7808 | GCUGCCAGUCGUAGGUGUAGG |
| 5205 | CUACCAUCUCAGCAUCUGUGG | 7809 | CUGCCAGUCGUAGGUGUAGGU |
| 5206 | UACCAUCUCAGCAUCUGUGGU | 7810 | UGCCAGUCGUAGGUGUAGGUU |
| 5207 | ACCAUCUCAGCAUCUGUGGUG | 7811 | GCCAGUCGUAGGUGUAGGUUU |
| 5208 | CCAUCUCAGCAUCUGUGGUGA | 7812 | CCAGUCGUAGGUGUAGGUUUC |
| 5209 | CAUCUCAGCAUCUGUGGUGAG | 7813 | CAGUCGUAGGUGUAGGUUUCU |
| 5210 | AUCUCAGCAUCUGUGGUGAGG | 7814 | AGUCGUAGGUGUAGGUUUCUC |
| 5211 | UCUCAGCAUCUGUGGUGAGGG | 7815 | GUCGUAGGUGUAGGUUUCUCC |
| 5212 | CUCAGCAUCUGUGGUGAGGGG | 7816 | UCGUAGGUGUAGGUUUCUCCU |
| 5213 | UCAGCAUCUGUGGUGAGGGGA | 7817 | CGUAGGUGUAGGUUUCUCCUU |
| 5214 | CAGCAUCUGUGGUGAGGGGAG | 7818 | GUAGGUGUAGGUUUCUCCUUU |
| 5215 | AGCAUCUGUGGUGAGGGGAGG | 7819 | UAGGUGUAGGUUUCUCCUUUA |
| 5216 | GCAUCUGUGGUGAGGGGAGGG | 7820 | AGGUGUAGGUUUCUCCUUUAG |
| 5217 | CAUCUGUGGUGAGGGGAGGGG | 7821 | GGUGUAGGUUUCUCCUUUAGG |
| 5218 | AUCUGUGGUGAGGGGAGGGGU | 7822 | GUGUAGGUUUCUCCUUUAGGU |
| 5219 | UCUGUGGUGAGGGGAGGGGUG | 7823 | UGUAGGUUUCUCCUUUAGGUG |
| 5220 | CUGUGGUGAGGGGAGGGGUGC | 7824 | GUAGGUUUCUCCUUUAGGUGG |
| 5221 | UGUGGUGAGGGGAGGGGUGCC | 7825 | UAGGUUUCUCCUUUAGGUGGU |
| 5222 | GUGGUGAGGGGAGGGGUGCCA | 7826 | AGGUUUCUCCUUUAGGUGGUU |
| 5223 | UGGUGAGGGGAGGGGUGCCAC | 7827 | GGUUUCUCCUUUAGGUGGUUC |
| 5224 | GGUGAGGGGAGGGGUGCCACU | 7828 | GUUUCUCCUUUAGGUGGUUCU |
| 5225 | GUGAGGGGAGGGGUGCCACUU | 7829 | UUUCUCCUUUAGGUGGUUCUU |
| 5226 | UGAGGGGAGGGGUGCCACUUC | 7830 | UUCUCCUUUAGGUGGUUCUUG |
| 5227 | GAGGGGAGGGGUGCCACUUCC | 7831 | UCUCCUUUAGGUGGUUCUUGG |
| 5228 | AGGGGAGGGGUGCCACUUCCU | 7832 | CUCCUUUAGGUGGUUCUUGGA |
| 5229 | GGGGAGGGGUGCCACUUCCUC | 7833 | UCCUUUAGGUGGUUCUUGGAG |
| 5230 | GGGAGGGGUGCCACUUCCUCU | 7834 | CCUUUAGGUGGUUCUUGGAGA |
| 5231 | GGAGGGGUGCCACUUCCUCUU | 7835 | CUUUAGGUGGUUCUUGGAGAA |
| 5232 | GAGGGGUGCCACUUCCUCUUU | 7836 | UUUAGGUGGUUCUUGGAGAAC |
| 5233 | AGGGGUGCCACUUCCUCUUUG | 7837 | UUAGGUGGUUCUUGGAGAACA |
| 5234 | GGGGUGCCACUUCCUCUUUGC | 7838 | UAGGUGGUUCUUGGAGAACAU |
| 5235 | GGGUGCCACUUCCUCUUUGCC | 7839 | AGGUGGUUCUUGGAGAACAUA |
| 5236 | GGUGCCACUUCCUCUUUGCCC | 7840 | GGUGGUUCUUGGAGAACAUAU |
| 5237 | GUGCCACUUCCUCUUUGCCCA | 7841 | GUGGUUCUUGGAGAACAUAUG |
| 5238 | UGCCACUUCCUCUUUGCCCAG | 7842 | UGGUUCUUGGAGAACAUAUGC |
| 5239 | GCCACUUCCUCUUUGCCCAGC | 7843 | GGUUCUUGGAGAACAUAUGCA |
| 5240 | CCACUUCCUCUUUGCCCAGCG | 7844 | GUUCUUGGAGAACAUAUGCAU |
| 5241 | CACUUCCUCUUUGCCCAGCGA | 7845 | UUCUUGGAGAACAUAUGCAUU |
| 5242 | ACUUCCUCUUUGCCCAGCGAG | 7846 | UCUUGGAGAACAUAUGCAUUU |
| 5243 | CUUCCUCUUUGCCCAGCGAGA | 7847 | CUUGGAGAACAUAUGCAUUUA |
| 5244 | UUCCUCUUUGCCCAGCGAGAG | 7848 | UUGGAGAACAUAUGCAUUUAA |
| 5245 | UCCUCUUUGCCCAGCGAGAGG | 7849 | UGGAGAACAUAUGCAUUUAAU |
| 5246 | CCUCUUUGCCCAGCGAGAGGG | 7850 | GGAGAACAUAUGCAUUUAAUU |
| 5247 | CUCUUUGCCCAGCGAGAGGGC | 7851 | GAGAACAUAUGCAUUUAAUUG |
| 5248 | UCUUUGCCCAGCGAGAGGGCG | 7852 | AGAACAUAUGCAUUUAAUUGA |
| 5249 | CUUUGCCCAGCGAGAGGGCGU | 7853 | GAACAUAUGCAUUUAAUUGAA |
| 5250 | UUUGCCCAGCGAGAGGGCGUA | 7854 | AACAUAUGCAUUUAAUUGAAC |
| 5251 | UUGCCCAGCGAGAGGGCGUAC | 7855 | ACAUAUGCAUUUAAUUGAACU |
| 5252 | UGCCCAGCGAGAGGGCGUACU | 7856 | CAUAUGCAUUUAAUUGAACUU |
| 5253 | GCCCAGCGAGAGGGCGUACUC | 7857 | AUAUGCAUUUAAUUGAACUUC |
| 5254 | CCCAGCGAGAGGGCGUACUCU | 7858 | UAUGCAUUUAAUUGAACUUCA |
| 5255 | CCAGCGAGAGGGCGUACUCUA | 7859 | AUGCAUUUAAUUGAACUUCAU |
| 5256 | CAGCGAGAGGGCGUACUCUAC | 7860 | UGCAUUUAAUUGAACUUCAUU |
| 5257 | AGCGAGAGGGCGUACUCUACC | 7861 | GCAUUUAAUUGAACUUCAUUC |
| 5258 | GCGAGAGGGCGUACUCUACCC | 7862 | CAUUUAAUUGAACUUCAUUCU |
| 5259 | CGAGAGGGCGUACUCUACCCC | 7863 | AUUUAAUUGAACUUCAUUCUU |
| 5260 | GAGAGGGCGUACUCUACCCCA | 7864 | UUUAAUUGAACUUCAUUCUUA |
| 5261 | AGAGGGCGUACUCUACCCCAG | 7865 | UUAAUUGAACUUCAUUCUUAG |
| 5262 | GAGGGCGUACUCUACCCCAGA | 7866 | UAAUUGAACUUCAUUCUUAGG |
| 5263 | AGGGCGUACUCUACCCCAGAG | 7867 | AAUUGAACUUCAUUCUUAGGC |
| 5264 | GGGCGUACUCUACCCCAGAGA | 7868 | AUUGAACUUCAUUCUUAGGCA |
| 5265 | GGCGUACUCUACCCCAGAGAG | 7869 | UUGAACUUCAUUCUUAGGCAG |
| 5266 | GCGUACUCUACCCCAGAGAGG | 7870 | UGAACUUCAUUCUUAGGCAGG |
| 5267 | CGUACUCUACCCCAGAGAGGG | 7871 | GAACUUCAUUCUUAGGCAGGG |
| 5268 | GUACUCUACCCCAGAGAGGGA | 7872 | AACUUCAUUCUUAGGCAGGGU |
| 5269 | UACUCUACCCCAGAGAGGGAA | 7873 | ACUUCAUUCUUAGGCAGGGUU |
| 5270 | ACUCUACCCCAGAGAGGGAAA | 7874 | CUUCAUUCUUAGGCAGGGUUA |
| 5271 | CUCUACCCCAGAGAGGGAAAC | 7875 | UUCAUUCUUAGGCAGGGUUAU |
| 5272 | UCUACCCCAGAGAGGGAAACA | 7876 | UCAUUCUUAGGCAGGGUUAUC |
| 5273 | CUACCCCAGAGAGGGAAACAC | 7877 | CAUUCUUAGGCAGGGUUAUCU |
| 5274 | UACCCCAGAGAGGGAAACACC | 7878 | AUUCUUAGGCAGGGUUAUCUG |
| 5275 | ACCCCAGAGAGGGAAACACCA | 7879 | UUCUUAGGCAGGGUUAUCUGG |
| 5276 | CCCCAGAGAGGGAAACACCAU | 7880 | UCUUAGGCAGGGUUAUCUGGA |
| 5277 | CCCAGAGAGGGAAACACCAUG | 7881 | CUUAGGCAGGGUUAUCUGGAC |
| 5278 | CCAGAGAGGGAAACACCAUGC | 7882 | UUAGGCAGGGUUAUCUGGACA |
| 5279 | CAGAGAGGGAAACACCAUGCC | 7883 | UAGGCAGGGUUAUCUGGACAC |
| 5280 | AGAGAGGGAAACACCAUGCCC | 7884 | AGGCAGGGUUAUCUGGACACU |
| 5281 | GAGAGGGAAACACCAUGCCCA | 7885 | GGCAGGGUUAUCUGGACACUC |
| 5282 | AGAGGGAAACACCAUGCCCAC | 7886 | GCAGGGUUAUCUGGACACUCU |
| 5283 | GAGGGAAACACCAUGCCCACA | 7887 | CAGGGUUAUCUGGACACUCUC |
| 5284 | AGGGAAACACCAUGCCCACAG | 7888 | AGGGUUAUCUGGACACUCUCU |
| 5285 | GGGAAACACCAUGCCCACAGU | 7889 | GGGUUAUCUGGACACUCUCUC |
| 5286 | GGAAACACCAUGCCCACAGUG | 7890 | GGUUAUCUGGACACUCUCUCC |
| 5287 | GAAACACCAUGCCCACAGUGC | 7891 | GUUAUCUGGACACUCUCUCCA |
| 5288 | AAACACCAUGCCCACAGUGCU | 7892 | UUAUCUGGACACUCUCUCCAG |
| 5289 | AACACCAUGCCCACAGUGCUU | 7893 | UAUCUGGACACUCUCUCCAGC |
| 5290 | ACACCAUGCCCACAGUGCUUG | 7894 | AUCUGGACACUCUCUCCAGCA |
| 5291 | CACCAUGCCCACAGUGCUUGG | 7895 | UCUGGACACUCUCUCCAGCAG |
| 5292 | ACCAUGCCCACAGUGCUUGGU | 7896 | CUGGACACUCUCUCCAGCAGA |
| 5293 | CCAUGCCCACAGUGCUUGGUU | 7897 | UGGACACUCUCUCCAGCAGAU |
| 5294 | CAUGCCCACAGUGCUUGGUUU | 7898 | GGACACUCUCUCCAGCAGAUA |
| 5295 | AUGCCCACAGUGCUUGGUUUU | 7899 | GACACUCUCUCCAGCAGAUAC |
| 5296 | UGCCCACAGUGCUUGGUUUUG | 7900 | ACACUCUCUCCAGCAGAUACC |
| 5297 | GCCCACAGUGCUUGGUUUUGC | 7901 | CACUCUCUCCAGCAGAUACCA |
| 5298 | CCCACAGUGCUUGGUUUUGCA | 7902 | ACUCUCUCCAGCAGAUACCAC |
| 5299 | CCACAGUGCUUGGUUUUGCAC | 7903 | CUCUCUCCAGCAGAUACCACC |
| 5300 | CACAGUGCUUGGUUUUGCACU | 7904 | UCUCUCCAGCAGAUACCACCA |
| 5301 | ACAGUGCUUGGUUUUGCACUC | 7905 | CUCUCCAGCAGAUACCACCAG |
| 5302 | CAGUGCUUGGUUUUGCACUCA | 7906 | UCUCCAGCAGAUACCACCAGU |
| 5303 | AGUGCUUGGUUUUGCACUCAG | 7907 | CUCCAGCAGAUACCACCAGUU |
| 5304 | GUGCUUGGUUUUGCACUCAGG | 7908 | UCCAGCAGAUACCACCAGUUC |
| 5305 | UGCUUGGUUUUGCACUCAGGU | 7909 | CCAGCAGAUACCACCAGUUCC |
| 5306 | GCUUGGUUUUGCACUCAGGUG | 7910 | CAGCAGAUACCACCAGUUCCU |
| 5307 | CUUGGUUUUGCACUCAGGUGU | 7911 | AGCAGAUACCACCAGUUCCUU |
| 5308 | UUGGUUUUGCACUCAGGUGUG | 7912 | GCAGAUACCACCAGUUCCUUU |
| 5309 | UGGUUUUGCACUCAGGUGUGC | 7913 | CAGAUACCACCAGUUCCUUUA |
| 5310 | GGUUUUGCACUCAGGUGUGCG | 7914 | AGAUACCACCAGUUCCUUUAU |
| 5311 | GUUUUGCACUCAGGUGUGCGG | 7915 | GAUACCACCAGUUCCUUUAUA |
| 5312 | UUUUGCACUCAGGUGUGCGGG | 7916 | AUACCACCAGUUCCUUUAUAA |
| 5313 | UUUGCACUCAGGUGUGCGGGC | 7917 | UACCACCAGUUCCUUUAUAAC |
| 5314 | UUGCACUCAGGUGUGCGGGCA | 7918 | ACCACCAGUUCCUUUAUAACU |
| 5315 | UGCACUCAGGUGUGCGGGCAG | 7919 | CCACCAGUUCCUUUAUAACUG |
| 5316 | GCACUCAGGUGUGCGGGCAGC | 7920 | CACCAGUUCCUUUAUAACUGG |
| 5317 | CACUCAGGUGUGCGGGCAGCA | 7921 | ACCAGUUCCUUUAUAACUGGG |
| 5318 | ACUCAGGUGUGCGGGCAGCAC | 7922 | CCAGUUCCUUUAUAACUGGGU |
| 5319 | CUCAGGUGUGCGGGCAGCACA | 7923 | CAGUUCCUUUAUAACUGGGUA |
| 5320 | UCAGGUGUGCGGGCAGCACAG | 7924 | AGUUCCUUUAUAACUGGGUAU |
| 5321 | CAGGUGUGCGGGCAGCACAGC | 7925 | GUUCCUUUAUAACUGGGUAUG |
| 5322 | AGGUGUGCGGGCAGCACAGCA | 7926 | UUCCUUUAUAACUGGGUAUGG |
| 5323 | GGUGUGCGGGCAGCACAGCAG | 7927 | UCCUUUAUAACUGGGUAUGGU |
| 5324 | GUGUGCGGGCAGCACAGCAGG | 7928 | CCUUUAUAACUGGGUAUGGUG |
| 5325 | UGUGCGGGCAGCACAGCAGGC | 7929 | CUUUAUAACUGGGUAUGGUGC |
| 5326 | GUGCGGGCAGCACAGCAGGCC | 7930 | UUUAUAACUGGGUAUGGUGCU |
| 5327 | UGCGGGCAGCACAGCAGGCCU | 7931 | UUAUAACUGGGUAUGGUGCUG |
| 5328 | GCGGGCAGCACAGCAGGCCUC | 7932 | UAUAACUGGGUAUGGUGCUGA |
| 5329 | CGGGCAGCACAGCAGGCCUCA | 7933 | AUAACUGGGUAUGGUGCUGAG |
| 5330 | GGGCAGCACAGCAGGCCUCAC | 7934 | UAACUGGGUAUGGUGCUGAGG |
| 5331 | GGCAGCACAGCAGGCCUCACC | 7935 | AACUGGGUAUGGUGCUGAGGU |
| 5332 | GCAGCACAGCAGGCCUCACCU | 7936 | ACUGGGUAUGGUGCUGAGGUG |
| 5333 | CAGCACAGCAGGCCUCACCUU | 7937 | CUGGGUAUGGUGCUGAGGUGC |
| 5334 | AGCACAGCAGGCCUCACCUUG | 7938 | UGGGUAUGGUGCUGAGGUGCU |
| 5335 | GCACAGCAGGCCUCACCUUGC | 7939 | GGGUAUGGUGCUGAGGUGCUC |
| 5336 | CACAGCAGGCCUCACCUUGCA | 7940 | GGUAUGGUGCUGAGGUGCUCU |
| 5337 | ACAGCAGGCCUCACCUUGCAG | 7941 | GUAUGGUGCUGAGGUGCUCUG |
| 5338 | CAGCAGGCCUCACCUUGCAGC | 7942 | UAUGGUGCUGAGGUGCUCUGG |
| 5339 | AGCAGGCCUCACCUUGCAGCA | 7943 | AUGGUGCUGAGGUGCUCUGGA |
| 5340 | GCAGGCCUCACCUUGCAGCAC | 7944 | UGGUGCUGAGGUGCUCUGGAA |
| 5341 | CAGGCCUCACCUUGCAGCACU | 7945 | GGUGCUGAGGUGCUCUGGAAA |
| 5342 | AGGCCUCACCUUGCAGCACUC | 7946 | GUGCUGAGGUGCUCUGGAAAG |
| 5343 | GGCCUCACCUUGCAGCACUCU | 7947 | UGCUGAGGUGCUCUGGAAAGA |
| 5344 | GCCUCACCUUGCAGCACUCUG | 7948 | GCUGAGGUGCUCUGGAAAGAG |
| 5345 | CCUCACCUUGCAGCACUCUGG | 7949 | CUGAGGUGCUCUGGAAAGAGG |
| 5346 | CUCACCUUGCAGCACUCUGGG | 7950 | UGAGGUGCUCUGGAAAGAGGC |
| 5347 | UCACCUUGCAGCACUCUGGGC | 7951 | GAGGUGCUCUGGAAAGAGGCC |
| 5348 | CACCUUGCAGCACUCUGGGCA | 7952 | AGGUGCUCUGGAAAGAGGCCU |
| 5349 | ACCUUGCAGCACUCUGGGCAC | 7953 | GGUGCUCUGGAAAGAGGCCUG |
| 5350 | CCUUGCAGCACUCUGGGCACA | 7954 | GUGCUCUGGAAAGAGGCCUGG |
| 5351 | CUUGCAGCACUCUGGGCACAA | 7955 | UGCUCUGGAAAGAGGCCUGGG |
| 5352 | UUGCAGCACUCUGGGCACAAU | 7956 | GCUCUGGAAAGAGGCCUGGGG |
| 5353 | UGCAGCACUCUGGGCACAAUG | 7957 | CUCUGGAAAGAGGCCUGGGGG |
| 5354 | GCAGCACUCUGGGCACAAUGA | 7958 | UCUGGAAAGAGGCCUGGGGGG |
| 5355 | CAGCACUCUGGGCACAAUGAC | 7959 | CUGGAAAGAGGCCUGGGGGGU |
| 5356 | AGCACUCUGGGCACAAUGACA | 7960 | UGGAAAGAGGCCUGGGGGGUA |
| 5357 | GCACUCUGGGCACAAUGACAC | 7961 | GGAAAGAGGCCUGGGGGGUAG |
| 5358 | CACUCUGGGCACAAUGACACU | 7962 | GAAAGAGGCCUGGGGGGUAGG |
| 5359 | ACUCUGGGCACAAUGACACUG | 7963 | AAAGAGGCCUGGGGGGUAGGG |
| 5360 | CUCUGGGCACAAUGACACUGU | 7964 | AAGAGGCCUGGGGGGUAGGGG |
| 5361 | UCUGGGCACAAUGACACUGUC | 7965 | AGAGGCCUGGGGGGUAGGGGU |
| 5362 | CUGGGCACAAUGACACUGUCC | 7966 | GAGGCCUGGGGGGUAGGGGUA |
| 5363 | UGGGCACAAUGACACUGUCCA | 7967 | AGGCCUGGGGGGUAGGGGUAG |
| 5364 | GGGCACAAUGACACUGUCCAC | 7968 | GGCCUGGGGGGUAGGGGUAGC |
| 5365 | GGCACAAUGACACUGUCCACU | 7969 | GCCUGGGGGGUAGGGGUAGCA |
| 5366 | GCACAAUGACACUGUCCACUG | 7970 | CCUGGGGGGUAGGGGUAGCAU |
| 5367 | CACAAUGACACUGUCCACUGG | 7971 | CUGGGGGGUAGGGGUAGCAUA |
| 5368 | ACAAUGACACUGUCCACUGGG | 7972 | UGGGGGGUAGGGGUAGCAUAA |
| 5369 | CAAUGACACUGUCCACUGGGG | 7973 | GGGGGGUAGGGGUAGCAUAAC |
| 5370 | AAUGACACUGUCCACUGGGGA | 7974 | GGGGGUAGGGGUAGCAUAACU |
| 5371 | AUGACACUGUCCACUGGGGAG | 7975 | GGGGUAGGGGUAGCAUAACUG |
| 5372 | UGACACUGUCCACUGGGGAGC | 7976 | GGGUAGGGGUAGCAUAACUGU |
| 5373 | GACACUGUCCACUGGGGAGCU | 7977 | GGUAGGGGUAGCAUAACUGUA |
| 5374 | ACACUGUCCACUGGGGAGCUG | 7978 | GUAGGGGUAGCAUAACUGUAG |
| 5375 | CACUGUCCACUGGGGAGCUGC | 7979 | UAGGGGUAGCAUAACUGUAGG |
| 5376 | ACUGUCCACUGGGGAGCUGCA | 7980 | AGGGGUAGCAUAACUGUAGGA |
| 5377 | CUGUCCACUGGGGAGCUGCAG | 7981 | GGGGUAGCAUAACUGUAGGAG |
| 5378 | UGUCCACUGGGGAGCUGCAGA | 7982 | GGGUAGCAUAACUGUAGGAGG |
| 5379 | GUCCACUGGGGAGCUGCAGAG | 7983 | GGUAGCAUAACUGUAGGAGGG |
| 5380 | UCCACUGGGGAGCUGCAGAGC | 7984 | GUAGCAUAACUGUAGGAGGGA |
| 5381 | CCACUGGGGAGCUGCAGAGCU | 7985 | UAGCAUAACUGUAGGAGGGAG |
| 5382 | CACUGGGGAGCUGCAGAGCUU | 7986 | AGCAUAACUGUAGGAGGGAGC |
| 5383 | ACUGGGGAGCUGCAGAGCUUA | 7987 | GCAUAACUGUAGGAGGGAGCC |
| 5384 | CUGGGGAGCUGCAGAGCUUAG | 7988 | CAUAACUGUAGGAGGGAGCCA |
| 5385 | UGGGGAGCUGCAGAGCUUAGC | 7989 | AUAACUGUAGGAGGGAGCCAC |
| 5386 | GGGGAGCUGCAGAGCUUAGCA | 7990 | UAACUGUAGGAGGGAGCCACU |
| 5387 | GGGAGCUGCAGAGCUUAGCAG | 7991 | AACUGUAGGAGGGAGCCACUG |
| 5388 | GGAGCUGCAGAGCUUAGCAGC | 7992 | ACUGUAGGAGGGAGCCACUGG |
| 5389 | GAGCUGCAGAGCUUAGCAGCU | 7993 | CUGUAGGAGGGAGCCACUGGC |
| 5390 | AGCUGCAGAGCUUAGCAGCUG | 7994 | UGUAGGAGGGAGCCACUGGCU |
| 5391 | GCUGCAGAGCUUAGCAGCUGG | 7995 | GUAGGAGGGAGCCACUGGCUG |
| 5392 | CUGCAGAGCUUAGCAGCUGGC | 7996 | UAGGAGGGAGCCACUGGCUGG |
| 5393 | UGCAGAGCUUAGCAGCUGGCU | 7997 | AGGAGGGAGCCACUGGCUGGG |
| 5394 | GCAGAGCUUAGCAGCUGGCUG | 7998 | GGAGGGAGCCACUGGCUGGGG |
| 5395 | CAGAGCUUAGCAGCUGGCUGG | 7999 | GAGGGAGCCACUGGCUGGGGG |
| 5396 | AGAGCUUAGCAGCUGGCUGGG | 8000 | AGGGAGCCACUGGCUGGGGGA |
| 5397 | GAGCUUAGCAGCUGGCUGGGU | 8001 | GGGAGCCACUGGCUGGGGGAC |
| 5398 | AGCUUAGCAGCUGGCUGGGUC | 8002 | GGAGCCACUGGCUGGGGGACA |
| 5399 | GCUUAGCAGCUGGCUGGGUCU | 8003 | GAGCCACUGGCUGGGGGACAG |
| 5400 | CUUAGCAGCUGGCUGGGUCUG | 8004 | AGCCACUGGCUGGGGGACAGC |
| 5401 | UUAGCAGCUGGCUGGGUCUGC | 8005 | GCCACUGGCUGGGGGACAGCA |
| 5402 | UAGCAGCUGGCUGGGUCUGCC | 8006 | CCACUGGCUGGGGGACAGCAA |
| 5403 | AGCAGCUGGCUGGGUCUGCCC | 8007 | CACUGGCUGGGGGACAGCAAU |
| 5404 | GCAGCUGGCUGGGUCUGCCCU | 8008 | ACUGGCUGGGGGACAGCAAUC |
| 5405 | CAGCUGGCUGGGUCUGCCCUC | 8009 | CUGGCUGGGGGACAGCAAUCU |
| 5406 | AGCUGGCUGGGUCUGCCCUCG | 8010 | UGGCUGGGGGACAGCAAUCUG |
| 5407 | GCUGGCUGGGUCUGCCCUCGG | 8011 | GGCUGGGGGACAGCAAUCUGG |
| 5408 | CUGGCUGGGUCUGCCCUCGGG | 8012 | GCUGGGGGACAGCAAUCUGGG |
| 5409 | UGGCUGGGUCUGCCCUCGGGG | 8013 | CUGGGGGACAGCAAUCUGGGU |
| 5410 | GGCUGGGUCUGCCCUCGGGGG | 8014 | UGGGGGACAGCAAUCUGGGUU |
| 5411 | GCUGGGUCUGCCCUCGGGGGA | 8015 | GGGGGACAGCAAUCUGGGUUU |
| 5412 | CUGGGUCUGCCCUCGGGGGAG | 8016 | GGGGACAGCAAUCUGGGUUUU |
| 5413 | UGGGUCUGCCCUCGGGGGAGG | 8017 | GGGACAGCAAUCUGGGUUUUC |
| 5414 | GGGUCUGCCCUCGGGGGAGGG | 8018 | GGACAGCAAUCUGGGUUUUCU |
| 5415 | GGUCUGCCCUCGGGGGAGGGG | 8019 | GACAGCAAUCUGGGUUUUCUC |
| 5416 | GUCUGCCCUCGGGGGAGGGGA | 8020 | ACAGCAAUCUGGGUUUUCUCA |
| 5417 | UCUGCCCUCGGGGGAGGGGAG | 8021 | CAGCAAUCUGGGUUUUCUCAG |
| 5418 | CUGCCCUCGGGGGAGGGGAGG | 8022 | AGCAAUCUGGGUUUUCUCAGA |
| 5419 | UGCCCUCGGGGGAGGGGAGGA | 8023 | GCAAUCUGGGUUUUCUCAGAA |
| 5420 | GCCCUCGGGGGAGGGGAGGAG | 8024 | CAAUCUGGGUUUUCUCAGAAC |
| 5421 | CCCUCGGGGGAGGGGAGGAGU | 8025 | AAUCUGGGUUUUCUCAGAACU |
| 5422 | CCUCGGGGGAGGGGAGGAGUU | 8026 | AUCUGGGUUUUCUCAGAACUU |
| 5423 | CUCGGGGGAGGGGAGGAGUUU | 8027 | UCUGGGUUUUCUCAGAACUUU |
| 5424 | UCGGGGGAGGGGAGGAGUUUG | 8028 | CUGGGUUUUCUCAGAACUUUU |
| 5425 | CGGGGGAGGGGAGGAGUUUGC | 8029 | UGGGUUUUCUCAGAACUUUUU |
| 5426 | GGGGGAGGGGAGGAGUUUGCA | 8030 | GGGUUUUCUCAGAACUUUUUA |
| 5427 | GGGGAGGGGAGGAGUUUGCAA | 8031 | GGUUUUCUCAGAACUUUUUAC |
| 5428 | GGGAGGGGAGGAGUUUGCAAA | 8032 | GUUUUCUCAGAACUUUUUACU |
| 5429 | GGAGGGGAGGAGUUUGCAAAA | 8033 | UUUUCUCAGAACUUUUUACUU |
| 5430 | GAGGGGAGGAGUUUGCAAAAA | 8034 | UUUCUCAGAACUUUUUACUUG |
| 5431 | AGGGGAGGAGUUUGCAAAAAA | 8035 | UUCUCAGAACUUUUUACUUGU |
| 5432 | GGGGAGGAGUUUGCAAAAAAA | 8036 | UCUCAGAACUUUUUACUUGUU |
| 5433 | GGGAGGAGUUUGCAAAAAAAG | 8037 | CUCAGAACUUUUUACUUGUUG |
| 5434 | GGAGGAGUUUGCAAAAAAAGG | 8038 | UCAGAACUUUUUACUUGUUGA |
| 5435 | GAGGAGUUUGCAAAAAAAGGA | 8039 | CAGAACUUUUUACUUGUUGAG |
| 5436 | AGGAGUUUGCAAAAAAAGGAG | 8040 | AGAACUUUUUACUUGUUGAGU |
| 5437 | GGAGUUUGCAAAAAAAGGAGG | 8041 | GAACUUUUUACUUGUUGAGUG |
| 5438 | GAGUUUGCAAAAAAAGGAGGC | 8042 | AACUUUUUACUUGUUGAGUGC |
| 5439 | AGUUUGCAAAAAAAGGAGGCC | 8043 | ACUUUUUACUUGUUGAGUGCU |
| 5440 | GUUUGCAAAAAAAGGAGGCCC | 8044 | CUUUUUACUUGUUGAGUGCUG |
| 5441 | UUUGCAAAAAAAGGAGGCCCU | 8045 | UUUUUACUUGUUGAGUGCUGG |
| 5442 | UUGCAAAAAAAGGAGGCCCUG | 8046 | UUUUACUUGUUGAGUGCUGGG |
| 5443 | UGCAAAAAAAGGAGGCCCUGA | 8047 | UUUACUUGUUGAGUGCUGGGC |
| 5444 | GCAAAAAAAGGAGGCCCUGAG | 8048 | UUACUUGUUGAGUGCUGGGCG |
| 5445 | CAAAAAAAGGAGGCCCUGAGG | 8049 | UACUUGUUGAGUGCUGGGCGU |
| 5446 | AAAAAAAGGAGGCCCUGAGGU | 8050 | ACUUGUUGAGUGCUGGGCGUA |
| 5447 | AAAAAAGGAGGCCCUGAGGUG | 8051 | CUUGUUGAGUGCUGGGCGUAG |
| 5448 | AAAAAGGAGGCCCUGAGGUGA | 8052 | UUGUUGAGUGCUGGGCGUAGU |
| 5449 | AAAAGGAGGCCCUGAGGUGAG | 8053 | UGUUGAGUGCUGGGCGUAGUA |
| 5450 | AAAGGAGGCCCUGAGGUGAGG | 8054 | GUUGAGUGCUGGGCGUAGUAG |
| 5451 | AAGGAGGCCCUGAGGUGAGGA | 8055 | UUGAGUGCUGGGCGUAGUAGC |
| 5452 | AGGAGGCCCUGAGGUGAGGAU | 8056 | UGAGUGCUGGGCGUAGUAGCA |
| 5453 | GGAGGCCCUGAGGUGAGGAUA | 8057 | GAGUGCUGGGCGUAGUAGCAA |
| 5454 | GAGGCCCUGAGGUGAGGAUAU | 8058 | AGUGCUGGGCGUAGUAGCAAG |
| 5455 | AGGCCCUGAGGUGAGGAUAUC | 8059 | GUGCUGGGCGUAGUAGCAAGA |
| 5456 | GGCCCUGAGGUGAGGAUAUCU | 8060 | UGCUGGGCGUAGUAGCAAGAC |
| 5457 | GCCCUGAGGUGAGGAUAUCUG | 8061 | GCUGGGCGUAGUAGCAAGACC |
| 5458 | CCCUGAGGUGAGGAUAUCUGG | 8062 | CUGGGCGUAGUAGCAAGACCC |
| 5459 | CCUGAGGUGAGGAUAUCUGGG | 8063 | UGGGCGUAGUAGCAAGACCCU |
| 5460 | CUGAGGUGAGGAUAUCUGGGG | 8064 | GGGCGUAGUAGCAAGACCCUC |
| 5461 | UGAGGUGAGGAUAUCUGGGGG | 8065 | GGCGUAGUAGCAAGACCCUCU |
| 5462 | GAGGUGAGGAUAUCUGGGGGC | 8066 | GCGUAGUAGCAAGACCCUCUG |
| 5463 | AGGUGAGGAUAUCUGGGGGCC | 8067 | CGUAGUAGCAAGACCCUCUGA |
| 5464 | GGUGAGGAUAUCUGGGGGCCC | 8068 | GUAGUAGCAAGACCCUCUGAU |
| 5465 | GUGAGGAUAUCUGGGGGCCCA | 8069 | UAGUAGCAAGACCCUCUGAUA |
| 5466 | UGAGGAUAUCUGGGGGCCCAC | 8070 | AGUAGCAAGACCCUCUGAUAU |
| 5467 | GAGGAUAUCUGGGGGCCCACC | 8071 | GUAGCAAGACCCUCUGAUAUU |
| 5468 | AGGAUAUCUGGGGGCCCACCA | 8072 | UAGCAAGACCCUCUGAUAUUU |
| 5469 | GGAUAUCUGGGGGCCCACCAG | 8073 | AGCAAGACCCUCUGAUAUUUC |
| 5470 | GAUAUCUGGGGGCCCACCAGA | 8074 | GCAAGACCCUCUGAUAUUUCA |
| 5471 | AUAUCUGGGGGCCCACCAGAC | 8075 | CAAGACCCUCUGAUAUUUCAG |
| 5472 | UAUCUGGGGGCCCACCAGACA | 8076 | AAGACCCUCUGAUAUUUCAGG |
| 5473 | AUCUGGGGGCCCACCAGACAG | 8077 | AGACCCUCUGAUAUUUCAGGU |
| 5474 | UCUGGGGGCCCACCAGACAGG | 8078 | GACCCUCUGAUAUUUCAGGUU |
| 5475 | CUGGGGGCCCACCAGACAGGU | 8079 | ACCCUCUGAUAUUUCAGGUUG |
| 5476 | UGGGGGCCCACCAGACAGGUU | 8080 | CCCUCUGAUAUUUCAGGUUGC |
| 5477 | GGGGGCCCACCAGACAGGUUU | 8081 | CCUCUGAUAUUUCAGGUUGCA |
| 5478 | GGGGCCCACCAGACAGGUUUA | 8082 | CUCUGAUAUUUCAGGUUGCAC |
| 5479 | GGGCCCACCAGACAGGUUUAA | 8083 | UCUGAUAUUUCAGGUUGCACU |
| 5480 | GGCCCACCAGACAGGUUUAAA | 8084 | CUGAUAUUUCAGGUUGCACUG |
| 5481 | GCCCACCAGACAGGUUUAAAG | 8085 | UGAUAUUUCAGGUUGCACUGA |
| 5482 | CCCACCAGACAGGUUUAAAGA | 8086 | GAUAUUUCAGGUUGCACUGAU |
| 5483 | CCACCAGACAGGUUUAAAGAG | 8087 | AUAUUUCAGGUUGCACUGAUA |
| 5484 | CACCAGACAGGUUUAAAGAGG | 8088 | UAUUUCAGGUUGCACUGAUAC |
| 5485 | ACCAGACAGGUUUAAAGAGGA | 8089 | AUUUCAGGUUGCACUGAUACA |
| 5486 | CCAGACAGGUUUAAAGAGGAA | 8090 | UUUCAGGUUGCACUGAUACAU |
| 5487 | CAGACAGGUUUAAAGAGGAAA | 8091 | UUCAGGUUGCACUGAUACAUU |
| 5488 | AGACAGGUUUAAAGAGGAAAC | 8092 | UCAGGUUGCACUGAUACAUUC |
| 5489 | GACAGGUUUAAAGAGGAAACC | 8093 | CAGGUUGCACUGAUACAUUCU |
| 5490 | ACAGGUUUAAAGAGGAAACCU | 8094 | AGGUUGCACUGAUACAUUCUU |
| 5491 | CAGGUUUAAAGAGGAAACCUC | 8095 | GGUUGCACUGAUACAUUCUUU |
| 5492 | AGGUUUAAAGAGGAAACCUCU | 8096 | GUUGCACUGAUACAUUCUUUG |
| 5493 | GGUUUAAAGAGGAAACCUCUU | 8097 | UUGCACUGAUACAUUCUUUGG |
| 5494 | GUUUAAAGAGGAAACCUCUUC | 8098 | UGCACUGAUACAUUCUUUGGC |
| 5495 | UUUAAAGAGGAAACCUCUUCA | 8099 | GCACUGAUACAUUCUUUGGCC |
| 5496 | UUAAAGAGGAAACCUCUUCAU | 8100 | CACUGAUACAUUCUUUGGCCC |
| 5497 | UAAAGAGGAAACCUCUUCAUU | 8101 | ACUGAUACAUUCUUUGGCCCA |
| 5498 | AAAGAGGAAACCUCUUCAUUC | 8102 | CUGAUACAUUCUUUGGCCCAC |
| 5499 | AAGAGGAAACCUCUUCAUUCA | 8103 | UGAUACAUUCUUUGGCCCACC |
| 5500 | AGAGGAAACCUCUUCAUUCAC | 8104 | GAUACAUUCUUUGGCCCACCA |
| 5501 | GAGGAAACCUCUUCAUUCACA | 8105 | AUACAUUCUUUGGCCCACCAG |
| 5502 | AGGAAACCUCUUCAUUCACAG | 8106 | UACAUUCUUUGGCCCACCAGA |
| 5503 | GGAAACCUCUUCAUUCACAGC | 8107 | ACAUUCUUUGGCCCACCAGAC |
| 5504 | GAAACCUCUUCAUUCACAGCU | 8108 | CAUUCUUUGGCCCACCAGACA |
| 5505 | AAACCUCUUCAUUCACAGCUU | 8109 | AUUCUUUGGCCCACCAGACAG |
| 5506 | AACCUCUUCAUUCACAGCUUC | 8110 | UUCUUUGGCCCACCAGACAGC |
| 5507 | ACCUCUUCAUUCACAGCUUCG | 8111 | UCUUUGGCCCACCAGACAGCU |
| 5508 | CCUCUUCAUUCACAGCUUCGU | 8112 | CUUUGGCCCACCAGACAGCUC |
| 5509 | CUCUUCAUUCACAGCUUCGUU | 8113 | UUUGGCCCACCAGACAGCUCU |
| 5510 | UCUUCAUUCACAGCUUCGUUG | 8114 | UUGGCCCACCAGACAGCUCUG |
| 5511 | CUUCAUUCACAGCUUCGUUGA | 8115 | UGGCCCACCAGACAGCUCUGC |
| 5512 | UUCAUUCACAGCUUCGUUGAG | 8116 | GGCCCACCAGACAGCUCUGCA |
| 5513 | UCAUUCACAGCUUCGUUGAGG | 8117 | GCCCACCAGACAGCUCUGCAG |
| 5514 | CAUUCACAGCUUCGUUGAGGG | 8118 | CCCACCAGACAGCUCUGCAGU |
| 5515 | AUUCACAGCUUCGUUGAGGGG | 8119 | CCACCAGACAGCUCUGCAGUC |
| 5516 | UUCACAGCUUCGUUGAGGGGU | 8120 | CACCAGACAGCUCUGCAGUCA |
| 5517 | UCACAGCUUCGUUGAGGGGUU | 8121 | ACCAGACAGCUCUGCAGUCAG |
| 5518 | CACAGCUUCGUUGAGGGGUUC | 8122 | CCAGACAGCUCUGCAGUCAGG |
| 5519 | ACAGCUUCGUUGAGGGGUUCC | 8123 | CAGACAGCUCUGCAGUCAGGU |
| 5520 | CAGCUUCGUUGAGGGGUUCCU | 8124 | AGACAGCUCUGCAGUCAGGUC |
| 5521 | AGCUUCGUUGAGGGGUUCCUG | 8125 | GACAGCUCUGCAGUCAGGUCU |
| 5522 | GCUUCGUUGAGGGGUUCCUGG | 8126 | ACAGCUCUGCAGUCAGGUCUG |
| 5523 | CUUCGUUGAGGGGUUCCUGGA | 8127 | CAGCUCUGCAGUCAGGUCUGU |
| 5524 | UUCGUUGAGGGGUUCCUGGAG | 8128 | AGCUCUGCAGUCAGGUCUGUG |
| 5525 | UCGUUGAGGGGUUCCUGGAGG | 8129 | GCUCUGCAGUCAGGUCUGUGG |
| 5526 | CGUUGAGGGGUUCCUGGAGGA | 8130 | CUCUGCAGUCAGGUCUGUGGU |
| 5527 | GUUGAGGGGUUCCUGGAGGAC | 8131 | UCUGCAGUCAGGUCUGUGGUU |
| 5528 | UUGAGGGGUUCCUGGAGGACG | 8132 | CUGCAGUCAGGUCUGUGGUUA |
| 5529 | UGAGGGGUUCCUGGAGGACGU | 8133 | UGCAGUCAGGUCUGUGGUUAG |
| 5530 | GAGGGGUUCCUGGAGGACGUC | 8134 | GCAGUCAGGUCUGUGGUUAGG |
| 5531 | AGGGGUUCCUGGAGGACGUCU | 8135 | CAGUCAGGUCUGUGGUUAGGG |
| 5532 | GGGGUUCCUGGAGGACGUCUC | 8136 | AGUCAGGUCUGUGGUUAGGGG |
| 5533 | GGGUUCCUGGAGGACGUCUCU | 8137 | GUCAGGUCUGUGGUUAGGGGA |
| 5534 | GGUUCCUGGAGGACGUCUCUG | 8138 | UCAGGUCUGUGGUUAGGGGAC |
| 5535 | GUUCCUGGAGGACGUCUCUGG | 8139 | CAGGUCUGUGGUUAGGGGACU |
| 5536 | UUCCUGGAGGACGUCUCUGGA | 8140 | AGGUCUGUGGUUAGGGGACUG |
| 5537 | UCCUGGAGGACGUCUCUGGAU | 8141 | GGUCUGUGGUUAGGGGACUGG |
| 5538 | CCUGGAGGACGUCUCUGGAUU | 8142 | GUCUGUGGUUAGGGGACUGGA |
| 5539 | CUGGAGGACGUCUCUGGAUUC | 8143 | UCUGUGGUUAGGGGACUGGAA |
| 5540 | UGGAGGACGUCUCUGGAUUCA | 8144 | CUGUGGUUAGGGGACUGGAAA |
| 5541 | GGAGGACGUCUCUGGAUUCAA | 8145 | UGUGGUUAGGGGACUGGAAAU |
| 5542 | GAGGACGUCUCUGGAUUCAAG | 8146 | GUGGUUAGGGGACUGGAAAUU |
| 5543 | AGGACGUCUCUGGAUUCAAGU | 8147 | UGGUUAGGGGACUGGAAAUUG |
| 5544 | GGACGUCUCUGGAUUCAAGUC | 8148 | GGUUAGGGGACUGGAAAUUGU |
| 5545 | GACGUCUCUGGAUUCAAGUCC | 8149 | GUUAGGGGACUGGAAAUUGUA |
| 5546 | ACGUCUCUGGAUUCAAGUCCC | 8150 | UUAGGGGACUGGAAAUUGUAA |
| 5547 | CGUCUCUGGAUUCAAGUCCCA | 8151 | UAGGGGACUGGAAAUUGUAAU |
| 5548 | GUCUCUGGAUUCAAGUCCCAG | 8152 | AGGGGACUGGAAAUUGUAAUC |
| 5549 | UCUCUGGAUUCAAGUCCCAGG | 8153 | GGGGACUGGAAAUUGUAAUCG |
| 5550 | CUCUGGAUUCAAGUCCCAGGG | 8154 | GGGACUGGAAAUUGUAAUCGC |
| 5551 | UCUGGAUUCAAGUCCCAGGGG | 8155 | GGACUGGAAAUUGUAAUCGCC |
| 5552 | CUGGAUUCAAGUCCCAGGGGU | 8156 | GACUGGAAAUUGUAAUCGCCU |
| 5553 | UGGAUUCAAGUCCCAGGGGUU | 8157 | ACUGGAAAUUGUAAUCGCCUU |
| 5554 | GGAUUCAAGUCCCAGGGGUUC | 8158 | CUGGAAAUUGUAAUCGCCUUG |
| 5555 | GAUUCAAGUCCCAGGGGUUCU | 8159 | UGGAAAUUGUAAUCGCCUUGU |
| 5556 | AUUCAAGUCCCAGGGGUUCUG | 8160 | GGAAAUUGUAAUCGCCUUGUG |
| 5557 | UUCAAGUCCCAGGGGUUCUGG | 8161 | GAAAUUGUAAUCGCCUUGUGG |
| 5558 | UCAAGUCCCAGGGGUUCUGGU | 8162 | AAAUUGUAAUCGCCUUGUGGA |
| 5559 | CAAGUCCCAGGGGUUCUGGUU | 8163 | AAUUGUAAUCGCCUUGUGGAC |
| 5560 | AAGUCCCAGGGGUUCUGGUUG | 8164 | AUUGUAAUCGCCUUGUGGACC |
| 5561 | AGUCCCAGGGGUUCUGGUUGG | 8165 | UUGUAAUCGCCUUGUGGACCU |
| 5562 | GUCCCAGGGGUUCUGGUUGGG | 8166 | UGUAAUCGCCUUGUGGACCUC |
| 5563 | UCCCAGGGGUUCUGGUUGGGA | 8167 | GUAAUCGCCUUGUGGACCUCU |
| 5564 | CCCAGGGGUUCUGGUUGGGAC | 8168 | UAAUCGCCUUGUGGACCUCUG |
| 5565 | CCAGGGGUUCUGGUUGGGACU | 8169 | AAUCGCCUUGUGGACCUCUGC |
| 5566 | CAGGGGUUCUGGUUGGGACUG | 8170 | AUCGCCUUGUGGACCUCUGCA |
| 5567 | AGGGGUUCUGGUUGGGACUGU | 8171 | UCGCCUUGUGGACCUCUGCAG |
| 5568 | GGGGUUCUGGUUGGGACUGUC | 8172 | CGCCUUGUGGACCUCUGCAGA |
| 5569 | GGGUUCUGGUUGGGACUGUCA | 8173 | GCCUUGUGGACCUCUGCAGAG |
| 5570 | GGUUCUGGUUGGGACUGUCAG | 8174 | CCUUGUGGACCUCUGCAGAGC |
| 5571 | GUUCUGGUUGGGACUGUCAGG | 8175 | CUUGUGGACCUCUGCAGAGCC |
| 5572 | UUCUGGUUGGGACUGUCAGGG | 8176 | UUGUGGACCUCUGCAGAGCCA |
| 5573 | UCUGGUUGGGACUGUCAGGGC | 8177 | UGUGGACCUCUGCAGAGCCAC |
| 5574 | CUGGUUGGGACUGUCAGGGCG | 8178 | GUGGACCUCUGCAGAGCCACU |
| 5575 | UGGUUGGGACUGUCAGGGCGA | 8179 | UGGACCUCUGCAGAGCCACUG |
| 5576 | GGUUGGGACUGUCAGGGCGAA | 8180 | GGACCUCUGCAGAGCCACUGG |
| 5577 | GUUGGGACUGUCAGGGCGAAA | 8181 | GACCUCUGCAGAGCCACUGGU |
| 5578 | UUGGGACUGUCAGGGCGAAAU | 8182 | ACCUCUGCAGAGCCACUGGUA |
| 5579 | UGGGACUGUCAGGGCGAAAUG | 8183 | CCUCUGCAGAGCCACUGGUAG |
| 5580 | GGGACUGUCAGGGCGAAAUGA | 8184 | CUCUGCAGAGCCACUGGUAGU |
| 5581 | GGACUGUCAGGGCGAAAUGAC | 8185 | UCUGCAGAGCCACUGGUAGUC |
| 5582 | GACUGUCAGGGCGAAAUGACC | 8186 | CUGCAGAGCCACUGGUAGUCA |
| 5583 | ACUGUCAGGGCGAAAUGACCA | 8187 | UGCAGAGCCACUGGUAGUCAG |
| 5584 | CUGUCAGGGCGAAAUGACCAG | 8188 | GCAGAGCCACUGGUAGUCAGA |
| 5585 | UGUCAGGGCGAAAUGACCAGC | 8189 | CAGAGCCACUGGUAGUCAGAC |
| 5586 | GUCAGGGCGAAAUGACCAGCA | 8190 | AGAGCCACUGGUAGUCAGACC |
| 5587 | UCAGGGCGAAAUGACCAGCAG | 8191 | GAGCCACUGGUAGUCAGACCA |
| 5588 | CAGGGCGAAAUGACCAGCAGA | 8192 | AGCCACUGGUAGUCAGACCAC |
| 5589 | AGGGCGAAAUGACCAGCAGAU | 8193 | GCCACUGGUAGUCAGACCACC |
| 5590 | GGGCGAAAUGACCAGCAGAUG | 8194 | CCACUGGUAGUCAGACCACCU |
| 5591 | GGCGAAAUGACCAGCAGAUGC | 8195 | CACUGGUAGUCAGACCACCUA |
| 5592 | GCGAAAUGACCAGCAGAUGCU | 8196 | ACUGGUAGUCAGACCACCUAA |
| 5593 | CGAAAUGACCAGCAGAUGCUG | 8197 | CUGGUAGUCAGACCACCUAAU |
| 5594 | GAAAUGACCAGCAGAUGCUGG | 8198 | UGGUAGUCAGACCACCUAAUU |
| 5595 | AAAUGACCAGCAGAUGCUGGG | 8199 | GGUAGUCAGACCACCUAAUUC |
| 5596 | AAUGACCAGCAGAUGCUGGGA | 8200 | GUAGUCAGACCACCUAAUUCG |
| 5597 | AUGACCAGCAGAUGCUGGGAC | 8201 | UAGUCAGACCACCUAAUUCGU |
| 5598 | UGACCAGCAGAUGCUGGGACA | 8202 | AGUCAGACCACCUAAUUCGUU |
| 5599 | GACCAGCAGAUGCUGGGACAG | 8203 | GUCAGACCACCUAAUUCGUUG |
| 5600 | ACCAGCAGAUGCUGGGACAGC | 8204 | UCAGACCACCUAAUUCGUUGG |
| 5601 | CCAGCAGAUGCUGGGACAGCA | 8205 | CAGACCACCUAAUUCGUUGGA |
| 5602 | CAGCAGAUGCUGGGACAGCAG | 8206 | AGACCACCUAAUUCGUUGGAG |
| 5603 | AGCAGAUGCUGGGACAGCAGC | 8207 | GACCACCUAAUUCGUUGGAGU |
| 5604 | GCAGAUGCUGGGACAGCAGCU | 8208 | ACCACCUAAUUCGUUGGAGUC |
| 5605 | CAGAUGCUGGGACAGCAGCUG | 8209 | CCACCUAAUUCGUUGGAGUCA |
| 5606 | AGAUGCUGGGACAGCAGCUGC | 8210 | CACCUAAUUCGUUGGAGUCAU |
| 5607 | GAUGCUGGGACAGCAGCUGCC | 8211 | ACCUAAUUCGUUGGAGUCAUU |
| 5608 | AUGCUGGGACAGCAGCUGCCC | 8212 | CCUAAUUCGUUGGAGUCAUUC |
| 5609 | UGCUGGGACAGCAGCUGCCCG | 8213 | CUAAUUCGUUGGAGUCAUUCA |
| 5610 | GCUGGGACAGCAGCUGCCCGC | 8214 | UAAUUCGUUGGAGUCAUUCAC |
| 5611 | CUGGGACAGCAGCUGCCCGCA | 8215 | AAUUCGUUGGAGUCAUUCACU |
| 5612 | UGGGACAGCAGCUGCCCGCAG | 8216 | AUUCGUUGGAGUCAUUCACUU |
| 5613 | GGGACAGCAGCUGCCCGCAGA | 8217 | UUCGUUGGAGUCAUUCACUUU |
| 5614 | GGACAGCAGCUGCCCGCAGAC | 8218 | UCGUUGGAGUCAUUCACUUUA |
| 5615 | GACAGCAGCUGCCCGCAGACU | 8219 | CGUUGGAGUCAUUCACUUUAG |
| 5616 | ACAGCAGCUGCCCGCAGACUC | 8220 | GUUGGAGUCAUUCACUUUAGA |
| 5617 | CAGCAGCUGCCCGCAGACUCG | 8221 | UUGGAGUCAUUCACUUUAGAA |
| 5618 | AGCAGCUGCCCGCAGACUCGG | 8222 | UGGAGUCAUUCACUUUAGAAU |
| 5619 | GCAGCUGCCCGCAGACUCGGG | 8223 | GGAGUCAUUCACUUUAGAAUG |
| 5620 | CAGCUGCCCGCAGACUCGGGA | 8224 | GAGUCAUUCACUUUAGAAUGC |
| 5621 | AGCUGCCCGCAGACUCGGGAG | 8225 | AGUCAUUCACUUUAGAAUGCU |
| 5622 | GCUGCCCGCAGACUCGGGAGG | 8226 | GUCAUUCACUUUAGAAUGCUG |
| 5623 | CUGCCCGCAGACUCGGGAGGU | 8227 | UCAUUCACUUUAGAAUGCUGU |
| 5624 | UGCCCGCAGACUCGGGAGGUA | 8228 | CAUUCACUUUAGAAUGCUGUG |
| 5625 | GCCCGCAGACUCGGGAGGUAG | 8229 | AUUCACUUUAGAAUGCUGUGU |
| 5626 | CCCGCAGACUCGGGAGGUAGG | 8230 | UUCACUUUAGAAUGCUGUGUC |
| 5627 | CCGCAGACUCGGGAGGUAGGA | 8231 | UCACUUUAGAAUGCUGUGUCA |
| 5628 | CGCAGACUCGGGAGGUAGGAG | 8232 | CACUUUAGAAUGCUGUGUCAC |
| 5629 | GCAGACUCGGGAGGUAGGAGG | 8233 | ACUUUAGAAUGCUGUGUCACU |
| 5630 | CAGACUCGGGAGGUAGGAGGA | 8234 | CUUUAGAAUGCUGUGUCACUA |
| 5631 | AGACUCGGGAGGUAGGAGGAC | 8235 | UUUAGAAUGCUGUGUCACUAU |
| 5632 | GACUCGGGAGGUAGGAGGACU | 8236 | UUAGAAUGCUGUGUCACUAUA |
| 5633 | ACUCGGGAGGUAGGAGGACUG | 8237 | UAGAAUGCUGUGUCACUAUAG |
| 5634 | CUCGGGAGGUAGGAGGACUGG | 8238 | AGAAUGCUGUGUCACUAUAGG |
| 5635 | UCGGGAGGUAGGAGGACUGGC | 8239 | GAAUGCUGUGUCACUAUAGGU |
| 5636 | CGGGAGGUAGGAGGACUGGCC | 8240 | AAUGCUGUGUCACUAUAGGUG |
| 5637 | GGGAGGUAGGAGGACUGGCCG | 8241 | AUGCUGUGUCACUAUAGGUGU |
| 5638 | GGAGGUAGGAGGACUGGCCGG | 8242 | UGCUGUGUCACUAUAGGUGUA |
| 5639 | GAGGUAGGAGGACUGGCCGGG | 8243 | GCUGUGUCACUAUAGGUGUAA |
| 5640 | AGGUAGGAGGACUGGCCGGGC | 8244 | CUGUGUCACUAUAGGUGUAAC |
| 5641 | GGUAGGAGGACUGGCCGGGCA | 8245 | UGUGUCACUAUAGGUGUAACU |
| 5642 | GUAGGAGGACUGGCCGGGCAG | 8246 | GUGUCACUAUAGGUGUAACUA |
| 5643 | UAGGAGGACUGGCCGGGCAGU | 8247 | UGUCACUAUAGGUGUAACUAC |
| 5644 | AGGAGGACUGGCCGGGCAGUG | 8248 | GUCACUAUAGGUGUAACUACG |
| 5645 | GGAGGACUGGCCGGGCAGUGU | 8249 | UCACUAUAGGUGUAACUACGU |
| 5646 | GAGGACUGGCCGGGCAGUGUG | 8250 | CACUAUAGGUGUAACUACGUC |
| 5647 | AGGACUGGCCGGGCAGUGUGC | 8251 | ACUAUAGGUGUAACUACGUCA |
| 5648 | GGACUGGCCGGGCAGUGUGCU | 8252 | CUAUAGGUGUAACUACGUCAC |
| 5649 | GACUGGCCGGGCAGUGUGCUG | 8253 | UAUAGGUGUAACUACGUCACU |
| 5650 | ACUGGCCGGGCAGUGUGCUGG | 8254 | AUAGGUGUAACUACGUCACUG |
| 5651 | CUGGCCGGGCAGUGUGCUGGG | 8255 | UAGGUGUAACUACGUCACUGG |
| 5652 | UGGCCGGGCAGUGUGCUGGGC | 8256 | AGGUGUAACUACGUCACUGGG |
| 5653 | GGCCGGGCAGUGUGCUGGGCC | 8257 | GGUGUAACUACGUCACUGGGA |
| 5654 | GCCGGGCAGUGUGCUGGGCCC | 8258 | GUGUAACUACGUCACUGGGAC |
| 5655 | CCGGGCAGUGUGCUGGGCCCU | 8259 | UGUAACUACGUCACUGGGACU |
| 5656 | CGGGCAGUGUGCUGGGCCCUG | 8260 | GUAACUACGUCACUGGGACUA |
| 5657 | GGGCAGUGUGCUGGGCCCUGC | 8261 | UAACUACGUCACUGGGACUAC |
| 5658 | GGCAGUGUGCUGGGCCCUGCC | 8262 | AACUACGUCACUGGGACUACC |
| 5659 | GCAGUGUGCUGGGCCCUGCCC | 8263 | ACUACGUCACUGGGACUACCU |
| 5660 | CAGUGUGCUGGGCCCUGCCCU | 8264 | CUACGUCACUGGGACUACCUC |
| 5661 | AGUGUGCUGGGCCCUGCCCUG | 8265 | UACGUCACUGGGACUACCUCU |
| 5662 | GUGUGCUGGGCCCUGCCCUGA | 8266 | ACGUCACUGGGACUACCUCUC |
| 5663 | UGUGCUGGGCCCUGCCCUGAG | 8267 | CGUCACUGGGACUACCUCUCU |
| 5664 | GUGCUGGGCCCUGCCCUGAGG | 8268 | GUCACUGGGACUACCUCUCUU |
| 5665 | UGCUGGGCCCUGCCCUGAGGA | 8269 | UCACUGGGACUACCUCUCUUC |
| 5666 | GCUGGGCCCUGCCCUGAGGAG | 8270 | CACUGGGACUACCUCUCUUCU |
| 5667 | CUGGGCCCUGCCCUGAGGAGA | 8271 | ACUGGGACUACCUCUCUUCUG |
| 5668 | UGGGCCCUGCCCUGAGGAGAC | 8272 | CUGGGACUACCUCUCUUCUGA |
| 5669 | GGGCCCUGCCCUGAGGAGACA | 8273 | UGGGACUACCUCUCUUCUGAA |
| 5670 | GGCCCUGCCCUGAGGAGACAG | 8274 | GGGACUACCUCUCUUCUGAAG |
| 5671 | GCCCUGCCCUGAGGAGACAGA | 8275 | GGACUACCUCUCUUCUGAAGC |
| 5672 | CCCUGCCCUGAGGAGACAGAC | 8276 | GACUACCUCUCUUCUGAAGCU |
| 5673 | CCUGCCCUGAGGAGACAGACC | 8277 | ACUACCUCUCUUCUGAAGCUU |
| 5674 | CUGCCCUGAGGAGACAGACCA | 8278 | CUACCUCUCUUCUGAAGCUUC |
| 5675 | UGCCCUGAGGAGACAGACCAG | 8279 | UACCUCUCUUCUGAAGCUUCC |
| 5676 | GCCCUGAGGAGACAGACCAGG | 8280 | ACCUCUCUUCUGAAGCUUCCU |
| 5677 | CCCUGAGGAGACAGACCAGGU | 8281 | CCUCUCUUCUGAAGCUUCCUG |
| 5678 | CCUGAGGAGACAGACCAGGUG | 8282 | CUCUCUUCUGAAGCUUCCUGA |
| 5679 | CUGAGGAGACAGACCAGGUGG | 8283 | UCUCUUCUGAAGCUUCCUGAU |
| 5680 | UGAGGAGACAGACCAGGUGGC | 8284 | CUCUUCUGAAGCUUCCUGAUU |
| 5681 | GAGGAGACAGACCAGGUGGCU | 8285 | UCUUCUGAAGCUUCCUGAUUA |
| 5682 | AGGAGACAGACCAGGUGGCUA | 8286 | CUUCUGAAGCUUCCUGAUUAA |
| 5683 | GGAGACAGACCAGGUGGCUAC | 8287 | UUCUGAAGCUUCCUGAUUAAG |
| 5684 | GAGACAGACCAGGUGGCUACA | 8288 | UCUGAAGCUUCCUGAUUAAGC |
| 5685 | AGACAGACCAGGUGGCUACAG | 8289 | CUGAAGCUUCCUGAUUAAGCU |
| 5686 | GACAGACCAGGUGGCUACAGG | 8290 | UGAAGCUUCCUGAUUAAGCUC |
| 5687 | ACAGACCAGGUGGCUACAGGA | 8291 | GAAGCUUCCUGAUUAAGCUCU |
| 5688 | CAGACCAGGUGGCUACAGGAU | 8292 | AAGCUUCCUGAUUAAGCUCUG |
| 5689 | AGACCAGGUGGCUACAGGAUC | 8293 | AGCUUCCUGAUUAAGCUCUGC |
| 5690 | GACCAGGUGGCUACAGGAUCU | 8294 | GCUUCCUGAUUAAGCUCUGCU |
| 5691 | ACCAGGUGGCUACAGGAUCUC | 8295 | CUUCCUGAUUAAGCUCUGCUG |
| 5692 | CCAGGUGGCUACAGGAUCUCC | 8296 | UUCCUGAUUAAGCUCUGCUGG |
| 5693 | CAGGUGGCUACAGGAUCUCCU | 8297 | UCCUGAUUAAGCUCUGCUGGU |
| 5694 | AGGUGGCUACAGGAUCUCCUC | 8298 | CCUGAUUAAGCUCUGCUGGUC |
| 5695 | GGUGGCUACAGGAUCUCCUCC | 8299 | CUGAUUAAGCUCUGCUGGUCA |
| 5696 | GUGGCUACAGGAUCUCCUCCC | 8300 | UGAUUAAGCUCUGCUGGUCAC |
| 5697 | UGGCUACAGGAUCUCCUCCCG | 8301 | GAUUAAGCUCUGCUGGUCACU |
| 5698 | GGCUACAGGAUCUCCUCCCGC | 8302 | AUUAAGCUCUGCUGGUCACUG |
| 5699 | GCUACAGGAUCUCCUCCCGCG | 8303 | UUAAGCUCUGCUGGUCACUGG |
| 5700 | CUACAGGAUCUCCUCCCGCGG | 8304 | UAAGCUCUGCUGGUCACUGGA |
| 5701 | UACAGGAUCUCCUCCCGCGGG | 8305 | AAGCUCUGCUGGUCACUGGAA |
| 5702 | ACAGGAUCUCCUCCCGCGGGC | 8306 | AGCUCUGCUGGUCACUGGAAG |
| 5703 | CAGGAUCUCCUCCCGCGGGCU | 8307 | GCUCUGCUGGUCACUGGAAGA |
| 5704 | AGGAUCUCCUCCCGCGGGCUC | 8308 | CUCUGCUGGUCACUGGAAGAU |
| 5705 | GGAUCUCCUCCCGCGGGCUCC | 8309 | UCUGCUGGUCACUGGAAGAUA |
| 5706 | GAUCUCCUCCCGCGGGCUCCU | 8310 | CUGCUGGUCACUGGAAGAUAC |
| 5707 | AUCUCCUCCCGCGGGCUCCUG | 8311 | UGCUGGUCACUGGAAGAUACA |
| 5708 | UCUCCUCCCGCGGGCUCCUGG | 8312 | GCUGGUCACUGGAAGAUACAG |
| 5709 | CUCCUCCCGCGGGCUCCUGGC | 8313 | CUGGUCACUGGAAGAUACAGC |
| 5710 | UCCUCCCGCGGGCUCCUGGCC | 8314 | UGGUCACUGGAAGAUACAGCA |
| 5711 | CCUCCCGCGGGCUCCUGGCCU | 8315 | GGUCACUGGAAGAUACAGCAG |
| 5712 | CUCCCGCGGGCUCCUGGCCUU | 8316 | GUCACUGGAAGAUACAGCAGG |
| 5713 | UCCCGCGGGCUCCUGGCCUUC | 8317 | UCACUGGAAGAUACAGCAGGU |
| 5714 | CCCGCGGGCUCCUGGCCUUCA | 8318 | CACUGGAAGAUACAGCAGGUC |
| 5715 | CCGCGGGCUCCUGGCCUUCAG | 8319 | ACUGGAAGAUACAGCAGGUCU |
| 5716 | CGCGGGCUCCUGGCCUUCAGA | 8320 | CUGGAAGAUACAGCAGGUCUG |
| 5717 | GCGGGCUCCUGGCCUUCAGAG | 8321 | UGGAAGAUACAGCAGGUCUGA |
| 5718 | CGGGCUCCUGGCCUUCAGAGG | 8322 | GGAAGAUACAGCAGGUCUGAG |
| 5719 | GGGCUCCUGGCCUUCAGAGGG | 8323 | GAAGAUACAGCAGGUCUGAGU |
| 5720 | GGCUCCUGGCCUUCAGAGGGG | 8324 | AAGAUACAGCAGGUCUGAGUG |
| 5721 | GCUCCUGGCCUUCAGAGGGGU | 8325 | AGAUACAGCAGGUCUGAGUGC |
| 5722 | CUCCUGGCCUUCAGAGGGGUC | 8326 | GAUACAGCAGGUCUGAGUGCA |
| 5723 | UCCUGGCCUUCAGAGGGGUCU | 8327 | AUACAGCAGGUCUGAGUGCAG |
| 5724 | CCUGGCCUUCAGAGGGGUCUG | 8328 | UACAGCAGGUCUGAGUGCAGC |
| 5725 | CUGGCCUUCAGAGGGGUCUGC | 8329 | ACAGCAGGUCUGAGUGCAGCU |
| 5726 | UGGCCUUCAGAGGGGUCUGCC | 8330 | CAGCAGGUCUGAGUGCAGCUC |
| 5727 | GGCCUUCAGAGGGGUCUGCCC | 8331 | AGCAGGUCUGAGUGCAGCUCU |
| 5728 | GCCUUCAGAGGGGUCUGCCCG | 8332 | GCAGGUCUGAGUGCAGCUCUG |
| 5729 | CCUUCAGAGGGGUCUGCCCGU | 8333 | CAGGUCUGAGUGCAGCUCUGG |
| 5730 | CUUCAGAGGGGUCUGCCCGUU | 8334 | AGGUCUGAGUGCAGCUCUGGG |
| 5731 | UUCAGAGGGGUCUGCCCGUUG | 8335 | GGUCUGAGUGCAGCUCUGGGU |
| 5732 | UCAGAGGGGUCUGCCCGUUGG | 8336 | GUCUGAGUGCAGCUCUGGGUG |
| 5733 | CAGAGGGGUCUGCCCGUUGGG | 8337 | UCUGAGUGCAGCUCUGGGUGG |
| 5734 | AGAGGGGUCUGCCCGUUGGGU | 8338 | CUGAGUGCAGCUCUGGGUGGG |
| 5735 | GAGGGGUCUGCCCGUUGGGUA | 8339 | UGAGUGCAGCUCUGGGUGGGC |
| 5736 | AGGGGUCUGCCCGUUGGGUAC | 8340 | GAGUGCAGCUCUGGGUGGGCU |
| 5737 | GGGGUCUGCCCGUUGGGUACA | 8341 | AGUGCAGCUCUGGGUGGGCUC |
| 5738 | GGGUCUGCCCGUUGGGUACAG | 8342 | GUGCAGCUCUGGGUGGGCUCU |
| 5739 | GGUCUGCCCGUUGGGUACAGA | 8343 | UGCAGCUCUGGGUGGGCUCUG |
| 5740 | GUCUGCCCGUUGGGUACAGAG | 8344 | GCAGCUCUGGGUGGGCUCUGC |
| 5741 | UCUGCCCGUUGGGUACAGAGC | 8345 | CAGCUCUGGGUGGGCUCUGCC |
| 5742 | CUGCCCGUUGGGUACAGAGCC | 8346 | AGCUCUGGGUGGGCUCUGCCU |
| 5743 | UGCCCGUUGGGUACAGAGCCA | 8347 | GCUCUGGGUGGGCUCUGCCUC |
| 5744 | GCCCGUUGGGUACAGAGCCAU | 8348 | CUCUGGGUGGGCUCUGCCUCC |
| 5745 | CCCGUUGGGUACAGAGCCAUU | 8349 | UCUGGGUGGGCUCUGCCUCCA |
| 5746 | CCGUUGGGUACAGAGCCAUUC | 8350 | CUGGGUGGGCUCUGCCUCCAA |
| 5747 | CGUUGGGUACAGAGCCAUUCU | 8351 | UGGGUGGGCUCUGCCUCCAAG |
| 5748 | GUUGGGUACAGAGCCAUUCUG | 8352 | GGGUGGGCUCUGCCUCCAAGA |
| 5749 | UUGGGUACAGAGCCAUUCUGA | 8353 | GGUGGGCUCUGCCUCCAAGAU |
| 5750 | UGGGUACAGAGCCAUUCUGAC | 8354 | GUGGGCUCUGCCUCCAAGAUG |
| 5751 | GGGUACAGAGCCAUUCUGACC | 8355 | UGGGCUCUGCCUCCAAGAUGC |
| 5752 | GGUACAGAGCCAUUCUGACCA | 8356 | GGGCUCUGCCUCCAAGAUGCC |
| 5753 | GUACAGAGCCAUUCUGACCAU | 8357 | GGCUCUGCCUCCAAGAUGCCC |
| 5754 | UACAGAGCCAUUCUGACCAUG | 8358 | GCUCUGCCUCCAAGAUGCCCA |
| 5755 | ACAGAGCCAUUCUGACCAUGC | 8359 | CUCUGCCUCCAAGAUGCCCAG |
| 5756 | CAGAGCCAUUCUGACCAUGCA | 8360 | UCUGCCUCCAAGAUGCCCAGU |
| 5757 | AGAGCCAUUCUGACCAUGCAG | 8361 | CUGCCUCCAAGAUGCCCAGUU |
| 5758 | GAGCCAUUCUGACCAUGCAGG | 8362 | UGCCUCCAAGAUGCCCAGUUC |
| 5759 | AGCCAUUCUGACCAUGCAGGA | 8363 | GCCUCCAAGAUGCCCAGUUCC |
| 5760 | GCCAUUCUGACCAUGCAGGAG | 8364 | CCUCCAAGAUGCCCAGUUCCA |
| 5761 | CCAUUCUGACCAUGCAGGAGU | 8365 | CUCCAAGAUGCCCAGUUCCAA |
| 5762 | CAUUCUGACCAUGCAGGAGUU | 8366 | UCCAAGAUGCCCAGUUCCAAC |
| 5763 | AUUCUGACCAUGCAGGAGUUU | 8367 | CCAAGAUGCCCAGUUCCAACC |
| 5764 | UUCUGACCAUGCAGGAGUUUG | 8368 | CAAGAUGCCCAGUUCCAACCU |
| 5765 | UCUGACCAUGCAGGAGUUUGC | 8369 | AAGAUGCCCAGUUCCAACCUA |
| 5766 | CUGACCAUGCAGGAGUUUGCC | 8370 | AGAUGCCCAGUUCCAACCUAG |
| 5767 | UGACCAUGCAGGAGUUUGCCC | 8371 | GAUGCCCAGUUCCAACCUAGC |
| 5768 | GACCAUGCAGGAGUUUGCCCU | 8372 | AUGCCCAGUUCCAACCUAGCC |
| 5769 | ACCAUGCAGGAGUUUGCCCUU | 8373 | UGCCCAGUUCCAACCUAGCCC |
| 5770 | CCAUGCAGGAGUUUGCCCUUC | 8374 | GCCCAGUUCCAACCUAGCCCC |
| 5771 | CAUGCAGGAGUUUGCCCUUCU | 8375 | CCCAGUUCCAACCUAGCCCCA |
| 5772 | AUGCAGGAGUUUGCCCUUCUC | 8376 | CCAGUUCCAACCUAGCCCCAG |
| 5773 | UGCAGGAGUUUGCCCUUCUCU | 8377 | CAGUUCCAACCUAGCCCCAGA |
| 5774 | GCAGGAGUUUGCCCUUCUCUC | 8378 | AGUUCCAACCUAGCCCCAGAA |
| 5775 | CAGGAGUUUGCCCUUCUCUCG | 8379 | GUUCCAACCUAGCCCCAGAAG |
| 5776 | AGGAGUUUGCCCUUCUCUCGG | 8380 | UUCCAACCUAGCCCCAGAAGA |
| 5777 | GGAGUUUGCCCUUCUCUCGGU | 8381 | UCCAACCUAGCCCCAGAAGAU |
| 5778 | GAGUUUGCCCUUCUCUCGGUC | 8382 | CCAACCUAGCCCCAGAAGAUG |
| 5779 | AGUUUGCCCUUCUCUCGGUCU | 8383 | CAACCUAGCCCCAGAAGAUGU |
| 5780 | GUUUGCCCUUCUCUCGGUCUG | 8384 | AACCUAGCCCCAGAAGAUGUG |
| 5781 | UUUGCCCUUCUCUCGGUCUGG | 8385 | ACCUAGCCCCAGAAGAUGUGG |
| 5782 | UUGCCCUUCUCUCGGUCUGGC | 8386 | CCUAGCCCCAGAAGAUGUGGU |
| 5783 | UGCCCUUCUCUCGGUCUGGCC | 8387 | CUAGCCCCAGAAGAUGUGGUA |
| 5784 | GCCCUUCUCUCGGUCUGGCCA | 8388 | UAGCCCCAGAAGAUGUGGUAC |
| 5785 | CCCUUCUCUCGGUCUGGCCAU | 8389 | AGCCCCAGAAGAUGUGGUACA |
| 5786 | CCUUCUCUCGGUCUGGCCAUG | 8390 | GCCCCAGAAGAUGUGGUACAU |
| 5787 | CUUCUCUCGGUCUGGCCAUGU | 8391 | CCCCAGAAGAUGUGGUACAUC |
| 5788 | UUCUCUCGGUCUGGCCAUGUA | 8392 | CCCAGAAGAUGUGGUACAUCA |
| 5789 | UCUCUCGGUCUGGCCAUGUAA | 8393 | CCAGAAGAUGUGGUACAUCAU |
| 5790 | CUCUCGGUCUGGCCAUGUAAA | 8394 | CAGAAGAUGUGGUACAUCAUC |
| 5791 | UCUCGGUCUGGCCAUGUAAAG | 8395 | AGAAGAUGUGGUACAUCAUCU |
| 5792 | CUCGGUCUGGCCAUGUAAAGA | 8396 | GAAGAUGUGGUACAUCAUCUU |
| 5793 | UCGGUCUGGCCAUGUAAAGAU | 8397 | AAGAUGUGGUACAUCAUCUUC |
| 5794 | CGGUCUGGCCAUGUAAAGAUG | 8398 | AGAUGUGGUACAUCAUCUUCA |
| 5795 | GGUCUGGCCAUGUAAAGAUGG | 8399 | GAUGUGGUACAUCAUCUUCAG |
| 5796 | GUCUGGCCAUGUAAAGAUGGC | 8400 | AUGUGGUACAUCAUCUUCAGG |
| 5797 | UCUGGCCAUGUAAAGAUGGCA | 8401 | UGUGGUACAUCAUCUUCAGGU |
| 5798 | CUGGCCAUGUAAAGAUGGCAU | 8402 | GUGGUACAUCAUCUUCAGGUA |
| 5799 | UGGCCAUGUAAAGAUGGCAUC | 8403 | UGGUACAUCAUCUUCAGGUAG |
| 5800 | GGCCAUGUAAAGAUGGCAUCA | 8404 | GGUACAUCAUCUUCAGGUAGA |
| 5801 | GCCAUGUAAAGAUGGCAUCAU | 8405 | GUACAUCAUCUUCAGGUAGAA |
| 5802 | CCAUGUAAAGAUGGCAUCAUC | 8406 | UACAUCAUCUUCAGGUAGAAA |
| 5803 | CAUGUAAAGAUGGCAUCAUCG | 8407 | ACAUCAUCUUCAGGUAGAAAG |
| 5804 | AUGUAAAGAUGGCAUCAUCGC | 8408 | CAUCAUCUUCAGGUAGAAAGC |
| 5805 | UGUAAAGAUGGCAUCAUCGCU | 8409 | AUCAUCUUCAGGUAGAAAGCC |
| 5806 | GUAAAGAUGGCAUCAUCGCUG | 8410 | UCAUCUUCAGGUAGAAAGCCC |
| 5807 | UAAAGAUGGCAUCAUCGCUGU | 8411 | CAUCUUCAGGUAGAAAGCCCA |
| 5808 | AAAGAUGGCAUCAUCGCUGUC | 8412 | AUCUUCAGGUAGAAAGCCCAA |
| 5809 | AAGAUGGCAUCAUCGCUGUCC | 8413 | UCUUCAGGUAGAAAGCCCAAA |
| 5810 | AGAUGGCAUCAUCGCUGUCCA | 8414 | CUUCAGGUAGAAAGCCCAAAU |
| 5811 | GAUGGCAUCAUCGCUGUCCAG | 8415 | UUCAGGUAGAAAGCCCAAAUC |
| 5812 | AUGGCAUCAUCGCUGUCCAGC | 8416 | UCAGGUAGAAAGCCCAAAUCA |
| 5813 | UGGCAUCAUCGCUGUCCAGCU | 8417 | CAGGUAGAAAGCCCAAAUCAU |
| 5814 | GGCAUCAUCGCUGUCCAGCUC | 8418 | AGGUAGAAAGCCCAAAUCAUC |
| 5815 | GCAUCAUCGCUGUCCAGCUCU | 8419 | GGUAGAAAGCCCAAAUCAUCU |
| 5816 | CAUCAUCGCUGUCCAGCUCUG | 8420 | GUAGAAAGCCCAAAUCAUCUG |
| 5817 | AUCAUCGCUGUCCAGCUCUGA | 8421 | UAGAAAGCCCAAAUCAUCUGC |
| 5818 | UCAUCGCUGUCCAGCUCUGAC | 8422 | AGAAAGCCCAAAUCAUCUGCA |
| 5819 | CAUCGCUGUCCAGCUCUGACU | 8423 | GAAAGCCCAAAUCAUCUGCAG |
| 5820 | AUCGCUGUCCAGCUCUGACUC | 8424 | AAAGCCCAAAUCAUCUGCAGU |
| 5821 | UCGCUGUCCAGCUCUGACUCG | 8425 | AAGCCCAAAUCAUCUGCAGUU |
| 5822 | CGCUGUCCAGCUCUGACUCGG | 8426 | AGCCCAAAUCAUCUGCAGUUU |
| 5823 | GCUGUCCAGCUCUGACUCGGA | 8427 | GCCCAAAUCAUCUGCAGUUUG |
| 5824 | CUGUCCAGCUCUGACUCGGAG | 8428 | CCCAAAUCAUCUGCAGUUUGG |
| 5825 | UGUCCAGCUCUGACUCGGAGU | 8429 | CCAAAUCAUCUGCAGUUUGGA |
| 5826 | GUCCAGCUCUGACUCGGAGUG | 8430 | CAAAUCAUCUGCAGUUUGGAA |
| 5827 | UCCAGCUCUGACUCGGAGUGC | 8431 | AAAUCAUCUGCAGUUUGGAAU |
| 5828 | CCAGCUCUGACUCGGAGUGCA | 8432 | AAUCAUCUGCAGUUUGGAAUU |
| 5829 | CAGCUCUGACUCGGAGUGCAU | 8433 | AUCAUCUGCAGUUUGGAAUUU |
| 5830 | AGCUCUGACUCGGAGUGCAUC | 8434 | UCAUCUGCAGUUUGGAAUUUU |
| 5831 | GCUCUGACUCGGAGUGCAUCA | 8435 | CAUCUGCAGUUUGGAAUUUUU |
| 5832 | CUCUGACUCGGAGUGCAUCAG | 8436 | AUCUGCAGUUUGGAAUUUUUU |
| 5833 | UCUGACUCGGAGUGCAUCAGG | 8437 | UCUGCAGUUUGGAAUUUUUUU |
| 5834 | CUGACUCGGAGUGCAUCAGGC | 8438 | CUGCAGUUUGGAAUUUUUUUA |
| 5835 | UGACUCGGAGUGCAUCAGGCU | 8439 | UGCAGUUUGGAAUUUUUUUAA |
| 5836 | GACUCGGAGUGCAUCAGGCUG | 8440 | GCAGUUUGGAAUUUUUUUAAA |
| 5837 | ACUCGGAGUGCAUCAGGCUGC | 8441 | CAGUUUGGAAUUUUUUUAAAA |
| 5838 | CUCGGAGUGCAUCAGGCUGCU | 8442 | AGUUUGGAAUUUUUUUAAAAA |
| 5839 | UCGGAGUGCAUCAGGCUGCUA | 8443 | GUUUGGAAUUUUUUUAAAAAC |
| 5840 | CGGAGUGCAUCAGGCUGCUAC | 8444 | UUUGGAAUUUUUUUAAAAACA |
| 5841 | GGAGUGCAUCAGGCUGCUACU | 8445 | UUGGAAUUUUUUUAAAAACAC |
| 5842 | GAGUGCAUCAGGCUGCUACUU | 8446 | UGGAAUUUUUUUAAAAACACC |
| 5843 | AGUGCAUCAGGCUGCUACUUA | 8447 | GGAAUUUUUUUAAAAACACCA |
| 5844 | GUGCAUCAGGCUGCUACUUAG | 8448 | GAAUUUUUUUAAAAACACCAG |
| 5845 | UGCAUCAGGCUGCUACUUAGC | 8449 | AAUUUUUUUAAAAACACCAGC |
| 5846 | GCAUCAGGCUGCUACUUAGCA | 8450 | AUUUUUUUAAAAACACCAGCA |
| 5847 | CAUCAGGCUGCUACUUAGCAA | 8451 | UUUUUUUAAAAACACCAGCAU |
| 5848 | AUCAGGCUGCUACUUAGCAAA | 8452 | UUUUUUAAAAACACCAGCAUG |
| 5849 | UCAGGCUGCUACUUAGCAAAA | 8453 | UUUUUAAAAACACCAGCAUGG |
| 5850 | CAGGCUGCUACUUAGCAAAAG | 8454 | UUUUAAAAACACCAGCAUGGA |
| 5851 | AGGCUGCUACUUAGCAAAAGG | 8455 | UUUAAAAACACCAGCAUGGAA |
| 5852 | GGCUGCUACUUAGCAAAAGGC | 8456 | UUAAAAACACCAGCAUGGAAU |
| 5853 | GCUGCUACUUAGCAAAAGGCC | 8457 | UAAAAACACCAGCAUGGAAUU |
| 5854 | CUGCUACUUAGCAAAAGGCCU | 8458 | AAAAACACCAGCAUGGAAUUG |
| 5855 | UGCUACUUAGCAAAAGGCCUU | 8459 | AAAACACCAGCAUGGAAUUGG |
| 5856 | GCUACUUAGCAAAAGGCCUUU | 8460 | AAACACCAGCAUGGAAUUGGA |
| 5857 | CUACUUAGCAAAAGGCCUUUC | 8461 | AACACCAGCAUGGAAUUGGAG |
| 5858 | UACUUAGCAAAAGGCCUUUCU | 8462 | ACACCAGCAUGGAAUUGGAGG |
| 5859 | ACUUAGCAAAAGGCCUUUCUG | 8463 | CACCAGCAUGGAAUUGGAGGA |
| 5860 | CUUAGCAAAAGGCCUUUCUGU | 8464 | ACCAGCAUGGAAUUGGAGGAG |
| 5861 | UUAGCAAAAGGCCUUUCUGUU | 8465 | CCAGCAUGGAAUUGGAGGAGU |
| 5862 | UAGCAAAAGGCCUUUCUGUUU | 8466 | CAGCAUGGAAUUGGAGGAGUG |
| 5863 | AGCAAAAGGCCUUUCUGUUUG | 8467 | AGCAUGGAAUUGGAGGAGUGU |
| 5864 | GCAAAAGGCCUUUCUGUUUGA | 8468 | GCAUGGAAUUGGAGGAGUGUG |
| 5865 | CAAAAGGCCUUUCUGUUUGAU | 8469 | CAUGGAAUUGGAGGAGUGUGU |
| 5866 | AAAAGGCCUUUCUGUUUGAUG | 8470 | AUGGAAUUGGAGGAGUGUGUC |
| 5867 | AAAGGCCUUUCUGUUUGAUGC | 8471 | UGGAAUUGGAGGAGUGUGUCC |
| 5868 | AAGGCCUUUCUGUUUGAUGCC | 8472 | GGAAUUGGAGGAGUGUGUCCU |
| 5869 | AGGCCUUUCUGUUUGAUGCCU | 8473 | GAAUUGGAGGAGUGUGUCCUA |
| 5870 | GGCCUUUCUGUUUGAUGCCUG | 8474 | AAUUGGAGGAGUGUGUCCUAA |
| 5871 | GCCUUUCUGUUUGAUGCCUGC | 8475 | AUUGGAGGAGUGUGUCCUAAA |
| 5872 | CCUUUCUGUUUGAUGCCUGCU | 8476 | UUGGAGGAGUGUGUCCUAAAA |
| 5873 | CUUUCUGUUUGAUGCCUGCUC | 8477 | UGGAGGAGUGUGUCCUAAAAG |
| 5874 | UUUCUGUUUGAUGCCUGCUCG | 8478 | GGAGGAGUGUGUCCUAAAAGC |
| 5875 | UUCUGUUUGAUGCCUGCUCGG | 8479 | GAGGAGUGUGUCCUAAAAGCC |
| 5876 | UCUGUUUGAUGCCUGCUCGGG | 8480 | AGGAGUGUGUCCUAAAAGCCC |
| 5877 | CUGUUUGAUGCCUGCUCGGGA | 8481 | GGAGUGUGUCCUAAAAGCCCG |
| 5878 | UGUUUGAUGCCUGCUCGGGAG | 8482 | GAGUGUGUCCUAAAAGCCCGG |
| 5879 | GUUUGAUGCCUGCUCGGGAGG | 8483 | AGUGUGUCCUAAAAGCCCGGC |
| 5880 | UUUGAUGCCUGCUCGGGAGGU | 8484 | GUGUGUCCUAAAAGCCCGGCA |
| 5881 | UUGAUGCCUGCUCGGGAGGUU | 8485 | UGUGUCCUAAAAGCCCGGCAG |
| 5882 | UGAUGCCUGCUCGGGAGGUUG | 8486 | GUGUCCUAAAAGCCCGGCAGC |
| 5883 | GAUGCCUGCUCGGGAGGUUGG | 8487 | UGUCCUAAAAGCCCGGCAGCU |
| 5884 | AUGCCUGCUCGGGAGGUUGGC | 8488 | GUCCUAAAAGCCCGGCAGCUC |
| 5885 | UGCCUGCUCGGGAGGUUGGCU | 8489 | UCCUAAAAGCCCGGCAGCUCU |
| 5886 | GCCUGCUCGGGAGGUUGGCUU | 8490 | CCUAAAAGCCCGGCAGCUCUG |
| 5887 | CCUGCUCGGGAGGUUGGCUUC | 8491 | CUAAAAGCCCGGCAGCUCUGG |
| 5888 | CUGCUCGGGAGGUUGGCUUCA | 8492 | UAAAAGCCCGGCAGCUCUGGG |
| 5889 | UGCUCGGGAGGUUGGCUUCAG | 8493 | AAAAGCCCGGCAGCUCUGGGG |
| 5890 | GCUCGGGAGGUUGGCUUCAGC | 8494 | AAAGCCCGGCAGCUCUGGGGC |
| 5891 | CUCGGGAGGUUGGCUUCAGCU | 8495 | AAGCCCGGCAGCUCUGGGGCC |
| 5892 | UCGGGAGGUUGGCUUCAGCUC | 8496 | AGCCCGGCAGCUCUGGGGCCU |
| 5893 | CGGGAGGUUGGCUUCAGCUCC | 8497 | GCCCGGCAGCUCUGGGGCCUG |
| 5894 | GGGAGGUUGGCUUCAGCUCCA | 8498 | CCCGGCAGCUCUGGGGCCUGC |
| 5895 | GGAGGUUGGCUUCAGCUCCAG | 8499 | CCGGCAGCUCUGGGGCCUGCU |
| 5896 | GAGGUUGGCUUCAGCUCCAGG | 8500 | CGGCAGCUCUGGGGCCUGCUG |
| 5897 | AGGUUGGCUUCAGCUCCAGGC | 8501 | GGCAGCUCUGGGGCCUGCUGC |
| 5898 | GGUUGGCUUCAGCUCCAGGCU | 8502 | GCAGCUCUGGGGCCUGCUGCA |
| 5899 | GUUGGCUUCAGCUCCAGGCUU | 8503 | CAGCUCUGGGGCCUGCUGCAG |
| 5900 | UUGGCUUCAGCUCCAGGCUUU | 8504 | AGCUCUGGGGCCUGCUGCAGU |
| 5901 | UGGCUUCAGCUCCAGGCUUUC | 8505 | GCUCUGGGGCCUGCUGCAGUC |
| 5902 | GGCUUCAGCUCCAGGCUUUCC | 8506 | CUCUGGGGCCUGCUGCAGUCU |
| 5903 | GCUUCAGCUCCAGGCUUUCCU | 8507 | UCUGGGGCCUGCUGCAGUCUG |
| 5904 | CUUCAGCUCCAGGCUUUCCUG | 8508 | CUGGGGCCUGCUGCAGUCUGC |
| 5905 | UUCAGCUCCAGGCUUUCCUGA | 8509 | UGGGGCCUGCUGCAGUCUGCC |
| 5906 | UCAGCUCCAGGCUUUCCUGAU | 8510 | GGGGCCUGCUGCAGUCUGCCU |
| 5907 | CAGCUCCAGGCUUUCCUGAUC | 8511 | GGGCCUGCUGCAGUCUGCCUG |
| 5908 | AGCUCCAGGCUUUCCUGAUCC | 8512 | GGCCUGCUGCAGUCUGCCUGA |
| 5909 | GCUCCAGGCUUUCCUGAUCCG | 8513 | GCCUGCUGCAGUCUGCCUGAA |
| 5910 | CUCCAGGCUUUCCUGAUCCGU | 8514 | CCUGCUGCAGUCUGCCUGAAU |
| 5911 | UCCAGGCUUUCCUGAUCCGUG | 8515 | CUGCUGCAGUCUGCCUGAAUG |
| 5912 | CCAGGCUUUCCUGAUCCGUGG | 8516 | UGCUGCAGUCUGCCUGAAUGC |
| 5913 | CAGGCUUUCCUGAUCCGUGGC | 8517 | GCUGCAGUCUGCCUGAAUGCA |
| 5914 | AGGCUUUCCUGAUCCGUGGCA | 8518 | CUGCAGUCUGCCUGAAUGCAC |
| 5915 | GGCUUUCCUGAUCCGUGGCAU | 8519 | UGCAGUCUGCCUGAAUGCACA |
| 5916 | GCUUUCCUGAUCCGUGGCAUC | 8520 | GCAGUCUGCCUGAAUGCACAU |
| 5917 | CUUUCCUGAUCCGUGGCAUCC | 8521 | CAGUCUGCCUGAAUGCACAUC |
| 5918 | UUUCCUGAUCCGUGGCAUCCA | 8522 | AGUCUGCCUGAAUGCACAUCC |
| 5919 | UUCCUGAUCCGUGGCAUCCAG | 8523 | GUCUGCCUGAAUGCACAUCCC |
| 5920 | UCCUGAUCCGUGGCAUCCAGG | 8524 | UCUGCCUGAAUGCACAUCCCU |
| 5921 | CCUGAUCCGUGGCAUCCAGGA | 8525 | CUGCCUGAAUGCACAUCCCUU |
| 5922 | CUGAUCCGUGGCAUCCAGGAU | 8526 | UGCCUGAAUGCACAUCCCUUC |
| 5923 | UGAUCCGUGGCAUCCAGGAUC | 8527 | GCCUGAAUGCACAUCCCUUCU |
| 5924 | GAUCCGUGGCAUCCAGGAUCU | 8528 | CCUGAAUGCACAUCCCUUCUA |
| 5925 | AUCCGUGGCAUCCAGGAUCUU | 8529 | CUGAAUGCACAUCCCUUCUAG |
| 5926 | UCCGUGGCAUCCAGGAUCUUG | 8530 | UGAAUGCACAUCCCUUCUAGC |
| 5927 | CCGUGGCAUCCAGGAUCUUGU | 8531 | GAAUGCACAUCCCUUCUAGCC |
| 5928 | CGUGGCAUCCAGGAUCUUGUA | 8532 | AAUGCACAUCCCUUCUAGCCA |
| 5929 | GUGGCAUCCAGGAUCUUGUAC | 8533 | AUGCACAUCCCUUCUAGCCAC |
| 5930 | UGGCAUCCAGGAUCUUGUACU | 8534 | UGCACAUCCCUUCUAGCCACC |
| 5931 | GGCAUCCAGGAUCUUGUACUU | 8535 | GCACAUCCCUUCUAGCCACCA |
| 5932 | GCAUCCAGGAUCUUGUACUUG | 8536 | CACAUCCCUUCUAGCCACCAA |
| 5933 | CAUCCAGGAUCUUGUACUUGC | 8537 | ACAUCCCUUCUAGCCACCAAA |
| 5934 | AUCCAGGAUCUUGUACUUGCU | 8538 | CAUCCCUUCUAGCCACCAAAA |
| 5935 | UCCAGGAUCUUGUACUUGCUU | 8539 | AUCCCUUCUAGCCACCAAAAG |
| 5936 | CCAGGAUCUUGUACUUGCUUU | 8540 | UCCCUUCUAGCCACCAAAAGA |
| 5937 | CAGGAUCUUGUACUUGCUUUU | 8541 | CCCUUCUAGCCACCAAAAGAC |
| 5938 | AGGAUCUUGUACUUGCUUUUC | 8542 | CCUUCUAGCCACCAAAAGACA |
| 5939 | GGAUCUUGUACUUGCUUUUCC | 8543 | CUUCUAGCCACCAAAAGACAU |
| 5940 | GAUCUUGUACUUGCUUUUCCU | 8544 | UUCUAGCCACCAAAAGACAUG |
| 5941 | AUCUUGUACUUGCUUUUCCUC | 8545 | UCUAGCCACCAAAAGACAUGG |
| 5942 | UCUUGUACUUGCUUUUCCUCU | 8546 | CUAGCCACCAAAAGACAUGGC |
| 5943 | CUUGUACUUGCUUUUCCUCUU | 8547 | UAGCCACCAAAAGACAUGGCA |
| 5944 | UUGUACUUGCUUUUCCUCUUG | 8548 | AGCCACCAAAAGACAUGGCAG |
| 5945 | UGUACUUGCUUUUCCUCUUGG | 8549 | GCCACCAAAAGACAUGGCAGG |
| 5946 | GUACUUGCUUUUCCUCUUGGG | 8550 | CCACCAAAAGACAUGGCAGGC |
| 5947 | UACUUGCUUUUCCUCUUGGGU | 8551 | CACCAAAAGACAUGGCAGGCA |
| 5948 | ACUUGCUUUUCCUCUUGGGUU | 8552 | ACCAAAAGACAUGGCAGGCAG |
| 5949 | CUUGCUUUUCCUCUUGGGUUU | 8553 | CCAAAAGACAUGGCAGGCAGA |
| 5950 | UUGCUUUUCCUCUUGGGUUUU | 8554 | CAAAAGACAUGGCAGGCAGAG |
| 5951 | UGCUUUUCCUCUUGGGUUUUC | 8555 | AAAAGACAUGGCAGGCAGAGU |
| 5952 | GCUUUUCCUCUUGGGUUUUCC | 8556 | AAAGACAUGGCAGGCAGAGUC |
| 5953 | CUUUUCCUCUUGGGUUUUCCU | 8557 | AAGACAUGGCAGGCAGAGUCC |
| 5954 | UUUUCCUCUUGGGUUUUCCUU | 8558 | AGACAUGGCAGGCAGAGUCCU |
| 5955 | UUUCCUCUUGGGUUUUCCUUU | 8559 | GACAUGGCAGGCAGAGUCCUG |
| 5956 | UUCCUCUUGGGUUUUCCUUUU | 8560 | ACAUGGCAGGCAGAGUCCUGG |
| 5957 | UCCUCUUGGGUUUUCCUUUUU | 8561 | CAUGGCAGGCAGAGUCCUGGC |
| 5958 | CCUCUUGGGUUUUCCUUUUUG | 8562 | AUGGCAGGCAGAGUCCUGGCA |
| 5959 | CUCUUGGGUUUUCCUUUUUGC | 8563 | UGGCAGGCAGAGUCCUGGCAG |
| 5960 | UCUUGGGUUUUCCUUUUUGCC | 8564 | GGCAGGCAGAGUCCUGGCAGC |
| 5961 | CUUGGGUUUUCCUUUUUGCCU | 8565 | GCAGGCAGAGUCCUGGCAGCA |
| 5962 | UUGGGUUUUCCUUUUUGCCUC | 8566 | CAGGCAGAGUCCUGGCAGCAG |
| 5963 | UGGGUUUUCCUUUUUGCCUCU | 8567 | AGGCAGAGUCCUGGCAGCAGG |
| 5964 | GGGUUUUCCUUUUUGCCUCUU | 8568 | GGCAGAGUCCUGGCAGCAGGC |
| 5965 | GGUUUUCCUUUUUGCCUCUUA | 8569 | GCAGAGUCCUGGCAGCAGGCA |
| 5966 | GUUUUCCUUUUUGCCUCUUAC | 8570 | CAGAGUCCUGGCAGCAGGCAG |
| 5967 | UUUUCCUUUUUGCCUCUUACA | 8571 | AGAGUCCUGGCAGCAGGCAGC |
| 5968 | UUUCCUUUUUGCCUCUUACAA | 8572 | GAGUCCUGGCAGCAGGCAGCC |
| 5969 | UUCCUUUUUGCCUCUUACAAC | 8573 | AGUCCUGGCAGCAGGCAGCCC |
| 5970 | UCCUUUUUGCCUCUUACAACA | 8574 | GUCCUGGCAGCAGGCAGCCCA |
| 5971 | CCUUUUUGCCUCUUACAACAA | 8575 | UCCUGGCAGCAGGCAGCCCAA |
| 5972 | CUUUUUGCCUCUUACAACAAC | 8576 | CCUGGCAGCAGGCAGCCCAAC |
| 5973 | UUUUUGCCUCUUACAACAACA | 8577 | CUGGCAGCAGGCAGCCCAACA |
| 5974 | UUUUGCCUCUUACAACAACAG | 8578 | UGGCAGCAGGCAGCCCAACAU |
| 5975 | UUUGCCUCUUACAACAACAGA | 8579 | GGCAGCAGGCAGCCCAACAUG |
| 5976 | UUGCCUCUUACAACAACAGAU | 8580 | GCAGCAGGCAGCCCAACAUGA |
| 5977 | UGCCUCUUACAACAACAGAUC | 8581 | CAGCAGGCAGCCCAACAUGAC |
| 5978 | GCCUCUUACAACAACAGAUCA | 8582 | AGCAGGCAGCCCAACAUGACU |
| 5979 | CCUCUUACAACAACAGAUCAC | 8583 | GCAGGCAGCCCAACAUGACUG |
| 5980 | CUCUUACAACAACAGAUCACA | 8584 | CAGGCAGCCCAACAUGACUGG |
| 5981 | UCUUACAACAACAGAUCACAG | 8585 | AGGCAGCCCAACAUGACUGGA |
| 5982 | CUUACAACAACAGAUCACAGU | 8586 | GGCAGCCCAACAUGACUGGAG |
| 5983 | UUACAACAACAGAUCACAGUC | 8587 | GCAGCCCAACAUGACUGGAGA |
| 5984 | UACAACAACAGAUCACAGUCC | 8588 | CAGCCCAACAUGACUGGAGAG |
| 5985 | ACAACAACAGAUCACAGUCCA | 8589 | AGCCCAACAUGACUGGAGAGA |
| 5986 | CAACAACAGAUCACAGUCCAA | 8590 | GCCCAACAUGACUGGAGAGAG |
| 5987 | AACAACAGAUCACAGUCCAAG | 8591 | CCCAACAUGACUGGAGAGAGG |
| 5988 | ACAACAGAUCACAGUCCAAGA | 8592 | CCAACAUGACUGGAGAGAGGG |
| 5989 | CAACAGAUCACAGUCCAAGAC | 8593 | CAACAUGACUGGAGAGAGGGG |
| 5990 | AACAGAUCACAGUCCAAGACA | 8594 | AACAUGACUGGAGAGAGGGGG |
| 5991 | ACAGAUCACAGUCCAAGACAG | 8595 | ACAUGACUGGAGAGAGGGGGU |
| 5992 | CAGAUCACAGUCCAAGACAGG | 8596 | CAUGACUGGAGAGAGGGGGUU |
| 5993 | AGAUCACAGUCCAAGACAGGA | 8597 | AUGACUGGAGAGAGGGGGUUC |
| 5994 | GAUCACAGUCCAAGACAGGAU | 8598 | UGACUGGAGAGAGGGGGUUCC |
| 5995 | AUCACAGUCCAAGACAGGAUU | 8599 | GACUGGAGAGAGGGGGUUCCU |
| 5996 | UCACAGUCCAAGACAGGAUUC | 8600 | ACUGGAGAGAGGGGGUUCCUU |
| 5997 | CACAGUCCAAGACAGGAUUCC | 8601 | CUGGAGAGAGGGGGUUCCUUC |
| 5998 | ACAGUCCAAGACAGGAUUCCC | 8602 | UGGAGAGAGGGGGUUCCUUCA |
| 5999 | CAGUCCAAGACAGGAUUCCCA | 8603 | GGAGAGAGGGGGUUCCUUCAA |
| 6000 | AGUCCAAGACAGGAUUCCCAA | 8604 | GAGAGAGGGGGUUCCUUCAAG |
| 6001 | GUCCAAGACAGGAUUCCCAAG | 8605 | AGAGAGGGGGUUCCUUCAAGA |
| 6002 | UCCAAGACAGGAUUCCCAAGG | 8606 | GAGAGGGGGUUCCUUCAAGAA |
| 6003 | CCAAGACAGGAUUCCCAAGGC | 8607 | AGAGGGGGUUCCUUCAAGAAG |
| 6004 | CAAGACAGGAUUCCCAAGGCA | 8608 | GAGGGGGUUCCUUCAAGAAGC |
| 6005 | AAGACAGGAUUCCCAAGGCAA | 8609 | AGGGGGUUCCUUCAAGAAGCC |
| 6006 | AGACAGGAUUCCCAAGGCAAC | 8610 | GGGGGUUCCUUCAAGAAGCCA |
| 6007 | GACAGGAUUCCCAAGGCAACA | 8611 | GGGGUUCCUUCAAGAAGCCAG |
| 6008 | ACAGGAUUCCCAAGGCAACAA | 8612 | GGGUUCCUUCAAGAAGCCAGA |
| 6009 | CAGGAUUCCCAAGGCAACAAC | 8613 | GGUUCCUUCAAGAAGCCAGAG |
| 6010 | AGGAUUCCCAAGGCAACAACA | 8614 | GUUCCUUCAAGAAGCCAGAGG |
| 6011 | GGAUUCCCAAGGCAACAACAA | 8615 | UUCCUUCAAGAAGCCAGAGGU |
| 6012 | GAUUCCCAAGGCAACAACAAU | 8616 | UCCUUCAAGAAGCCAGAGGUG |
| 6013 | AUUCCCAAGGCAACAACAAUG | 8617 | CCUUCAAGAAGCCAGAGGUGA |
| 6014 | UUCCCAAGGCAACAACAAUGA | 8618 | CUUCAAGAAGCCAGAGGUGAU |
| 6015 | UCCCAAGGCAACAACAAUGAC | 8619 | UUCAAGAAGCCAGAGGUGAUU |
| 6016 | CCCAAGGCAACAACAAUGACA | 8620 | UCAAGAAGCCAGAGGUGAUUU |
| 6017 | CCAAGGCAACAACAAUGACAA | 8621 | CAAGAAGCCAGAGGUGAUUUU |
| 6018 | CAAGGCAACAACAAUGACAAA | 8622 | AAGAAGCCAGAGGUGAUUUUC |
| 6019 | AAGGCAACAACAAUGACAAAG | 8623 | AGAAGCCAGAGGUGAUUUUCU |
| 6020 | AGGCAACAACAAUGACAAAGG | 8624 | GAAGCCAGAGGUGAUUUUCUC |
| 6021 | GGCAACAACAAUGACAAAGGU | 8625 | AAGCCAGAGGUGAUUUUCUCC |
| 6022 | GCAACAACAAUGACAAAGGUA | 8626 | AGCCAGAGGUGAUUUUCUCCC |
| 6023 | CAACAACAAUGACAAAGGUAG | 8627 | GCCAGAGGUGAUUUUCUCCCC |
| 6024 | AACAACAAUGACAAAGGUAGC | 8628 | CCAGAGGUGAUUUUCUCCCCC |
| 6025 | ACAACAAUGACAAAGGUAGCA | 8629 | CAGAGGUGAUUUUCUCCCCCA |
| 6026 | CAACAAUGACAAAGGUAGCAA | 8630 | AGAGGUGAUUUUCUCCCCCAG |
| 6027 | AACAAUGACAAAGGUAGCAAU | 8631 | GAGGUGAUUUUCUCCCCCAGA |
| 6028 | ACAAUGACAAAGGUAGCAAUG | 8632 | AGGUGAUUUUCUCCCCCAGAU |
| 6029 | CAAUGACAAAGGUAGCAAUGA | 8633 | GGUGAUUUUCUCCCCCAGAUC |
| 6030 | AAUGACAAAGGUAGCAAUGAU | 8634 | GUGAUUUUCUCCCCCAGAUCU |
| 6031 | AUGACAAAGGUAGCAAUGAUA | 8635 | UGAUUUUCUCCCCCAGAUCUC |
| 6032 | UGACAAAGGUAGCAAUGAUAA | 8636 | GAUUUUCUCCCCCAGAUCUCA |
| 6033 | GACAAAGGUAGCAAUGAUAAC | 8637 | AUUUUCUCCCCCAGAUCUCAG |
| 6034 | ACAAAGGUAGCAAUGAUAACA | 8638 | UUUUCUCCCCCAGAUCUCAGG |
| 6035 | CAAAGGUAGCAAUGAUAACAU | 8639 | UUUCUCCCCCAGAUCUCAGGC |
| 6036 | AAAGGUAGCAAUGAUAACAUA | 8640 | UUCUCCCCCAGAUCUCAGGCC |
| 6037 | AAGGUAGCAAUGAUAACAUAU | 8641 | UCUCCCCCAGAUCUCAGGCCA |
| 6038 | AGGUAGCAAUGAUAACAUAUA | 8642 | CUCCCCCAGAUCUCAGGCCAA |
| 6039 | GGUAGCAAUGAUAACAUAUAA | 8643 | UCCCCCAGAUCUCAGGCCAAC |
| 6040 | GUAGCAAUGAUAACAUAUAAC | 8644 | CCCCCAGAUCUCAGGCCAACU |
| 6041 | UAGCAAUGAUAACAUAUAACA | 8645 | CCCCAGAUCUCAGGCCAACUC |
| 6042 | AGCAAUGAUAACAUAUAACAC | 8646 | CCCAGAUCUCAGGCCAACUCC |
| 6043 | GCAAUGAUAACAUAUAACACG | 8647 | CCAGAUCUCAGGCCAACUCCA |
| 6044 | CAAUGAUAACAUAUAACACGC | 8648 | CAGAUCUCAGGCCAACUCCAA |
| 6045 | AAUGAUAACAUAUAACACGCU | 8649 | AGAUCUCAGGCCAACUCCAAA |
| 6046 | AUGAUAACAUAUAACACGCUC | 8650 | GAUCUCAGGCCAACUCCAAAU |
| 6047 | UGAUAACAUAUAACACGCUCC | 8651 | AUCUCAGGCCAACUCCAAAUU |
| 6048 | GAUAACAUAUAACACGCUCCA | 8652 | UCUCAGGCCAACUCCAAAUUG |
| 6049 | AUAACAUAUAACACGCUCCAC | 8653 | CUCAGGCCAACUCCAAAUUGU |
| 6050 | UAACAUAUAACACGCUCCACU | 8654 | UCAGGCCAACUCCAAAUUGUG |
| 6051 | AACAUAUAACACGCUCCACUC | 8655 | CAGGCCAACUCCAAAUUGUGU |
| 6052 | ACAUAUAACACGCUCCACUCA | 8656 | AGGCCAACUCCAAAUUGUGUC |
| 6053 | CAUAUAACACGCUCCACUCAC | 8657 | GGCCAACUCCAAAUUGUGUCU |
| 6054 | AUAUAACACGCUCCACUCACA | 8658 | GCCAACUCCAAAUUGUGUCUU |
| 6055 | UAUAACACGCUCCACUCACAG | 8659 | CCAACUCCAAAUUGUGUCUUC |
| 6056 | AUAACACGCUCCACUCACAGU | 8660 | CAACUCCAAAUUGUGUCUUCC |
| 6057 | UAACACGCUCCACUCACAGUU | 8661 | AACUCCAAAUUGUGUCUUCCC |
| 6058 | AACACGCUCCACUCACAGUUG | 8662 | ACUCCAAAUUGUGUCUUCCCC |
| 6059 | ACACGCUCCACUCACAGUUGC | 8663 | CUCCAAAUUGUGUCUUCCCCU |
| 6060 | CACGCUCCACUCACAGUUGCU | 8664 | UCCAAAUUGUGUCUUCCCCUG |
| 6061 | ACGCUCCACUCACAGUUGCUG | 8665 | CCAAAUUGUGUCUUCCCCUGC |
| 6062 | CGCUCCACUCACAGUUGCUGU | 8666 | CAAAUUGUGUCUUCCCCUGCU |
| 6063 | GCUCCACUCACAGUUGCUGUC | 8667 | AAAUUGUGUCUUCCCCUGCUG |
| 6064 | CUCCACUCACAGUUGCUGUCU | 8668 | AAUUGUGUCUUCCCCUGCUGG |
| 6065 | UCCACUCACAGUUGCUGUCUC | 8669 | AUUGUGUCUUCCCCUGCUGGC |
| 6066 | CCACUCACAGUUGCUGUCUCC | 8670 | UUGUGUCUUCCCCUGCUGGCA |
| 6067 | CACUCACAGUUGCUGUCUCCA | 8671 | UGUGUCUUCCCCUGCUGGCAC |
| 6068 | ACUCACAGUUGCUGUCUCCAU | 8672 | GUGUCUUCCCCUGCUGGCACC |
| 6069 | CUCACAGUUGCUGUCUCCAUC | 8673 | UGUCUUCCCCUGCUGGCACCU |
| 6070 | UCACAGUUGCUGUCUCCAUCC | 8674 | GUCUUCCCCUGCUGGCACCUG |
| 6071 | CACAGUUGCUGUCUCCAUCCC | 8675 | UCUUCCCCUGCUGGCACCUGC |
| 6072 | ACAGUUGCUGUCUCCAUCCCU | 8676 | CUUCCCCUGCUGGCACCUGCU |
| 6073 | CAGUUGCUGUCUCCAUCCCUC | 8677 | UUCCCCUGCUGGCACCUGCUC |
| 6074 | AGUUGCUGUCUCCAUCCCUCA | 8678 | UCCCCUGCUGGCACCUGCUCU |
| 6075 | GUUGCUGUCUCCAUCCCUCAG | 8679 | CCCCUGCUGGCACCUGCUCUC |
| 6076 | UUGCUGUCUCCAUCCCUCAGC | 8680 | CCCUGCUGGCACCUGCUCUCA |
| 6077 | UGCUGUCUCCAUCCCUCAGCU | 8681 | CCUGCUGGCACCUGCUCUCAC |
| 6078 | GCUGUCUCCAUCCCUCAGCUG | 8682 | CUGCUGGCACCUGCUCUCACU |
| 6079 | CUGUCUCCAUCCCUCAGCUGC | 8683 | UGCUGGCACCUGCUCUCACUG |
| 6080 | UGUCUCCAUCCCUCAGCUGCA | 8684 | GCUGGCACCUGCUCUCACUGG |
| 6081 | GUCUCCAUCCCUCAGCUGCAC | 8685 | CUGGCACCUGCUCUCACUGGC |
| 6082 | UCUCCAUCCCUCAGCUGCACC | 8686 | UGGCACCUGCUCUCACUGGCA |
| 6083 | CUCCAUCCCUCAGCUGCACCU | 8687 | GGCACCUGCUCUCACUGGCAU |
| 6084 | UCCAUCCCUCAGCUGCACCUU | 8688 | GCACCUGCUCUCACUGGCAUC |
| 6085 | CCAUCCCUCAGCUGCACCUUG | 8689 | CACCUGCUCUCACUGGCAUCU |
| 6086 | CAUCCCUCAGCUGCACCUUGA | 8690 | ACCUGCUCUCACUGGCAUCUG |
| 6087 | AUCCCUCAGCUGCACCUUGAU | 8691 | CCUGCUCUCACUGGCAUCUGU |
| 6088 | UCCCUCAGCUGCACCUUGAUG | 8692 | CUGCUCUCACUGGCAUCUGUU |
| 6089 | CCCUCAGCUGCACCUUGAUGA | 8693 | UGCUCUCACUGGCAUCUGUUG |
| 6090 | CCUCAGCUGCACCUUGAUGAA | 8694 | GCUCUCACUGGCAUCUGUUGA |
| 6091 | CUCAGCUGCACCUUGAUGAAA | 8695 | CUCUCACUGGCAUCUGUUGAC |
| 6092 | UCAGCUGCACCUUGAUGAAAU | 8696 | UCUCACUGGCAUCUGUUGACA |
| 6093 | CAGCUGCACCUUGAUGAAAUU | 8697 | CUCACUGGCAUCUGUUGACAA |
| 6094 | AGCUGCACCUUGAUGAAAUUC | 8698 | UCACUGGCAUCUGUUGACAAC |
| 6095 | GCUGCACCUUGAUGAAAUUCU | 8699 | CACUGGCAUCUGUUGACAACC |
| 6096 | CUGCACCUUGAUGAAAUUCUC | 8700 | ACUGGCAUCUGUUGACAACCA |
| 6097 | UGCACCUUGAUGAAAUUCUCC | 8701 | CUGGCAUCUGUUGACAACCAC |
| 6098 | GCACCUUGAUGAAAUUCUCCA | 8702 | UGGCAUCUGUUGACAACCACA |
| 6099 | CACCUUGAUGAAAUUCUCCAU | 8703 | GGCAUCUGUUGACAACCACAG |
| 6100 | ACCUUGAUGAAAUUCUCCAUC | 8704 | GCAUCUGUUGACAACCACAGA |
| 6101 | CCUUGAUGAAAUUCUCCAUCC | 8705 | CAUCUGUUGACAACCACAGAA |
| 6102 | CUUGAUGAAAUUCUCCAUCCA | 8706 | AUCUGUUGACAACCACAGAAC |
| 6103 | UUGAUGAAAUUCUCCAUCCAA | 8707 | UCUGUUGACAACCACAGAACG |
| 6104 | UGAUGAAAUUCUCCAUCCAAA | 8708 | CUGUUGACAACCACAGAACGC |
| 6105 | GAUGAAAUUCUCCAUCCAAAA | 8709 | UGUUGACAACCACAGAACGCU |
| 6106 | AUGAAAUUCUCCAUCCAAAAA | 8710 | GUUGACAACCACAGAACGCUG |
| 6107 | UGAAAUUCUCCAUCCAAAAAG | 8711 | UUGACAACCACAGAACGCUGA |
| 6108 | GAAAUUCUCCAUCCAAAAAGG | 8712 | UGACAACCACAGAACGCUGAA |
| 6109 | AAAUUCUCCAUCCAAAAAGGG | 8713 | GACAACCACAGAACGCUGAAG |
| 6110 | AAUUCUCCAUCCAAAAAGGGU | 8714 | ACAACCACAGAACGCUGAAGC |
| 6111 | AUUCUCCAUCCAAAAAGGGUC | 8715 | CAACCACAGAACGCUGAAGCA |
| 6112 | UUCUCCAUCCAAAAAGGGUCA | 8716 | AACCACAGAACGCUGAAGCAA |
| 6113 | UCUCCAUCCAAAAAGGGUCAC | 8717 | ACCACAGAACGCUGAAGCAAA |
| 6114 | CUCCAUCCAAAAAGGGUCACA | 8718 | CCACAGAACGCUGAAGCAAAA |
| 6115 | UCCAUCCAAAAAGGGUCACAG | 8719 | CACAGAACGCUGAAGCAAAAG |
| 6116 | CCAUCCAAAAAGGGUCACAGA | 8720 | ACAGAACGCUGAAGCAAAAGC |
| 6117 | CAUCCAAAAAGGGUCACAGAU | 8721 | CAGAACGCUGAAGCAAAAGCA |
| 6118 | AUCCAAAAAGGGUCACAGAUA | 8722 | AGAACGCUGAAGCAAAAGCAA |
| 6119 | UCCAAAAAGGGUCACAGAUAC | 8723 | GAACGCUGAAGCAAAAGCAAG |
| 6120 | CCAAAAAGGGUCACAGAUACA | 8724 | AACGCUGAAGCAAAAGCAAGU |
| 6121 | CAAAAAGGGUCACAGAUACAG | 8725 | ACGCUGAAGCAAAAGCAAGUA |
| 6122 | AAAAAGGGUCACAGAUACAGC | 8726 | CGCUGAAGCAAAAGCAAGUAU |
| 6123 | AAAAGGGUCACAGAUACAGCG | 8727 | GCUGAAGCAAAAGCAAGUAUA |
| 6124 | AAAGGGUCACAGAUACAGCGU | 8728 | CUGAAGCAAAAGCAAGUAUAA |
| 6125 | AAGGGUCACAGAUACAGCGUU | 8729 | UGAAGCAAAAGCAAGUAUAAA |
| 6126 | AGGGUCACAGAUACAGCGUUU | 8730 | GAAGCAAAAGCAAGUAUAAAA |
| 6127 | GGGUCACAGAUACAGCGUUUG | 8731 | AAGCAAAAGCAAGUAUAAAAC |
| 6128 | GGUCACAGAUACAGCGUUUGG | 8732 | AGCAAAAGCAAGUAUAAAACA |
| 6129 | GUCACAGAUACAGCGUUUGGU | 8733 | GCAAAAGCAAGUAUAAAACAG |
| 6130 | UCACAGAUACAGCGUUUGGUG | 8734 | CAAAAGCAAGUAUAAAACAGG |
| 6131 | CACAGAUACAGCGUUUGGUGA | 8735 | AAAAGCAAGUAUAAAACAGGU |
| 6132 | ACAGAUACAGCGUUUGGUGAA | 8736 | AAAGCAAGUAUAAAACAGGUA |
| 6133 | CAGAUACAGCGUUUGGUGAAC | 8737 | AAGCAAGUAUAAAACAGGUAC |
| 6134 | AGAUACAGCGUUUGGUGAACG | 8738 | AGCAAGUAUAAAACAGGUACA |
| 6135 | GAUACAGCGUUUGGUGAACGA | 8739 | GCAAGUAUAAAACAGGUACAG |
| 6136 | AUACAGCGUUUGGUGAACGAG | 8740 | CAAGUAUAAAACAGGUACAGG |
| 6137 | UACAGCGUUUGGUGAACGAGU | 8741 | AAGUAUAAAACAGGUACAGGC |
| 6138 | ACAGCGUUUGGUGAACGAGUC | 8742 | AGUAUAAAACAGGUACAGGCU |
| 6139 | CAGCGUUUGGUGAACGAGUCA | 8743 | GUAUAAAACAGGUACAGGCUU |
| 6140 | AGCGUUUGGUGAACGAGUCAC | 8744 | UAUAAAACAGGUACAGGCUUC |
| 6141 | GCGUUUGGUGAACGAGUCACA | 8745 | AUAAAACAGGUACAGGCUUCU |
| 6142 | CGUUUGGUGAACGAGUCACAG | 8746 | UAAAACAGGUACAGGCUUCUC |
| 6143 | GUUUGGUGAACGAGUCACAGU | 8747 | AAAACAGGUACAGGCUUCUCA |
| 6144 | UUUGGUGAACGAGUCACAGUG | 8748 | AAACAGGUACAGGCUUCUCAA |
| 6145 | UUGGUGAACGAGUCACAGUGG | 8749 | AACAGGUACAGGCUUCUCAAC |
| 6146 | UGGUGAACGAGUCACAGUGGC | 8750 | ACAGGUACAGGCUUCUCAACC |
| 6147 | GGUGAACGAGUCACAGUGGCC | 8751 | CAGGUACAGGCUUCUCAACCA |
| 6148 | GUGAACGAGUCACAGUGGCCA | 8752 | AGGUACAGGCUUCUCAACCAC |
| 6149 | UGAACGAGUCACAGUGGCCAU | 8753 | GGUACAGGCUUCUCAACCACU |
| 6150 | GAACGAGUCACAGUGGCCAUG | 8754 | GUACAGGCUUCUCAACCACUU |
| 6151 | AACGAGUCACAGUGGCCAUGG | 8755 | UACAGGCUUCUCAACCACUUC |
| 6152 | ACGAGUCACAGUGGCCAUGGU | 8756 | ACAGGCUUCUCAACCACUUCG |
| 6153 | CGAGUCACAGUGGCCAUGGUC | 8757 | CAGGCUUCUCAACCACUUCGC |
| 6154 | GAGUCACAGUGGCCAUGGUCG | 8758 | AGGCUUCUCAACCACUUCGCA |
| 6155 | AGUCACAGUGGCCAUGGUCGG | 8759 | GGCUUCUCAACCACUUCGCAG |
| 6156 | GUCACAGUGGCCAUGGUCGGA | 8760 | GCUUCUCAACCACUUCGCAGA |
| 6157 | UCACAGUGGCCAUGGUCGGAA | 8761 | CUUCUCAACCACUUCGCAGAU |
| 6158 | CACAGUGGCCAUGGUCGGAAC | 8762 | UUCUCAACCACUUCGCAGAUG |
| 6159 | ACAGUGGCCAUGGUCGGAACA | 8763 | UCUCAACCACUUCGCAGAUGU |
| 6160 | CAGUGGCCAUGGUCGGAACAG | 8764 | CUCAACCACUUCGCAGAUGUC |
| 6161 | AGUGGCCAUGGUCGGAACAGU | 8765 | UCAACCACUUCGCAGAUGUCU |
| 6162 | GUGGCCAUGGUCGGAACAGUU | 8766 | CAACCACUUCGCAGAUGUCUG |
| 6163 | UGGCCAUGGUCGGAACAGUUC | 8767 | AACCACUUCGCAGAUGUCUGC |
| 6164 | GGCCAUGGUCGGAACAGUUCA | 8768 | ACCACUUCGCAGAUGUCUGCC |
| 6165 | GCCAUGGUCGGAACAGUUCAG | 8769 | CCACUUCGCAGAUGUCUGCCA |
| 6166 | CCAUGGUCGGAACAGUUCAGC | 8770 | CACUUCGCAGAUGUCUGCCAA |
| 6167 | CAUGGUCGGAACAGUUCAGCU | 8771 | ACUUCGCAGAUGUCUGCCAAU |
| 6168 | AUGGUCGGAACAGUUCAGCUG | 8772 | CUUCGCAGAUGUCUGCCAAUA |
| 6169 | UGGUCGGAACAGUUCAGCUGA | 8773 | UUCGCAGAUGUCUGCCAAUAA |
| 6170 | GGUCGGAACAGUUCAGCUGAC | 8774 | UCGCAGAUGUCUGCCAAUAAU |
| 6171 | GUCGGAACAGUUCAGCUGACA | 8775 | CGCAGAUGUCUGCCAAUAAUA |
| 6172 | UCGGAACAGUUCAGCUGACAU | 8776 | GCAGAUGUCUGCCAAUAAUAU |
| 6173 | CGGAACAGUUCAGCUGACAUG | 8777 | CAGAUGUCUGCCAAUAAUAUC |
| 6174 | GGAACAGUUCAGCUGACAUGU | 8778 | AGAUGUCUGCCAAUAAUAUCC |
| 6175 | GAACAGUUCAGCUGACAUGUG | 8779 | GAUGUCUGCCAAUAAUAUCCU |
| 6176 | AACAGUUCAGCUGACAUGUGA | 8780 | AUGUCUGCCAAUAAUAUCCUG |
| 6177 | ACAGUUCAGCUGACAUGUGAC | 8781 | UGUCUGCCAAUAAUAUCCUGA |
| 6178 | CAGUUCAGCUGACAUGUGACA | 8782 | GUCUGCCAAUAAUAUCCUGAU |
| 6179 | AGUUCAGCUGACAUGUGACAG | 8783 | UCUGCCAAUAAUAUCCUGAUA |
| 6180 | GUUCAGCUGACAUGUGACAGU | 8784 | CUGCCAAUAAUAUCCUGAUAA |
| 6181 | UUCAGCUGACAUGUGACAGUG | 8785 | UGCCAAUAAUAUCCUGAUAAA |
| 6182 | UCAGCUGACAUGUGACAGUGU | 8786 | GCCAAUAAUAUCCUGAUAAAA |
| 6183 | CAGCUGACAUGUGACAGUGUU | 8787 | CCAAUAAUAUCCUGAUAAAAU |
| 6184 | AGCUGACAUGUGACAGUGUUG | 8788 | CAAUAAUAUCCUGAUAAAAUC |
| 6185 | GCUGACAUGUGACAGUGUUGA | 8789 | AAUAAUAUCCUGAUAAAAUCC |
| 6186 | CUGACAUGUGACAGUGUUGAC | 8790 | AUAAUAUCCUGAUAAAAUCCA |
| 6187 | UGACAUGUGACAGUGUUGACU | 8791 | UAAUAUCCUGAUAAAAUCCAG |
| 6188 | GACAUGUGACAGUGUUGACUU | 8792 | AAUAUCCUGAUAAAAUCCAGG |
| 6189 | ACAUGUGACAGUGUUGACUUC | 8793 | AUAUCCUGAUAAAAUCCAGGA |
| 6190 | CAUGUGACAGUGUUGACUUCC | 8794 | UAUCCUGAUAAAAUCCAGGAA |
| 6191 | AUGUGACAGUGUUGACUUCCA | 8795 | AUCCUGAUAAAAUCCAGGAAG |
| 6192 | UGUGACAGUGUUGACUUCCAA | 8796 | UCCUGAUAAAAUCCAGGAAGC |
| 6193 | GUGACAGUGUUGACUUCCAAG | 8797 | CCUGAUAAAAUCCAGGAAGCA |
| 6194 | UGACAGUGUUGACUUCCAAGG | 8798 | CUGAUAAAAUCCAGGAAGCAG |
| 6195 | GACAGUGUUGACUUCCAAGGC | 8799 | UGAUAAAAUCCAGGAAGCAGG |
| 6196 | ACAGUGUUGACUUCCAAGGCU | 8800 | GAUAAAAUCCAGGAAGCAGGA |
| 6197 | CAGUGUUGACUUCCAAGGCUC | 8801 | AUAAAAUCCAGGAAGCAGGAU |
| 6198 | AGUGUUGACUUCCAAGGCUCU | 8802 | UAAAAUCCAGGAAGCAGGAUU |
| 6199 | GUGUUGACUUCCAAGGCUCUG | 8803 | AAAAUCCAGGAAGCAGGAUUU |
| 6200 | UGUUGACUUCCAAGGCUCUGA | 8804 | AAAUCCAGGAAGCAGGAUUUG |
| 6201 | GUUGACUUCCAAGGCUCUGAA | 8805 | AAUCCAGGAAGCAGGAUUUGG |
| 6202 | UUGACUUCCAAGGCUCUGAAU | 8806 | AUCCAGGAAGCAGGAUUUGGC |
| 6203 | UGACUUCCAAGGCUCUGAAUA | 8807 | UCCAGGAAGCAGGAUUUGGCU |
| 6204 | GACUUCCAAGGCUCUGAAUAU | 8808 | CCAGGAAGCAGGAUUUGGCUU |
| 6205 | ACUUCCAAGGCUCUGAAUAUC | 8809 | CAGGAAGCAGGAUUUGGCUUG |
| 6206 | CUUCCAAGGCUCUGAAUAUCA | 8810 | AGGAAGCAGGAUUUGGCUUGA |
| 6207 | UUCCAAGGCUCUGAAUAUCAA | 8811 | GGAAGCAGGAUUUGGCUUGAC |
| 6208 | UCCAAGGCUCUGAAUAUCAAA | 8812 | GAAGCAGGAUUUGGCUUGACU |
| 6209 | CCAAGGCUCUGAAUAUCAAAA | 8813 | AAGCAGGAUUUGGCUUGACUC |
| 6210 | CAAGGCUCUGAAUAUCAAAAA | 8814 | AGCAGGAUUUGGCUUGACUCC |
| 6211 | AAGGCUCUGAAUAUCAAAAAG | 8815 | GCAGGAUUUGGCUUGACUCCC |
| 6212 | AGGCUCUGAAUAUCAAAAAGU | 8816 | CAGGAUUUGGCUUGACUCCCA |
| 6213 | GGCUCUGAAUAUCAAAAAGUC | 8817 | AGGAUUUGGCUUGACUCCCAG |
| 6214 | GCUCUGAAUAUCAAAAAGUCU | 8818 | GGAUUUGGCUUGACUCCCAGC |
| 6215 | CUCUGAAUAUCAAAAAGUCUG | 8819 | GAUUUGGCUUGACUCCCAGCC |
| 6216 | UCUGAAUAUCAAAAAGUCUGC | 8820 | AUUUGGCUUGACUCCCAGCCU |
| 6217 | CUGAAUAUCAAAAAGUCUGCC | 8821 | UUUGGCUUGACUCCCAGCCUC |
| 6218 | UGAAUAUCAAAAAGUCUGCCU | 8822 | UUGGCUUGACUCCCAGCCUCU |
| 6219 | GAAUAUCAAAAAGUCUGCCUU | 8823 | UGGCUUGACUCCCAGCCUCUU |
| 6220 | AAUAUCAAAAAGUCUGCCUUU | 8824 | GGCUUGACUCCCAGCCUCUUC |
| 6221 | AUAUCAAAAAGUCUGCCUUUU | 8825 | GCUUGACUCCCAGCCUCUUCU |
| 6222 | UAUCAAAAAGUCUGCCUUUUG | 8826 | CUUGACUCCCAGCCUCUUCUC |
| 6223 | AUCAAAAAGUCUGCCUUUUGC | 8827 | UUGACUCCCAGCCUCUUCUCC |
| 6224 | UCAAAAAGUCUGCCUUUUGCU | 8828 | UGACUCCCAGCCUCUUCUCCA |
| 6225 | CAAAAAGUCUGCCUUUUGCUU | 8829 | GACUCCCAGCCUCUUCUCCAU |
| 6226 | AAAAAGUCUGCCUUUUGCUUC | 8830 | ACUCCCAGCCUCUUCUCCAUG |
| 6227 | AAAAGUCUGCCUUUUGCUUCC | 8831 | CUCCCAGCCUCUUCUCCAUGG |
| 6228 | AAAGUCUGCCUUUUGCUUCCG | 8832 | UCCCAGCCUCUUCUCCAUGGC |
| 6229 | AAGUCUGCCUUUUGCUUCCGC | 8833 | CCCAGCCUCUUCUCCAUGGCC |
| 6230 | AGUCUGCCUUUUGCUUCCGCA | 8834 | CCAGCCUCUUCUCCAUGGCCC |
| 6231 | GUCUGCCUUUUGCUUCCGCAG | 8835 | CAGCCUCUUCUCCAUGGCCCU |
| 6232 | UCUGCCUUUUGCUUCCGCAGC | 8836 | AGCCUCUUCUCCAUGGCCCUC |
| 6233 | CUGCCUUUUGCUUCCGCAGCU | 8837 | GCCUCUUCUCCAUGGCCCUCC |
| 6234 | UGCCUUUUGCUUCCGCAGCUC | 8838 | CCUCUUCUCCAUGGCCCUCCA |
| 6235 | GCCUUUUGCUUCCGCAGCUCA | 8839 | CUCUUCUCCAUGGCCCUCCAG |
| 6236 | CCUUUUGCUUCCGCAGCUCAC | 8840 | UCUUCUCCAUGGCCCUCCAGA |
| 6237 | CUUUUGCUUCCGCAGCUCACU | 8841 | CUUCUCCAUGGCCCUCCAGAC |
| 6238 | UUUUGCUUCCGCAGCUCACUC | 8842 | UUCUCCAUGGCCCUCCAGACA |
| 6239 | UUUGCUUCCGCAGCUCACUCU | 8843 | UCUCCAUGGCCCUCCAGACAG |
| 6240 | UUGCUUCCGCAGCUCACUCUU | 8844 | CUCCAUGGCCCUCCAGACAGG |
| 6241 | UGCUUCCGCAGCUCACUCUUG | 8845 | UCCAUGGCCCUCCAGACAGGC |
| 6242 | GCUUCCGCAGCUCACUCUUGA | 8846 | CCAUGGCCCUCCAGACAGGCA |
| 6243 | CUUCCGCAGCUCACUCUUGAG | 8847 | CAUGGCCCUCCAGACAGGCAG |
| 6244 | UUCCGCAGCUCACUCUUGAGC | 8848 | AUGGCCCUCCAGACAGGCAGA |
| 6245 | UCCGCAGCUCACUCUUGAGCA | 8849 | UGGCCCUCCAGACAGGCAGAA |
| 6246 | CCGCAGCUCACUCUUGAGCAU | 8850 | GGCCCUCCAGACAGGCAGAAC |
| 6247 | CGCAGCUCACUCUUGAGCAUC | 8851 | GCCCUCCAGACAGGCAGAACC |
| 6248 | GCAGCUCACUCUUGAGCAUCG | 8852 | CCCUCCAGACAGGCAGAACCA |
| 6249 | CAGCUCACUCUUGAGCAUCGC | 8853 | CCUCCAGACAGGCAGAACCAG |
| 6250 | AGCUCACUCUUGAGCAUCGCU | 8854 | CUCCAGACAGGCAGAACCAGU |
| 6251 | GCUCACUCUUGAGCAUCGCUG | 8855 | UCCAGACAGGCAGAACCAGUA |
| 6252 | CUCACUCUUGAGCAUCGCUGC | 8856 | CCAGACAGGCAGAACCAGUAC |
| 6253 | UCACUCUUGAGCAUCGCUGCC | 8857 | CAGACAGGCAGAACCAGUACA |
| 6254 | CACUCUUGAGCAUCGCUGCCA | 8858 | AGACAGGCAGAACCAGUACAC |
| 6255 | ACUCUUGAGCAUCGCUGCCAC | 8859 | GACAGGCAGAACCAGUACACG |
| 6256 | CUCUUGAGCAUCGCUGCCACC | 8860 | ACAGGCAGAACCAGUACACGG |
| 6257 | UCUUGAGCAUCGCUGCCACCU | 8861 | CAGGCAGAACCAGUACACGGG |
| 6258 | CUUGAGCAUCGCUGCCACCUC | 8862 | AGGCAGAACCAGUACACGGGG |
| 6259 | UUGAGCAUCGCUGCCACCUCA | 8863 | GGCAGAACCAGUACACGGGGC |
| 6260 | UGAGCAUCGCUGCCACCUCAU | 8864 | GCAGAACCAGUACACGGGGCU |
| 6261 | GAGCAUCGCUGCCACCUCAUG | 8865 | CAGAACCAGUACACGGGGCUC |
| 6262 | AGCAUCGCUGCCACCUCAUGG | 8866 | AGAACCAGUACACGGGGCUCA |
| 6263 | GCAUCGCUGCCACCUCAUGGC | 8867 | GAACCAGUACACGGGGCUCAG |
| 6264 | CAUCGCUGCCACCUCAUGGCC | 8868 | AACCAGUACACGGGGCUCAGG |
| 6265 | AUCGCUGCCACCUCAUGGCCU | 8869 | ACCAGUACACGGGGCUCAGGG |
| 6266 | UCGCUGCCACCUCAUGGCCUU | 8870 | CCAGUACACGGGGCUCAGGGC |
| 6267 | CGCUGCCACCUCAUGGCCUUU | 8871 | CAGUACACGGGGCUCAGGGCC |
| 6268 | GCUGCCACCUCAUGGCCUUUG | 8872 | AGUACACGGGGCUCAGGGCCA |
| 6269 | CUGCCACCUCAUGGCCUUUGA | 8873 | GUACACGGGGCUCAGGGCCAC |
| 6270 | UGCCACCUCAUGGCCUUUGAA | 8874 | UACACGGGGCUCAGGGCCACA |
| 6271 | GCCACCUCAUGGCCUUUGAAG | 8875 | ACACGGGGCUCAGGGCCACAU |
| 6272 | CCACCUCAUGGCCUUUGAAGA | 8876 | CACGGGGCUCAGGGCCACAUA |
| 6273 | CACCUCAUGGCCUUUGAAGAU | 8877 | ACGGGGCUCAGGGCCACAUAG |
| 6274 | ACCUCAUGGCCUUUGAAGAUC | 8878 | CGGGGCUCAGGGCCACAUAGC |
| 6275 | CCUCAUGGCCUUUGAAGAUCU | 8879 | GGGGCUCAGGGCCACAUAGCG |
| 6276 | CUCAUGGCCUUUGAAGAUCUG | 8880 | GGGCUCAGGGCCACAUAGCGG |
| 6277 | UCAUGGCCUUUGAAGAUCUGG | 8881 | GGCUCAGGGCCACAUAGCGGG |
| 6278 | CAUGGCCUUUGAAGAUCUGGU | 8882 | GCUCAGGGCCACAUAGCGGGG |
| 6279 | AUGGCCUUUGAAGAUCUGGUG | 8883 | CUCAGGGCCACAUAGCGGGGC |
| 6280 | UGGCCUUUGAAGAUCUGGUGG | 8884 | UCAGGGCCACAUAGCGGGGCC |
| 6281 | GGCCUUUGAAGAUCUGGUGGG | 8885 | CAGGGCCACAUAGCGGGGCCC |
| 6282 | GCCUUUGAAGAUCUGGUGGGG | 8886 | AGGGCCACAUAGCGGGGCCCC |
| 6283 | CCUUUGAAGAUCUGGUGGGGA | 8887 | GGGCCACAUAGCGGGGCCCCG |
| 6284 | CUUUGAAGAUCUGGUGGGGAG | 8888 | GGCCACAUAGCGGGGCCCCGG |
| 6285 | UUUGAAGAUCUGGUGGGGAGG | 8889 | GCCACAUAGCGGGGCCCCGGC |
| 6286 | UUGAAGAUCUGGUGGGGAGGC | 8890 | CCACAUAGCGGGGCCCCGGCU |
| 6287 | UGAAGAUCUGGUGGGGAGGCU | 8891 | CACAUAGCGGGGCCCCGGCUC |
| 6288 | GAAGAUCUGGUGGGGAGGCUC | 8892 | ACAUAGCGGGGCCCCGGCUCU |
| 6289 | AAGAUCUGGUGGGGAGGCUCG | 8893 | CAUAGCGGGGCCCCGGCUCUC |
| 6290 | AGAUCUGGUGGGGAGGCUCGU | 8894 | AUAGCGGGGCCCCGGCUCUCG |
| 6291 | GAUCUGGUGGGGAGGCUCGUU | 8895 | UAGCGGGGCCCCGGCUCUCGG |
| 6292 | AUCUGGUGGGGAGGCUCGUUU | 8896 | AGCGGGGCCCCGGCUCUCGGC |
| 6293 | UCUGGUGGGGAGGCUCGUUUU | 8897 | GCGGGGCCCCGGCUCUCGGCG |
| 6294 | CUGGUGGGGAGGCUCGUUUUG | 8898 | CGGGGCCCCGGCUCUCGGCGG |
| 6295 | UGGUGGGGAGGCUCGUUUUGA | 8899 | GGGGCCCCGGCUCUCGGCGGC |
| 6296 | GGUGGGGAGGCUCGUUUUGAA | 8900 | GGGCCCCGGCUCUCGGCGGCC |
| 6297 | GUGGGGAGGCUCGUUUUGAAC | 8901 | GGCCCCGGCUCUCGGCGGCCU |
| 6298 | UGGGGAGGCUCGUUUUGAACA | 8902 | GCCCCGGCUCUCGGCGGCCUC |
| 6299 | GGGGAGGCUCGUUUUGAACAA | 8903 | CCCCGGCUCUCGGCGGCCUCC |
| 6300 | GGGAGGCUCGUUUUGAACAAA | 8904 | CCCGGCUCUCGGCGGCCUCCG |
| 6301 | GGAGGCUCGUUUUGAACAAAA | 8905 | CCGGCUCUCGGCGGCCUCCGC |
| 6302 | GAGGCUCGUUUUGAACAAAAA | 8906 | CGGCUCUCGGCGGCCUCCGCC |
| 6303 | AGGCUCGUUUUGAACAAAAAA | 8907 | GGCUCUCGGCGGCCUCCGCCU |
| 6304 | GGCUCGUUUUGAACAAAAAAU | 8908 | GCUCUCGGCGGCCUCCGCCUC |
| 6305 | GCUCGUUUUGAACAAAAAAUA | 8909 | CUCUCGGCGGCCUCCGCCUCC |
| 6306 | CUCGUUUUGAACAAAAAAUAC | 8910 | UCUCGGCGGCCUCCGCCUCCU |
| 6307 | UCGUUUUGAACAAAAAAUACC | 8911 | CUCGGCGGCCUCCGCCUCCUC |
| 6308 | CGUUUUGAACAAAAAAUACCA | 8912 | UCGGCGGCCUCCGCCUCCUCC |
| 6309 | GUUUUGAACAAAAAAUACCAU | 8913 | CGGCGGCCUCCGCCUCCUCCU |
| 6310 | UUUUGAACAAAAAAUACCAUU | 8914 | GGCGGCCUCCGCCUCCUCCUG |
| 6311 | UUUGAACAAAAAAUACCAUUU | 8915 | GCGGCCUCCGCCUCCUCCUGG |
| 6312 | UUGAACAAAAAAUACCAUUUU | 8916 | CGGCCUCCGCCUCCUCCUGGU |
| 6313 | UGAACAAAAAAUACCAUUUUG | 8917 | GGCCUCCGCCUCCUCCUGGUC |
| 6314 | GAACAAAAAAUACCAUUUUGG | 8918 | GCCUCCGCCUCCUCCUGGUCC |
| 6315 | AACAAAAAAUACCAUUUUGGU | 8919 | CCUCCGCCUCCUCCUGGUCCA |
| 6316 | ACAAAAAAUACCAUUUUGGUG | 8920 | CUCCGCCUCCUCCUGGUCCAU |
| 6317 | CAAAAAAUACCAUUUUGGUGC | 8921 | UCCGCCUCCUCCUGGUCCAUG |
| 6318 | AAAAAAUACCAUUUUGGUGCU | 8922 | CCGCCUCCUCCUGGUCCAUGG |
| 6319 | AAAAAUACCAUUUUGGUGCUC | 8923 | CGCCUCCUCCUGGUCCAUGGG |
| 6320 | AAAAUACCAUUUUGGUGCUCU | 8924 | GCCUCCUCCUGGUCCAUGGGC |
| 6321 | AAAUACCAUUUUGGUGCUCUG | 8925 | CCUCCUCCUGGUCCAUGGGCU |
| 6322 | AAUACCAUUUUGGUGCUCUGC | 8926 | CUCCUCCUGGUCCAUGGGCUC |
| 6323 | AUACCAUUUUGGUGCUCUGCU | 8927 | UCCUCCUGGUCCAUGGGCUCG |
| 6324 | UACCAUUUUGGUGCUCUGCUC | 8928 | CCUCCUGGUCCAUGGGCUCGG |
| 6325 | ACCAUUUUGGUGCUCUGCUCC | 8929 | CUCCUGGUCCAUGGGCUCGGG |
| 6326 | CCAUUUUGGUGCUCUGCUCCG | 8930 | UCCUGGUCCAUGGGCUCGGGG |
| 6327 | CAUUUUGGUGCUCUGCUCCGU | 8931 | CCUGGUCCAUGGGCUCGGGGA |
| 6328 | AUUUUGGUGCUCUGCUCCGUG | 8932 | CUGGUCCAUGGGCUCGGGGAC |
| 6329 | UUUUGGUGCUCUGCUCCGUGU | 8933 | UGGUCCAUGGGCUCGGGGACC |
| 6330 | UUUGGUGCUCUGCUCCGUGUA | 8934 | GGUCCAUGGGCUCGGGGACCC |
| 6331 | UUGGUGCUCUGCUCCGUGUAC | 8935 | GUCCAUGGGCUCGGGGACCCC |
| 6332 | UGGUGCUCUGCUCCGUGUACG | 8936 | UCCAUGGGCUCGGGGACCCCC |
| 6333 | GGUGCUCUGCUCCGUGUACGG | 8937 | CCAUGGGCUCGGGGACCCCCA |
| 6334 | GUGCUCUGCUCCGUGUACGGC | 8938 | CAUGGGCUCGGGGACCCCCAA |
| 6335 | UGCUCUGCUCCGUGUACGGCU | 8939 | AUGGGCUCGGGGACCCCCAAC |
| 6336 | GCUCUGCUCCGUGUACGGCUG | 8940 | UGGGCUCGGGGACCCCCAACC |
| 6337 | CUCUGCUCCGUGUACGGCUGA | 8941 | GGGCUCGGGGACCCCCAACCU |
| 6338 | UCUGCUCCGUGUACGGCUGAA | 8942 | GGCUCGGGGACCCCCAACCUU |
| 6339 | CUGCUCCGUGUACGGCUGAAU | 8943 | GCUCGGGGACCCCCAACCUUC |
| 6340 | UGCUCCGUGUACGGCUGAAUC | 8944 | CUCGGGGACCCCCAACCUUCG |
| 6341 | GCUCCGUGUACGGCUGAAUCU | 8945 | UCGGGGACCCCCAACCUUCGC |
| 6342 | CUCCGUGUACGGCUGAAUCUU | 8946 | CGGGGACCCCCAACCUUCGCU |
| 6343 | UCCGUGUACGGCUGAAUCUUU | 8947 | GGGGACCCCCAACCUUCGCUC |
| 6344 | CCGUGUACGGCUGAAUCUUUU | 8948 | GGGACCCCCAACCUUCGCUCC |
| 6345 | CGUGUACGGCUGAAUCUUUUG | 8949 | GGACCCCCAACCUUCGCUCCC |
| 6346 | GUGUACGGCUGAAUCUUUUGC | 8950 | GACCCCCAACCUUCGCUCCCC |
| 6347 | UGUACGGCUGAAUCUUUUGCA | 8951 | ACCCCCAACCUUCGCUCCCCU |
| 6348 | GUACGGCUGAAUCUUUUGCAC | 8952 | CCCCCAACCUUCGCUCCCCUC |
| 6349 | UACGGCUGAAUCUUUUGCACA | 8953 | CCCCAACCUUCGCUCCCCUCA |
| 6350 | ACGGCUGAAUCUUUUGCACAA | 8954 | CCCAACCUUCGCUCCCCUCAC |
| 6351 | CGGCUGAAUCUUUUGCACAAU | 8955 | CCAACCUUCGCUCCCCUCACC |
| 6352 | GGCUGAAUCUUUUGCACAAUG | 8956 | CAACCUUCGCUCCCCUCACCC |
| 6353 | GCUGAAUCUUUUGCACAAUGA | 8957 | AACCUUCGCUCCCCUCACCCG |
| 6354 | CUGAAUCUUUUGCACAAUGAU | 8958 | ACCUUCGCUCCCCUCACCCGG |
| 6355 | UGAAUCUUUUGCACAAUGAUG | 8959 | CCUUCGCUCCCCUCACCCGGA |
| 6356 | GAAUCUUUUGCACAAUGAUGU | 8960 | CUUCGCUCCCCUCACCCGGAG |
| 6357 | AAUCUUUUGCACAAUGAUGUC | 8961 | UUCGCUCCCCUCACCCGGAGG |
| 6358 | AUCUUUUGCACAAUGAUGUCG | 8962 | UCGCUCCCCUCACCCGGAGGA |
| 6359 | UCUUUUGCACAAUGAUGUCGG | 8963 | CGCUCCCCUCACCCGGAGGAG |
| 6360 | CUUUUGCACAAUGAUGUCGGA | 8964 | GCUCCCCUCACCCGGAGGAGG |
| 6361 | UUUUGCACAAUGAUGUCGGAA | 8965 | CUCCCCUCACCCGGAGGAGGA |
| 6362 | UUUGCACAAUGAUGUCGGAAU | 8966 | UCCCCUCACCCGGAGGAGGAG |
| 6363 | UUGCACAAUGAUGUCGGAAUC | 8967 | CCCCUCACCCGGAGGAGGAGG |
| 6364 | UGCACAAUGAUGUCGGAAUCC | 8968 | CCCUCACCCGGAGGAGGAGGA |
| 6365 | GCACAAUGAUGUCGGAAUCCA | 8969 | CCUCACCCGGAGGAGGAGGAG |
| 6366 | CACAAUGAUGUCGGAAUCCAG | 8970 | CUCACCCGGAGGAGGAGGAGG |
| 6367 | ACAAUGAUGUCGGAAUCCAGC | 8971 | UCACCCGGAGGAGGAGGAGGA |
| 6368 | CAAUGAUGUCGGAAUCCAGCA | 8972 | CACCCGGAGGAGGAGGAGGAA |
| 6369 | AAUGAUGUCGGAAUCCAGCAC | 8973 | ACCCGGAGGAGGAGGAGGAAG |
| 6370 | AUGAUGUCGGAAUCCAGCACC | 8974 | CCCGGAGGAGGAGGAGGAAGA |
| 6371 | UGAUGUCGGAAUCCAGCACCC | 8975 | CCGGAGGAGGAGGAGGAAGAG |
| 6372 | GAUGUCGGAAUCCAGCACCCC | 8976 | CGGAGGAGGAGGAGGAAGAGG |
| 6373 | AUGUCGGAAUCCAGCACCCCC | 8977 | GGAGGAGGAGGAGGAAGAGGA |
| 6374 | UGUCGGAAUCCAGCACCCCCA | 8978 | GAGGAGGAGGAGGAAGAGGAA |
| 6375 | GUCGGAAUCCAGCACCCCCAG | 8979 | AGGAGGAGGAGGAAGAGGAAG |
| 6376 | UCGGAAUCCAGCACCCCCAGG | 8980 | GGAGGAGGAGGAAGAGGAAGA |
| 6377 | CGGAAUCCAGCACCCCCAGGA | 8981 | GAGGAGGAGGAAGAGGAAGAA |
| 6378 | GGAAUCCAGCACCCCCAGGAG | 8982 | AGGAGGAGGAAGAGGAAGAAG |
| 6379 | GAAUCCAGCACCCCCAGGAGG | 8983 | GGAGGAGGAAGAGGAAGAAGG |
| 6380 | AAUCCAGCACCCCCAGGAGGA | 8984 | GAGGAGGAAGAGGAAGAAGGU |
| 6381 | AUCCAGCACCCCCAGGAGGAC | 8985 | AGGAGGAAGAGGAAGAAGGUA |
| 6382 | UCCAGCACCCCCAGGAGGACC | 8986 | GGAGGAAGAGGAAGAAGGUAG |
| 6383 | CCAGCACCCCCAGGAGGACCC | 8987 | GAGGAAGAGGAAGAAGGUAGU |
| 6384 | CAGCACCCCCAGGAGGACCCC | 8988 | AGGAAGAGGAAGAAGGUAGUG |
| 6385 | AGCACCCCCAGGAGGACCCCA | 8989 | GGAAGAGGAAGAAGGUAGUGC |
| 6386 | GCACCCCCAGGAGGACCCCAA | 8990 | GAAGAGGAAGAAGGUAGUGCG |
| 6387 | CACCCCCAGGAGGACCCCAAU | 8991 | AAGAGGAAGAAGGUAGUGCGG |
| 6388 | ACCCCCAGGAGGACCCCAAUC | 8992 | AGAGGAAGAAGGUAGUGCGGG |
| 6389 | CCCCCAGGAGGACCCCAAUCU | 8993 | GAGGAAGAAGGUAGUGCGGGC |
| 6390 | CCCCAGGAGGACCCCAAUCUG | 8994 | AGGAAGAAGGUAGUGCGGGCU |
| 6391 | CCCAGGAGGACCCCAAUCUGG | 8995 | GGAAGAAGGUAGUGCGGGCUC |
| 6392 | CCAGGAGGACCCCAAUCUGGC | 8996 | GAAGAAGGUAGUGCGGGCUCC |
| 6393 | CAGGAGGACCCCAAUCUGGCG | 8997 | AAGAAGGUAGUGCGGGCUCCC |
| 6394 | AGGAGGACCCCAAUCUGGCGG | 8998 | AGAAGGUAGUGCGGGCUCCCC |
| 6395 | GGAGGACCCCAAUCUGGCGGA | 8999 | GAAGGUAGUGCGGGCUCCCCA |
| 6396 | GAGGACCCCAAUCUGGCGGAU | 9000 | AAGGUAGUGCGGGCUCCCCAC |
| 6397 | AGGACCCCAAUCUGGCGGAUG | 9001 | AGGUAGUGCGGGCUCCCCACC |
| 6398 | GGACCCCAAUCUGGCGGAUGA | 9002 | GGUAGUGCGGGCUCCCCACCC |
| 6399 | GACCCCAAUCUGGCGGAUGAA | 9003 | GUAGUGCGGGCUCCCCACCCG |
| 6400 | ACCCCAAUCUGGCGGAUGAAC | 9004 | UAGUGCGGGCUCCCCACCCGG |
| 6401 | CCCCAAUCUGGCGGAUGAACA | 9005 | AGUGCGGGCUCCCCACCCGGA |
| 6402 | CCCAAUCUGGCGGAUGAACAU | 9006 | GUGCGGGCUCCCCACCCGGAC |
| 6403 | CCAAUCUGGCGGAUGAACAUC | 9007 | UGCGGGCUCCCCACCCGGACA |
| 6404 | CAAUCUGGCGGAUGAACAUCC | 9008 | GCGGGCUCCCCACCCGGACAG |
| 6405 | AAUCUGGCGGAUGAACAUCCC | 9009 | CGGGCUCCCCACCCGGACAGC |
| 6406 | AUCUGGCGGAUGAACAUCCCC | 9010 | GGGCUCCCCACCCGGACAGCU |
| 6407 | UCUGGCGGAUGAACAUCCCCU | 9011 | GGCUCCCCACCCGGACAGCUA |
| 6408 | CUGGCGGAUGAACAUCCCCUU | 9012 | GCUCCCCACCCGGACAGCUAC |
| 6409 | UGGCGGAUGAACAUCCCCUUC | 9013 | CUCCCCACCCGGACAGCUACC |
| 6410 | GGCGGAUGAACAUCCCCUUCA | 9014 | UCCCCACCCGGACAGCUACCU |
| 6411 | GCGGAUGAACAUCCCCUUCAG | 9015 | CCCCACCCGGACAGCUACCUC |
| 6412 | CGGAUGAACAUCCCCUUCAGC | 9016 | CCCACCCGGACAGCUACCUCU |
| 6413 | GGAUGAACAUCCCCUUCAGCC | 9017 | CCACCCGGACAGCUACCUCUC |
| 6414 | GAUGAACAUCCCCUUCAGCCU | 9018 | CACCCGGACAGCUACCUCUCG |
| 6415 | AUGAACAUCCCCUUCAGCCUC | 9019 | ACCCGGACAGCUACCUCUCGC |
| 6416 | UGAACAUCCCCUUCAGCCUCU | 9020 | CCCGGACAGCUACCUCUCGCC |
| 6417 | GAACAUCCCCUUCAGCCUCUC | 9021 | CCGGACAGCUACCUCUCGCCU |
| 6418 | AACAUCCCCUUCAGCCUCUCA | 9022 | CGGACAGCUACCUCUCGCCUC |
| 6419 | ACAUCCCCUUCAGCCUCUCAG | 9023 | GGACAGCUACCUCUCGCCUCA |
| 6420 | CAUCCCCUUCAGCCUCUCAGU | 9024 | GACAGCUACCUCUCGCCUCAG |
| 6421 | AUCCCCUUCAGCCUCUCAGUU | 9025 | ACAGCUACCUCUCGCCUCAGC |
| 6422 | UCCCCUUCAGCCUCUCAGUUA | 9026 | CAGCUACCUCUCGCCUCAGCC |
| 6423 | CCCCUUCAGCCUCUCAGUUAG | 9027 | AGCUACCUCUCGCCUCAGCCU |
| 6424 | CCCUUCAGCCUCUCAGUUAGC | 9028 | GCUACCUCUCGCCUCAGCCUC |
| 6425 | CCUUCAGCCUCUCAGUUAGCU | 9029 | CUACCUCUCGCCUCAGCCUCC |
| 6426 | CUUCAGCCUCUCAGUUAGCUG | 9030 | UACCUCUCGCCUCAGCCUCCC |
| 6427 | UUCAGCCUCUCAGUUAGCUGA | 9031 | ACCUCUCGCCUCAGCCUCCCU |
| 6428 | UCAGCCUCUCAGUUAGCUGAC | 9032 | CCUCUCGCCUCAGCCUCCCUG |
| 6429 | CAGCCUCUCAGUUAGCUGACU | 9033 | CUCUCGCCUCAGCCUCCCUGG |
| 6430 | AGCCUCUCAGUUAGCUGACUG | 9034 | UCUCGCCUCAGCCUCCCUGGA |
| 6431 | GCCUCUCAGUUAGCUGACUGA | 9035 | CUCGCCUCAGCCUCCCUGGAC |
| 6432 | CCUCUCAGUUAGCUGACUGAC | 9036 | UCGCCUCAGCCUCCCUGGACA |
| 6433 | CUCUCAGUUAGCUGACUGACG | 9037 | CGCCUCAGCCUCCCUGGACAG |
| 6434 | UCUCAGUUAGCUGACUGACGU | 9038 | GCCUCAGCCUCCCUGGACAGC |
| 6435 | CUCAGUUAGCUGACUGACGUU | 9039 | CCUCAGCCUCCCUGGACAGCG |
| 6436 | UCAGUUAGCUGACUGACGUUG | 9040 | CUCAGCCUCCCUGGACAGCGA |
| 6437 | CAGUUAGCUGACUGACGUUGA | 9041 | UCAGCCUCCCUGGACAGCGAC |
| 6438 | AGUUAGCUGACUGACGUUGAU | 9042 | CAGCCUCCCUGGACAGCGACG |
| 6439 | GUUAGCUGACUGACGUUGAUA | 9043 | AGCCUCCCUGGACAGCGACGG |
| 6440 | UUAGCUGACUGACGUUGAUAU | 9044 | GCCUCCCUGGACAGCGACGGC |
| 6441 | UAGCUGACUGACGUUGAUAUC | 9045 | CCUCCCUGGACAGCGACGGCG |
| 6442 | AGCUGACUGACGUUGAUAUCC | 9046 | CUCCCUGGACAGCGACGGCGG |
| 6443 | GCUGACUGACGUUGAUAUCCA | 9047 | UCCCUGGACAGCGACGGCGGC |
| 6444 | CUGACUGACGUUGAUAUCCAA | 9048 | CCCUGGACAGCGACGGCGGCC |
| 6445 | UGACUGACGUUGAUAUCCAAG | 9049 | CCUGGACAGCGACGGCGGCCG |
| 6446 | GACUGACGUUGAUAUCCAAGA | 9050 | CUGGACAGCGACGGCGGCCGG |
| 6447 | ACUGACGUUGAUAUCCAAGAU | 9051 | UGGACAGCGACGGCGGCCGGA |
| 6448 | CUGACGUUGAUAUCCAAGAUG | 9052 | GGACAGCGACGGCGGCCGGAA |
| 6449 | UGACGUUGAUAUCCAAGAUGA | 9053 | GACAGCGACGGCGGCCGGAAA |
| 6450 | GACGUUGAUAUCCAAGAUGAU | 9054 | ACAGCGACGGCGGCCGGAAAC |
| 6451 | ACGUUGAUAUCCAAGAUGAUC | 9055 | AGGUUGGUGUUCUAACACCCA |
| 6452 | CGUUGAUAUCCAAGAUGAUCU | 9056 | GGUUGGUGUUCUAACACCCAG |
| 6453 | GUUGAUAUCCAAGAUGAUCUC | 9057 | GUUGGUGUUCUAACACCCAGU |
| 6454 | UUGAUAUCCAAGAUGAUCUCC | 9058 | UUGGUGUUCUAACACCCAGUU |
| 6455 | UGAUAUCCAAGAUGAUCUCCA | 9059 | UGGUGUUCUAACACCCAGUUC |
| 6456 | GAUAUCCAAGAUGAUCUCCAC | 9060 | GGUGUUCUAACACCCAGUUCA |
| 6457 | AUAUCCAAGAUGAUCUCCACC | 9061 | GUGUUCUAACACCCAGUUCAG |
| 6458 | UAUCCAAGAUGAUCUCCACCA | 9062 | UGUUCUAACACCCAGUUCAGC |
| 6459 | AUCCAAGAUGAUCUCCACCAG | 9063 | GUUCUAACACCCAGUUCAGCA |
| 6460 | UCCAAGAUGAUCUCCACCAGG | 9064 | UUCUAACACCCAGUUCAGCAU |
| 6461 | CCAAGAUGAUCUCCACCAGGU | 9065 | UCUAACACCCAGUUCAGCAUG |
| 6462 | CAAGAUGAUCUCCACCAGGUU | 9066 | CUAACACCCAGUUCAGCAUGU |
| 6463 | AAGAUGAUCUCCACCAGGUUG | 9067 | UAACACCCAGUUCAGCAUGUG |
| 6464 | AGAUGAUCUCCACCAGGUUGU | 9068 | AACACCCAGUUCAGCAUGUGA |
| 6465 | GAUGAUCUCCACCAGGUUGUU | 9069 | ACACCCAGUUCAGCAUGUGAG |
| 6466 | AUGAUCUCCACCAGGUUGUUU | 9070 | CACCCAGUUCAGCAUGUGAGU |
| 6467 | UGAUCUCCACCAGGUUGUUUU | 9071 | ACCCAGUUCAGCAUGUGAGUG |
| 6468 | GAUCUCCACCAGGUUGUUUUU | 9072 | CCCAGUUCAGCAUGUGAGUGA |
| 6469 | AUCUCCACCAGGUUGUUUUUC | 9073 | CCAGUUCAGCAUGUGAGUGAA |
| 6470 | UCUCCACCAGGUUGUUUUUCC | 9074 | CAGUUCAGCAUGUGAGUGAAU |
| 6471 | CUCCACCAGGUUGUUUUUCCU | 9075 | AGUUCAGCAUGUGAGUGAAUA |
| 6472 | UCCACCAGGUUGUUUUUCCUG | 9076 | GUUCAGCAUGUGAGUGAAUAU |
| 6473 | CCACCAGGUUGUUUUUCCUGG | 9077 | UUCAGCAUGUGAGUGAAUAUG |
| 6474 | CACCAGGUUGUUUUUCCUGGG | 9078 | UCAGCAUGUGAGUGAAUAUGG |
| 6475 | ACCAGGUUGUUUUUCCUGGGA | 9079 | CAGCAUGUGAGUGAAUAUGGA |
| 6476 | CCAGGUUGUUUUUCCUGGGAU | 9080 | AGCAUGUGAGUGAAUAUGGAG |
| 6477 | CAGGUUGUUUUUCCUGGGAUC | 9081 | GCAUGUGAGUGAAUAUGGAGU |
| 6478 | AGGUUGUUUUUCCUGGGAUCA | 9082 | CAUGUGAGUGAAUAUGGAGUC |
| 6479 | GGUUGUUUUUCCUGGGAUCAG | 9083 | AUGUGAGUGAAUAUGGAGUCA |
| 6480 | GUUGUUUUUCCUGGGAUCAGG | 9084 | UGUGAGUGAAUAUGGAGUCAG |
| 6481 | UUGUUUUUCCUGGGAUCAGGU | 9085 | GUGAGUGAAUAUGGAGUCAGA |
| 6482 | UGUUUUUCCUGGGAUCAGGUU | 9086 | UGAGUGAAUAUGGAGUCAGAU |
| 6483 | GUUUUUCCUGGGAUCAGGUUU | 9087 | GAGUGAAUAUGGAGUCAGAUA |
| 6484 | UUUUUCCUGGGAUCAGGUUUC | 9088 | AGUGAAUAUGGAGUCAGAUAU |
| 6485 | UUUUCCUGGGAUCAGGUUUCA | 9089 | GUGAAUAUGGAGUCAGAUAUC |
| 6486 | UUUCCUGGGAUCAGGUUUCAC | 9090 | UGAAUAUGGAGUCAGAUAUCA |
| 6487 | UUCCUGGGAUCAGGUUUCACC | 9091 | GAAUAUGGAGUCAGAUAUCAU |
| 6488 | UCCUGGGAUCAGGUUUCACCU | 9092 | AAUAUGGAGUCAGAUAUCAUG |
| 6489 | CCUGGGAUCAGGUUUCACCUC | 9093 | AUAUGGAGUCAGAUAUCAUGA |
| 6490 | CUGGGAUCAGGUUUCACCUCC | 9094 | UAUGGAGUCAGAUAUCAUGAG |
| 6491 | UGGGAUCAGGUUUCACCUCCA | 9095 | AUGGAGUCAGAUAUCAUGAGG |
| 6492 | GGGAUCAGGUUUCACCUCCAC | 9096 | UGGAGUCAGAUAUCAUGAGGC |
| 6493 | GGAUCAGGUUUCACCUCCACA | 9097 | GGAGUCAGAUAUCAUGAGGCA |
| 6494 | GAUCAGGUUUCACCUCCACAG | 9098 | GAGUCAGAUAUCAUGAGGCAC |
| 6495 | AUCAGGUUUCACCUCCACAGU | 9099 | AGUCAGAUAUCAUGAGGCACC |
| 6496 | UCAGGUUUCACCUCCACAGUG | 9100 | GUCAGAUAUCAUGAGGCACCU |
| 6497 | CAGGUUUCACCUCCACAGUGG | 9101 | UCAGAUAUCAUGAGGCACCUU |
| 6498 | AGGUUUCACCUCCACAGUGGU | 9102 | CAGAUAUCAUGAGGCACCUUG |
| 6499 | GGUUUCACCUCCACAGUGGUC | 9103 | AGAUAUCAUGAGGCACCUUGC |
| 6500 | GUUUCACCUCCACAGUGGUCC | 9104 | GAUAUCAUGAGGCACCUUGCU |
| 6501 | UUUCACCUCCACAGUGGUCCG | 9105 | AUAUCAUGAGGCACCUUGCUA |
| 6502 | UUCACCUCCACAGUGGUCCGG | 9106 | UAUCAUGAGGCACCUUGCUAA |
| 6503 | UCACCUCCACAGUGGUCCGGU | 9107 | AUCAUGAGGCACCUUGCUAAG |
| 6504 | CACCUCCACAGUGGUCCGGUC | 9108 | UCAUGAGGCACCUUGCUAAGG |
| 6505 | ACCUCCACAGUGGUCCGGUCU | 9109 | CAUGAGGCACCUUGCUAAGGG |
| 6506 | CCUCCACAGUGGUCCGGUCUG | 9110 | AUGAGGCACCUUGCUAAGGGU |
| 6507 | CUCCACAGUGGUCCGGUCUGU | 9111 | UGAGGCACCUUGCUAAGGGUU |
| 6508 | UCCACAGUGGUCCGGUCUGUG | 9112 | GAGGCACCUUGCUAAGGGUUU |
| 6509 | CCACAGUGGUCCGGUCUGUGU | 9113 | AGGCACCUUGCUAAGGGUUUC |
| 6510 | CACAGUGGUCCGGUCUGUGUC | 9114 | GGCACCUUGCUAAGGGUUUCC |
| 6511 | ACAGUGGUCCGGUCUGUGUCA | 9115 | GCACCUUGCUAAGGGUUUCCC |
| 6512 | CAGUGGUCCGGUCUGUGUCAC | 9116 | CACCUUGCUAAGGGUUUCCCC |
| 6513 | AGUGGUCCGGUCUGUGUCACU | 9117 | ACCUUGCUAAGGGUUUCCCCC |
| 6514 | GUGGUCCGGUCUGUGUCACUC | 9118 | CCUUGCUAAGGGUUUCCCCCU |
| 6515 | UGGUCCGGUCUGUGUCACUCU | 9119 | CUUGCUAAGGGUUUCCCCCUU |
| 6516 | GGUCCGGUCUGUGUCACUCUC | 9120 | UUGCUAAGGGUUUCCCCCUUC |
| 6517 | GUCCGGUCUGUGUCACUCUCA | 9121 | UGCUAAGGGUUUCCCCCUUCA |
| 6518 | UCCGGUCUGUGUCACUCUCAC | 9122 | GCUAAGGGUUUCCCCCUUCAA |
| 6519 | CCGGUCUGUGUCACUCUCACC | 9123 | CUAAGGGUUUCCCCCUUCAAG |
| 6520 | CGGUCUGUGUCACUCUCACCC | 9124 | UAAGGGUUUCCCCCUUCAAGG |
| 6521 | GGUCUGUGUCACUCUCACCCU | 9125 | AAGGGUUUCCCCCUUCAAGGA |
| 6522 | GUCUGUGUCACUCUCACCCUU | 9126 | AGGGUUUCCCCCUUCAAGGAA |
| 6523 | UCUGUGUCACUCUCACCCUUU | 9127 | GGGUUUCCCCCUUCAAGGAAA |
| 6524 | CUGUGUCACUCUCACCCUUUG | 9128 | GGUUUCCCCCUUCAAGGAAAC |
| 6525 | UGUGUCACUCUCACCCUUUGC | 9129 | GUUUCCCCCUUCAAGGAAACA |
| 6526 | GUGUCACUCUCACCCUUUGCA | 9130 | UUUCCCCCUUCAAGGAAACAA |
| 6527 | UGUCACUCUCACCCUUUGCAU | 9131 | UUCCCCCUUCAAGGAAACAAC |
| 6528 | GUCACUCUCACCCUUUGCAUC | 9132 | UCCCCCUUCAAGGAAACAACC |
| 6529 | UCACUCUCACCCUUUGCAUCG | 9133 | CCCCCUUCAAGGAAACAACCC |
| 6530 | CACUCUCACCCUUUGCAUCGG | 9134 | CCCCUUCAAGGAAACAACCCU |
| 6531 | ACUCUCACCCUUUGCAUCGGU | 9135 | CCCUUCAAGGAAACAACCCUC |
| 6532 | CUCUCACCCUUUGCAUCGGUC | 9136 | CCUUCAAGGAAACAACCCUCU |
| 6533 | UCUCACCCUUUGCAUCGGUCA | 9137 | CUUCAAGGAAACAACCCUCUC |
| 6534 | CUCACCCUUUGCAUCGGUCAC | 9138 | UUCAAGGAAACAACCCUCUCU |
| 6535 | UCACCCUUUGCAUCGGUCACU | 9139 | UCAAGGAAACAACCCUCUCUG |
| 6536 | CACCCUUUGCAUCGGUCACUU | 9140 | CAAGGAAACAACCCUCUCUGA |
| 6537 | ACCCUUUGCAUCGGUCACUUU | 9141 | AAGGAAACAACCCUCUCUGAC |
| 6538 | CCCUUUGCAUCGGUCACUUUC | 9142 | AGGAAACAACCCUCUCUGACA |
| 6539 | CCUUUGCAUCGGUCACUUUCA | 9143 | GGAAACAACCCUCUCUGACAC |
| 6540 | CUUUGCAUCGGUCACUUUCAG | 9144 | GAAACAACCCUCUCUGACACA |
| 6541 | UUUGCAUCGGUCACUUUCAGG | 9145 | AAACAACCCUCUCUGACACAG |
| 6542 | UUGCAUCGGUCACUUUCAGGU | 9146 | AACAACCCUCUCUGACACAGC |
| 6543 | UGCAUCGGUCACUUUCAGGUG | 9147 | ACAACCCUCUCUGACACAGCA |
| 6544 | GCAUCGGUCACUUUCAGGUGA | 9148 | AGACAGGCAGAACCAGUACAA |
| 6545 | CAUCGGUCACUUUCAGGUGAA | 9149 | GACAGGCAGAACCAGUACAAA |
| 6546 | AUCGGUCACUUUCAGGUGAAA | 9150 | ACAGGCAGAACCAGUACAAAG |
| 6547 | UCGGUCACUUUCAGGUGAAAA | 9151 | CAGGCAGAACCAGUACAAAGC |
| 6548 | CGGUCACUUUCAGGUGAAAAG | 9152 | AGGCAGAACCAGUACAAAGCG |
| 6549 | GGUCACUUUCAGGUGAAAAGU | 9153 | GGCAGAACCAGUACAAAGCGA |
| 6550 | GUCACUUUCAGGUGAAAAGUG | 9154 | GCAGAACCAGUACAAAGCGAA |
| 6551 | UCACUUUCAGGUGAAAAGUGU | 9155 | CAGAACCAGUACAAAGCGAAG |
| 6552 | CACUUUCAGGUGAAAAGUGUA | 9156 | AGAACCAGUACAAAGCGAAGG |
| 6553 | ACUUUCAGGUGAAAAGUGUAG | 9157 | GAACCAGUACAAAGCGAAGGA |
| 6554 | CUUUCAGGUGAAAAGUGUAGG | 9158 | AACCAGUACAAAGCGAAGGAA |
| 6555 | UUUCAGGUGAAAAGUGUAGGU | 9159 | ACCAGUACAAAGCGAAGGAAU |
| 6556 | UUCAGGUGAAAAGUGUAGGUU | 9160 | CCAGUACAAAGCGAAGGAAUC |
| 6557 | UCAGGUGAAAAGUGUAGGUUC | 9161 | CAGUACAAAGCGAAGGAAUCU |
| 6558 | CAGGUGAAAAGUGUAGGUUCC | 9162 | AGUACAAAGCGAAGGAAUCUG |
| 6559 | AGGUGAAAAGUGUAGGUUCCC | 9163 | GUACAAAGCGAAGGAAUCUGG |
| 6560 | GGUGAAAAGUGUAGGUUCCCU | 9164 | UACAAAGCGAAGGAAUCUGGG |
| 6561 | GUGAAAAGUGUAGGUUCCCUC | 9165 | ACAAAGCGAAGGAAUCUGGGC |
| 6562 | UGAAAAGUGUAGGUUCCCUCA | 9166 | CAAAGCGAAGGAAUCUGGGCC |
| 6563 | GAAAAGUGUAGGUUCCCUCAA | 9167 | AAAGCGAAGGAAUCUGGGCCC |
| 6564 | AAAAGUGUAGGUUCCCUCAAC | 9168 | AAGCGAAGGAAUCUGGGCCCC |
| 6565 | AAAGUGUAGGUUCCCUCAACC | 9169 | AGCGAAGGAAUCUGGGCCCCC |
| 6566 | AAGUGUAGGUUCCCUCAACCA | 9170 | GCGAAGGAAUCUGGGCCCCCA |
| 6567 | AGUGUAGGUUCCCUCAACCAG | 9171 | CGAAGGAAUCUGGGCCCCCAG |
| 6568 | GUGUAGGUUCCCUCAACCAGG | 9172 | GAAGGAAUCUGGGCCCCCAGC |
| 6569 | UGUAGGUUCCCUCAACCAGGU | 9173 | AAGGAAUCUGGGCCCCCAGCC |
| 6570 | GUAGGUUCCCUCAACCAGGUU | 9174 | AGGAAUCUGGGCCCCCAGCCU |
| 6571 | UAGGUUCCCUCAACCAGGUUU | 9175 | GGAAUCUGGGCCCCCAGCCUC |
| 6572 | AGGUUCCCUCAACCAGGUUUG | 9176 | GAAUCUGGGCCCCCAGCCUCU |
| 6573 | GGUUCCCUCAACCAGGUUUGA | 9177 | AAUCUGGGCCCCCAGCCUCUC |
| 6574 | GUUCCCUCAACCAGGUUUGAA | 9178 | AUCUGGGCCCCCAGCCUCUCG |
| 6575 | UUCCCUCAACCAGGUUUGAAA | 9179 | UCUGGGCCCCCAGCCUCUCGC |
| 6576 | UCCCUCAACCAGGUUUGAAAG | 9180 | CUGGGCCCCCAGCCUCUCGCC |
| 6577 | CCCUCAACCAGGUUUGAAAGA | 9181 | UGGGCCCCCAGCCUCUCGCCG |
| 6578 | CCUCAACCAGGUUUGAAAGAA | 9182 | GGGCCCCCAGCCUCUCGCCGC |
| 6579 | CUCAACCAGGUUUGAAAGAAA | 9183 | GGCCCCCAGCCUCUCGCCGCC |
| 6580 | UCAACCAGGUUUGAAAGAAAA | 9184 | GCCCCCAGCCUCUCGCCGCCC |
| 6581 | CAACCAGGUUUGAAAGAAAAA | 9185 | CCCCCAGCCUCUCGCCGCCCG |
| 6582 | AACCAGGUUUGAAAGAAAAAG | 9186 | CCCCAGCCUCUCGCCGCCCGC |
| 6583 | ACCAGGUUUGAAAGAAAAAGG | 9187 | CCCAGCCUCUCGCCGCCCGCU |
| 6584 | CCAGGUUUGAAAGAAAAAGGA | 9188 | CCAGCCUCUCGCCGCCCGCUC |
| 6585 | CAGGUUUGAAAGAAAAAGGAU | 9189 | CAGCCUCUCGCCGCCCGCUCU |
| 6586 | AGGUUUGAAAGAAAAAGGAUA | 9190 | AGCCUCUCGCCGCCCGCUCUC |
| 6587 | GGUUUGAAAGAAAAAGGAUAG | 9191 | GCCUCUCGCCGCCCGCUCUCC |
| 6588 | GUUUGAAAGAAAAAGGAUAGG | 9192 | CCUCUCGCCGCCCGCUCUCCA |
| 6589 | UUUGAAAGAAAAAGGAUAGGG | 9193 | CUCUCGCCGCCCGCUCUCCAG |
| 6590 | UUGAAAGAAAAAGGAUAGGGU | 9194 | UCUCGCCGCCCGCUCUCCAGA |
| 6591 | UGAAAGAAAAAGGAUAGGGUG | 9195 | CUCGCCGCCCGCUCUCCAGAG |
| 6592 | GAAAGAAAAAGGAUAGGGUGA | 9196 | UCGCCGCCCGCUCUCCAGAGG |
| 6593 | AAAGAAAAAGGAUAGGGUGAU | 9197 | CGCCGCCCGCUCUCCAGAGGC |
| 6594 | AAGAAAAAGGAUAGGGUGAUG | 9198 | GCCGCCCGCUCUCCAGAGGCA |
| 6595 | AGAAAAAGGAUAGGGUGAUGG | 9199 | CCGCCCGCUCUCCAGAGGCAG |
| 6596 | GAAAAAGGAUAGGGUGAUGGU | 9200 | CGCCCGCUCUCCAGAGGCAGU |
| 6597 | AAAAAGGAUAGGGUGAUGGUC | 9201 | GCCCGCUCUCCAGAGGCAGUC |
| 6598 | AAAAGGAUAGGGUGAUGGUCA | 9202 | CCCGCUCUCCAGAGGCAGUCU |
| 6599 | AAAGGAUAGGGUGAUGGUCAG | 9203 | CCGCUCUCCAGAGGCAGUCUG |
| 6600 | AAGGAUAGGGUGAUGGUCAGA | 9204 | CGCUCUCCAGAGGCAGUCUGC |
| 6601 | AGGAUAGGGUGAUGGUCAGAG | 9205 | GCUCUCCAGAGGCAGUCUGCA |
| 6602 | GGAUAGGGUGAUGGUCAGAGU | 9206 | CUCUCCAGAGGCAGUCUGCAC |
| 6603 | GAUAGGGUGAUGGUCAGAGUG | 9207 | UCUCCAGAGGCAGUCUGCACC |
| 6604 | AUAGGGUGAUGGUCAGAGUGA | 9208 | CUCCAGAGGCAGUCUGCACCU |
| 6605 | UAGGGUGAUGGUCAGAGUGAU | 9209 | UCCAGAGGCAGUCUGCACCUU |
| 6606 | AGGGUGAUGGUCAGAGUGAUU | 9210 | CCAGAGGCAGUCUGCACCUUG |
| 6607 | GGGUGAUGGUCAGAGUGAUUU | 9211 | CAGAGGCAGUCUGCACCUUGC |
| 6608 | GGUGAUGGUCAGAGUGAUUUA | 9212 | AGAGGCAGUCUGCACCUUGCC |
| 6609 | GUGAUGGUCAGAGUGAUUUAA | 9213 | GAGGCAGUCUGCACCUUGCCU |
| 6610 | UGAUGGUCAGAGUGAUUUAAC | 9214 | AGGCAGUCUGCACCUUGCCUC |
| 6611 | GAUGGUCAGAGUGAUUUAACA | 9215 | GGCAGUCUGCACCUUGCCUCC |
| 6612 | AUGGUCAGAGUGAUUUAACAC | 9216 | GCAGUCUGCACCUUGCCUCCU |
| 6613 | UGGUCAGAGUGAUUUAACACC | 9217 | CAGUCUGCACCUUGCCUCCUU |
| 6614 | GGUCAGAGUGAUUUAACACCU | 9218 | AGUCUGCACCUUGCCUCCUUC |
| 6615 | GUCAGAGUGAUUUAACACCUC | 9219 | GUCUGCACCUUGCCUCCUUCG |
| 6616 | UCAGAGUGAUUUAACACCUCC | 9220 | UCUGCACCUUGCCUCCUUCGC |
| 6617 | CAGAGUGAUUUAACACCUCCC | 9221 | CUGCACCUUGCCUCCUUCGCU |
| 6618 | AGAGUGAUUUAACACCUCCCC | 9222 | UGCACCUUGCCUCCUUCGCUC |
| 6619 | GAGUGAUUUAACACCUCCCCU | 9223 | GCACCUUGCCUCCUUCGCUCG |
| 6620 | AGUGAUUUAACACCUCCCCUG | 9224 | CACCUUGCCUCCUUCGCUCGA |
| 6621 | GUGAUUUAACACCUCCCCUGC | 9225 | ACCUUGCCUCCUUCGCUCGAG |
| 6622 | UGAUUUAACACCUCCCCUGCU | 9226 | CCUUGCCUCCUUCGCUCGAGC |
| 6623 | GAUUUAACACCUCCCCUGCUG | 9227 | CUUGCCUCCUUCGCUCGAGCC |
| 6624 | AUUUAACACCUCCCCUGCUGC | 9228 | UUGCCUCCUUCGCUCGAGCCC |
| 6625 | UUUAACACCUCCCCUGCUGCU | 9229 | UGCCUCCUUCGCUCGAGCCCC |
| 6626 | UUAACACCUCCCCUGCUGCUG | 9230 | GCCUCCUUCGCUCGAGCCCCA |
| 6627 | UAACACCUCCCCUGCUGCUGG | 9231 | CCUCCUUCGCUCGAGCCCCAG |
| 6628 | AACACCUCCCCUGCUGCUGGG | 9232 | CUCCUUCGCUCGAGCCCCAGC |
| 6629 | ACACCUCCCCUGCUGCUGGGC | 9233 | UCCUUCGCUCGAGCCCCAGCC |
| 6630 | CACCUCCCCUGCUGCUGGGCU | 9234 | CCUUCGCUCGAGCCCCAGCCC |
| 6631 | ACCUCCCCUGCUGCUGGGCUC | 9235 | CUUCGCUCGAGCCCCAGCCCC |
| 6632 | CCUCCCCUGCUGCUGGGCUCC | 9236 | UUCGCUCGAGCCCCAGCCCCC |
| 6633 | CUCCCCUGCUGCUGGGCUCCC | 9237 | UCGCUCGAGCCCCAGCCCCCA |
| 6634 | UCCCCUGCUGCUGGGCUCCCC | 9238 | CGCUCGAGCCCCAGCCCCCAG |
| 6635 | CCCCUGCUGCUGGGCUCCCCU | 9239 | GCUCGAGCCCCAGCCCCCAGA |
| 6636 | CCCUGCUGCUGGGCUCCCCUC | 9240 | CUCGAGCCCCAGCCCCCAGAC |
| 6637 | CCUGCUGCUGGGCUCCCCUCA | 9241 | UCGAGCCCCAGCCCCCAGACU |
| 6638 | CUGCUGCUGGGCUCCCCUCAU | 9242 | CGAGCCCCAGCCCCCAGACUC |
| 6639 | UGCUGCUGGGCUCCCCUCAUC | 9243 | GAGCCCCAGCCCCCAGACUCG |
| 6640 | GCUGCUGGGCUCCCCUCAUCU | 9244 | AGCCCCAGCCCCCAGACUCGG |
| 6641 | CUGCUGGGCUCCCCUCAUCUC | 9245 | GCCCCAGCCCCCAGACUCGGG |
| 6642 | UGCUGGGCUCCCCUCAUCUCG | 9246 | CCCCAGCCCCCAGACUCGGGC |
| 6643 | GCUGGGCUCCCCUCAUCUCGA | 9247 | CCCAGCCCCCAGACUCGGGCA |
| 6644 | CUGGGCUCCCCUCAUCUCGAG | 9248 | CCAGCCCCCAGACUCGGGCAA |
| 6645 | UGGGCUCCCCUCAUCUCGAGU | 9249 | CAGCCCCCAGACUCGGGCAAU |
| 6646 | GGGCUCCCCUCAUCUCGAGUC | 9250 | AGCCCCCAGACUCGGGCAAUA |
| 6647 | GGCUCCCCUCAUCUCGAGUCC | 9251 | GCCCCCAGACUCGGGCAAUAC |
| 6648 | GCUCCCCUCAUCUCGAGUCCA | 9252 | CCCCCAGACUCGGGCAAUACC |
| 6649 | CUCCCCUCAUCUCGAGUCCAG | 9253 | CCCCAGACUCGGGCAAUACCC |
| 6650 | UCCCCUCAUCUCGAGUCCAGA | 9254 | AGACAGGCAGAACCAGUACAU |
| 6651 | CCCCUCAUCUCGAGUCCAGAG | 9255 | GACAGGCAGAACCAGUACAUU |
| 6652 | CCCUCAUCUCGAGUCCAGAGG | 9256 | ACAGGCAGAACCAGUACAUUU |
| 6653 | CCUCAUCUCGAGUCCAGAGGU | 9257 | CAGGCAGAACCAGUACAUUUU |
| 6654 | CUCAUCUCGAGUCCAGAGGUA | 9258 | AGGCAGAACCAGUACAUUUUG |
| 6655 | UCAUCUCGAGUCCAGAGGUAG | 9259 | GGCAGAACCAGUACAUUUUGA |
| 6656 | CAUCUCGAGUCCAGAGGUAGC | 9260 | GCAGAACCAGUACAUUUUGAG |
| 6657 | AUCUCGAGUCCAGAGGUAGCU | 9261 | CAGAACCAGUACAUUUUGAGG |
| 6658 | UCUCGAGUCCAGAGGUAGCUG | 9262 | AGAACCAGUACAUUUUGAGGA |
| 6659 | CUCGAGUCCAGAGGUAGCUGA | 9263 | GAACCAGUACAUUUUGAGGAG |
| 6660 | UCGAGUCCAGAGGUAGCUGAC | 9264 | AACCAGUACAUUUUGAGGAGA |
| 6661 | CGAGUCCAGAGGUAGCUGACU | 9265 | ACCAGUACAUUUUGAGGAGAA |
| 6662 | GAGUCCAGAGGUAGCUGACUA | 9266 | CCAGUACAUUUUGAGGAGAAG |
| 6663 | AGUCCAGAGGUAGCUGACUAU | 9267 | CAGUACAUUUUGAGGAGAAGA |
| 6664 | GUCCAGAGGUAGCUGACUAUU | 9268 | AGUACAUUUUGAGGAGAAGAG |
| 6665 | UCCAGAGGUAGCUGACUAUUC | 9269 | GUACAUUUUGAGGAGAAGAGG |
| 6666 | CCAGAGGUAGCUGACUAUUCC | 9270 | UACAUUUUGAGGAGAAGAGGU |
| 6667 | CAGAGGUAGCUGACUAUUCCC | 9271 | ACAUUUUGAGGAGAAGAGGUU |
| 6668 | AGAGGUAGCUGACUAUUCCCU | 9272 | CAUUUUGAGGAGAAGAGGUUG |
| 6669 | GAGGUAGCUGACUAUUCCCUU | 9273 | AUUUUGAGGAGAAGAGGUUGC |
| 6670 | AGGUAGCUGACUAUUCCCUUG | 9274 | UUUUGAGGAGAAGAGGUUGCA |
| 6671 | GGUAGCUGACUAUUCCCUUGU | 9275 | UUUGAGGAGAAGAGGUUGCAC |
| 6672 | GUAGCUGACUAUUCCCUUGUC | 9276 | UUGAGGAGAAGAGGUUGCACC |
| 6673 | UAGCUGACUAUUCCCUUGUCA | 9277 | UGAGGAGAAGAGGUUGCACCC |
| 6674 | AGCUGACUAUUCCCUUGUCAU | 9278 | GAGGAGAAGAGGUUGCACCCU |
| 6675 | GCUGACUAUUCCCUUGUCAUC | 9279 | AGGAGAAGAGGUUGCACCCUC |
| 6676 | CUGACUAUUCCCUUGUCAUCU | 9280 | GGAGAAGAGGUUGCACCCUCA |
| 6677 | UGACUAUUCCCUUGUCAUCUG | 9281 | GAGAAGAGGUUGCACCCUCAG |
| 6678 | GACUAUUCCCUUGUCAUCUGA | 9282 | AGAAGAGGUUGCACCCUCAGA |
| 6679 | ACUAUUCCCUUGUCAUCUGAG | 9283 | GAAGAGGUUGCACCCUCAGAG |
| 6680 | CUAUUCCCUUGUCAUCUGAGG | 9284 | AAGAGGUUGCACCCUCAGAGG |
| 6681 | UAUUCCCUUGUCAUCUGAGGA | 9285 | AGAGGUUGCACCCUCAGAGGU |
| 6682 | AUUCCCUUGUCAUCUGAGGAC | 9286 | GAGGUUGCACCCUCAGAGGUA |
| 6683 | UUCCCUUGUCAUCUGAGGACU | 9287 | AGGUUGCACCCUCAGAGGUAA |
| 6684 | UCCCUUGUCAUCUGAGGACUU | 9288 | GGUUGCACCCUCAGAGGUAAA |
| 6685 | CCCUUGUCAUCUGAGGACUUA | 9289 | GUUGCACCCUCAGAGGUAAAG |
| 6686 | CCUUGUCAUCUGAGGACUUAG | 9290 | UUGCACCCUCAGAGGUAAAGU |
| 6687 | CUUGUCAUCUGAGGACUUAGA | 9291 | UGCACCCUCAGAGGUAAAGUA |
| 6688 | UUGUCAUCUGAGGACUUAGAG | 9292 | GCACCCUCAGAGGUAAAGUAA |
| 6689 | UGUCAUCUGAGGACUUAGAGC | 9293 | CACCCUCAGAGGUAAAGUAAC |
| 6690 | GUCAUCUGAGGACUUAGAGCC | 9294 | ACCCUCAGAGGUAAAGUAACC |
| 6691 | UCAUCUGAGGACUUAGAGCCA | 9295 | CCCUCAGAGGUAAAGUAACCU |
| 6692 | CAUCUGAGGACUUAGAGCCAU | 9296 | CCUCAGAGGUAAAGUAACCUG |
| 6693 | AUCUGAGGACUUAGAGCCAUC | 9297 | CUCAGAGGUAAAGUAACCUGA |
| 6694 | UCUGAGGACUUAGAGCCAUCC | 9298 | UCAGAGGUAAAGUAACCUGAG |
| 6695 | CUGAGGACUUAGAGCCAUCCA | 9299 | CAGAGGUAAAGUAACCUGAGG |
| 6696 | UGAGGACUUAGAGCCAUCCAG | 9300 | AGAGGUAAAGUAACCUGAGGG |
| 6697 | GAGGACUUAGAGCCAUCCAGC | 9301 | GAGGUAAAGUAACCUGAGGGU |
| 6698 | AGGACUUAGAGCCAUCCAGCU | 9302 | AGGUAAAGUAACCUGAGGGUU |
| 6699 | GGACUUAGAGCCAUCCAGCUC | 9303 | GGUAAAGUAACCUGAGGGUUA |
| 6700 | GACUUAGAGCCAUCCAGCUCU | 9304 | GUAAAGUAACCUGAGGGUUAU |
| 6701 | ACUUAGAGCCAUCCAGCUCUG | 9305 | UAAAGUAACCUGAGGGUUAUG |
| 6702 | CUUAGAGCCAUCCAGCUCUGC | 9306 | AAAGUAACCUGAGGGUUAUGU |
| 6703 | UUAGAGCCAUCCAGCUCUGCU | 9307 | AAGUAACCUGAGGGUUAUGUC |
| 6704 | UAGAGCCAUCCAGCUCUGCUG | 9308 | AGUAACCUGAGGGUUAUGUCU |
| 6705 | AGAGCCAUCCAGCUCUGCUGU | 9309 | GUAACCUGAGGGUUAUGUCUG |
| 6706 | GAGCCAUCCAGCUCUGCUGUG | 9310 | UAACCUGAGGGUUAUGUCUGG |
| 6707 | AGCCAUCCAGCUCUGCUGUGC | 9311 | AACCUGAGGGUUAUGUCUGGC |
| 6708 | GCCAUCCAGCUCUGCUGUGCU | 9312 | ACCUGAGGGUUAUGUCUGGCC |
| 6709 | CCAUCCAGCUCUGCUGUGCUC | 9313 | CCUGAGGGUUAUGUCUGGCCC |
| 6710 | CAUCCAGCUCUGCUGUGCUCG | 9314 | CUGAGGGUUAUGUCUGGCCCA |
| 6711 | AUCCAGCUCUGCUGUGCUCGU | 9315 | UGAGGGUUAUGUCUGGCCCAG |
| 6712 | UCCAGCUCUGCUGUGCUCGUG | 9316 | GAGGGUUAUGUCUGGCCCAGU |
| 6713 | CCAGCUCUGCUGUGCUCGUGG | 9317 | AGGGUUAUGUCUGGCCCAGUA |
| 6714 | CAGCUCUGCUGUGCUCGUGGG | 9318 | GGGUUAUGUCUGGCCCAGUAU |
| 6715 | AGCUCUGCUGUGCUCGUGGGU | 9319 | GGUUAUGUCUGGCCCAGUAUU |
| 6716 | GCUCUGCUGUGCUCGUGGGUA | 9320 | GUUAUGUCUGGCCCAGUAUUU |
| 6717 | CUCUGCUGUGCUCGUGGGUAG | 9321 | UUAUGUCUGGCCCAGUAUUUU |
| 6718 | UCUGCUGUGCUCGUGGGUAGG | 9322 | UAUGUCUGGCCCAGUAUUUUU |
| 6719 | CUGCUGUGCUCGUGGGUAGGG | 9323 | AUGUCUGGCCCAGUAUUUUUU |
| 6720 | UGCUGUGCUCGUGGGUAGGGU | 9324 | UGUCUGGCCCAGUAUUUUUUA |
| 6721 | GCUGUGCUCGUGGGUAGGGUA | 9325 | GUCUGGCCCAGUAUUUUUUAA |
| 6722 | CUGUGCUCGUGGGUAGGGUAA | 9326 | UCUGGCCCAGUAUUUUUUAAA |
| 6723 | UGUGCUCGUGGGUAGGGUAAU | 9327 | CUGGCCCAGUAUUUUUUAAAU |
| 6724 | GUGCUCGUGGGUAGGGUAAUC | 9328 | UGGCCCAGUAUUUUUUAAAUU |
| 6725 | UGCUCGUGGGUAGGGUAAUCA | 9329 | GGCCCAGUAUUUUUUAAAUUU |
| 6726 | GCUCGUGGGUAGGGUAAUCAC | 9330 | GCCCAGUAUUUUUUAAAUUUC |
| 6727 | CUCGUGGGUAGGGUAAUCACC | 9331 | CCCAGUAUUUUUUAAAUUUCU |
| 6728 | UCGUGGGUAGGGUAAUCACCA | 9332 | CCAGUAUUUUUUAAAUUUCUG |
| 6729 | CGUGGGUAGGGUAAUCACCAC | 9333 | CAGUAUUUUUUAAAUUUCUGA |
| 6730 | GUGGGUAGGGUAAUCACCACA | 9334 | AGUAUUUUUUAAAUUUCUGAA |
| 6731 | UGGGUAGGGUAAUCACCACAU | 9335 | GUAUUUUUUAAAUUUCUGAAU |
| 6732 | GGGUAGGGUAAUCACCACAUU | 9336 | UAUUUUUUAAAUUUCUGAAUG |
| 6733 | GGUAGGGUAAUCACCACAUUC | 9337 | AUUUUUUAAAUUUCUGAAUGC |
| 6734 | GUAGGGUAAUCACCACAUUCC | 9338 | UUUUUUAAAUUUCUGAAUGCA |
| 6735 | UAGGGUAAUCACCACAUUCCC | 9339 | UUUUUAAAUUUCUGAAUGCAA |
| 6736 | AGGGUAAUCACCACAUUCCCA | 9340 | UUUUAAAUUUCUGAAUGCAAA |
| 6737 | GGGUAAUCACCACAUUCCCAG | 9341 | UUUAAAUUUCUGAAUGCAAAU |
| 6738 | GGUAAUCACCACAUUCCCAGU | 9342 | CGUAGGUGUAGGUUUCUCCUG |
| 6739 | GUAAUCACCACAUUCCCAGUU | 9343 | GUAGGUGUAGGUUUCUCCUGG |
| 6740 | UAAUCACCACAUUCCCAGUUA | 9344 | UAGGUGUAGGUUUCUCCUGGG |
| 6741 | AAUCACCACAUUCCCAGUUAU | 9345 | AGGUGUAGGUUUCUCCUGGGU |
| 6742 | AUCACCACAUUCCCAGUUAUC | 9346 | GGUGUAGGUUUCUCCUGGGUA |
| 6743 | UCACCACAUUCCCAGUUAUCU | 9347 | GUGUAGGUUUCUCCUGGGUAU |
| 6744 | CACCACAUUCCCAGUUAUCUU | 9348 | UGUAGGUUUCUCCUGGGUAUG |
| 6745 | ACCACAUUCCCAGUUAUCUUG | 9349 | GUAGGUUUCUCCUGGGUAUGG |
| 6746 | CCACAUUCCCAGUUAUCUUGG | 9350 | UAGGUUUCUCCUGGGUAUGGU |
| 6747 | CACAUUCCCAGUUAUCUUGGC | 9351 | AGGUUUCUCCUGGGUAUGGUG |
| 6748 | ACAUUCCCAGUUAUCUUGGCU | 9352 | GGUUUCUCCUGGGUAUGGUGC |
| 6749 | CAUUCCCAGUUAUCUUGGCUA | 9353 | GUUUCUCCUGGGUAUGGUGCU |
| 6750 | AUUCCCAGUUAUCUUGGCUAU | 9354 | UUUCUCCUGGGUAUGGUGCUG |
| 6751 | UUCCCAGUUAUCUUGGCUAUA | 9355 | UUCUCCUGGGUAUGGUGCUGA |
| 6752 | UCCCAGUUAUCUUGGCUAUAG | 9356 | UCUCCUGGGUAUGGUGCUGAG |
| 6753 | CCCAGUUAUCUUGGCUAUAGG | 9357 | CUCCUGGGUAUGGUGCUGAGG |
| 6754 | CCAGUUAUCUUGGCUAUAGGU | 9358 | UCCUGGGUAUGGUGCUGAGGU |
| 6755 | CAGUUAUCUUGGCUAUAGGUG | 9359 | CCUGGGUAUGGUGCUGAGGUG |
| 6756 | AGUUAUCUUGGCUAUAGGUGG | 9360 | GACAGGCAGAACCAGUACACU |
| 6757 | GUUAUCUUGGCUAUAGGUGGU | 9361 | ACAGGCAGAACCAGUACACUC |
| 6758 | UUAUCUUGGCUAUAGGUGGUU | 9362 | CAGGCAGAACCAGUACACUCU |
| 6759 | UAUCUUGGCUAUAGGUGGUUU | 9363 | AGGCAGAACCAGUACACUCUC |
| 6760 | AUCUUGGCUAUAGGUGGUUUG | 9364 | GGCAGAACCAGUACACUCUCG |
| 6761 | UCUUGGCUAUAGGUGGUUUGU | 9365 | GCAGAACCAGUACACUCUCGC |
| 6762 | CUUGGCUAUAGGUGGUUUGUU | 9366 | CAGAACCAGUACACUCUCGCC |
| 6763 | UUGGCUAUAGGUGGUUUGUUU | 9367 | AGAACCAGUACACUCUCGCCU |
| 6764 | UGGCUAUAGGUGGUUUGUUUA | 9368 | GAACCAGUACACUCUCGCCUC |
| 6765 | GGCUAUAGGUGGUUUGUUUAU | 9369 | AACCAGUACACUCUCGCCUCA |
| 6766 | GCUAUAGGUGGUUUGUUUAUU | 9370 | ACCAGUACACUCUCGCCUCAG |
| 6767 | CUAUAGGUGGUUUGUUUAUUU | 9371 | CCAGUACACUCUCGCCUCAGC |
| 6768 | UAUAGGUGGUUUGUUUAUUUC | 9372 | CAGUACACUCUCGCCUCAGCC |
| 6769 | AUAGGUGGUUUGUUUAUUUCU | 9373 | AGUACACUCUCGCCUCAGCCU |
| 6770 | UAGGUGGUUUGUUUAUUUCUU | 9374 | GUACACUCUCGCCUCAGCCUC |
| 6771 | AGGUGGUUUGUUUAUUUCUUC | 9375 | UACACUCUCGCCUCAGCCUCC |
| 6772 | GGUGGUUUGUUUAUUUCUUCU | 9376 | ACACUCUCGCCUCAGCCUCCC |
| 6773 | GUGGUUUGUUUAUUUCUUCUU | 9377 | CACUCUCGCCUCAGCCUCCCU |
| 6774 | UGGUUUGUUUAUUUCUUCUUU | 9378 | ACUCUCGCCUCAGCCUCCCUG |
| 6775 | GGUUUGUUUAUUUCUUCUUUG | 9379 | ACAGGCAGAACCAGUACACUA |
| 6776 | GUUUGUUUAUUUCUUCUUUGA | 9380 | CAGGCAGAACCAGUACACUAU |
| 6777 | UUUGUUUAUUUCUUCUUUGAC | 9381 | AGGCAGAACCAGUACACUAUG |
| 6778 | UUGUUUAUUUCUUCUUUGACA | 9382 | GGCAGAACCAGUACACUAUGU |
| 6779 | UGUUUAUUUCUUCUUUGACAA | 9383 | GCAGAACCAGUACACUAUGUG |
| 6780 | GUUUAUUUCUUCUUUGACAAU | 9384 | CAGAACCAGUACACUAUGUGG |
| 6781 | UUUAUUUCUUCUUUGACAAUG | 9385 | AGAACCAGUACACUAUGUGGU |
| 6782 | UUAUUUCUUCUUUGACAAUGA | 9386 | GAACCAGUACACUAUGUGGUA |
| 6783 | UAUUUCUUCUUUGACAAUGAC | 9387 | AACCAGUACACUAUGUGGUAG |
| 6784 | AUUUCUUCUUUGACAAUGACA | 9388 | ACCAGUACACUAUGUGGUAGA |
| 6785 | UUUCUUCUUUGACAAUGACAU | 9389 | CCAGUACACUAUGUGGUAGAA |
| 6786 | UUCUUCUUUGACAAUGACAUU | 9390 | CAGUACACUAUGUGGUAGAAU |
| 6787 | UCUUCUUUGACAAUGACAUUC | 9391 | AGUACACUAUGUGGUAGAAUG |
| 6788 | CUUCUUUGACAAUGACAUUCA | 9392 | GUACACUAUGUGGUAGAAUGG |
| 6789 | UUCUUUGACAAUGACAUUCAC | 9393 | UACACUAUGUGGUAGAAUGGA |
| 6790 | UCUUUGACAAUGACAUUCACA | 9394 | ACACUAUGUGGUAGAAUGGAA |
| 6791 | CUUUGACAAUGACAUUCACAG | 9395 | CACUAUGUGGUAGAAUGGAAA |
| 6792 | UUUGACAAUGACAUUCACAGA | 9396 | ACUAUGUGGUAGAAUGGAAAG |
| 6793 | UUGACAAUGACAUUCACAGAG | 9397 | CUAUGUGGUAGAAUGGAAAGA |
| 6794 | UGACAAUGACAUUCACAGAGC | 9398 | UAUGUGGUAGAAUGGAAAGAG |
| 6795 | GACAAUGACAUUCACAGAGCU | 9399 | AUGUGGUAGAAUGGAAAGAGU |
| 6796 | ACAAUGACAUUCACAGAGCUC | 9400 | UGUGGUAGAAUGGAAAGAGUA |
| 6797 | CAAUGACAUUCACAGAGCUCU | 9401 | GUGGUAGAAUGGAAAGAGUAC |
| 6798 | AAUGACAUUCACAGAGCUCUG | 9402 | UGGUAGAAUGGAAAGAGUACC |
| 6799 | AUGACAUUCACAGAGCUCUGG | 9403 | GGUAGAAUGGAAAGAGUACCA |
| 6800 | UGACAUUCACAGAGCUCUGGC | 9404 | GUAGAAUGGAAAGAGUACCAG |
| 6801 | GACAUUCACAGAGCUCUGGCU | 9405 | UAGAAUGGAAAGAGUACCAGC |
| 6802 | ACAUUCACAGAGCUCUGGCUU | 9406 | AGAAUGGAAAGAGUACCAGCC |
| 6803 | CAUUCACAGAGCUCUGGCUUU | 9407 | GAAUGGAAAGAGUACCAGCCC |
| 6804 | AUUCACAGAGCUCUGGCUUUG | 9408 | AAUGGAAAGAGUACCAGCCCG |
| 6805 | UUCACAGAGCUCUGGCUUUGC | 9409 | AUGGAAAGAGUACCAGCCCGC |
| 6806 | UCACAGAGCUCUGGCUUUGCA | 9410 | UGGAAAGAGUACCAGCCCGCA |
| 6807 | CACAGAGCUCUGGCUUUGCAG | 9411 | GGAAAGAGUACCAGCCCGCAG |
| 6808 | ACAGAGCUCUGGCUUUGCAGG | 9412 | GAAAGAGUACCAGCCCGCAGA |
| 6809 | CAGAGCUCUGGCUUUGCAGGU | 9413 | AAAGAGUACCAGCCCGCAGAC |
| 6810 | AGAGCUCUGGCUUUGCAGGUU | 9414 | AAGAGUACCAGCCCGCAGACA |
| 6811 | GAGCUCUGGCUUUGCAGGUUC | 9415 | AGAGUACCAGCCCGCAGACAA |
| 6812 | AGCUCUGGCUUUGCAGGUUCC | 9416 | GAGUACCAGCCCGCAGACAAG |
| 6813 | GCUCUGGCUUUGCAGGUUCCU | 9417 | AGUACCAGCCCGCAGACAAGA |
| 6814 | CUCUGGCUUUGCAGGUUCCUC | 9418 | GUACCAGCCCGCAGACAAGAA |
| 6815 | UCUGGCUUUGCAGGUUCCUCU | 9419 | UACCAGCCCGCAGACAAGAAG |
| 6816 | CUGGCUUUGCAGGUUCCUCUC | 9420 | ACCAGCCCGCAGACAAGAAGA |
| 6817 | UGGCUUUGCAGGUUCCUCUCA | 9421 | CCAGCCCGCAGACAAGAAGAU |
| 6818 | GGCUUUGCAGGUUCCUCUCAU | 9422 | CAGCCCGCAGACAAGAAGAUC |
| 6819 | GCUUUGCAGGUUCCUCUCAUC | 9423 | AGCCCGCAGACAAGAAGAUCU |
| 6820 | CUUUGCAGGUUCCUCUCAUCU | 9424 | GCCCGCAGACAAGAAGAUCUC |
| 6821 | UUUGCAGGUUCCUCUCAUCUU | 9425 | CCCGCAGACAAGAAGAUCUCU |
| 6822 | UUGCAGGUUCCUCUCAUCUUU | 9426 | CCGCAGACAAGAAGAUCUCUG |
| 6823 | UGCAGGUUCCUCUCAUCUUUG | 9427 | CGCAGACAAGAAGAUCUCUGA |
| 6824 | GCAGGUUCCUCUCAUCUUUGA | 9428 | GCAGACAAGAAGAUCUCUGAG |
| 6825 | CAGGUUCCUCUCAUCUUUGAC | 9429 | CAGACAAGAAGAUCUCUGAGU |
| 6826 | AGGUUCCUCUCAUCUUUGACA | 9430 | AGACAAGAAGAUCUCUGAGUU |
| 6827 | GGUUCCUCUCAUCUUUGACAG | 9431 | GACAAGAAGAUCUCUGAGUUU |
| 6828 | GUUCCUCUCAUCUUUGACAGU | 9432 | ACAAGAAGAUCUCUGAGUUUU |
| 6829 | UUCCUCUCAUCUUUGACAGUC | 9433 | CAAGAAGAUCUCUGAGUUUUA |
| 6830 | UCCUCUCAUCUUUGACAGUCA | 9434 | AAGAAGAUCUCUGAGUUUUAG |
| 6831 | CCUCUCAUCUUUGACAGUCAA | 9435 | AGAAGAUCUCUGAGUUUUAGU |
| 6832 | CUCUCAUCUUUGACAGUCAAG | 9436 | GAAGAUCUCUGAGUUUUAGUU |
| 6833 | UCUCAUCUUUGACAGUCAAGG | 9437 | AAGAUCUCUGAGUUUUAGUUG |
| 6834 | CUCAUCUUUGACAGUCAAGGU | 9438 | AGAUCUCUGAGUUUUAGUUGC |
| 6835 | UCAUCUUUGACAGUCAAGGUG | 9439 | GAUCUCUGAGUUUUAGUUGCA |
| 6836 | CAUCUUUGACAGUCAAGGUGA | 9440 | AUCUCUGAGUUUUAGUUGCAG |
| 6837 | AUCUUUGACAGUCAAGGUGAA | 9441 | UCUCUGAGUUUUAGUUGCAGU |
| 6838 | UCUUUGACAGUCAAGGUGAAC | 9442 | CUCUGAGUUUUAGUUGCAGUU |
| 6839 | CUUUGACAGUCAAGGUGAACA | 9443 | UCUGAGUUUUAGUUGCAGUUC |
| 6840 | UUUGACAGUCAAGGUGAACAC | 9444 | CUGAGUUUUAGUUGCAGUUCU |
| 6841 | UUGACAGUCAAGGUGAACACA | 9445 | UGAGUUUUAGUUGCAGUUCUG |
| 6842 | UGACAGUCAAGGUGAACACAU | 9446 | GAGUUUUAGUUGCAGUUCUGC |
| 6843 | GACAGUCAAGGUGAACACAUA | 9447 | AGUUUUAGUUGCAGUUCUGCU |
| 6844 | ACAGUCAAGGUGAACACAUAG | 9448 | GUUUUAGUUGCAGUUCUGCUA |
| 6845 | CAGUCAAGGUGAACACAUAGG | 9449 | UUUUAGUUGCAGUUCUGCUAC |
| 6846 | AGUCAAGGUGAACACAUAGGU | 9450 | UUUAGUUGCAGUUCUGCUACU |
| 6847 | GUCAAGGUGAACACAUAGGUC | 9451 | UUAGUUGCAGUUCUGCUACUA |
| 6848 | UCAAGGUGAACACAUAGGUCC | 9452 | UAGUUGCAGUUCUGCUACUAA |
| 6849 | CAAGGUGAACACAUAGGUCCC | 9453 | AGUUGCAGUUCUGCUACUAAC |
| 6850 | AAGGUGAACACAUAGGUCCCC | 9454 | GUUGCAGUUCUGCUACUAACA |
| 6851 | AGGUGAACACAUAGGUCCCCA | 9455 | UUGCAGUUCUGCUACUAACAG |
| 6852 | GGUGAACACAUAGGUCCCCAC | 9456 | UGCAGUUCUGCUACUAACAGC |
| 6853 | GUGAACACAUAGGUCCCCACU | 9457 | GCAGUUCUGCUACUAACAGCU |
| 6854 | UGAACACAUAGGUCCCCACUU | 9458 | CAGUUCUGCUACUAACAGCUU |
| 6855 | GAACACAUAGGUCCCCACUUG | 9459 | AGUUCUGCUACUAACAGCUUU |
| 6856 | AACACAUAGGUCCCCACUUGC | 9460 | GUUCUGCUACUAACAGCUUUG |
| 6857 | ACACAUAGGUCCCCACUUGCA | 9461 | UUCUGCUACUAACAGCUUUGU |
| 6858 | CACAUAGGUCCCCACUUGCAG | 9462 | UCUGCUACUAACAGCUUUGUG |
| 6859 | ACAUAGGUCCCCACUUGCAGC | 9463 | CUGCUACUAACAGCUUUGUGA |
| 6860 | CAUAGGUCCCCACUUGCAGCC | 9464 | UGCUACUAACAGCUUUGUGAC |
| 6861 | AUAGGUCCCCACUUGCAGCCC | 9465 | GCUACUAACAGCUUUGUGACU |
| 6862 | UAGGUCCCCACUUGCAGCCCA | 9466 | CUACUAACAGCUUUGUGACUU |
| 6863 | AGGUCCCCACUUGCAGCCCAG | 9467 | UACUAACAGCUUUGUGACUUU |
| 6864 | GGUCCCCACUUGCAGCCCAGU | 9468 | ACUAACAGCUUUGUGACUUUG |
| 6865 | GUCCCCACUUGCAGCCCAGUC | 9469 | CUAACAGCUUUGUGACUUUGA |
| 6866 | UCCCCACUUGCAGCCCAGUCA | 9470 | UAACAGCUUUGUGACUUUGAC |
| 6867 | CCCCACUUGCAGCCCAGUCAC | 9471 | AACAGCUUUGUGACUUUGACC |
| 6868 | CCCACUUGCAGCCCAGUCACA | 9472 | ACAGCUUUGUGACUUUGACCA |
| 6869 | CCACUUGCAGCCCAGUCACAG | 9473 | CAGCUUUGUGACUUUGACCAA |
| 6870 | CACUUGCAGCCCAGUCACAGU | 9474 | AGCUUUGUGACUUUGACCAAA |
| 6871 | ACUUGCAGCCCAGUCACAGUA | 9475 | GCUUUGUGACUUUGACCAAAC |
| 6872 | CUUGCAGCCCAGUCACAGUAG | 9476 | CUUUGUGACUUUGACCAAACA |
| 6873 | UUGCAGCCCAGUCACAGUAGC | 9477 | UUUGUGACUUUGACCAAACAC |
| 6874 | UGCAGCCCAGUCACAGUAGCA | 9478 | UUGUGACUUUGACCAAACACA |
| 6875 | GCAGCCCAGUCACAGUAGCAA | 9479 | UGUGACUUUGACCAAACACAC |
| 6876 | CAGCCCAGUCACAGUAGCAAC | 9480 | GUGACUUUGACCAAACACACU |
| 6877 | AGCCCAGUCACAGUAGCAACA | 9481 | UGACUUUGACCAAACACACUU |
| 6878 | GCCCAGUCACAGUAGCAACAC | 9482 | GACUUUGACCAAACACACUUU |
| 6879 | CCCAGUCACAGUAGCAACACU | 9483 | ACUUUGACCAAACACACUUUC |
| 6880 | CCAGUCACAGUAGCAACACUG | 9484 | CUUUGACCAAACACACUUUCU |
| 6881 | CAGUCACAGUAGCAACACUGC | 9485 | UUUGACCAAACACACUUUCUU |
| 6882 | AGUCACAGUAGCAACACUGCU | 9486 | UUGACCAAACACACUUUCUUA |
| 6883 | GUCACAGUAGCAACACUGCUG | 9487 | UGACCAAACACACUUUCUUAC |
| 6884 | UCACAGUAGCAACACUGCUGU | 9488 | GACCAAACACACUUUCUUACU |
| 6885 | CACAGUAGCAACACUGCUGUU | 9489 | ACCAAACACACUUUCUUACUU |
| 6886 | ACAGUAGCAACACUGCUGUUA | 9490 | CCAAACACACUUUCUUACUUU |
| 6887 | CAGUAGCAACACUGCUGUUAG | 9491 | CAAACACACUUUCUUACUUUU |
| 6888 | AGUAGCAACACUGCUGUUAGC | 9492 | AAACACACUUUCUUACUUUUG |
| 6889 | GUAGCAACACUGCUGUUAGCA | 9493 | AACACACUUUCUUACUUUUGA |
| 6890 | UAGCAACACUGCUGUUAGCAU | 9494 | ACACACUUUCUUACUUUUGAG |
| 6891 | AGCAACACUGCUGUUAGCAUU | 9495 | CACACUUUCUUACUUUUGAGG |
| 6892 | GCAACACUGCUGUUAGCAUUC | 9496 | ACACUUUCUUACUUUUGAGGA |
| 6893 | CAACACUGCUGUUAGCAUUCU | 9497 | CACUUUCUUACUUUUGAGGAG |
| 6894 | AACACUGCUGUUAGCAUUCUC | 9498 | ACUUUCUUACUUUUGAGGAGA |
| 6895 | ACACUGCUGUUAGCAUUCUCG | 9499 | CUUUCUUACUUUUGAGGAGAA |
| 6896 | CACUGCUGUUAGCAUUCUCGA | 9500 | UUUCUUACUUUUGAGGAGAAG |
| 6897 | ACUGCUGUUAGCAUUCUCGAG | 9501 | UUCUUACUUUUGAGGAGAAGA |
| 6898 | CUGCUGUUAGCAUUCUCGAGC | 9502 | UCUUACUUUUGAGGAGAAGAG |
| 6899 | UGCUGUUAGCAUUCUCGAGCU | 9503 | CUUACUUUUGAGGAGAAGAGG |
| 6900 | GCUGUUAGCAUUCUCGAGCUG | 9504 | UUACUUUUGAGGAGAAGAGGU |
| 6901 | CUGUUAGCAUUCUCGAGCUGC | 9505 | UACUUUUGAGGAGAAGAGGUU |
| 6902 | UGUUAGCAUUCUCGAGCUGCA | 9506 | ACUUUUGAGGAGAAGAGGUUG |
| 6903 | GUUAGCAUUCUCGAGCUGCAC | 9507 | CUUUUGAGGAGAAGAGGUUGC |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A composition comprising an agent that blocks AAV binding to an AAV receptor (AAVR) and a delivery molecule that delivers the agent to the liver.

2. The composition of claim 1, wherein the agent comprises (a) a small-interfering RNA (siRNA); (b) an anti-AAVR antibody; (c) a small molecule; or (d) an antisense oligonucleotide (ASO).

3. The composition of claim 2, wherein the siRNA comprises;
(a) at least 19 contiguous nucleotides of any of SEQ ID Nos: 4300-9527;
(b) any of the sequences of SEQ ID NOs: 4300-9527; and/or
(c) wherein the siRNA is no more than 21, 25, or 31 nucleotides in length.

4.-8. (canceled)

9. The composition of claim 2, wherein the ASO comprises any of the sequences of SEQ ID Nos: 9600-9623, or comprises at least 14 consecutive nucleotides of any of SEQ ID Nos: 9600-9623.

10. The composition of claim 1, wherein the delivery molecule comprises one or more of the following: lactose, galactose, N-acetylgalactosamine (GalNAc), galactosamine, N-formylgalactosamine, N-acetylgalactosamine, N-propionylgalactosamine, N-butanoylgalactosamine, N-isobutanoyl-galactosamine, and cholesterol, or a derivative thereof.

11. (canceled)

12. The composition of claim 1, wherein the delivery molecule comprises a lipid nanoparticle (LNP) or an AAV.

13. (canceled)

14. A method comprising: (a) administering to a subject an agent that blocks AAV binding to an AAV receptor (AAVR) in the liver, and then (b) administering an AAV vector to the subject.

15. The method of claim 14, wherein the AAV vector further comprises a payload, wherein the payload comprises (a) a therapeutic agent to prevent or treat disease; and/or (b) a guide RNA, an endonuclease, a tRNA, a small molecule, an antisense oligonucleotide (ASO), an antibody, a small-interfering RNA (siRNA), or an RNAi agent.

16. (canceled)

17. (canceled)

18. A method of increasing the percentage of AAV delivered to a non-liver target in a subject, comprising (a) a pre-conditioning step comprising administering to the subject a composition comprising an agent that blocks AAV binding to an AAV receptor (AAVR) in the liver, and then (b) administering an AAV vector targeting a non-liver tissue.

19. The method of claim 14, wherein step (a) comprises administering to the subject a composition comprising the agent that blocks AAV binding to an AAVR and a delivery molecule that delivers the agent to the liver.

20. A method of decreasing tropism of AAV to the liver in a subject comprising (a) administering to the subject a composition of claim 1, and then (b) administering an AAV vector targeting a non-liver tissue.

21. The method of claim 14, wherein:
(a) administering to the subject agent that blocks AAV binding to an AAV receptor (AAVR) in the liver temporarily blocks AAV binding to AAV receptors in the liver;
(b) administering to the subject the agent that blocks AAV binding to an AAVR in the liver is part of a pre-conditioning step; and/or
(c) administering to the subject the agent that blocks AAV binding to an AAVR in the liver occurs about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the AAV vector.

22.-25. (canceled)

26. The method of claim 19, wherein (a) administering the composition immediately precedes administering the AAV vector; or (b) the composition and the AAV vector are co-administered.

27. (canceled)

28. The method of claim 14, wherein step (a) comprises administering to the subject a composition comprising the agent, wherein the agent comprises a small-interfering RNA (siRNA) or an antisense oligonucleotide (ASO).

29. The method of claim 28, wherein the composition comprises:
(a) an siRNA that is conjugated to a liver-targeting moiety;
(b) an N-acetylgalactosamine (GalNAc)-conjugated siRNA; and/or
(c) an siRNA encapsulated in a lipid nanoparticle (LNP).

30.-32. (canceled)

33. The method of claim 18, wherein the composition comprises a pharmaceutically acceptable carrier.

34. The method of claim 33, wherein the method comprises administering the composition to the subject by intraperitoneal injection.

35.-41. (canceled)

42. The method of claim 14, wherein the AAV vector targets a non-liver tissue, wherein the non-liver tissue is the brain; central nervous system; spinal cord; eye; retina; bone; cardiac muscle, skeletal muscle, and/or smooth muscle; lung; pancreas; heart; and/or kidney.

43. (canceled)

44. The method of claim 18, wherein administering the composition in step (a) increases the percentage of AAV delivered to a non-liver target.

45. The method of claim 14, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of AAV in brain; central nervous system; spinal cord; eye; retina; bone; cardiac muscle, skeletal muscle, and/or smooth muscle; lung; pancreas; heart; and/or kidney as compared to the AAV in the corresponding tissue of a control subject that received the AAV but did not receive the agent that blocks AAV binding to an AAV receptor (AAVR) in the liver.

46. (canceled)

47. The method of claim 14, wherein the AAV vector further comprises one or more molecules for enhancing tropism for the target host cells or tissue.

48. The method of claim 14, wherein the subject is a human subject.

49. The method of claim 14, wherein the AAV vector comprises a single nucleic acid molecule encoding one or more guide RNAs and a Cas9, wherein the single nucleic acid molecule comprises:
a. a first nucleic acid encoding one or more spacer sequences selected from any one of SEQ ID NOs: 1-35, 1000-1078, or 3000-3069, and a second nucleic acid encoding a Staphylococcus aureus Cas9 (SaCas9); or
b. a first nucleic acid encoding one or more spacer sequences selected from any one of SEQ ID NOs: 100-225, 2000-2116, or 4000-4251, and a second nucleic acid encoding a Staphylococcus lugdunensis (SluCas9); or
c. a first nucleic acid encoding one or more spacer sequences comprising at least 20 contiguous nucleotides of a spacer sequence selected from any one of SEQ ID NOs: 1-35, 1000-1078, or 3000-3069, and a second nucleic acid encoding a Staphylococcus aureus Cas9 (SaCas9); or
d. a first nucleic acid encoding one or more spacer sequences comprising at least 20 contiguous nucleotides of a spacer sequence selected from any one of SEQ ID NOs: 100-225, 2000-2116, or 4000-4251, and a second nucleic acid encoding a Staphylococcus lugdunensis (SluCas9); or
e. a first nucleic acid encoding one or more spacer sequences that is at least 90% identical to any one of SEQ ID NOs: 1-35, 1000-1078, or 3000-3069, and a second nucleic acid encoding a Staphylococcus aureus Cas9 (SaCas9); or
f. a first nucleic acid encoding one or more spacer sequences that is at least 90% identical to any one of SEQ ID NOs: 100-225, 2000-2116, or 4000-4251, and a second nucleic acid encoding a Staphylococcus lugdunensis (SluCas9); or
g. a first nucleic acid encoding a pair of guide RNAs comprising a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
h. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
i. a first nucleic acid encoding a pair of guide RNAs that is at least 90% identical to a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
j. a first nucleic acid encoding a pair of guide RNAs comprising a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
k. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
l. a first nucleic acid encoding a pair of guide RNAs that is at least 90% identical to a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
m. a first nucleic acid encoding a pair of guide RNAs that is at least 90% identical to a first and second spacer sequence selected from any one of the following pairs of spacer sequences:
i. SEQ ID NOS: 148 and 134,
ii. SEQ ID Nos: 145 and 131,
iii. SEQ ID Nos: 144 and 149;
iv. SEQ ID Nos: 144 and 150;
v. SEQ ID Nos: 146 and 148;
and a second nucleic acid encoding a Staphylococcus lugdunensis (SluCas9); or
n. a first nucleic acid encoding a pair of guide RNAs that is at least 90% identical to a first and second spacer sequence selected from any one of the following pairs of spacer sequences:
i. SEQ ID NOs: 12 and 1013; and
ii. SEQ ID Nos: 12 and 1016;
and a second nucleic acid encoding a SaCas9-KKH.

50. (canceled)

51. The method of claim 14, wherein the AAV vector is an AAV9 vector.

52. The method of claim 14, wherein the agent comprises an siRNA, an anti-AAVR antibody, a small molecule, or an antisense oligonucleotide (ASO).

53. The method of claim 52, wherein the siRNA comprises;
(a) at least 19 contiguous nucleotides of any of SEQ ID Nos: 4300-9527;
(b) any of the sequences of SEQ ID NOs: 4300-9527;
(c) at least 19 contiguous nucleotides of any of SEQ ID NOs: 9508-9531; or
(d) any of the sequences of SEQ ID NOs: 9508-9531.

54.-56. (canceled)

57. The method of claim 52 wherein the siRNA is no more than 21, 25, or 31 nucleotides in length.

58.-60. (canceled)

61. The method of claim 52, wherein the ASO
(a) comprises any of the sequences of SEQ ID Nos: 9600-9623, or comprises at least 14 consecutive nucleotides of any of SEQ ID Nos: 9600-9623; and/or
(b) is between 14-35, 15-30, or 15-25 nucleotides in length.

62. (canceled)
